SEQUENCE LISTING (122 sequences)

SEQ ID NO: 1 - protein, 955 aa, synthetic construct
ETGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 832 aa, synthetic construct
ETGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 330 aa, Homo sapiens [sequence omitted]

SEQ ID NO: 4 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 9 - protein, 110 aa, synthetic construct
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 10 - protein, 131 aa, synthetic construct
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 11 - protein, 128 aa, synthetic construct
AAQAPVKLSLNLLASSDPPEAASWLLCEVSGFSPPNILLMWLEDQREVNTSGFAPAPMPEPQAPGRYFAHSILTVSEEEWNTGETYTCVVAHEALPNRVTERTVDKSTGKPTLYNVSLVMSDTAGTCY

SEQ ID NO: 12 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1353 bp, synthetic construct
  source 1..1353: mol_type = other DNA
gaagtgcagctggttgaatctggcggcggactggttcaaccaggcggatctctgagactgagctgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgtagtagatggggaggcgacggcttctacgctatggattattggggccagggcaccctggtcacagttagctctgcgtcgacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 14 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 645 bp, synthetic construct
  source 1..645: mol_type = other DNA
gatatccagatgacacagagccccagcagcctgtctgcctctgtgggagacagagtgaccatcacctgtagagccagccaggacgtgaacacagccgtggcttggtatcagcagaagcctggcaaggcccctaagctgctgatctacagcgccagctttctgtacagcggcgtgcccagcagattcagcggaagcagaagcggcaccgacttcaccctgaccataagcagtctgcagcccgaggacttcgccacctactactgtcagcagcactacaccacacctccaaccttcggccagggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 16 - protein, 452 aa, Homo sapiens
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1359 bp, Homo sapiens
  source 1..1359: mol_type = other DNA
gaagtgcagctggttgaatctggcggcggactggttcagcctggcagatctctgagactgagctgtgccgccagcggcttcaccttcaacgattacgccatgcactgggtccgacaggcccctggaaaaggccttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacactggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 18 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 645 bp, Homo sapiens
  source 1..645: mol_type = other DNA
gaaattgtgctgacacagagccccgccacactgtcactttctccaggcgaaagagccacactgagctgcagagccagccagagcgtgtcctcttacctggcctggtatcagcagaagcctggacaggctccccggctgctgatctacgatgccagcaatagagccaccggcattcccgccagattttctggctctggaagcggcaccgacttcaccctgaccataagcagcctggaacctgaggacttcgccgtgtactactgccagcagagaagcaactggcccatcacctttggccagggtaccagactggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 20 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1359 bp, synthetic construct
  source 1..1359: mol_type = other DNA
gaagttcagctgctggaatctggcggcggactggttcaaccaggcggatctctgagactgagctgtgccgtgtccggcttcaccttcaacagcttcgccatgagctgggtccgacaggcccctggaaaaggccttgaatgggtgtccgccatctctggctctggcggaggcacatattacgccgactctgtgaagggcagattcaccatcagccgggacaacagcaagaacaccctgtacctgcagatgaacagcctgagagccgaggataccgccgtgtacttctgcgccaaggacaagatcctttggttcggcgagcccgtgttcgattattggggccagggcaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 22 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 645 bp, synthetic construct
  source 1..645: mol_type = other DNA
gaaattgtgctgacacagagccccgccacactgtcactttctccaggcgaaagagccacactgagctgcagagccagccagagcgtgtcctcttacctggcctggtatcagcagaagcctggacaggctccccggctgctgatctacgatgccagcaatagagccaccggcattcccgccagattttctggctctggaagcggcaccgacttcaccctgaccataagcagcctggaacctgaggacttcgccgtgtactactgccagcagagaagcaactggcctcctacctttggccagggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 24 - protein, 449 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1350 bp, synthetic construct
  source 1..1350: mol_type = other DNA
gaagtgcagctggttgaatctggcggcggactggttcaaccaggcggatctctgagactgagctgtgccgccagcggcttcaccttcaccgactacacaatggactgggtccgacaggcccctggcaaaggacttgaatgggtcgccgacgtgaaccccaatagcggcggcagcatctacaaccagcggttcaagggcagattcaccctgagcgtggacagaagcaagaacaccctgtacctgcagatgaacagcctgagagccgaggacaccgccgtgtactactgcgccagaaatctgggccccagcttctacttcgattattggggccagggcaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 26 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 645 bp, synthetic construct
  source 1..645: mol_type = other DNA
gatatccagatgacacagagccccagcagcctgtctgcctctgtgggagacagagtgaccatcacctgtaaagccagccaggacgtgtccatcggcgtggcatggtatcagcagaagcctggcaaggcccctaagctgctgatctacagcgccagctacagatacaccggcgtgcccagcagattttctggcagcggctctggcaccgacttcaccctgaccatatctagcctgcagcctgaggacttcgccacctactactgccagcagtactacatctacccctacaccttcggccagggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 28 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1356 bp, synthetic construct
  source 1..1356: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtttctgccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 30 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 642 bp, synthetic construct
  source 1..642: mol_type = other DNA
caaattgtgctgtctcagagccccgccatcctgagtgcttctccaggcgagaaagtgaccatgacctgcagagccagcagcagcgtgtcctacatccactggttccagcagaagcctggcagcagccctaagccttggatctacgccacaagcaatctggccagcggcgtgccagtcagattttctggctctggcagcggcaccagctacagcctgacaatcagcagagtggaagccgaggatgccgccacctactactgtcagcagtggaccagcaatcctcctacctttggcggcggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 32 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1341 bp, synthetic construct
  source 1..1341: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 442 aa, synthetic construct
EVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 35 - DNA, 1329 bp, synthetic construct
  source 1..1329: mol_type = other DNA
gaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatga

SEQ ID NO: 36 - protein, 220 aa, synthetic construct
DIVMSQSPSSLAVSVGEKVTMSCKSSQSLLYSSNQKNYLAWYQRKPGQSPKLLIYWASTRESGVPDRFTGSGSGTDFTLTISSVKAEDLAVYYCQHYYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 660 bp, synthetic construct
  source 1..660: mol_type = other DNA
gacatagtgatgtcacagagcccaagcagccttgcagtgtctgttggtgagaaggtgaccatgagctgcaagtcaagccagagcctgctctatagcagcaatcagaagaactatttggcctggtatcagagaaaacccggccagagccctaagctccttatttattgggctagcacacgcgagtccggcgtgccggacagatttaccggcagcggtagcggcaccgattttactttgaccatctcgtctgtaaaggccgaagacctggcagtgtattactgtcaacattattactcatctccctacactttcggaggggggaccaaactggaaattaagcgtacggttgctgccccttctgtcttcatcttccctcccagcgatgaacagctgaaaagtgggacagcgagtgtagtgtgcctgctaaacaatttttacccacgggaggccaaagtgcagtggaaagtggacaacgctctgcaaagtggaaattctcaggagtccgtcacagagcaggactcgaaggacagcacttacagcttgtcaagcaccctgacgctgagcaaagccgattacgagaagcacaaggtgtacgcatgcgaagttacgcaccaaggactgagtagcccagtcacaaagagcttcaacaggggggaatgt

SEQ ID NO: 38 - protein, 498 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKTKGQPREPQVYTLPPSREEMTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 39 - DNA, 1494 bp, synthetic construct
  source 1..1494: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttctagaagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatacctgcaatgtcaatcataaacccagtaacacaaaggtagacaagcgggtcgaactgaaaacccccctgggcgacaccacccacacctgtcctagatgccccgagcccaagtcctgcgatacccctccaccttgccccaggtgccctgagcctaagagctgtgataccccccctccctgccctcggtgtccagagcctaagtcttgtgacaccccacccccatgtccaaggtgccccgcccccgaactcctggggggaccttcggtttttctttttccacctaaaccgaaagacaccctgatgatcagcaggacaccagaagtgacatgtgtcgtcgtggacgtatcccatgaggacccggaggtgcagttcaagtggtatgttgatggtgtggaagtgcataatgcaaaaactaagctgcgggaggaacagtacaatagcacattcagagtagttagcgttctgactgtcctgcaccaagattggctgaacgggaaggaatacaagtgtaaggtgagcaacaaagccctgcccgctccaattgagaaaacaatttctaagaccaaaggacagccccgggagccacaggtgtatactctgccgcctagcagggaagagatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacatcttcagttgctcagtgatgcacgaagcgctgcacaatcattatactcagaaaagcctgagcttgagcccgggaaaa

SEQ ID NO: 40 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttccagaagcacctctgaaagtactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcaccaagacctatacatgcaatgtcgaccataaacccagtaacacaaaggtagacaagcgagtcgaaagcaagtacggccccccctgccctagctgccccgcccccgaatttctggggggaccttcggtttttctttttccacctaaaccgaaagacaccctgatgatcagcaggacaccagaagtgacatgtgtcgtcgtggacgtatcccaggaggacccggaggtgcagttcaactggtatgttgatggtgtggaagtgcataatgcaaaaactaagccacgggaggaacagttcaatagcacatacagagtagttagcgttctgactgtcctgcaccaagattggctgaacgggaaggaatacaagtgtaaggtgagcaacaaaggcctgcccagtagcattgagaaaacaatttctaaggccaaaggacagccccgggagccacaggtgtatactctgccgcctagccaagaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcacggctgaccgtggacaaaagcagatggcaggagggaaacgttttcagttgctcagtgatgcacgaagcgctgcacaatcattatactcagaaaagcctgagcttgagcttaggaaaa

SEQ ID NO: 42 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggcacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 44 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 645 bp, synthetic construct
  source 1..645: mol_type = other DNA
gatatcgtgatgacacagagccacctgagcatgagcaccagcctgggagatcccgtgtccatcacctgtaaagccagccaggatgtgtccaccgtggtggcctggtatcagcagaagcctggccagtctcctcggcggctgatctactctgccagctacagatatatcggcgtgcccgacagattcaccggatctggcgctggcaccgacttcacctttaccatcagctctgtgcaggccgaggacctggccgtgtactattgtcagcagcactacagccctccatacacctttggcggcggtaccaagctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 46 - protein, 447 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - DNA, 1341 bp, Homo sapiens
  source 1..1341: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgattattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 48 - protein, 214 aa, Homo sapiens
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 642 bp, Homo sapiens
  source 1..642: mol_type = other DNA
gagattgtgatgacacagagccccgccacactgtccgtttctccaggcgaaagagccacactgagctgcagagccagccagagcatcagcaacaacctgcactggtatcagcagaagcccggacaggctcccagactgctgatcaagtttgccagccagtccatcacaggcatccccgccagattttctggcagcggctctggcaccgagttcaccctgacaatcagcagcctgcagagcgaggacttcgccgtgtactattgccagcagggcaacagctggccctacaccttcggccagggtaccaagctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 50 - protein, 443 aa, Homo sapiens
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 51 - DNA, 1329 bp, Homo sapiens
  source 1..1329: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctggtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 52 - protein, 447 aa, Homo sapiens
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - DNA, 1341 bp, Homo sapiens
  source 1..1341: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 54 - protein, 213 aa, Homo sapiens
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 639 bp, Homo sapiens
  source 1..639: mol_type = other DNA
gatatccagctgacacagagccctagcagcctgtctgcctctgtgggcgacagagtgaccatcacatgtagcgccagcagcagcgtgtcctacatgtactggtatcagcagaagcccggcaaggcccctaagccttggatctacagaaccagcaatctggccagcggcgtgcccagcagattttctggttctggcagcggcaccgacttcaccctgaccatatctagcctgcagcctgaggacttcgccacctactactgccagcagtaccacagctaccctccaacctttggcggcggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 56 - protein, 443 aa, Homo sapiens
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 57 - DNA, 1329 bp, Homo sapiens
  source 1..1329: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 58 - protein, 439 aa, Homo sapiens
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 59 - DNA, 1317 bp, Homo sapiens
  source 1..1317: mol_type = other DNA
gaagttcagctgcaacagtctggcgccgaggttgtgaaacctggcgcctctgtgaagctgagctgtaaagccagcggcttcaacatcaaggacaccttcatccactgggtcaagcaggcccctggacagggactcgagtggatcggaagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggacacgaagcgctgcccctggctttcacccagaaaaccatcgatagactggccggc

SEQ ID NO: 60 - protein, 439 aa, Homo sapiens
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 61 - DNA, 1317 bp, Homo sapiens
  source 1..1317: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggacacgaagcgctgcccctggctttcacccagaaaaccatcgatagactggccggc

SEQ ID NO: 62 - protein, 447 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 63 - DNA, 1341 bp, synthetic construct
  source 1..1341: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgattattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 64 - protein, 443 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 65 - DNA, 1329 bp, synthetic construct
  source 1..1329: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctggtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 66 - protein, 447 aa, Homo sapiens
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 67 - DNA, 1341 bp, Homo sapiens
  source 1..1341: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 68 - protein, 443 aa, Homo sapiens
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 69 - DNA, 1329 bp, Homo sapiens
  source 1..1329: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 70 - protein, 439 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGFEALPLAFTQKTIDRLAG

SEQ ID NO: 71 - DNA, 1317 bp, synthetic construct
  source 1..1317: mol_type = other DNA
gaagttcagctgcaacagtctggcgccgaggttgtgaaacctggcgcctctgtgaagctgagctgtaaagccagcggcttcaacatcaaggacaccttcatccactgggtcaagcaggcccctggacagggactcgagtggatcggaagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggattcgaagcgctgcccctggctttcacccagaaaaccatcgatagactggccggc

SEQ ID NO: 72 - protein, 439 aa, Homo sapiens
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGFEALPLAFTQKTIDRLAG

SEQ ID NO: 73 - DNA, 1317 bp, Homo sapiens
  source 1..1317: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggattcgaagcgctgcccctggctttcacccagaaaaccatcgatagactggccggc

SEQ ID NO: 74 - protein, 453 aa, Homo sapiens
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1359 bp, Homo sapiens
  source 1..1359: mol_type = other DNA
gaagtgcagctgctggaatctggcggcggacttgttcaaccaggcggctctctgagactgagctgtgccgcttccggcttcaccttcgacgattacggcatgagctgggtccgacaggcccctggaaaaggccttgaatgggtgtccgacatcagctggaatggcggcaagacccactacgtggactctgtgaagggccagttcaccatcagccgggacaacagcaagaacaccctgtacctgcagatgaacagcctgagagccgaggacaccgccgtgtactattgtgccagaggcagcctgttccacgacagcagcggcttctattttggccactggggccagggcacactggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 76 - protein, 216 aa, synthetic construct
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 77 - DNA, 648 bp, synthetic construct
  source 1..648: mol_type = other DNA
caatctgttctgacacagcctcctagcgcctctggcacacctggacagagagtgaccatcagctgtagcggcagcagcagcaacatcggcgacaactacgtgtcctggtatcagcagctgcctggcacagcccctaaactgctgatctaccgggatagccagaggccaagcggcgtgccagatagattttccggcagcaagagcggcacatctgccagcctggctatcagcggactgagatctgaggacgaggccgactactactgccagagctacgatagcagcctgagcggctctgtgtttggcggaggtaccaagctgaccgtgctgggccaacctaaagccaatcctaccgtgacactgttccctccaagcagcgaagaactgcaggccaacaaggccacactcgtgtgcctgatcagcgacttttatcctggcgccgtgaccgtggcctggaaggctgatggatctcctgtgaaagccggcgtggaaaccaccaagcctagcaagcagagcaacaacaaatacgccgccagcagctacctgagcctgacacctgagcagtggaagtcccacagatcctacagctgccaagtgacccacgagggcagcaccgtggaaaagacagtggcccctaccgagtgcagc

SEQ ID NO: 78 - protein, 448 aa, Homo sapiens
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1344 bp, Homo sapiens
  source 1..1344: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggcacactggtcaccgtgtcctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 80 - protein, 217 aa, Homo sapiens
EIVMMQSPGTLSLSPGERATLSCRASQSVRSNYLAWYQQKPGQAPRLLIYGASSRATGIPDRFSGSGSGTDFTLTISRLEPEDFAVYYCQQYGSSPPRFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 651 bp, Homo sapiens
  source 1..651: mol_type = other DNA
gagatcgtcatgatgcagagccccggcacactgtcactgtctccaggcgaaagagccacactgagctgtagagcctctcagagcgtgcggagcaattacctggcctggtatcagcagaagcccggacaggctcctcggctgttgatctatggcgcctctagcagagccactggcatccccgatagattttctggcagcggctccggcaccgacttcaccctgacaatcagcagactggaacccgaggacttcgccgtgtactactgtcagcagtacggcagcagccctcctcggttcacatttggccctggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 82 - protein, 447 aa, Homo sapiens
QVQEVQLLESGGGLVQPGGSLRLSCAASGFTFSSYVMTWARQAPGKGLEWVSAISGTGYTYYADSVKGRFTVSRDNSKNTLFLQMSSLRAEDTAVYYCAITMAPVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 455 aa, Homo sapiens
QVQLVESGAEVKKPGESLKISCKGSGYTFTRYWIGWVRQMPGKGLEWMGIIYPGDSDTRYSPSFQGHVTISADKSISTAYLQWNSLKASDTAMYYCARLPQYCSNGVCQRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1341 bp, Homo sapiens
  source 1..1341: mol_type = other DNA
caagtgcaggaagtgcagctgctggaatctggcggaggactggttcaaccaggcggctctctgagactgtcttgtgccgccagcggcttcaccttcagcagctacgttatgacctgggccagacaggcccctggcaaaggacttgaatgggtgtccgccatcagcggcacaggctacacctactacgccgactctgtgaagggcagattcaccgtgtccagagacaacagcaagaataccctgttcctgcagatgagcagcctgagagccgaggataccgccgtgtactactgcgccattacaatggcccctgtcgtgtggggccagggcacaacagtgacagtcagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 85 - DNA, 1365 bp, Homo sapiens
  source 1..1365: mol_type = other DNA
caagtgcagctggttgaatctggcgccgaagtgaagaagcctggcgagagcctgaagatcagctgcaaaggcagcggctacaccttcaccagatactggatcggctgggtccgacagatgcctggcaaaggccttgagtggatgggcatcatctaccccggcgacagcgataccagatacagccctagctttcagggccacgtgaccatcagcgccgacaagtctatcagcaccgcctacctgcagtggaacagcctgaaggccagcgacaccgccatgtactactgtgccagactgccccagtactgcagcaatggcgtttgccagcgttggttcgatccttggggacagggcacactggtcaccgttagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 86 - protein, 216 aa, Homo sapiens
SYELTQPPSVSVSPGQTARITCSGDALPKRYAYWYQQKSGQAPILVIYEDKKRPSGIPERLSGSKSGTVATLTISGAQVEDEADYYCYSTDSSGNHAVFGGGTQLTVLGQPKAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 214 aa, Homo sapiens
EIVLTQSPSSVSASVGDRVTITCRASQGISSWLAWYQQKPGKAPKLLIYAASSLQSGVPSRFSGSGSGTDFTLTISSLQPEDFATYYCQQGNSFPYTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 648 bp, Homo sapiens
  source 1..648: mol_type = other DNA
agctatgaactgacacagcctccaagcgtgtccgtgtctcctggacagaccgccagaatcacatgtagcggagatgccctgcctaagagatacgcctactggtatcagcagaagtccggacaggcccctatcctggtcatctacgaggacaagaagcggccctctggcatccctgagagactgagcggaagcaagtctggcacagtggccacactgacaatctctggcgcccaggtggaagatgaggccgactactactgctacagcaccgacagctctggcaaccacgctgtgtttggcggaggtacccaactgaccgtgctgggccaacctaaagccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 89 - DNA, 642 bp, Homo sapiens
  source 1..642: mol_type = other DNA
gaaattgtgctgacacagagccccagcagcgtgtcagcctctgtgggagacagagtgaccatcacctgtagagccagccagggcatctcttcttggctggcctggtatcagcagaagcctggcaaggcccctaagctgctgatctatgccgctagctctctgcagtctggcgtgcccagcagattttctggcagcggctctggcaccgacttcaccctgaccatatctagcctgcagcctgaggacttcgccacctactattgccagcagggcaacagcttcccctacacctttggccagggtaccaacctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 90 - protein, 448 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 91 - DNA, 1344 bp, synthetic construct
  source 1..1344: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggcacactggtcaccgtgtcctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaagtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 92 - protein, 447 aa, synthetic construct
QVQEVQLLESGGGLVQPGGSLRLSCAASGFTFSSYVMTWARQAPGKGLEWVSAISGTGYTYYADSVKGRFTVSRDNSKNTLFLQMSSLRAEDTAVYYCAITMAPVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 93 - DNA, 1341 bp, synthetic construct
  source 1..1341: mol_type = other DNA
caagtgcaggaagtgcagctgctggaatctggcggaggactggttcaaccaggcggctctctgagactgtcttgtgccgccagcggcttcaccttcagcagctacgttatgacctgggccagacaggcccctggcaaaggacttgaatgggtgtccgccatcagcggcacaggctacacctactacgccgactctgtgaagggcagattcaccgtgtccagagacaacagcaagaataccctgttcctgcagatgagcagcctgagagccgaggataccgccgtgtactactgcgccattacaatggcccctgtcgtgtggggccagggcacaacagtgacagtcagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 94 - protein, 455 aa, synthetic construct
QVQLVESGAEVKKPGESLKISCKGSGYTFTRYWIGWVRQMPGKGLEWMGIIYPGDSDTRYSPSFQGHVTISADKSISTAYLQWNSLKASDTAMYYCARLPQYCSNGVCQRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 95 - DNA, 1365 bp, synthetic construct
  source 1..1365: mol_type = other DNA
caagtgcagctggttgaatctggcgccgaagtgaagaagcctggcgagagcctgaagatcagctgcaaaggcagcggctacaccttcaccagatactggatcggctgggtccgacagatgcctggcaaaggccttgagtggatgggcatcatctaccccggcgacagcgataccagatacagccctagctttcagggccacgtgaccatcagcgccgacaagtctatcagcaccgcctacctgcagtggaacagcctgaaggccagcgacaccgccatgtactactgtgccagactgccccagtactgcagcaatggcgtttgccagcgttggttcgatccttggggacagggcacactggtcaccgttagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 96 - protein, 19 aa, Homo sapiens
HEALHNHYTQKSLSLSPGK

SEQ ID NO: 97 - DNA, 57 bp, Homo sapiens
  source 1..57: mol_type = other DNA
cacgaagcgctgcacaatcattatactcagaaaagcctgagcttgagcccgggaaaa

SEQ ID NO: 98 - protein, 19 aa, synthetic construct
FEALHNHYTQKSLSLSPGK

SEQ ID NO: 99 - DNA, 57 bp, synthetic construct
  source 1..57: mol_type = other DNA
ttcgaggcgctgcacaatcattatactcagaaaagcctgagcttgagcccgggaaaa

SEQ ID NO: 100 - protein, 19 aa, synthetic construct
FEALHNHYTQESLSLSPGK

SEQ ID NO: 101 - DNA, 57 bp, synthetic construct
  source 1..57: mol_type = other DNA
ttcgaagcgctgcacaatcattatactcaggagagcctgagcttgagcccgggaaaa

SEQ ID NO: 102 - protein, 19 aa, synthetic construct
FEALHNHYTQKKLSLSPGK

SEQ ID NO: 103 - DNA, 57 bp, synthetic construct
  source 1..57: mol_type = other DNA
ttcgaagcgctgcacaatcattatactcagaaaaagctgagcttgagcccgggaaaa

SEQ ID NO: 104 - protein, 1065 aa, Homo sapiens
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 3195 bp, Homo sapiens
  source 1..3195: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggatccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgagctcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 106 - protein, 842 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - DNA, 2526 bp, Homo sapiens
  source 1..2526: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggctcgaggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 108 - protein, 1065 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 109 - DNA, 3195 bp, synthetic construct
  source 1..3195: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggatccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgagctcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 110 - protein, 447 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 111 - DNA, 1341 bp, synthetic construct
  source 1..1341: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgattattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaagccgcggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 112 - protein, 447 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 113 - DNA, 1341 bp, synthetic construct
  source 1..1341: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaagccgcggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 114 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 107 aa, synthetic construct
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 116 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - protein, 131 aa, synthetic construct
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 119 - protein, 131 aa, synthetic construct; identical to SEQ ID NO: 118

SEQ ID NO: 120 - protein, 31 aa, synthetic construct
MGILPSPGMPALLSLVSLLSVLLMGCVAETG

SEQ ID NO: 121 - protein, 5 aa, synthetic construct
GGGGS

SEQ ID NO: 122 - protein, 7 aa, synthetic construct
GSGSGSG

(19) United States
(12) Patent Application Publication
(10) Pub. No.: US 2026/0158160 A1
(43) Pub. Date: Jun. 11, 2026

(54) Immunotherapeutic Proteins Comprising an Fc Region Component With a Mutation at Position 429

(71) Applicant: Macfarlane Burnet Institute for Medical Research and Public Health Limited, Melbourne, Victoria (AU)

(72) Inventors: Phillip Mark HOGARTH, Williamstown, Victoria (AU); Bruce David WINES, Heidelberg, Victoria (AU)

(73) Assignee: Macfarlane Burnet Institute for Medical Research and Public Health Limited, Melbourne, Victoria (AU)

(21) Appl. No.: 18/708,757

(22) PCT Filed: Oct. 26, 2022
(86) PCT No.: PCT/AU2022/051287

§ 371 Date: May 9, 2024

(30) Foreign Application Priority Data
Nov. 11, 2021 (AU) 2021903609
Jun. 15, 2022 (AU) 2022901632

(51) Int. Cl. A61K 47/68; C07K 16/10; C07K 16/102; C07K 16/104; C07K 16/28; C12N 9/48

(52) U.S. Cl. CPC A61K 47/6835; C07K 16/10; C07K 16/102; C07K 16/104; C07K 16/2803; C07K 16/2818; C07K 16/2827; C07K 16/2875; C07K 16/2878; C07K 16/2887; C07K 16/2893; C07K 16/2896; C12N 9/485; C12Y 304/17023; C07K 2319/30

(57) ABSTRACT

An immunotherapeutic protein and methods of use and production thereof are disclosed, wherein the immunotherapeutic protein comprises one or more immunoglobulin heavy chain polypeptide comprising an Fc region component comprising at least a constant heavy chain domain 3 (CH3) domain (or at least a constant heavy domain 4 (CH4) domain), wherein said one or more polypeptide includes an amino acid substitution at a position corresponding to H429 of the amino acid sequence of human IgG1 heavy chain (Eu numbering) such as H429Y and H429F. The immunotherapeutic protein may be in the form of, for example, an antibody, antibody-like molecule or fusion protein. The amino acid substitution may have the effect of enabling the immunotherapeutic protein to oligomerise (eg into a hexameric form) either in solution or upon binding to a relevant target.

### TECHNICAL FIELD

The present disclosure relates to immunotherapeutic proteins comprising, for example, modified immunoglobulin molecules (i.e. antibodies) for use in methods of treating diseases or conditions.

### PRIORITY DOCUMENT

The present application claims priority from Australian Provisional Patent Application No 2021903609 titled “Modified immunoglobulin and method of use thereof (3)” and filed on 11 Nov. 2021, as well as from Australian Provisional Patent Application No 2022901632 also titled “Modified immunoglobulin and method of use thereof (3)” but filed on 15 Jun. 2022. The content of both of these applications is hereby incorporated by reference in their entirety.

### BACKGROUND

Monoclonal antibodies (mAbs) have become one of the most important and successful types of therapeutics, revolutionizing the treatment of cancer and inflammatory diseases such as autoimmune diseases. Many mAbs engineered on an IgG antibody class backbone specifically harness the powerful effector functions of the immune system by engaging both the target antigen via their variable Fab domains and Fc γ receptors (Fc γ Rs) via their heavy chains including the constant Fc portion (Fc or Fc fragment), leading to, inter alia, activation of inflammatory or killer cells. In addition, the Fc of antibodies may also activate blood proteins called complement (involving the coming together of antibodies in a process known as “self-association” or “on-target assembly”) to bring about complement-dependent effector functions such as, for example, complement-dependent cytotoxicity (CDC). These various actions work separately, but in a complementary manner, to enable natural antibodies to destroy “targets” such as cancer cells and virus-infected cells (or the virus itself), as well as bacteria, parasites and other pathogens. However, they can also be advantageously harnessed through the action of engineered mAbs intended for various medical treatments (i.e. therapeutic mAbs) such as, for example, cancer therapies, and treatments of infection and inflammatory diseases and disorders.

Given the above, it is perhaps unsurprising that considerable research effort has been applied to elucidate the details of Fc interactions (e.g. with Fc γ Rs and complement proteins such as C1q and the SUBSTITUTE SHEETS (RULE 26) membrane attack complex (MAC) proteins C4-C9) and identify modifications (e.g. mutations) that may be made to the Fc in order to potentially enhance the potency of therapeutic mAbs. However, very few such Fc mutations have been included to date in therapeutic mAbs; the best known being antibodies wherein the heavy chain glycan has been engineered to lack fucose and thereby result in enhanced FcγRIII interaction, for example obinituzumab and margetuximab (which comprises six point mutations of the heavy chain of an IgG1 antibody), and others that are undergoing clinical trials but yet to be approved such as various HexaBody™ mAbs (GenMab BV; Copenhagen, Denmark) which comprise a triple mutation of the Fc fragment of IgG1, namely amino acid substitutions at positions 345 (i.e. E345R), 430 (i.e. E430G) and 440 (i.e. S440Y) ((known as “IgG-RGY”) (see International patent publication no. WO 2014/006217)), or a single mutation only at position 430 (i.e. E430G) of the Fc of IgG1 (de Jong R N et al., PLoS Biol 14(1):e1002344, 2016). This E430G mutation has been shown to enhance the ability of IgG1 antibodies to form hexamers on target molecules, and IgG1 mutants including the E430G mutation have been reported as displaying strongly enhanced CDC conditional on antigen binding at the target cell (de Jong et al., 2016 supra). It will therefore be apparent to those skilled in the art that there is a need in the art for modifications which enhance the therapeutic potency of immunotherapeutic proteins (e.g., mAbs).

### SUMMARY

In work leading to the present disclosure, the inventors produced a series of mutant antibodies (particularly mutant immunoglobulin G (IgG) molecules) comprising, for example, point mutations at various positions in and around the interface of the CH2 and CH3 domains of the heavy chain polypeptides. Some molecules including a mutation at position 429, considered to be a “buried” or inaccessible site within the IgG1 structure (see FIG. 1) and occupied by a histidine (His/H) residue, were found to show, for example, enhanced activation of the complement system by the classical complement pathway including binding of C1q, formation of the membrane attack complex and complement-dependent cytotoxicity in assays of complement function. In addition, the inventors obtained evidence showing that some molecules comprising an Fc region component with a mutation at position 429 are capable of forming oligomers, either in solution or upon binding to a relevant target. The present disclosure is directed to immunoglobulins and other molecules comprising an Fc region component (e.g. fusion proteins comprising a partner polypeptide with a beneficial function and/or characteristic), which comprise a mutation at position 429 of the Fc region component (or a position corresponding thereto), and their potential use in therapies for, for example, cancer (e.g. acute lymphoblastic leukaemia (ALL) and adenocarcinoma), infections (e.g. COVID-19, emerging viral infections, and parasitic infections such as infections by drug-resistant malaria) and inflammatory diseases and disorders (e.g. such as autoimmune diseases including systemic lupus erythematosus (SLE) and type 1 diabetes (T1D)).

Thus, in a first aspect, the present disclosure provides an immunotherapeutic protein comprising one or more immunoglobulin heavy chain polypeptide comprising an Fc region component comprising at least a CH3 domain (or at least a CH4 domain), wherein said one or more polypeptide includes an amino acid substitution at a position corresponding to H429 of the amino acid sequence of human IgG1 heavy chain (Eu numbering).

The immunotherapeutic protein may comprise a dimeric immunotherapeutic protein such as an immunoglobulin molecule (e.g. antibody) comprising first and second immunoglobulin heavy chain polypeptides comprising an Fc region component comprising CH2 and CH3 domains (so that the first and second polypeptides may form, by dimerisation, an Fc fragment or Fc-like fragment). In some embodiments, such a dimeric immunotherapeutic protein may be provided in an oligomeric form such as, for example, a hexamer comprising 6 copies of the dimeric immunotherapeutic protein.

In other embodiments, the immunotherapeutic protein comprises a partner polypeptide linked to an Fc region component comprising at least a CH3 domain (or at least a CH4 domain), such that the immunotherapeutic protein may be in the form of a fusion protein or protein conjugate. Such a fusion protein or protein conjugate may, in some embodiments, comprise a dimeric immunotherapeutic protein comprising first and second immunoglobulin heavy chain polypeptides comprising an Fc region component comprising CH2 and CH3 domains (so that the first and second polypeptides may form, by dimerisation, an Fc fragment or Fc-like fragment). In some embodiments, such a dimeric immunotherapeutic protein may be provided in an oligomeric form such as, for example, a hexamer comprising 6 copies of the dimeric immunotherapeutic protein.

In some preferred embodiments, the immunotherapeutic protein comprises an H→Y or H→F amino acid substitution at the position corresponding to H429 of the amino acid sequence of the human IgG1 heavy chain.

In a second aspect, the present disclosure provides the use of an immunotherapeutic protein as defined in the first aspect, for treating or preventing a disease or condition in a subject, wherein the disease or condition may be selected from, for example, autoimmune diseases and conditions, other inflammatory diseases, infectious diseases and proliferative diseases.

In a third aspect, the present disclosure provides the use of an immunotherapeutic protein as defined in the first aspect, in the manufacture of a medicament for treating or preventing a disease or condition, wherein the disease or condition is selected from autoimmune diseases and conditions, other inflammatory diseases, infectious diseases and proliferative diseases.

In a fourth aspect, the present disclosure provides a method for treating or preventing a disease or condition, comprising administering to the subject an effective amount of an immunotherapeutic protein as defined in the first aspect, wherein the disease or condition is selected from autoimmune diseases and conditions, other inflammatory diseases, infectious diseases and proliferative diseases.

In a fifth aspect, the present disclosure provides a pharmaceutical composition or medicament comprising an immunotherapeutic protein as defined in the first aspect, and a pharmaceutically acceptable carrier, diluent and/or excipient.

### BRIEF DESCRIPTION OF FIGURES

FIG. 1 provides diagrammatic representations of the structure of the Fc of human IgG1 showing that the H429 residue is located in an inaccessible site within the Fc fragment. The representations are of the Human IgG1-Fc (PDB: 1Fc1) reproduced from Deisenhofer J., Biochemistry 20:2361-2370, 1981 with space filled rendering. (A) Side view of the Fc with the A-chain shown in black and B-chain in light grey with N-linked glycan shown in dark grey. The boxed area of the A-chain indicates the region enlarged in panels B and C; (B) the enlarged boxed area of panel A indicating in grey, solvent accessible amino acids of chain-B labelled according to the Eu numbering of human IgG1: methionine at position 428, (M428), glutamic acid 430 (E430), alanine 431 (A431), leucine 432 (L432), histidine H435 which sit above and obscure histidine 429 (H429) which is buried in the Fc; (C) The view of panel B after the solvent accessible residues shown in panel B are not rendered i.e. removed from view. Only now does the buried H429 shown in grey CPK, become apparent beneath these overlying neighbouring solvent accessible residues shown in panel B. Accessible surface area (ASA) for residues calculated using PISA (ie “Protein interfaces, surfaces and assemblies” service PISA; http://www.ebi. ac.uk/pdbe/prot_int/pistart.htm), and Krissinel E and K Henrick. J Mol Biol 372:774-797, 2007) indicates that H429 is inaccessible, recording a value of 0.0;

FIG. 2 provides representations of immunoglobulin (antibodies) and antibody-like (Ab-like) molecules showing the modular nature of the antibody: Left panel: Provides representations of the prototypical immunoglobulin structure—Immunoglobulins are comprised of chains of variable (V) and constant (C) domains arranged in heavy (H) chains and, optionally, light (L) chains that self-assemble. Domains are further identified by chain (e.g. CH3 is the third constant domain of the heavy chain). Heavy chains (H) normally further include a distinct linking, or hinge sequence, between the Fab and Fc segments. Some H chains comprise three constant H domains, as shown in the panel (with residue H429 indicated in the CH3 domain), and others comprise four constant H domains (e.g. IgM and IgE) where CH4 is the equivalent domain to the CH3 of other antibodies (e.g. IgG and IgA). Antibodies from many species include a light (L) chain. In antibodies that lack a light chain, two H chains dimerise (H₂) and normally comprise the antibody. On the other hand, in antibodies that include a light (L) chain, the two antibody H chains will normally dimerise and a L chain is associated with each H chain (i.e. H₂L₂). Some antibodies lacking strong H chain interactions (e.g. such as human IgG4) may at times be antibody “half-molecules” comprising a monomeric H chain and one L chain (H₁L₁). The Fc fragment (or Fc) of an immunoglobulin is a dimer formed by covalent and/or non-covalent interactions between parts of each H chain (i.e. two Fc regions of the H chain, which each comprise a hinge, CH2 and CH3 domain of a heavy chain).

Right panel. Provides representations showing that the modular nature of immunoglobulins allows flexibility in the production of Ab-like molecules and fusion proteins (including immunoglobulin parts). The molecules shown all include an “H-like” chain, comprising at least a CH3 domain. In particular, the Ab-like molecules depicted each consist of a V domain for target antigen recognition joined by a linking sequence to a CH3 domain of an Fc region from, for example, an IgG (which, in variations of the molecules shown, could be an equivalent CH4 domain from an IgE or IgM). The CH3 domain can carry mutations such as those described herein at position 429 (Eu numbering). The V and CH3 domains may be linked by a sequence that may comprise heterologous linking sequences, synthetic linking sequences and/or comprise other homologous or heterologous sequences or domains linking the V and CH3 domains (e.g. as depicted in the examples shown in the figure, the V and CH3 domains can be linked by CH1-CH2 or CH2 with or without a further short polypeptide sequence such as a heterologous or synthetic linking sequence or an immunoglobulin hinge sequence), and the CH1, CH2 and hinge sequence modules may be provided in varied combinations and orders, not limited to the examples shown. The molecules shown are each in a monomeric form, which may dimerise to form homodimeric or heterodimeric forms. Similarly for the fusion proteins shown, these each consist of a target recognition moiety (depicted as “X”) joined by a linking sequence to at least a CH3 domain of an Fc region from, for example, an IgG (or an equivalent CH4 domain of an IgE or IgM). Again, the CH3 domain may include mutations such as those at position 429 (Eu numbering), and the target recognition moiety and H chain modules (e.g. the CH3 domain, CH2-CH3 or CH3-CH2 components) are linked by a sequence that may comprise heterologous or synthetic linking sequences and/or comprise other homologous or heterologous sequences or domains linking the V and CH3 domains (e.g. CH1-CH2 or CH2 with or without a further short polypeptide sequence such as a heterologous or synthetic linking sequence or an immunoglobulin hinge sequence);

FIG. 3 illustrates the conservation of the domain structure and sequence homology in immunoglobulin G (IgG) and immunoglobulin A (IgA) molecules: The sequence comparison of the hinges and constant domains of human immunoglobulin heavy (H) chain sequences are provided depicting the domain-based structure of the H chain (i.e. CH1-Hinge-CH2-CH3 defined by IgG1; Eu numbering indicated (inverted triangle) for the first amino acid of each IgG1 domain and hinge is shown thus: CH1 domain, amino acids 118 to 215; hinge amino acids 216 to 230; CH2 domain amino acids 231 to 340; and CH3 domain amino acids 341 to 447). The other IgG subclasses IgG2, IgG3, IgG4, as well as IgA subclasses, IgA1 and IgA2, have a corresponding domain structure with amino acid sequence homology. Also shown is the conservation of the histidine residue at position 429 of human IgG1 across the other IgG subclasses and IgA subclasses (indicated by the arrow). Amino acid sequences were derived from translation of open reading frames of sequences from the European Nucleotide Archive (https://www.ebi.ac.uk/ena/browser/). Accession numbers of the H chain sequences are: IgG1 H chain, J00228-IGHG1; IgG3 H chain, X03604-IGHG3; IgG4 H chain, K01316-IGHG; IgG2 H chain, J00230-IGHG2; IgA1 H chain, J00220-IGHA1; and IgA2 H chain, J00221-IGHA2. Alignments were performed using Clustal except for the hinges which were aligned manually;

FIG. 4 shows that the histidine of position 429 in IgG1 is conserved in all immunoglobulin classes. Comparison of the amino acid sequence of the IgG1 CH3 domain and the corresponding domains of other human immunoglobulin classes shows that Histidine 429, indicated by the arrow, is conserved in the CH3 domains of the other IgG subclasses, IgG2, IgG3 and IgG4 and the IgA subclasses IgA1 and IgA2, IgD and in the equivalent domain (CH4) of IgE and IgM. The IgG1 CH3 domain is defined and numbered according to the Eu numbering and the first amino acid in the CH3 domain is indicated by the inverted triangle above glycine. The amino acid sequences shown were derived from translation of open reading frames of sequences from the European Nucleotide Archive (https://www.ebi.ac.uk/ena/browser/). Accession numbers of the H chain sequences are: IgG1 CH3 domain, J00228-IGHG1; IgG3 CH3 domain, X03604-IGHG3; IgG4 CH3 domain, K01316-IGHG4; IgG2 CH3 domain, J00230-IGHG2; IgA1 CH3 domain, J00220-IGHA1; IgA2 CH3 domain, J00221-IGHA2; IgE CH4 domain, IGHE-CH4-J00222; IgM CH4 domain, IGHM-CH4-X57331; and IgD CH3 domain, IGHD-K02879;

FIG. 5 shows the results of the purification of an ACE2-Fc H429Y fusion protein according to the present disclosure: (A) Anion exchange (IEX) chromatography of flACE2-Fc-WT (flow through (ft), eluted fractions, and wash) with the ACE2-Fc-containing peak highlighted by *; (B) SDS-PAGE of flow through (ft) and IEX peak fractions with the flACE2-Fc H429Y migrating above the 250 kDa marker and low molecular weight (mw) impurities marked t; (C) Size-exclusion chromatography (SEC) of IEX fractions containing flACE2-Fc-WT (comparator) using a Superose 6 column, with oligomeric (oli), monomeric (mn) and low mw impurities (t) indicated; and (D) SEC of IEX fractions containing flACE2-Fc H429Y, showing the high proportion of oligomeric species. The monomeric (mn) species are considered to be single molecules (i.e. monomer molecules) comprising two copies of the respective ACE2-Fc fusion polypeptide self-associated through the Fc region components;

FIG. 6 provides graphical results showing SARS-CoV-2 RBD binding activities of ACE2-Fc fusion proteins according to the present disclosure. ACE2-Fc fusion protein binding to immobilised RBD-Ig was determined by ELISA for: (A) trACE2-Fc-WT, (B) flACE2-Fc-WT and (C) and EflACE2-Fc-WT and for the following variants thereof:
F ACE2-Fc proteins including a mutated H429F Fc region component
Yₒₗᵢ SEC purified ACE2-Fc proteins including a mutated H429Y Fc region component (oligomeric proteins)
Ymn SEC purified ACE2-Fc proteins including a mutated H429Y Fc region component (monomer proteins; comprising two copies of the ACE2-Fc fusion protein dimerised through the Fc region components)
kif trACE2-Fc-WT proteins produced in the presence of the mannosidase inhibitor, kifunensine. (D) Summary of EC₅₀ binding constants for the trACE2-Fc-WT, flACE2-Fc-WT and variant proteins demonstrating a higher apparent RBD binding affinity for the flACE2-Fc-WT over trACE2-Fc-WT and a weaker binding affinity for the flACE2-Fc-H429Y monomer (Ymn; i.e. comprising two copies of the flACE2-Fc-H429Y fusion protein dimerised through the Fc region components). Welch's unpaired t test p=0.0332 (*), <0.0001 (****);

FIG. 7 provides results showing that the flACE2-Fc H429Y fusion protein according to the present disclosure forms pH-dependant oligomers: SEC of flACE2-Fc H429Y purified by IEX was dialysed against (A) PBS 7.4 or (B) 100 mM citrate, 100 mM NaCl pH 5 and then SEC was performed in the same buffers. SEC at pH 5 yielded a greater proportion of monomeric (mn) fusion protein, than separation at pH 7.4. (C) Native PAGE (1 μg) of ACE2-Fc H429Y. Lane 1, SEC pH 5 oligomers (oli); Lane 2, SEC pH 5 monomer (mn); Lane 3, SEC pH 7.4 oligomers; Lane 4, SEC pH 7.4 monomer; Lane 5, SEC pH 5 monomer re-dialysed against PBS pH 7.4 and purified by SEC and the mn fractions collected as in panel D. (D) SEC chromatogram of pH 5.0 oligomers analysed in lane 1 of panel C, re-dialysed against PBS pH 7.4 and SEC performed in PBS pH 7.4. (E) Improved RBD-Ig binding activity of flACE2-Fc-H429Y monomer (Yₘₙ) prepared at pH 5. The monomers are considered to be single molecules (i.e. monomer molecules) comprising two copies of the respective ACE2-Fc fusion protein dimerised through the Fc region components;

FIG. 8 provides results showing that the SARS-CoV-2 neutralisation potency of ACE2-Fc fusion proteins according to the present disclosure is affected by the ACE2 scaffold (i.e. truncated or full-length) and Fc mutation. Neutralisation potencies of the ACE2 polypeptide and the three groups of ACE2-Fc-WT fusion and variant proteins were determined by titration to cytopathic effect (CPE) endpoint in a micro-neutralisation assay. The fusion proteins are trACE2-Fc, flACE2-Fc and EflACE2-Fc WT, and Fc variant, H429F, F; H429Y oligomers on SEC, Yₒₗᵢ and H429Y monomers on SEC, Yₘₙ (comprising two copies of the fusion protein dimerised through the Fc region components). The trACE2-Fc fusion proteins include the glycan-modified trACE2-Fc-kif. Neutralisation endpoint mean±SEM, ANOVA with Dunnett's multiple comparisons test compared to ACE2 and ACE2-Fc WT. p=0.1234 (ns), 0.0332 (*), 0.0021 (**), 0.0002 (***), <0.0001 (****);

FIG. 9 provides the results of assays to assess the interaction of ACE2-Fc fusion proteins according to the present disclosure with FcγR. Ramos-S cells were opsonised with the ACE2-Fc WT fusion protein and variant proteins (5 μg/ml). Biotinylated (A) dimeric rsFcγRIIa or (B) dimeric rsFcγRIIIa probes, followed by streptavidin-APC were bound to the opsonised cells and binding described as median fluorescence intensity (Median FI) determined by flow cytometry. FcR binding activity of the ACE2-Fc fusion proteins was readily detected with the exception of ACE2-Fc H429Y fusion proteins which was greatly diminished (3 replicates, mean±SEM);

FIG. 10 provides the results of assays to assess the capacity of ACE2-Fc fusion proteins according to the present disclosure to mediate cell activation via FcγRIIIa. The results demonstrate that flACE2-Fc proteins are potent activators of Fc γ RIIIa with the exception of the Fc H429Y mutants in any ACE2 format which fail to stimulate. The afucosylated trACE2-Fc-kif is also a potent activator of FcγRIIIa. Ramos-S target cells were opsonised with (A) trACE2-Fc, (B) flACE2-Fc and (C) EflACE2-Fc, WT and variant proteins, including H429F, F; H429Y, oligomers, Yoli; H429Y monomer, Ymn (comprising two copies of the fusion protein dimerised through the Fc region components); or trACE2-Fc kifproduced from trACE2-Fc WT in Expi293 cells in the presence of the mannosidase inhibitor, kifunensine. In some experiments, Ramos-S target cells were separately opsonised with anti-CD20 mAb rituximab, RIT. These opsonised targets were incubated with FcγRIIIa-NF—KB-RE nanoluciferase reporter cells, and FcγRIIIa activation measured by the induction of nanoluciferase (RLU). Data was fitted to agonist response curves to estimate EC₅₀. (D) EC₅₀ (nM) values from the curve fits are shown. Mean±SEM, n>4, ANOVA with Dunnett's multiple comparisons test comparing to trACE2-Fc WT. p=0.0021 (**), 0.0002 (***), <0.0001 (****);

FIG. 11 provides results showing that ACE2-Fc fusion proteins according to the present disclosure, comprising Fc region components with the H429F and H429Y mutations, strongly fix complement and direct complement dependent cytotoxicity killing of Ramos-S target cells; as determined using ELISA analysis of the complement fixing activity of trACE2-Fc (A, C, E) or flACE2-Fc (B, D, F) bound to SARS-CoV-2 RBD-biotin captured by plate bound avidin: C1q binding (A, B) to ACE2-Fc fusion protein variants (2.0 μg/ml) at different concentrations of avidin-captured RBD on the plate; titration of C1q (C, D) or C5b-9 (E, F) binding by serially diluted ACE2-Fc fusion protein variants bound to avidin-captured RBD-biotin (2.5 μg/ml) (mean±SEM); two independent ELISA experiments. (G) Flow cytometric analysis of complement dependent cytotoxicity (CDC) (% killing) of opsonised Ramos-S cells was determined using the presence of a 1/3 dilution of normal human serum as a source of complement (EC₅₀ (nM) values from the curve fits are shown);

FIG. 12 provides the results of assays showing that IgG1 antibodies comprising Fc region components with the H429F mutation of the CH3 domain strongly fix complement and show identical antigen binding. (A,B) TNP-BSA antigen was adsorbed at different concentrations (20 μg/ml-0.625 g/ml) to the wells of ELISA plates and were reacted with a single concentration (2 μg/ml) of chimeric anti-TNP human IgG1 and IgG2 mAbs comprising unmodified wildtype (WT) heavy chains or with 2 g/ml anti-TNP mAbs comprising IgG1 heavy chains comprising the Fc component mutation H429F (TNP-IgG1-H429F). The antibody-opsonised TNP-BSA coated wells were treated with (A) purified human C1q and the fixation of C1q detected with anti-C1q rabbit polyclonal antibody and (B) with human serum as a source of complement and the formation of the membrane attack complex (C5b-C9) was detected with anti-C5b-C9 rabbit polyclonal antibody. (C) provides the results from an ELISA showing the antigen binding activity of the chimeric anti-TNP mAbs used in panels A and B including the unmodified (TNP-IgG1-WT and TNP-IgG2-WT mAbs) and H429 modified mAb, namely TNP-IgG1-H429F. ELISA plates were coated with TNP-BSA then anti-TNP mAbs titrated and antibody binding detected with HRP-conjugated anti-human IgG. All mAbs showed similar antigen binding activity;

FIG. 13 provides results showing C1q fixation and MAC (C5b-9) formation by mAbs comprising IgG1 heavy chains with glutamic acid, glutamine or serine modifications at position 429 of the heavy chain. TNP-BSA (20 μg/ml) was adsorbed to the wells and reacted with chimeric human anti-TNP mAbs, titrated across the concentration range 4 μg/ml-0.125 μg/ml. The mAbs tested comprised unmodified wild type (WT) heavy chains of IgG1 (TNP-IgG1-WT) (panels A-D) or IgG2 (TNP-IgG2-WT) (panels A-C) or IgG1 H chains comprising mutations TNP-IgG1-H429Q or TNP-IgG1-H429E (A,C) and TNP-IgG1-H429S (B,D). Antibody opsonised TNP-BSA coated wells were treated with human serum as a source of complement. The fixation of C1q (A,B) was detected with anti-C1q rabbit polyclonal antibody and (C,D) the formation of the membrane attack complex (C5b-C9) was detected with rabbit anti-C5b-C9 polyclonal antibody;

FIG. 14 provides the results of flow cytometric quantitation of SEC purified mAb binding to target cell surface antigens. The binding activity of the unmodified (WT) rituximab, daratumumab-WT, 11B8-WT mAbs and their CH3-modified mutants carrying the mutations H429F or H429Y (SEC (IgG H₂L2) peak fraction p1 and IgGₒₗᵢ peak fraction p2) was evaluated by flow cytometry on Ramos lymphoma cells expressing CD20 and CD38. The binding activity of the unmodified trastuzumab-WT, pertuzumab-WT and their CH3 heavy chain mutants carrying the mutation H429F was evaluated on SK-OV-3 cells expressing HER2;

FIG. 15 provides elution chromotagrams of Protein A affinity purification characteristics of rituximab-WT, trastuzumab-WT and their mutants bearing modification in the CH3 of the heavy chain. The IgG was recovered from the column following elution with sodium citrate buffer pH 3.0: (A) Elution profiles of anti-CD20 rituximab-based mAbs. The rituximab-WT IgG and each mutant IgG eluted as a single homogenous peak; (B) Elution profiles of anti-HER2 trastuzumab-based mAbs. The trastuzumab-WT IgG and each mutant IgG eluted as a single homogenous peak;

FIG. 16 provides size exclusion chromatography (SEC) profiles revealing that H429 mutation can alter the physical properties of IgG. Following Protein A affinity purification, the mAbs were further purified by SEC at pH 7.2: (A) The SEC profiles of the unmodified rituximab-WT (WT) and the rituximab-H429F (H429F) mutant consisted of a single homogenous IgG peak (fractions right of the vertical dashed line) with minimal oligomeric species, (fractions left of the vertical dashed line). Thus, for example, the SEC profile of unmodified rituximab-WT showed a single major species corresponding, as expected, to IgG (H₂L2) as confirmed by SDS-PAGE analysis (FIG. 17A) where it migrated at the expected ˜150 kDa mass of unreduced IgG and which resolved, after reduction in DTT, to its ˜50 kDa heavy (H) chain and ˜25 kDa light (L) chain species. The rituximab-H429Y mAb on the other hand comprised both non-oligomeric IgG and oligomeric IgG; (B) The SEC profiles of Protein A purified anti-HER2 trastuzumab wild-type (WT) (that is, an unmodified form of trastuzumab) and its mutants including the amino acid substitution of H429F or H429Y. A single peak of non-oligomeric IgG, to the right of the dashed vertical line, was observed for the trastuzumab-WT, and the H429F mutant mAb, but the trastuzumab-H429Y mutant IgG (H429Y) contained both non-oligomeric IgG and oligomeric IgG. The non-oligomeric IgG peak of the trastuzumab-H429Y was coincident with that of the unmodified trastuzumab-WT, or its H429F variant and the equivalent rituximab-based mAbs in panel (A). The SEC profile of the oligomeric IgG in trastuzumab-H429Y was coincident with the oligomeric IgG in rituximab-H429Y in panel (A);

FIG. 17 provides images obtained from SDS-PAGE of SEC purified mAbs. mAbs purified by SEC were analysed by SDS-PAGE in 5-15% gradient gels with or without reduction of disulphide bonds: (A) Under non-reducing conditions, the non-oligomeric IgG peak of rituximab-WT (WT IgG) and rituximab-H429F (HF IgG) migrated as a single species at the expected ˜150 kDa molecular size of IgG (i.e H₂L₂). Following reduction in DTT, the 150 kDa IgG resolved as expected into −50 kDa heavy (H) chain and ˜25 kDa light (L) chains. Prior to reduction, the non-oligomeric IgG (HY IgG (H₂L₂)) and oligomeric (HY IgG(oli)) forms of rituximab-H429Y mAb (see FIG. 16), both migrated identically as a single 150 kDa IgG i.e. H₂L₂ species. Following reduction, both forms migrated as ˜50 kDa heavy (H) chain and ˜25 kDa light (L) chain; WT=wild-type, HF=H429F, HY=H429Y; M=molecular-weight markers, their masses (kD) are shown on the left side; (B) Under non-reducing conditions, the IgG peak of trastuzumab-WT (WT IgG), and trastuzumab-H429F (HF IgG) migrated as a single species at the expected ˜150 kD molecular size of IgG i.e. H₂L2. Following reduction with dithiothreitol (DTT), the 150 kDa IgG resolved as expected into ˜50 kDa heavy (H) chain and ˜25 kDa light (L) chains. Prior to reduction, the non-oligomeric IgG (HY IgG (H₂L2)) and oligomeric (HY IgG(oli)) forms of trastuzumab-H429Y mAb both migrated identically as a single 150 kDa IgG species under non-reducing conditions and following reduction, as the expected ˜50 kDa heavy (H) chain and ˜25 kDa light (L) chain; WT=wild-type, HF=H429F, HY=H429Y; M=molecular-weight markers, their masses (kD) are shown on the left side;

FIG. 18 provides the results of experimentation showing that oligomeric and non-oligomeric forms of H429Y-modified IgG antibodies show equivalent CDC potency and that their formation is pH sensitive: (A) SEC of the rituximab-H429Y at pH7.2 showing the presence of two major IgG forms (see also FIG. 16A). The non-oligomeric IgG (H₂L2) (right of the vertical dashed line, indicated as p1) and the oligomeric form of IgG (IgG(oli)), (left of the vertical dashed line indicated as p2) were separately collected for further evaluation of complement-dependent cytotoxicity (CDC) potency; (B) The oligomeric (p2) and non-oligomeric (p1) IgG forms from panel A, and the SEC purified unmodified rituximab-WT IgG were titrated and CDC potency was determined by flow cytometry using Ramos lymphoma cells and normal human serum diluted 1/3 as the source of complement. The extent of killing of the Ramos cells opsonised with the non-oligomeric rituximab H429Y p1 (open squares), oligomeric rituximab-H429Y p2 (triangles) or unmodified rituximab-WT (filled circles) was determined. The control background CDC of complement in the absence of mAb (no mAb C′ only) is shown as a filled diamond; and (C) The formation of IgG oligomers is sensitive to pH. Protein A purified trastuzumab-H429Y was analysed by size exclusion chromatography (SEC) either at pH 7.2 (left panel) or at pH 5.0 (right panel). At pH 7.2, both oligomeric (IgG (oli)) and non-oligomeric IgG were present, however at pH 5.0, only the single peak corresponding to non-oligomeric IgG was present;

FIG. 19 provides results which show that an H429F mutation in the CH3 domain of the IgG H chain potently promotes C1q binding and complement dependent cell-mediated cytotoxicity. Flow cytometric detection of C1q binding (fluorescence intensity) to Ramos lymphoma cells opsonised with unmodified rituximab-WT (A) or with the modified rituximab-H429F mAb (B), or to SK-OV-3 ovarian cancer cells opsonised with trastuzumab-WT (C) or with the modified trastuzumab-H429F mAb (D) is depicted. The cells opsonised with the mAbs were treated with normal human serum as a source of complement and the binding of C1q was detected by staining with anti-C1q rabbit polyclonal antibody. The C1q binding to the mAb opsonised cells is shown in the un-shaded histograms and the C1q background binding control (i.e. cells treated with serum complement in the absence of mAbs), is shown in the grey-shaded histograms. The median fluorescence intensity (MFI) values for each histogram are indicated in parentheses. (E) The complement-dependent cytotoxicity (CDC; i.e. % killing detected using Zombie Green) of Ramos lymphoma cells opsonised with rituximab-WT (filled circles) or its mutated variant mAb rituximab-H429F (filled squares), was determined by flow cytometry using normal human serum as the source of complement. CDC is greatly enhanced by mutation at position 429 in the rituximab-H429F to compared rituximab-WT mAb;

FIG. 20 shows the results of Protein A affinity chromatography of Type-II anti-CD20 11B8-WT mAb and mutant mAb including an H429 substitution. The mAbs were 11B8-WT mAb (WT) produced with a wild-type human IgG1 heavy chain. The 11B8-H429F (H429F) was produced with a modified IgG1 heavy chain wherein amino acid histidine at position 429 in the CH3 domain of the Ig heavy chain was replaced with phenylalanine;

FIG. 21 shows the results of size exclusion chromatography (SEC) purification and SDS PAGE analysis of the 11B8-WT mAb and the 11B8-H429F (H429F) mutant. (A) Chromatograms of the mAbs purified by SEC at pH 7.2; monomeric non-oligomeric IgG is shown to the right of the vertical dotted line and (B) SDS-PAGE analysis (5-15% gradient gel) of the SEC-purified monomeric IgG mAbs from panel A. Prior to reduction (Non-Reduced), all SEC purified antibodies migrated at the expected 150 kDa molecular size of IgG i.e H₂L2. Following reduction with DTT (Reduced), all of the antibodies resolved as expected into ˜50 kDa heavy (H) chain and ˜25 kDa light (L) chain. WT=wild-type, HF=H429F, M=molecular-weight markers, their masses (kD) are shown on the left side;

FIG. 22 provides results showing that H429 modification of the CH3 domain confers CDC potency on a type II anti-CD20 mAb. CDC by the 11B8-WT mAb (solid circles) or CH3 mutated 11B8-H429F mAb (solid squares). Complement-dependent lysis was determined using Ramos lymphoma cells opsonised with the mAbs at the indicated concentrations and normal human serum diluted 1/3 as the source of complement. CDC (% killing) was determined by flow cytometry using Zombie Green. The control background CDC of complement in the absence of mAb (no mAb C′ only) is shown as a filled diamond. The 11B8-WT mAb failed to mediate CDC whereas the 11B8-H429F mutant mAb mediated potent complement-dependent lysis of lymphoma cells (WT=wild type);

FIG. 23 shows the results of Protein A affinity chromatography of the anti-CD38 daratumumab-WT mAb and the daratumumab-H429F mAb. Elution chromatograms showed that each of the antibodies eluted as a single coincident homogenous peak;

FIG. 24 shows the results of size exclusion chromatography (SEC) purification and SDS-PAGE analysis of the daratumumab-WT mAb as well as the mutant mAb, daratumumab-H429F. (A) Chromatograms of the SEC purified mAbs. In each case, the mAbs contained a single non-oligomeric IgG peak (fractions right of the vertical dashed line) and the absence of oligomeric species (fractions left of the vertical dashed line); (B) SDS-PAGE analysis (5-15% gradient gel) of SEC purified IgG from panel A. Prior to reduction (Non-Reduced), all of the antibodies migrated at the expected ˜150 kDa molecular size of IgG (i.e. H₂L₂), and following reduction with DTT (Reduced), all of the antibodies resolved as expected into ˜50 kDa heavy (H) chain and ˜25 kDa light (L) chain. WT=wild-type, HF=H429F, M=molecular-weight markers, their masses (kD) are shown on the left side;

FIG. 25 provides results which show that the H429F substitution profoundly enhances complement-dependent lysis of lymphoma cells by daratumumab-WT. The graph shows the level of CDC (% killing) by the daratumumab-WT mAb (solid circles) and daratumumab-H429F (solid squares). Background lysis (solid triangle) was determined in the absence of mAb, but in the presence of only complement. CDC was determined by flow cytometry using Zombie Green. The daratumumab-H429F mAb mediated more potent complement-dependent lysis of lymphoma cells than the unmodified daratumumab-WT;

FIG. 26 provides results showing that the H429F substitution confers complement-dependent lysis of myeloma and leukaemia cells resistant to lysis by anti-CD38 mAb. CDC by complement-dependent lysis was determined using: (A) KMS-12-PE myeloma cells opsonised with the daratumumab-WT mAb (solid circles) or daratumumab-H429F mutant mAb (solid squares) at the indicated concentrations. Background lysis (solid triangle) was determined in the absence of mAb, but in the presence of only complement; and (B) SUP-15 acute lymphoblastic leukaemia (ALL) cells which were opsonised with the daratumumab-WT mAb (solid circles) and daratumumab-H429F mutant mAb (solid squares) at the indicated concentrations. Background lysis (solid triangle) was determined in the absence of mAb but in the presence of only complement. The CDC (% killing) was determined by flow cytometry using Zombie Green;

FIG. 27 shows the results of Protein A affinity chromatography of the anti-HER2 mAb pertuzumab-WT and CH3 variant. Elution chromatograms showed that each of the antibodies eluted as a single coincident homogenous peak;

FIG. 28 shows the results of size exclusion chromatography (SEC) purification and SDS-PAGE analysis of the anti-HER2 mAb pertuzumab-WT and CH3 H429F variant. (A) Chromatograms of SEC purified mAbs. Following Protein A affinity chromatography, the mAbs were further purified by size exclusion chromatography (SEC) at pH 7.2. The chromatographic profiles are shown for unmodified pertuzumab-WT and for an Fc modified variant, pertuzumab-H429F (H429F). In each case, the Protein A-purified mAbs contained a single non-oligomeric IgG peak (fractions right of the vertical dashed line) and the absence of oligomeric species (fractions left of the vertical dashed line). (B) SDS-PAGE analysis (5-15% gradient gel) of SEC purified IgG from panel A. Prior to reduction (Non-Reduced), all of the SEC purified antibodies migrated at the expected ˜150 kDa molecular size of IgG (i.e. H₂L₂). Following reduction with DTT (Reduced), all of the antibodies resolved as expected into ˜50 kDa heavy (H) chain and ˜25 kDa light (L) chain. WT=wild-type, HF=H429F, M=molecular-weight markers shown in kilodaltons (kD);

FIG. 29 provides results showing the co-operation and functional synergy in mixtures of mAbs including amino acid substitution at position H429; in particular, the results show that H429F-modified mAbs can combine to enhance C1q binding to target cells: (A) Flow cytometric histograms of C1q binding to HER2 expressing SK-OV-3 cells opsonised (unfilled histograms) with trastuzumab-H429F alone, pertuzumab-H429F alone or a 1:1 mixture of trastuzumab-H429F and pertuzumab-H429F. Filled histograms show the background binding of C1q to non-opsonised cells. The Median Fluorescence Intensity (MFI) values for each histogram is also shown. C1q binding was detected using an anti-C1q-specific polyclonal rabbit antibody; (B) Titration of enhanced functional cooperation. SK-OV-3 cells were opsonised with titrated individual anti-HER2 mAbs, trastuzumab-WT, pertuzumab-WT, trastuzumab-H429F, pertuzumab-H429F or with 1:1 mixtures of trastuzumab-WT with pertuzumab-WT or trastuzumab-H429F with pertuzumab-H429F. The double-headed vertical arrow exemplifies extensive cooperation in the mixture of trastuzumab-H429F with pertuzumab-H429F where antibody concentrations are limiting compared to C1q binding of either mAb alone;

FIG. 30 provides graphical results showing that the co-operation and functional synergy of H429-modified mAbs enhances complement dependent killing (CDC) of target cells. The indicated rituximab-based mAbs were titrated alone (light grey columns) on Ramos cells or titrated in the presence of a fixed concentration of the indicated anti-CD38 or anti-CD20 mAbs (dark columns): (A) rituximab-WT titrated in the presence of 0.025 μg/ml, daratumumab-WT, (B) rituximab-HF titrated in the presence 0.025 μg/ml daratumumab-H429F, (C) rituximab-HF titrated in the presence of 0.5 μg/ml, 11B8-H429F. CDC (% killing) was determined by flow cytometry using Zombie Green. The dotted horizontal line in (B, C) indicates the percentage of CDC killing obtained in the absence (0 μg/ml) of rituximab-based mAbs, but in the presence only of (B) 0.025 μg/ml daratumumab-H429F or (C) 0.5 μg/ml 11B8-H429F. The black arrows in (B, C) indicate the enhanced CDC of the mixtures of mAbs above that of the individual mAbs (ie the CDC of either the rituximab-H429F alone at the indicated concentrations (light columns) or of 0.025 μg/ml daratumumab-H429F or 0.5 μg/ml 11B8-H429F (dark column, 0 μg/ml rituximab));

FIG. 31 provides the results of flow cytometry analysis of CDC potency of a rituximab antibody comprising an H429F mutation on normal peripheral blood CD19⁺ B lymphocytes. The results indicate that the rituximab-H429F mAb exhibits more potent CDC killing of normal peripheral blood B cells compared to unmodified rituximab-WT. Following treatment of peripheral blood mononuclear cells with the indicated mAbs and human complement, the proportion of live or dead B cells was determined by staining with anti-CD19 to identify B cells and also with Zombie Green (ZG) to identify dead cells. The cytograms show Zombie Green median fluorescence intensity (ZG MFI) of gated CD19 B cells (CD19 median fluorescence intensity (CD19 MFI). In each cytogram, the % of dead cells (ZG positive) is shown in the upper section, Q1, of each cytogram for rituximab-WT (30.4% ZG positive), rituximab-H429F (85.6% ZG positive) and the corresponding negative control mAbs trastuzumab-WT (3.05% ZG positive) and trastuzumab-H429F (3.11% ZG positive); the % live B cells are shown in the lower section, Q2;

FIG. 32 provides results showing that the effects of modification of H429 on the function of antibodies are not restricted to IgG1 and extend to other immunoglobulin types. Here, CDC potency on Ramos lymphoma cells of rituximab antibodies formatted with wild-type (WT) or H429F (HF) modified heavy chains of (A) IgG3 or (B) IgG4 subclasses were assessed;

FIG. 33 provides results showing that potent CDC of target cells mediated by mAbs is dependent on the presence of both the monoclonal antibody and serum complement. CDC (% killing) of Ramos cells by rituximab-H429F, daratumumab-H429F or 11B8-H429F, or of SUP-15 cells by daratumumab-H429F is shown. A=percent killing in the presence of both the indicated mAb and human serum as a source of complement; B=lysis in the presence of mAb only at the same concentration as in A; and C=lysis in the presence of complement only;

FIG. 34 provides results showing that the H429F substitution confers complement-dependent lysis of leukaemia cells resistant to lysis by the unmodified anti-CD38 mAb isatuximab. CDC by complement-dependent lysis was determined using SUP-15 acute lymphoblastic leukaemia (ALL) cells which were opsonised with the isatuximab-WT mAb (dashed line with filled circles) and isatuximab-H429F mutant mAb (solid line with filled squares) at the indicated concentrations. Background lysis (unfilled circle) was determined in the absence of mAb but in the presence of only complement (C′ only, no mAb). Background lysis induced by the mAbs at 5 μg/ml in the absence of complement was determined for isatuximab-WT mAb (inverted unfilled triangle) and isatuximab-H429F mutant mAb (open unfilled square). The CDC (% killing) was determined by flow cytometry using Zombie Green;

FIG. 35 provides the results of flow cytometric quantitation of the binding to Colo205 colorectal cells of purified DR5-specific mAbs that comprised unmodified wild-type (WT) H chains of the human IgG1 or IgG2 subclasses or comprised human IgG1 or human IgG2 subclass heavy chains that had been modified by replacement of histidine 429 with phenylalanine (H429F). The panels show: (A) binding of BDR5-1WT which comprised wild-type H chains of the human IgG1 subclass or binding of the BDR5-1HF mAb which comprised IgG1 heavy chains containing the H429F modification, (B) binding of BDR5-2WT which comprised unmodified heavy chains of the human IgG2 subclass or binding of the BDR5-2HF mAb which comprised IgG2 heavy chains containing the H429F modification, (C) the binding of the TDR5-1WT mAb which comprised unmodified heavy chains of the human IgG1 subclass or the TDR5-1HF mAb which comprised IgG1 heavy chains containing the H429F modification, and (D) binding of TDR5-2WT which comprised unmodified heavy chains of the human IgG2 subclass or binding the TDR5-2HF mAb which comprised IgG2 heavy chains containing the H429F modification. The mAbs were serially 2-fold titrated and the binding activity on Colo205 cells was quantified by flow cytometry using an anti-IgG secondary reagent labelled with goat anti-hIgG Fc FITC. In all panels, the level of non-specific binding of fluorescent conjugate to cells is shown (▴ conj); MFI=Median Fluorescence Intensity;

FIG. 36 provides the results of flow cytometric quantitation of the binding to target Ramos lymphoma cells of purified DR5-specific mAbs that comprised unmodified wild-type (WT) H chains of the human IgG1 or human IgG2 subclasses or comprised human IgG1 or human IgG2 heavy chains that had been modified by replacement of histidine 429 with phenylalanine (H429F). The panels show: (A) binding of BDR5-1WT which comprised unmodified heavy chains of the human IgG1 subclass or binding of the BDR5-1HF mAb which comprised IgG1 heavy chains containing the H429F modification; (B) binding of BDR5-2WT which comprised unmodified heavy chains of the human IgG2 subclass or binding of the BDR5-2HF mAb which comprised IgG2 heavy chains containing the H429F modification; (C) binding of TDR5-1WT which comprised unmodified heavy chains of the human IgG1 subclass or binding of the TDR5-1HF mAb which comprised IgG1 heavy chains containing the H429F modification; and (D) binding of TDR5-2WT which comprised unmodified heavy chains of the human IgG2 subclass or binding of the TDR5-2HF mAb which comprised IgG2 heavy chains containing the H429F modification. The mAbs were serially 2-fold titrated and the binding to Ramos cells was quantified by flow cytometry using an anti-IgG secondary reagent labelled with goat anti-hIgG Fc FITC. In all panels, the level of non-specific binding to cells was determined using an irrelevant IgG antibody (negative IgG) and the background binding of the fluorescence anti-IgG conjugate to cells is shown (▴ conj); MFI=Median Fluorescence Intensity;

FIG. 37 provides the results of flow cytometric quantitation of the binding to target KMS-12-PE myeloma cells of purified DR5-specific mAbs that comprised unmodified wild-type (WT) H chains of the human IgG1 or human IgG2 subclasses or comprised human IgG1 or human IgG2 heavy chains that had been modified by replacement of histidine 429 with phenylalanine (H429F). The binding activity is shown for the DR5-specific mAbs, BDR5 and TDR5, which comprised unmodified, wild-type, H chains of the human IgG1 subclass (BDR5-1WT, TDR5-1WT) or human IgG2 subclass (BDR5-2WT, TDR5-2WT) or comprised Fc mutated H chains of the IgG1 subclass (BDR5-1HF, TDR5-1HF) or IgG2 subclass (BDR5-2HF, TDR5-2HF) containing the H249F mutation. KMS12-PE cells were incubated with mAbs at 5 μg/ml. Binding activity was quantified by flow cytometry using an anti-IgG secondary reagent labelled with FITC goat anti-hIgG Fc. The background control of anti-IgG conjugate only binding is shown (conj.) along with the background fluorescence of cells only (cells);

FIG. 38 provides results showing the survival of Colo205 colorectal cells in the presence of purified DR5-specific mAbs comprised of unmodified wild-type H chains of the human IgG1 or human IgG2 subclasses, or comprised of Fc mutated H chains of the human IgG1 or human IgG2 subclasses containing the H429F mutation. Ten thousand cells were cultured in each well of a 96 well plate in the presence of 20 μg/ml of each of the following mAbs: TDR5-1WT mAb which comprised unmodified wild-type H chains of the human IgG1 subclass, or TDR5-1HF mAb which comprised H chains of the human IgG1 subclass containing the H429F mutation, or TDR5-2WT mAb which comprised unmodified wild-type H chains of the human IgG2 subclass, or TDR5-2HF mAb which comprised H chain of the human IgG2 subclass containing the H429F mutation. Additionally, Colo205 cells were separately cultured in the presence of a mixture of 10 μg/ml TDR5-2HF and 10 μg/ml BDR5-2HF mAbs (TDR5-2HF+BDR5-2HF) both of which comprised Fc mutated human IgG2 H chains carrying H429F mutations. Cell viability was quantitated using a CCK8 colorimetric cell viability assay where absorbance (Abs450 nm) of cell culture supernatant is a measure of cell survival and viability. Maximum cell viability was determined by the culture of cells in the absence of antibody (no ab). Survival is represented by: [Abs450 nm of mAb or control treatment determined experimentally−background Abs450 of cell culture medium], the mean Abs450 and the four replicate values are shown;

FIG. 39 provides results showing the survival of Ramos lymphoma cells in the presence of purified DR5-specific mAbs comprised of unmodified wild-type or Fc mutated H chains of the human IgG1 or IgG2 subclasses containing the H429F mutation. Ten thousand cells were cultured in each well of a 96 well plate in the presence of 20 μg/ml of each of the following mAbs: TDR5-1WT mAb which comprised unmodified wild-type H chains of the human IgG1 subclass, or TDR5-1HF mAb which comprised H chain of the human IgG1 subclass containing the H429F mutation, TDR5-2WT mAb which comprised unmodified wild-type H chains of the human IgG2 subclass, or TDR5-2HF mAb which comprised H chains of the human IgG2 subclass containing the H429F mutation. Additionally, Ramos cells were separately cultured in the presence of a mixture of 10 μg/ml TDR5-2HF and 10 μg/ml BDR5-2HF mAbs (TDR5-2HF+BDR5-2HF) both of which comprised Fc mutated human IgG2 H chains carrying H429F mutations. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab). Survival is represented by: [Abs450 nm of mAb or control treatment determined experimentally−background Abs450 of cell culture medium], the mean Abs450 and the four replicate values are shown;

FIG. 40 provides results showing the survival of Colo205 colorectal cells in the presence of mixtures of purified DR5-specific mAbs wherein the mAbs comprised wild-type or Fc modified H chains of the same IgG subclass. The BDR5 and TDR5 mAbs were comprised of unmodified wild-type H chains of the human IgG1 or human IgG2 subclasses or were comprised of heavy chains of the IgG1 subclass or IgG2 subclasses containing the H429F modification. Colo205 cells, 30,000 cells per well of 96-well plate, were cultured in the presence of serial 2-fold dilutions of 1:1 mixtures of mAbs comprised of identical H chains. The starting concentration of 1 μg/ml was comprised of 0.5 μg/ml of each mAb in the mixture. Thus, the mixtures used were: BDR5-1WT mAb comprising unmodified wild-type IgG1 H chain mAb mixed with TDR5-1WT comprising unmodified wild-type IgG1 H chain (BDR5-1WT+TDR5-1WT); BDR5-2WT mAb comprising unmodified wild-type IgG2 H chain mAb mixed with TDR5-2WT comprising unmodified wild-type IgG2 H chain (BDR5-2WT+TDR5-2WT); BDR5-1HF mAb comprising IgG1 H chains containing H429F modification mixed with TDR5-1HF mAb comprising IgG1 H chains containing H429F modification (BDR5-1HF+TDR5-1HF); BDR5-2HF mAb comprising IgG2 H chains containing H429F modification mixed with TDR5-2HF mAb comprising IgG2 H chains containing H429F modification (BDR5-2HF+TDR5-2HF). Cell viability was quantitated using a CCK8 colorimetric cell viability assay where absorbance (Abs450 nm) of cell culture supernatant is a measure of cell survival and viability. Maximum cell viability was determined by the culture of cells in the absence of antibody (no ab) and maximum death determined by culture of cells with sodium dodecyl sulphate (SDS). Survival is represented by: [Abs450 nm of mAb or control treatment determined experimentally−background Abs450 of cell culture medium];

FIG. 41 provides results showing the survival of Colo205 colorectal cells in the presence of different pairwise combinations of purified DR5-specific mAbs wherein the mAbs comprised wild-type or Fc mutated H chains of different IgG subclasses. MAbs comprised H chains of human IgG1 subclass or human IgG2 subclass comprising wild-type heavy chains or comprising heavy chains modified by H429F modification. Colo205 cells, 10,000 cells per well of 96-well tissue culture plate, were cultured in the presence of 1:1 mixtures of mAbs serially diluted 2-fold from 1 μg/ml. The starting concentration of 1 g/ml was comprised of 0.5 μg/ml of each mAb in the mixture. The mixtures of wild-type mAbs used were: BDR5-2WT comprising wild-type human IgG2 H chain mixed with TDR5-1WT comprising unmodified wild-type human IgG1 H chain (BDR5-2WT+TDR5-1WT); BDR5-1WT comprising wild-type human IgG1 H chain mixed with TDR5-2WT comprising unmodified wild-type human IgG2 H chain (BDR5-1WT+TDR5-2WT); and a control mixture of BDR5-2WT comprising unmodified wild-type human IgG2 H chain mixed with TDR5-2WT comprising unmodified wild-type human IgG2 H chain (BDR5-2WT+TDR5-2WT) as observed in FIG. 40. The mixtures of DR5 mAbs comprising H429F Fc-mutated H chains were: BDR5-2HF comprising H429F Fc-mutated human IgG2 H chain mixed with TDR5-1HF comprising H429F Fc-mutated human IgG1 H chain (BDR5-2HF+TDR5-1HF); BDR5-1HF comprising H429F Fc-mutated human IgG1 H chain mixed with TDR5-2HF comprising H429F Fc-mutated human IgG2 H chain (BDR5-2HF+TDR5-2HF); positive killing control mixture of BDR5-2HF comprising H429F Fc-mutated human IgG2 H chain mixed with TDR5-2HF comprising H429F Fc-mutated IgG2 H chain (BDR5 2HF+TDR5-2HF) as observed in FIG. 38 and FIG. 40. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab) and maximum death determined by culture of cells with sodium dodecyl sulphate (SDS). Survival is represented by: [Abs450 nm of mAb or control treatment determined experimentally−background Abs450 of cell culture medium];

FIG. 42 provides results showing the survival of Colo205 cells in the presence of pairwise mixtures of BDR5 and TDR5 mAbs at different ratios. Ten thousand Colo205 cells were cultured for 48 hours in the presence of individual mAbs BDR5-1WT or TDR5-1WT comprising wild-type IgG1 H chains or individual mAbs BDR5-1HF or TDR5-1HF comprising H chains with the H429F modification or mixtures of BDR5-1HF with TDR5-1HF mAbs at the following ratios, 90:10, 75:25, 50:50, 25:75, and 10:90. In the mixes shown in the plot, the BDR5-1HF mAb is designated as B-1HF and TDR5-1HF is designated as T-1HF. Controls include maximum cell survival in the absence of mAbs (no ab) or maximum death control in the presence of sodium dodecyl sulphate (SDS). Cell viability was quantitated using a CCK8 colorimetric cell viability assay where absorbance (Abs450 nm) of cell culture supernatant is a measure of cell survival and viability. Survival is represented by: [Abs450 nm of mAb or control treatment determined experimentally−background Abs450 of cell culture medium];

FIG. 43 provides results showing the survival of Ramos lymphoma cells in the presence of pairwise mixtures of BDR5 and TDR5 mAbs at different ratios. 10,000 Ramos cells were cultured for 48 hours in the presence of individual mAbs BDR5-1WT or TDR5-1WT comprising wild-type H chains or individual mAbs BDR5-1HF or TDR5-1HF comprising H chains with the H429F modification or mixtures of BDR5-1HF with TDR5-1HF mAbs at the following ratios, 90:10, 75:25, 50:50, 25:75, and 10:90. In the mixes shown in the plot, the BDR5-1HF mAb is designated as B-1HF and TDR5-1HF is designated as T-1HF. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab) and maximum death determined by culture of cells with sodium dodecyl sulphate (SDS). Survival is represented by: [Abs450 nm of mAb or control treatment determined experimentally−background Abs450 of cell culture medium];

FIG. 44 provides results quantitating the binding to Colo205 cells of DR5-specific mAbs that detect distinct epitopes and comprise wild-type or Fc mutated heavy chains of the human IgA2 subclass. The mAbs, used as tissue culture supernatants from appropriately transfected Expi293 cells, comprised unmodified wild-type IgA2 heavy chain (BDR5-A2WT) and (TDR5-A2WT) or Fc mutated IgA2 H chains carrying the H429F mutation (BDR5-A2HF) and (TDR5-A2HF) of the human IgA2 subclass. Background fluorescence determined by measuring binding of fluorescence conjugate only (no ab) is shown along with the non-specific fluorescence of cells only (cells);

FIG. 45 provides results showing the Colo205 cell survival in the presence of two distinct DR5-specific mAbs comprising heavy chains of the human IgA2 subclass. Colo205 cells, 10,000 cells per well of 96-well plate, were cultured for 48 hours in the presence of serial 2-fold dilutions of tissue culture supernatants from Expi293 cells producing the DR5 IgA mAbs used in FIG. 44. The mAbs used were: BDR5-A2WT which comprised wild-type heavy chain of the human IgA2 subclass; TDR5-A2WT which comprised wild-type heavy chain of human IgA2 subclass; BDR5-A2HF which comprised heavy chains of the human IgA2 subclass carrying the H429F modification; TDR5-A2HF which comprised heavy chains of the human IgA2 subclass carrying the H429F modification. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab) and maximum death determined by culture of cells with sodium dodecyl sulphate (SDS). Survival is represented by: [experimentally determined Abs450 nm−background Abs450 of cell culture medium];

FIG. 46 provides results showing the enhanced killing of Ramos lymphoma cells by mixtures of H429F-modified mAbs detecting distinct molecular targets. Ramos cells at 10,000 per well were separately cultured in the presence of anti-CD38 mAb isatuximab comprising IgG1 WT H Chains (Isa-WT), or anti-DR5 mAb BDR5-1 comprising IgG1 WT H chains (BDR5-1WT), or a 1:1 mixture of both mAbs (Isa-WT+BDR5-1WT) or cultured with their Fc modified counterparts, isatuximab comprising H chains carrying the H429F modification (Isa-HF) as described in Example 7 and BDR5-1 comprising H chains carrying the H429F modification (BDR5-1HF) or a 1:1 mixture of both (Isa-HF+BDR5-1HF). All mAbs were present in the cultures at 10 μg/ml and, for the mixtures, at 10 μg/ml of each mAb for a total mAb concentration of 20 μg/ml. After culture for 48 h, cell viability was quantitated using a CCK8 colorimetric cell viability assay where absorbance (Abs450 nm) of the supernatant of the CCK8 treated cells is a measure of cell survival and viability. Maximum cell viability was determined by the culture of cells in the absence of antibody (no Ab) and maximum cell death by cell incubation with SDS before CCK8 treatment of the cells. Survival is represented by: [experimentally determined Abs450 nm−background Abs450 of cell culture medium], the mean Abs450 and the four replicate values are shown;

FIG. 47 provides results showing that H429F substitution confers complement-dependent lysis (CDC) of leukaemia cells resistant to lysis by the unmodified anti-CD38 mAb mezagitamab. CDC by complement-dependent lysis was determined using Ramos lymphoma cells which were opsonised especially with the mezagitamab-WT mAb or mezagitamab-H429F mutant mAb at the indicated concentrations. Background lysis was determined in the absence of mAb but in the presence of only complement (C′ only). The CDC (% killing) was determined by flow cytometry using Zombie Green;

FIG. 48 provides results showing that additional mutations of K439E or S440K in the Fc region component may suppress the enhanced CDC of H429F-modified rituximab and that the combination of rituximab-H429F/K439E with rituximab-H429F/S440K restores efficient CDC killing of Ramos cells. (A) Ramos lymphoma cells were opsonised with the individual rituximab-WT (WT) or rituximab-H429F (H429F) mAbs or with rituximab-H429F comprising the additional H chain mutation K439E (H429F/K439E) or with rituximab-H429F comprising the additional H chain mutation S440K (H429F/S440K), or with pairwise mixtures of rituximab-H429F/S440K with rituximab-H429F/K439E (H429F/K439E+H429F/S440K). Background lysis was determined in the absence of mAb but in the presence of only complement (no mAb C′ only). The CDC (% killing) of the mAbs was measured in the presence of a 1/3 dilution of human serum by flow cytometry using Zombie green. The enhanced CDC mediated by H429F modification of rituximab was supressed by either Fc:Fc interactions inhibiting the K439E or S440K mutations, but was found to be fully recovered by the mixture of both IgG mutants. (B) For the binding analysis, the proteins were serially two-fold titrated and binding activity (on Ramos cells) quantified by flow cytometry using a FITC-conjugated anti-hIgG-Fc secondary reagent. The level of non-specific background binding of fluorescent conjugate to cells is shown (conj only); MFI=Median Fluorescence Intensity. All mAbs gave near identical binding to CD20;

FIG. 49 provides the results of a flow cytometric analysis of binding (A, C) and CDC (B, D) of purified flACE2-Fc fusion protein (A, B) and SARS-CoV spike-specific mAbs (C, D) using Ramos-S cells. For the binding analysis, the proteins were serially two-fold titrated and binding activity (on Ramos-S cells) quantified by flow cytometry using a FITC-conjugated anti-hIgG-Fc secondary reagent. The level of non-specific background binding of fluorescent conjugate to cells is shown (conj); MFI=Median Fluorescence Intensity. For CDC analysis, the proteins were serially two-fold titrated and human serum used as a source of complement. The CDC lysis was quantitated using Zombie Green. Background lysis by complement in the absence antibodies was determined and is shown (no mAb C′ only); and

FIG. 50 provides graphical results from flow cytometric analysis showing that cooperative and functional synergy of the H429F-modified flACE2-Fc fusion protein and H429F-modified anti-SARS-CoV-2 mAbs further enhances complement dependent killing (CDC). (A) The mAbs S2P6-H429F (S2P6-HF) or S2P6-WT were titrated alone or titrated in the presence of a fixed concentration of the flACE2-Fc-H429 fusion protein (1 μg/ml final concentration) (S2P6-HF+flACE2-Fc-HF) that, when used alone, mediated CDC killing of 23.4% (indicated by open diamond symbol), or were titrated in the presence of fixed concentration of flACE2-Fc-WT (1 μg/ml final) (S2P6-WT+flACE2-Fc-WT) which, when used alone, mediated a % kill of 5.0% (filled diamond). The background lysis by complement in the absence of mAb or Fc fusion protein is shown (C′ only); Arrow shows example of greatest synergy; (B) CDC killing potency of H429F-modified flACE2-Fc was evaluated on Ramos-S cells. The mAbs CC40.8-H429F (CC40.8-HF) and CV3-25-H429F (CV3-25-HF) were used alone (2.5 μg/ml final concentration) or mixed with flACE2-Fc-H429F (CC40.8-HF+flACE2-Fc-HF; CV3-25-HF+flACE2-Fc-HF; note that the final concentrations of the mAbs was 2.5 μg/ml and of the flACE2-Fc-H429F was 1 μg/ml). Killing potency was evaluated in the flow cytometric assay using Zombie Green. The % CDC killing mediated by flACE-2-Fc-WT and background lysis by complement in the absence of mAb or Fc fusion protein (C′ only) is also shown. Four replicate values and SEM are shown. Mean % kill value is shown above each column;

FIG. 51 provides results showing that ACE2-Fc fusion proteins in three formats, namely trACE2-Fc, flACE2-Fc and EflACE2-Fc, comprising Fc region components with the wild type sequence or the H429F mutation bind equivalently to Ramos-S target cells; as determined using flow cytometric analysis using an anti-IgG secondary reagent labelled with goat anti-hIgG Fc FITC: (A) trACE2-Fc (mean±SEM, n=3); (B) flACE2-Fc (mean±SEM, n=3); and (C) EflACE2-Fc (WT n=3, H429F n=1). In all panels, the level of non-specific binding of fluorescent conjugate to cells is shown (conj only); MFI=Median Fluorescence Intensity. The EC50 (nM) values from the curve fits are shown;

FIG. 52 provides results showing that the ACE2-Fc fusion proteins in three formats (trACE2-Fc, flACE2-Fc and EflACE2-Fc) comprising Fc region components with the wild type sequence or the H429F mutation, strongly fix complement and direct complement dependent cytotoxicity (CDC) of Ramos-S target cells; as determined using flow cytometric analysis of CDC of opsonised Ramos-S cells using diluted normal human serum as a source of complement: (A) trACE2-Fc (mean±SEM, n=3); (B) flACE2-Fc (mean±SEM, n=3); (C) EflACE2-Fc (WT n=3, H429F n=1). In all panels, the level of non-specific complement killing in the absence of ACE2-Fc fusion proteins is shown complement only (C′ only). The EC50 (nM) values from the curve fits are shown for the H429F proteins. Fits for the WT proteins could not be determined (nd);

FIG. 53 provides representations of immunoglobulin (antibodies) and immunoglobulin (antibody)-like molecules showing the modular nature of the antibody:

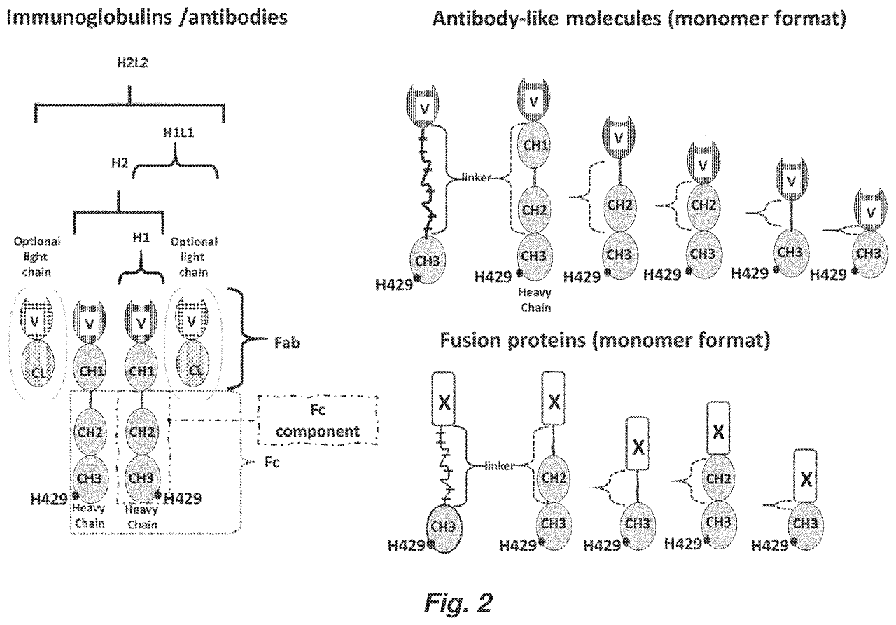

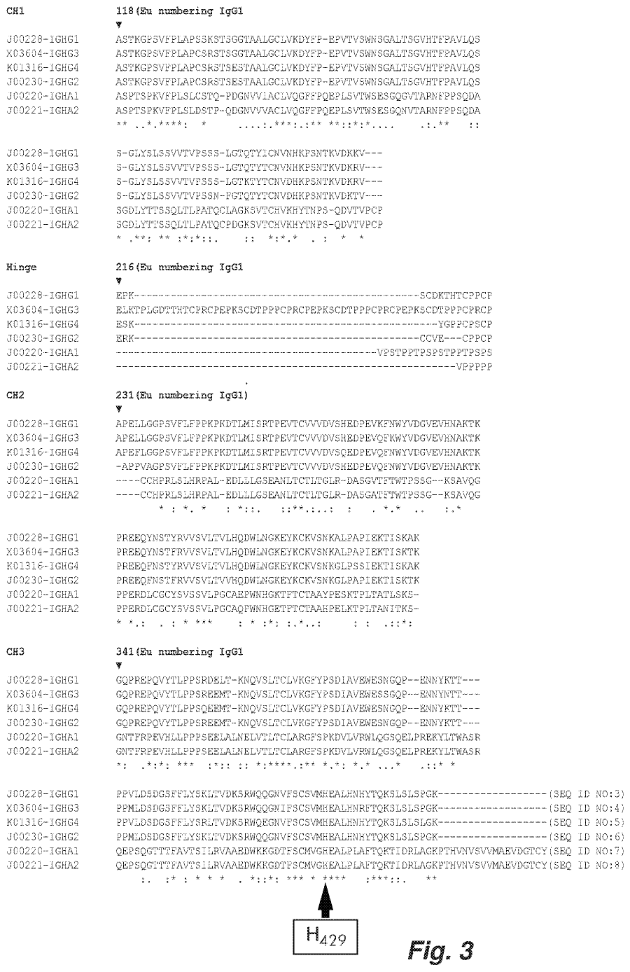

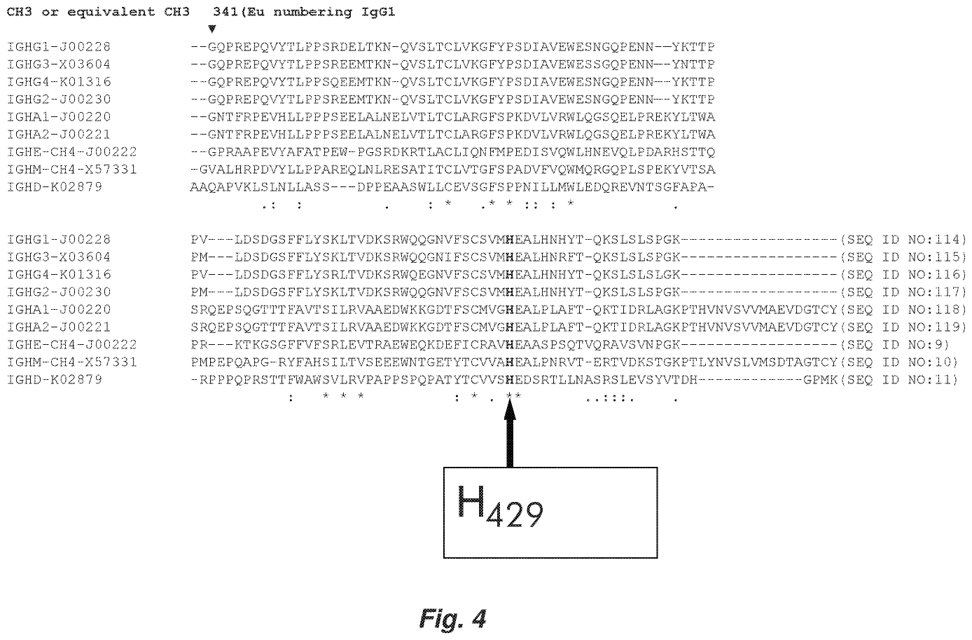

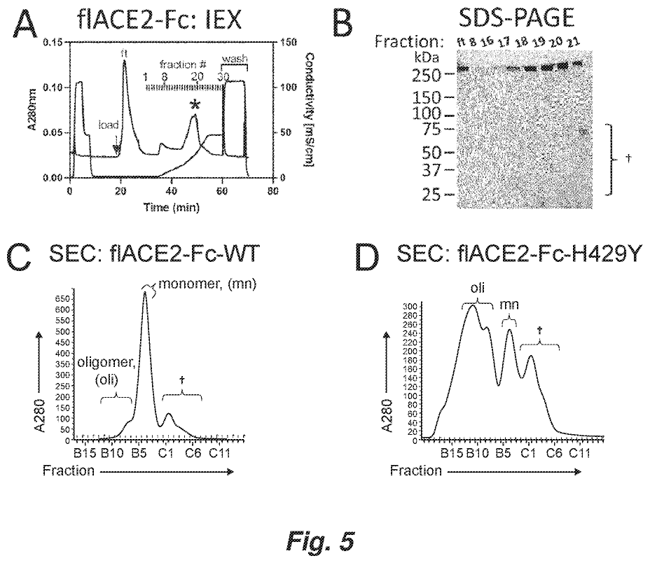

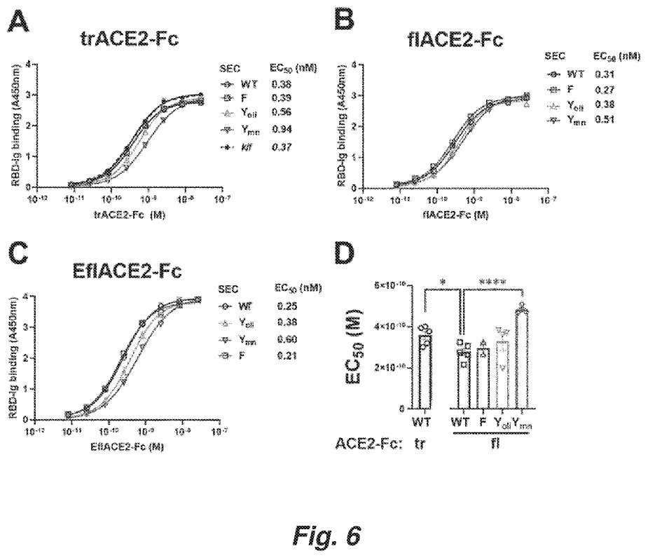

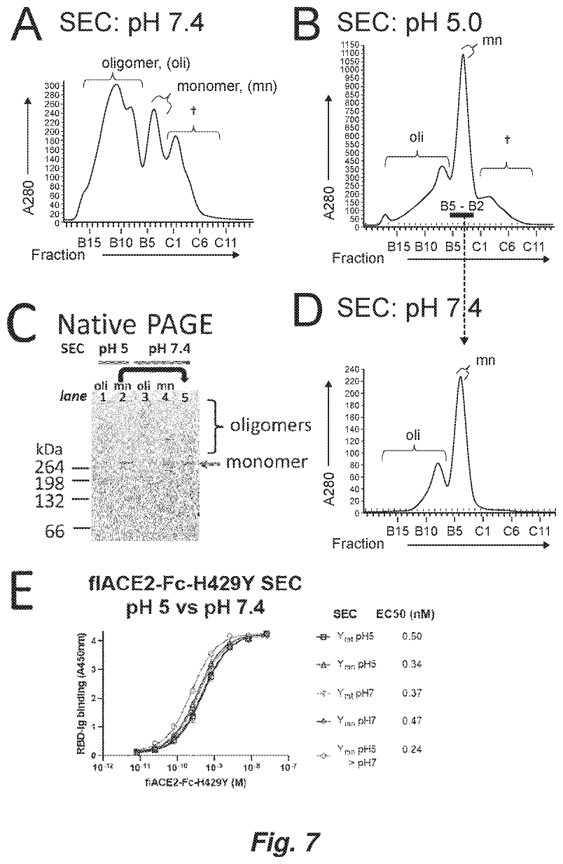

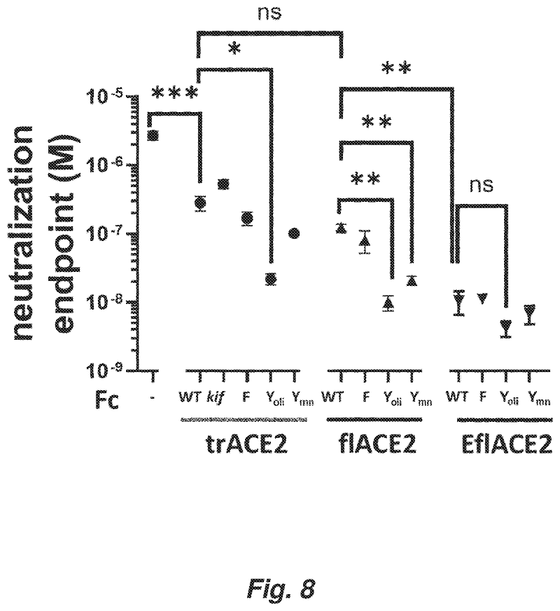

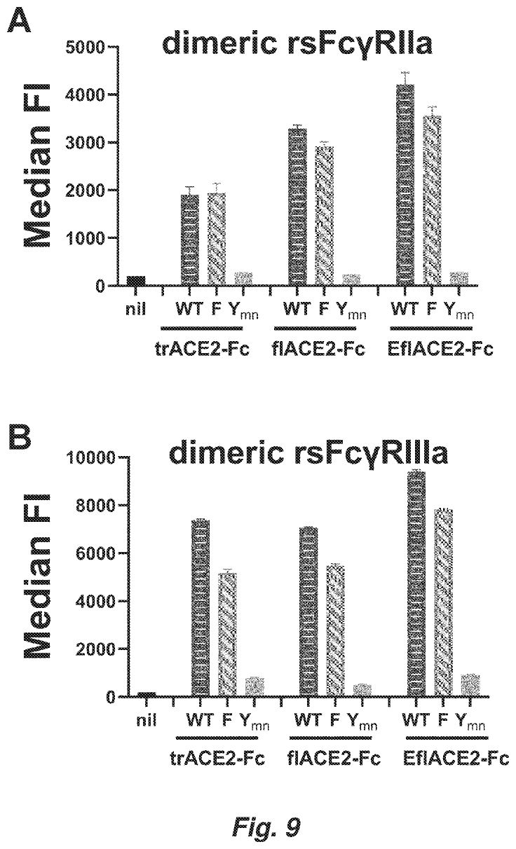

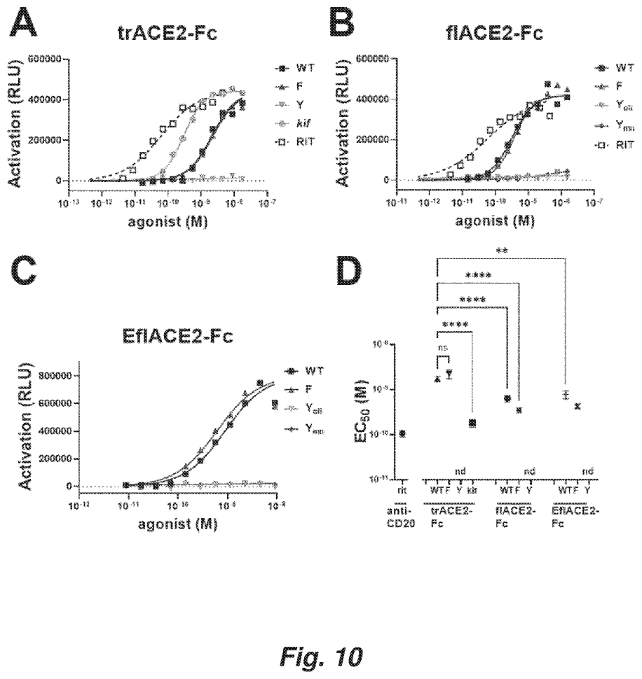

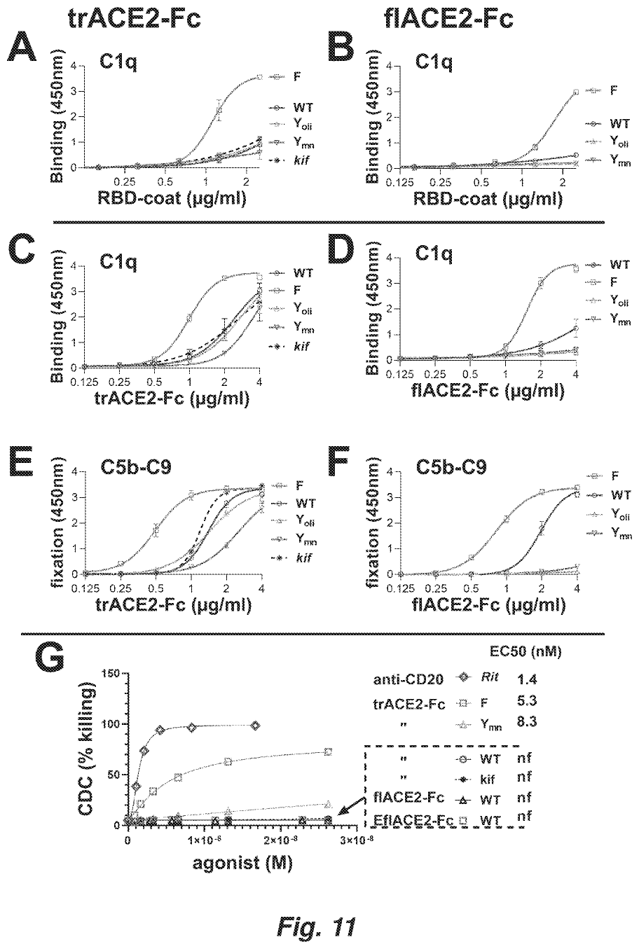

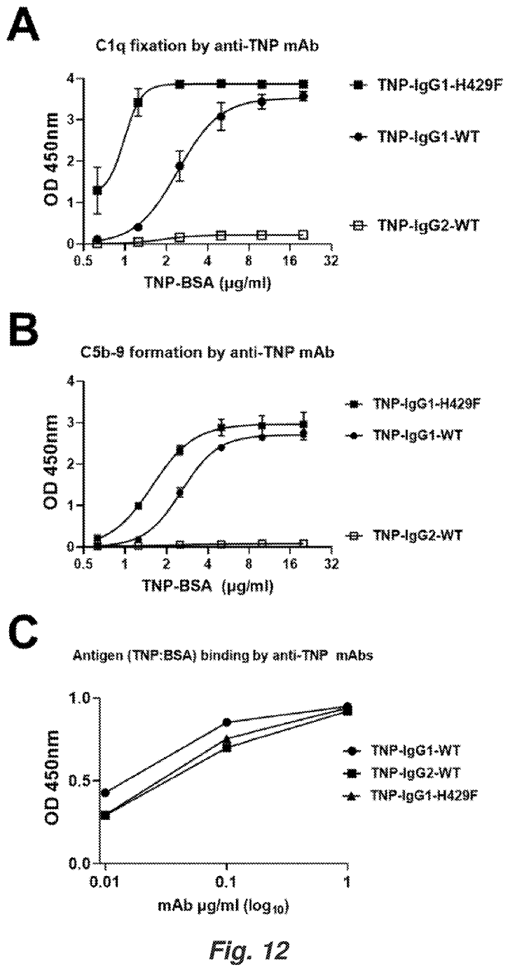

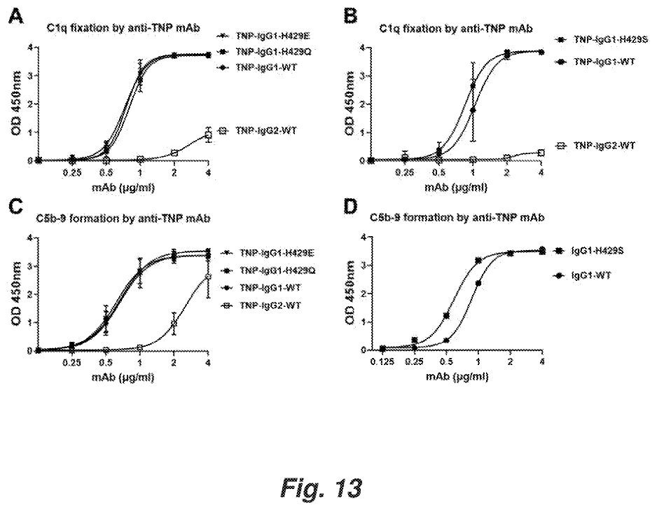

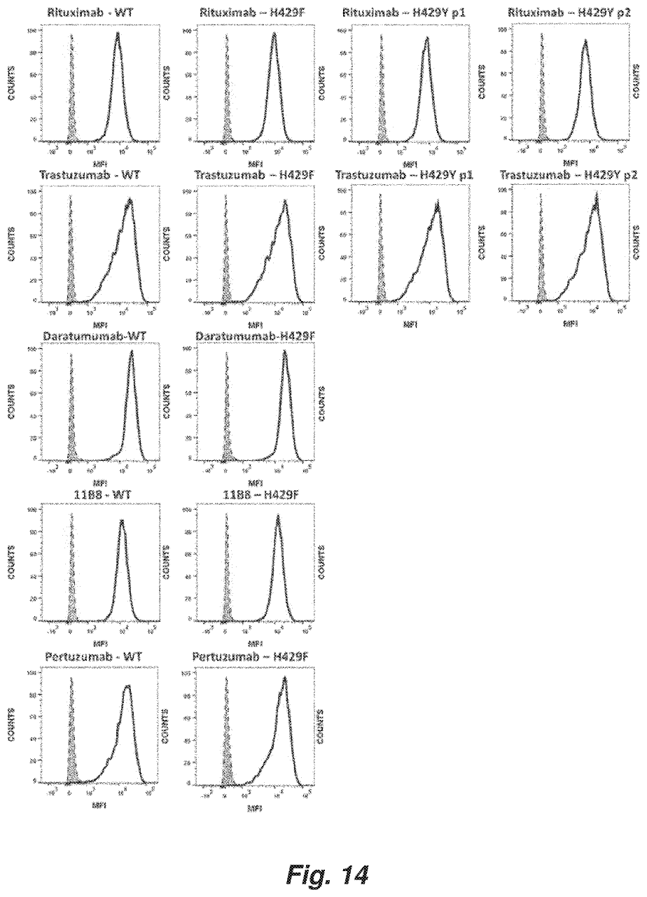

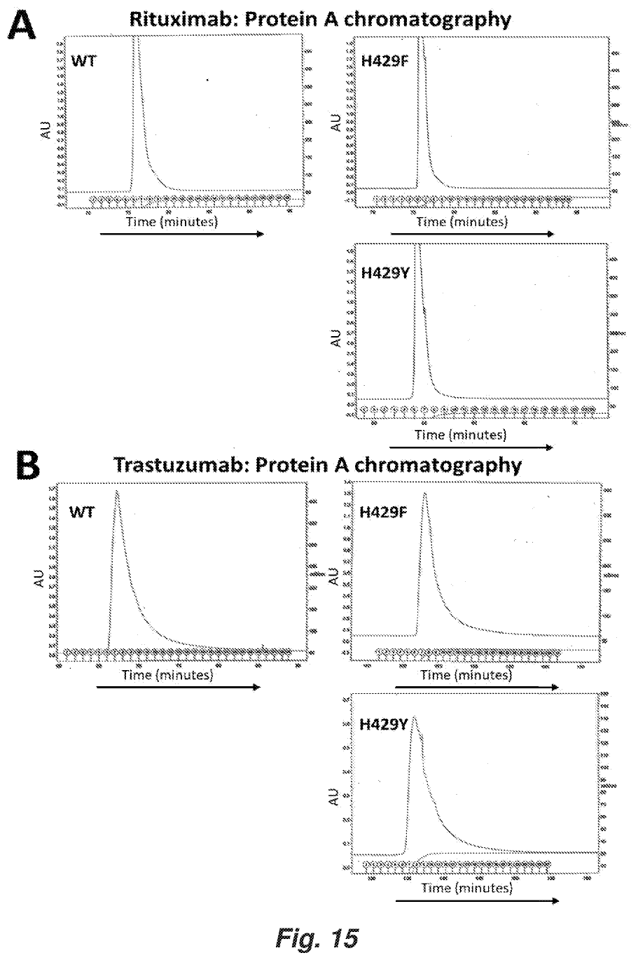

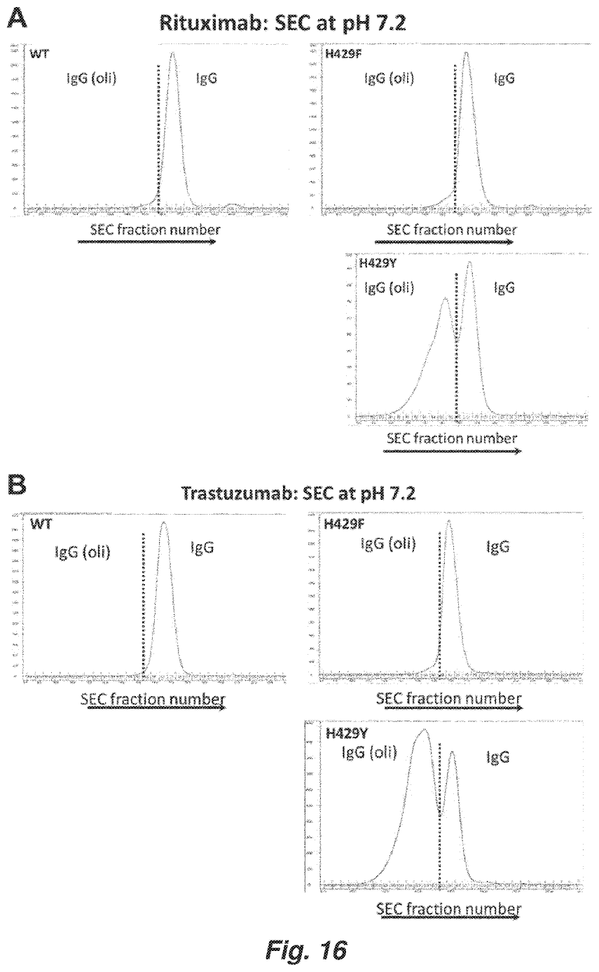

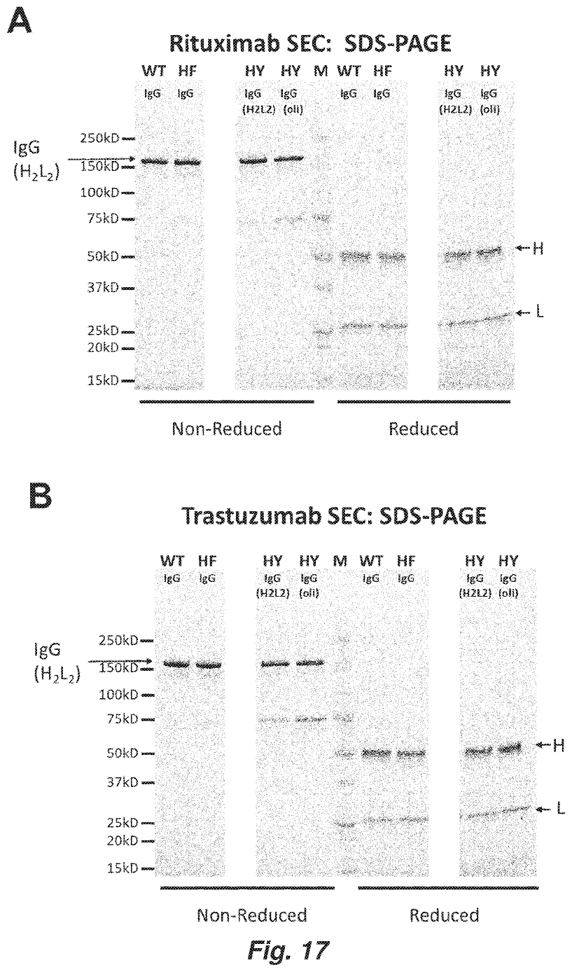

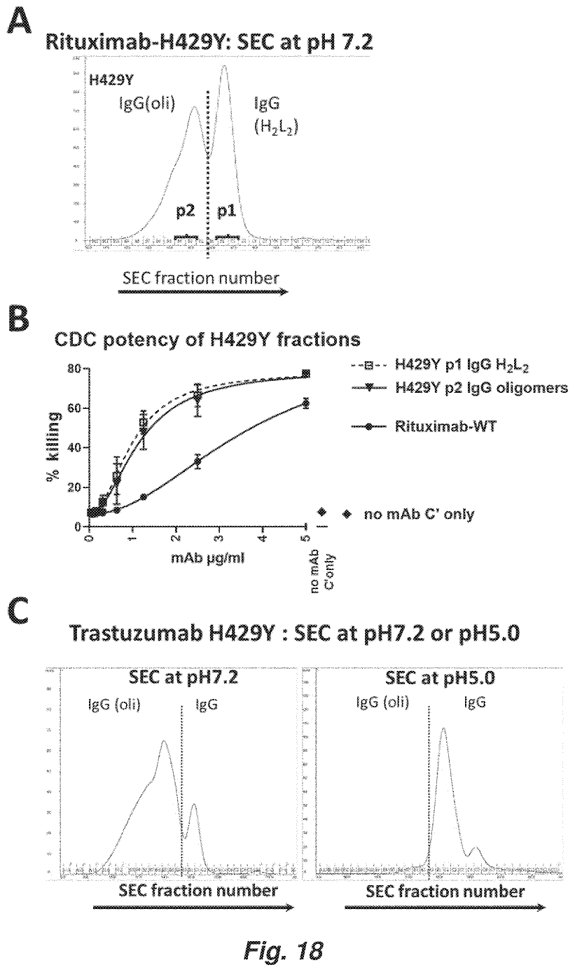

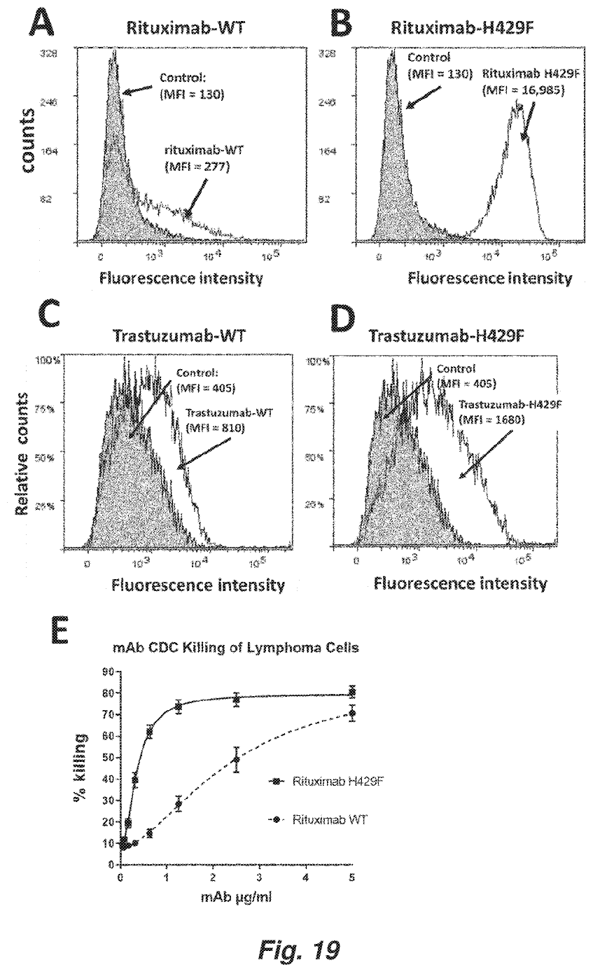

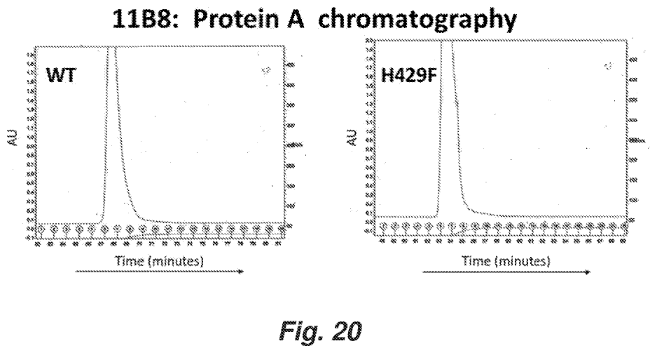

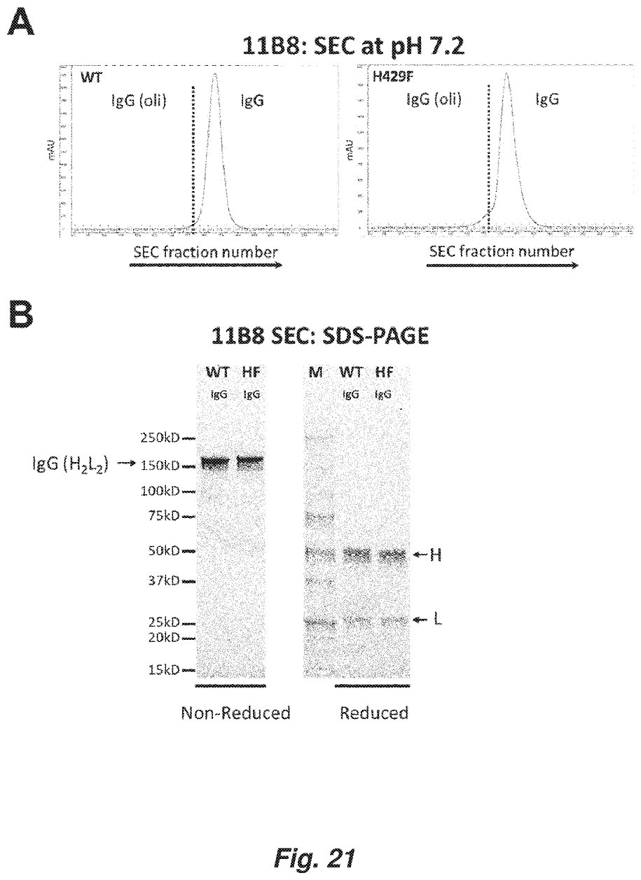

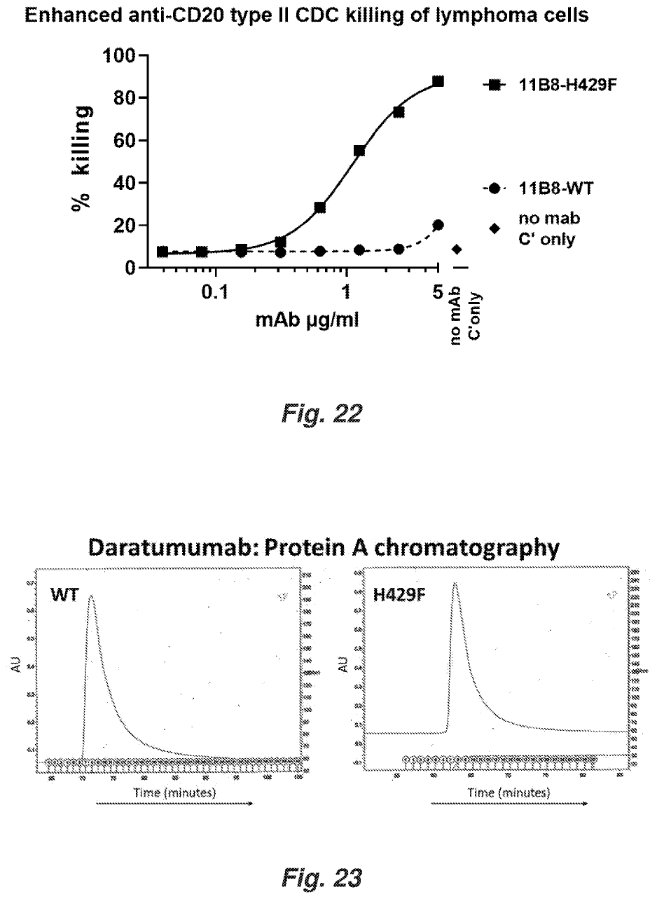

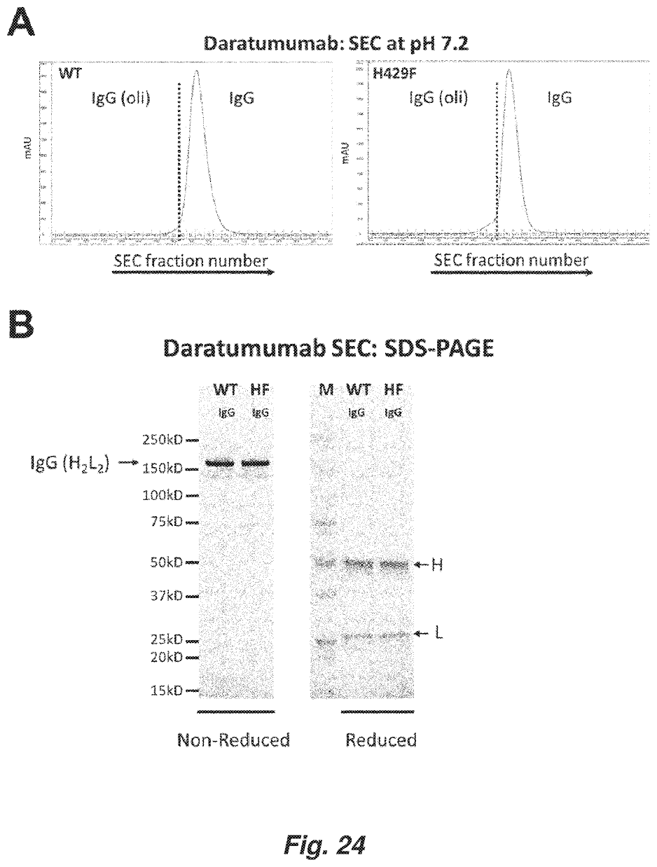

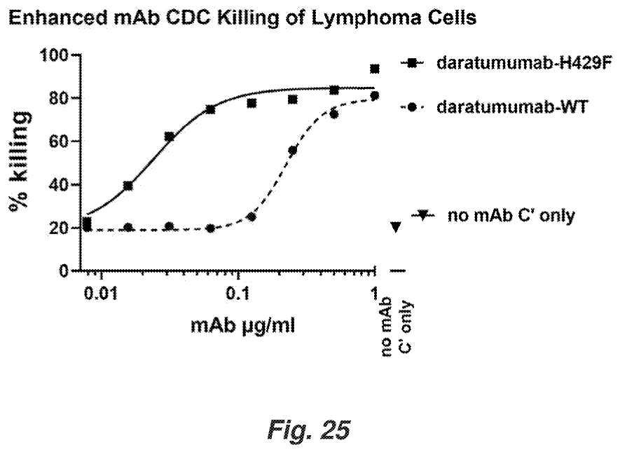

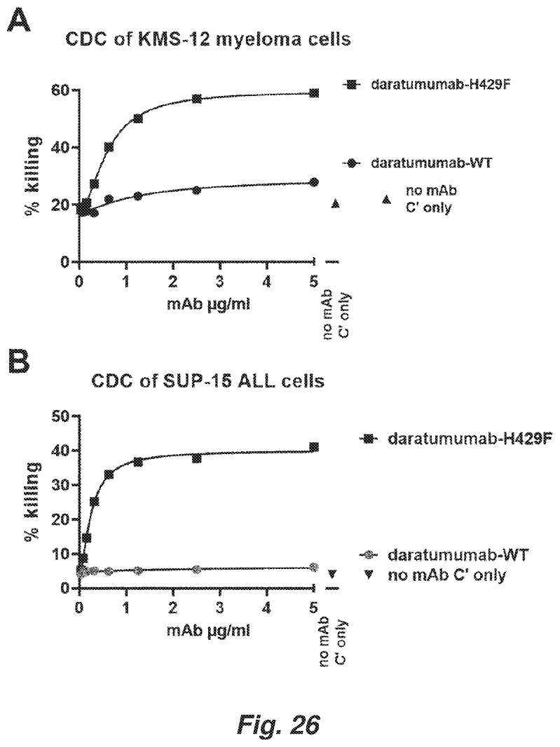

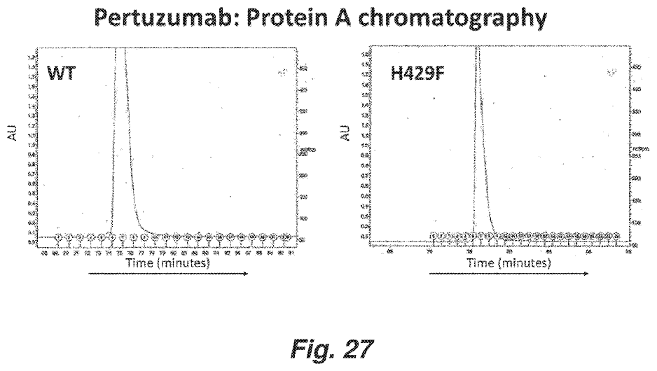

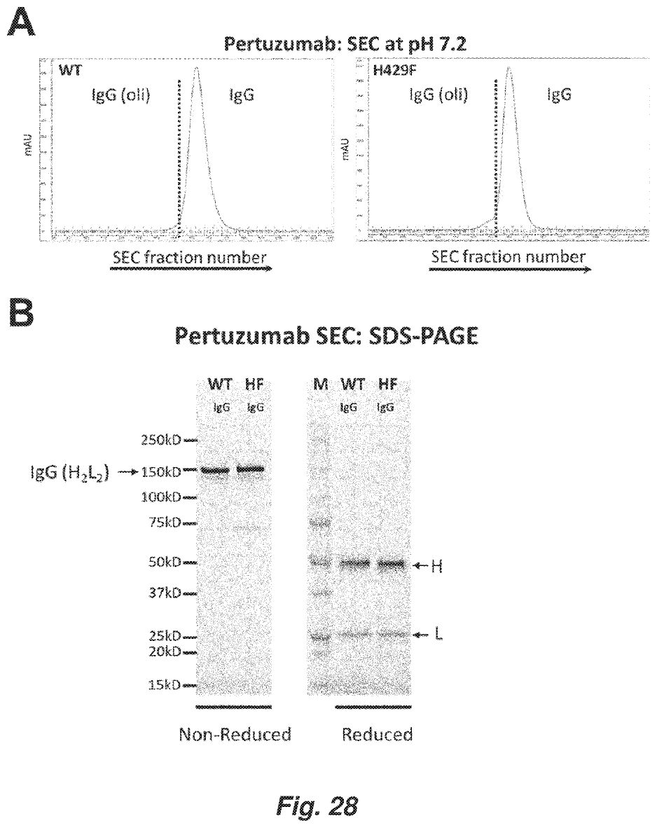

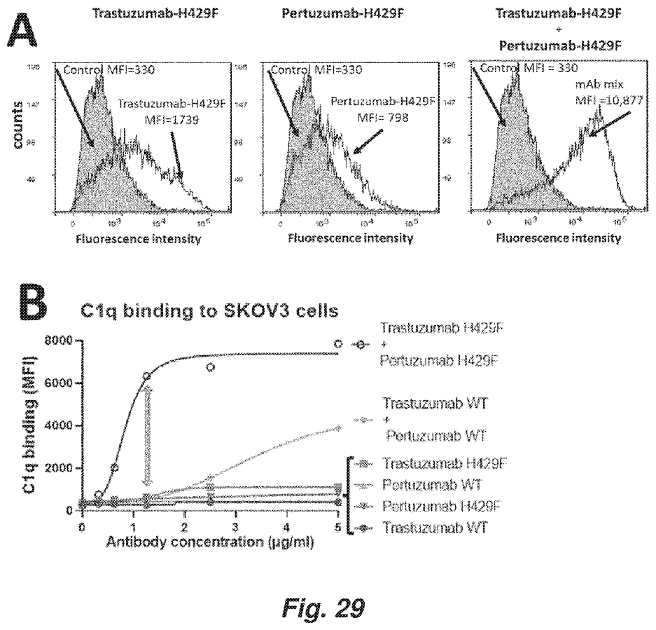

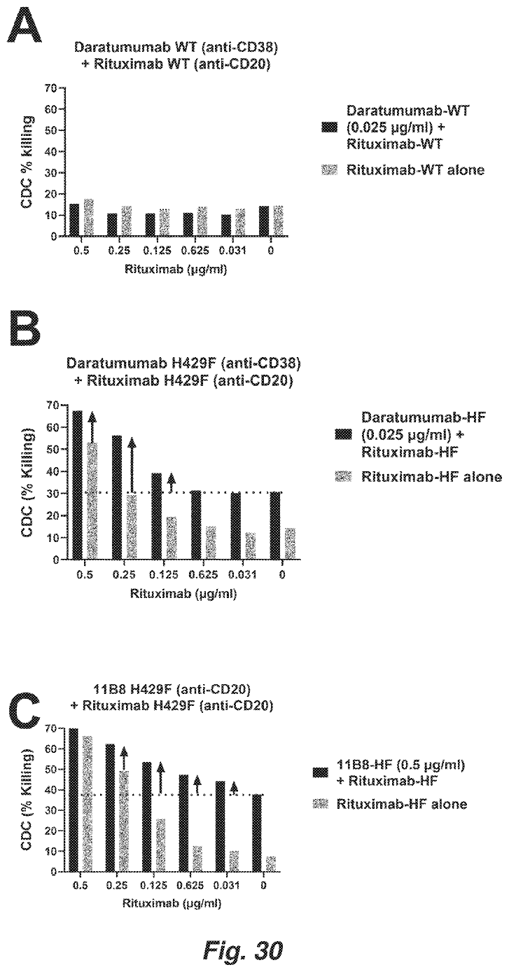

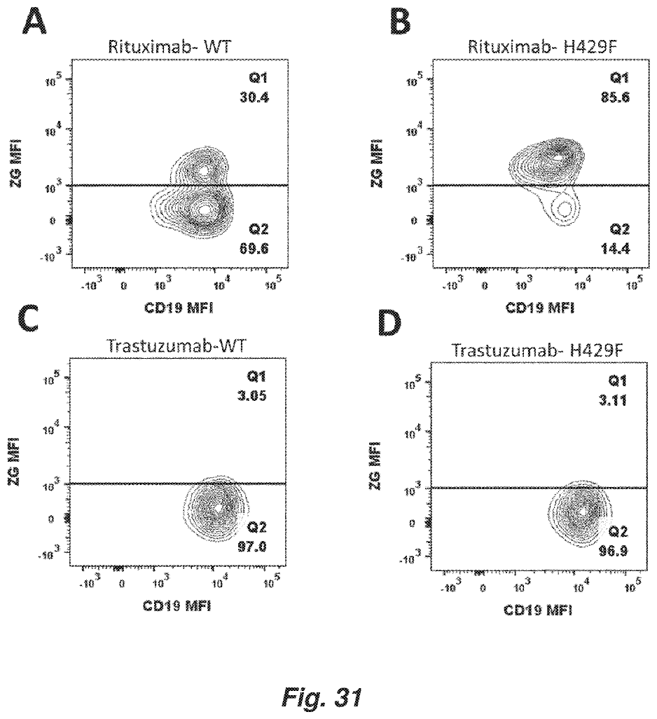

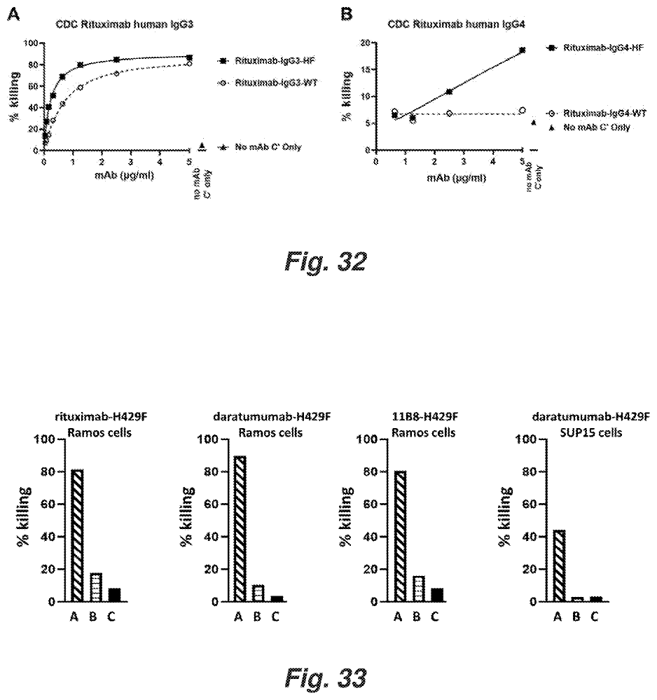

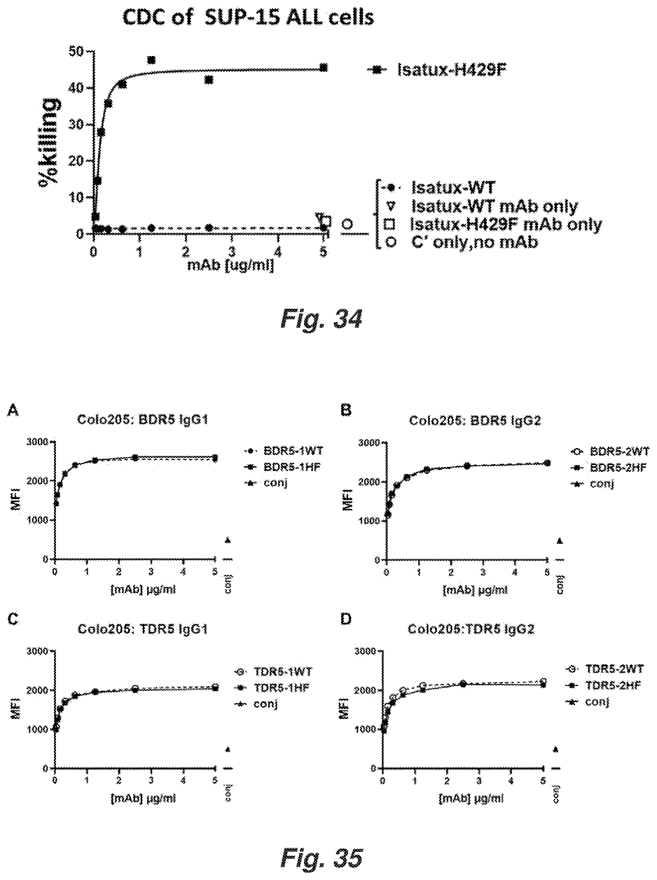

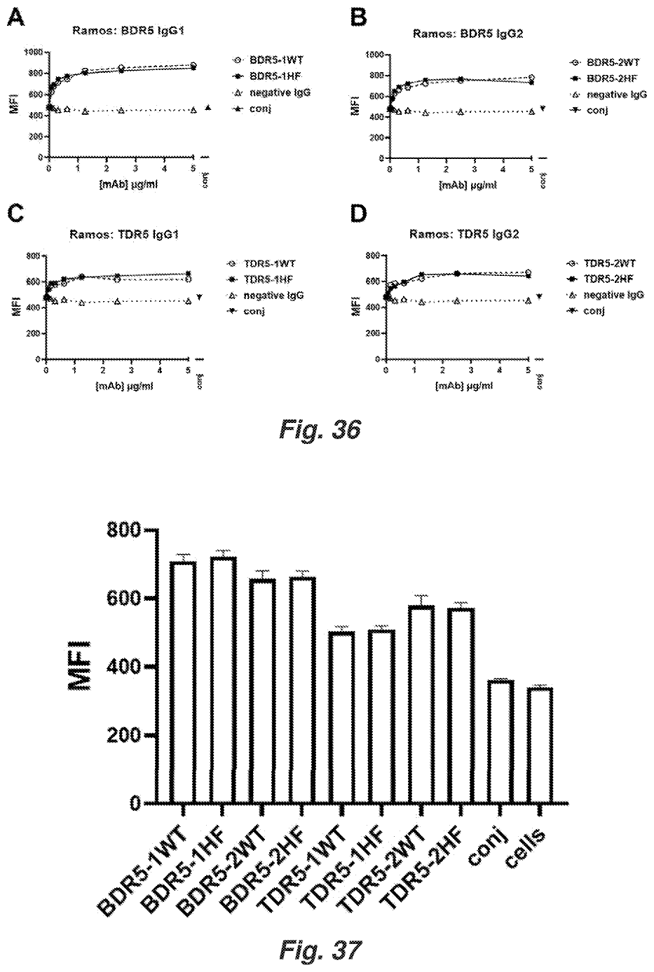

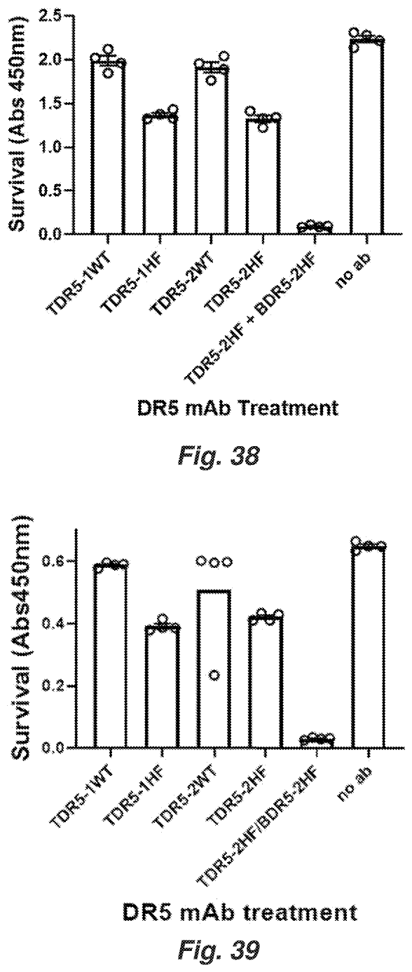

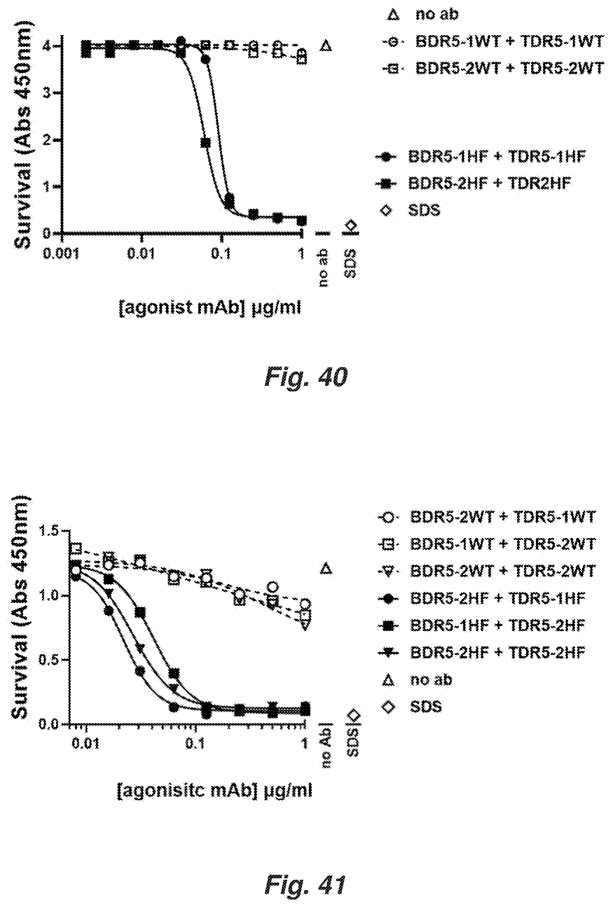

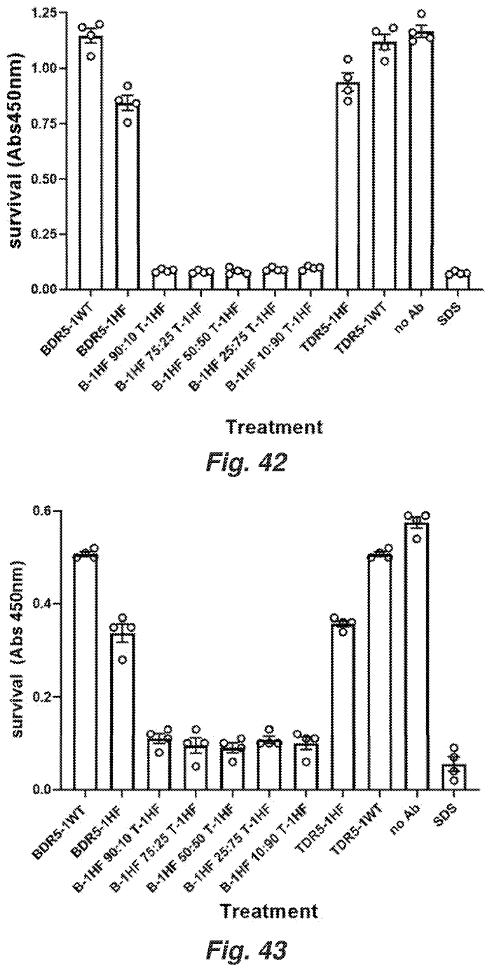

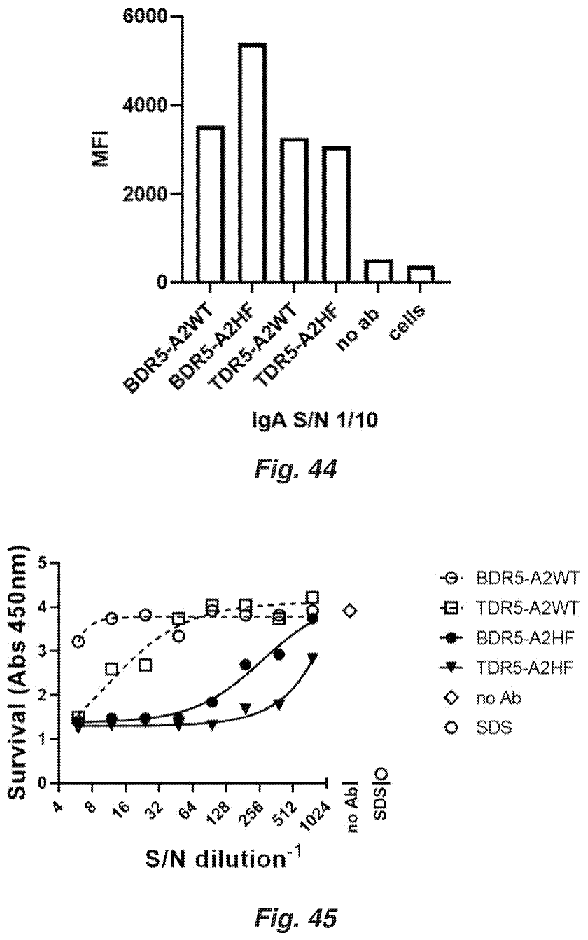

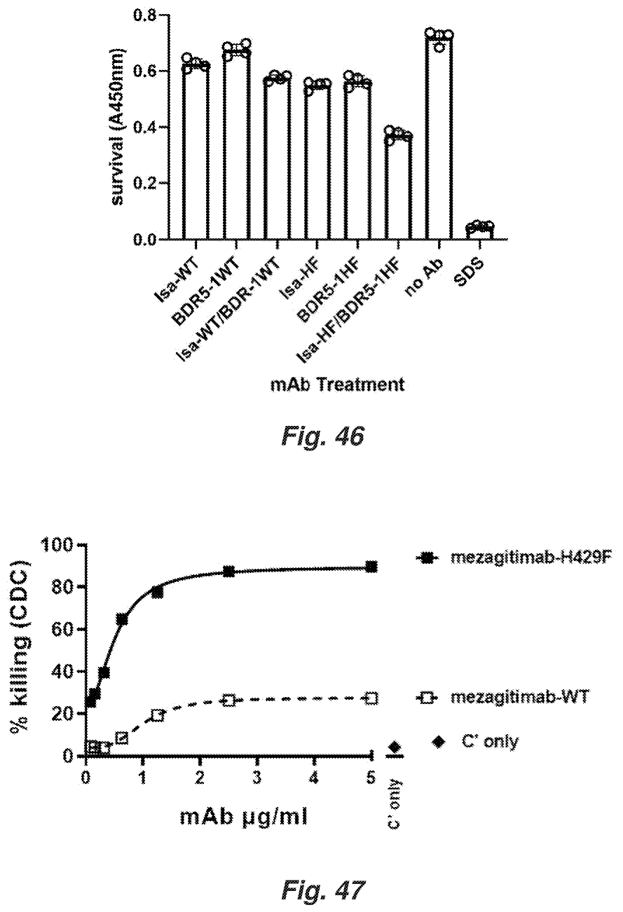

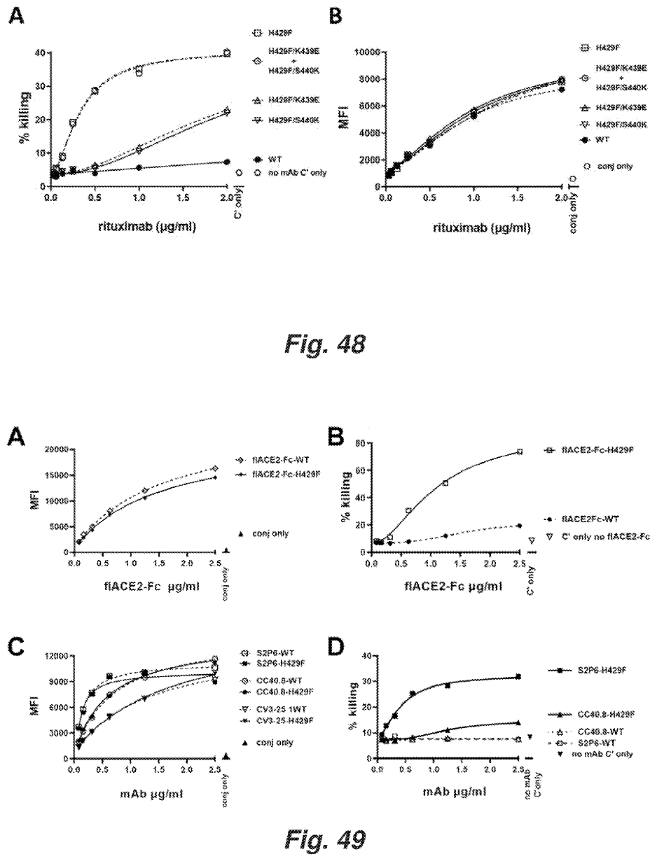

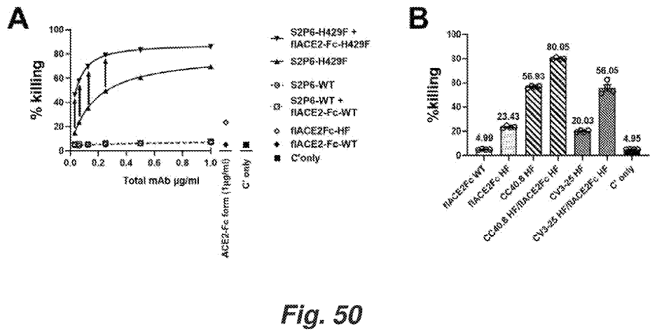

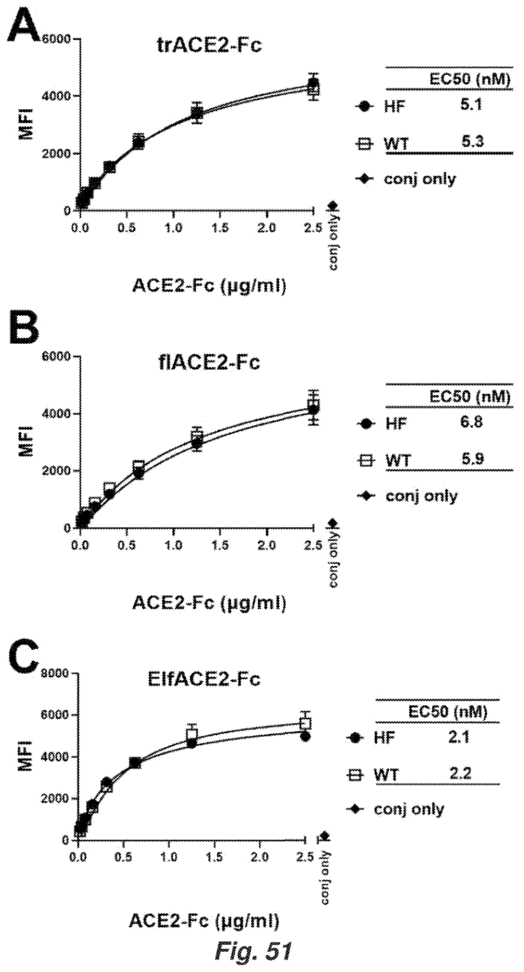

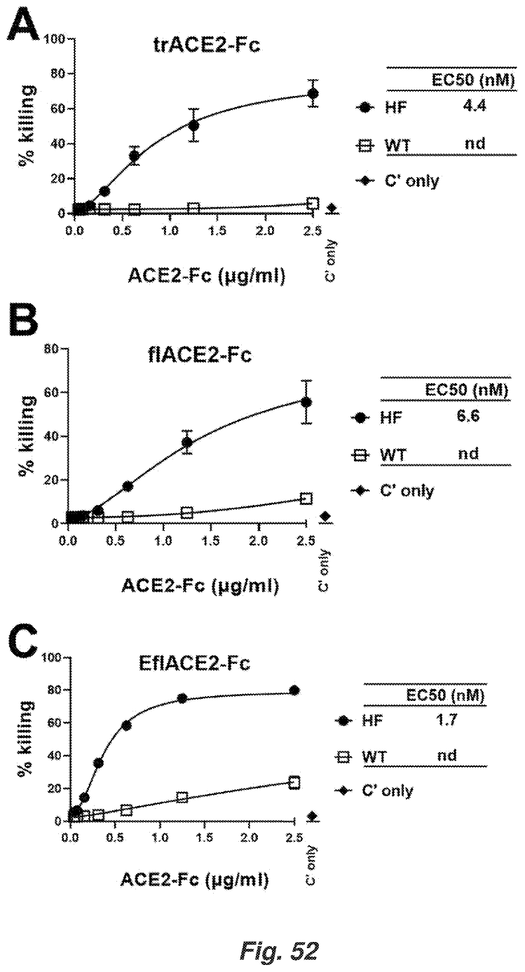

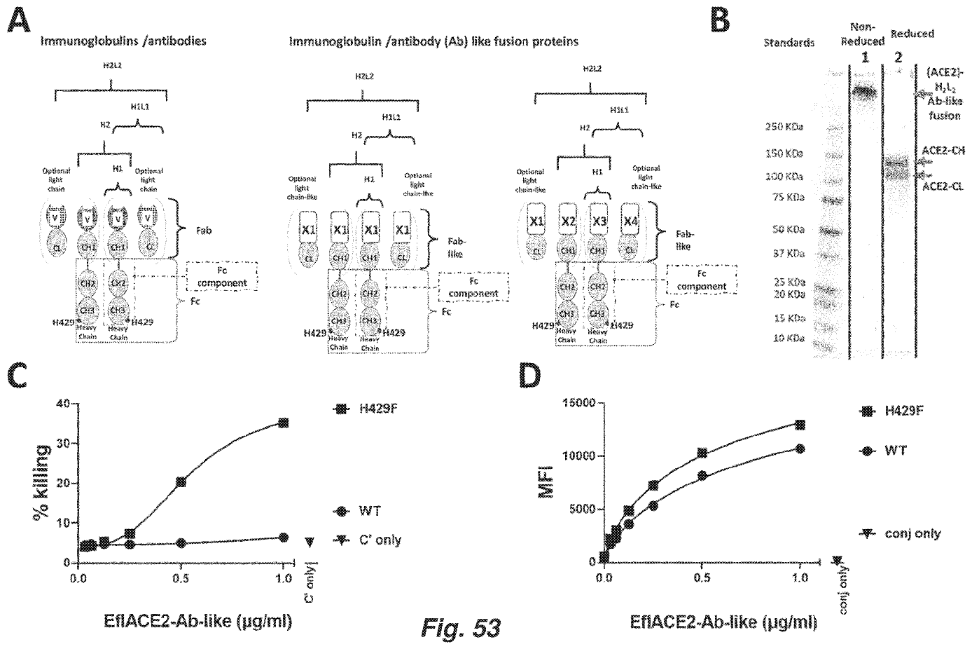

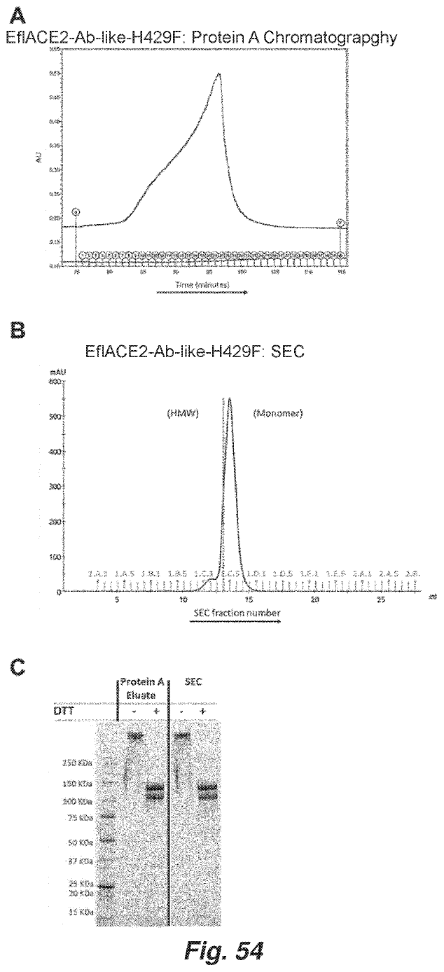

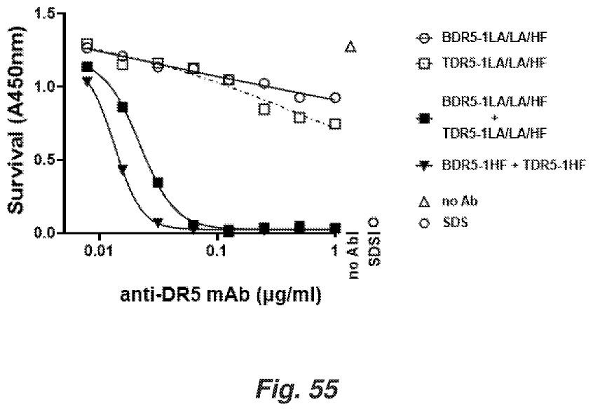

(A) Left panel: The definition of the immunoglobulin molecule chains and components are as indicated and as also defined in the left panel of FIG. 2.

Middle panel: Provides one example of an Ab-like fusion protein, showing that the modular nature of immunoglobulins allows flexibility in the production of Ab-like molecules (as provided in FIG. 2). In the particular depicted Ab-like fusion protein, the target recognition structure (shown as X1) is the same in all chains, as is the case of the Ab-like molecule described in Example 14 (where an EflACE2 polypeptide is separately linked to both the H chain at the CH1 domain and the L chain constant domain, enabling assembly into an H₂L₂ Ab-like fusion protein).

Right panel: Depicts possible Ab-like fusion proteins comprising fusions to different target recognition structures (or enzymes and/or reporter molecules) in any combination of specificities (e.g. “X1 X1 X1 X1”, “X1 X1 X1 X2”, “X1 X1 X2 X2, “X1 X1 X2 X3”, “X1 X2 X3 X4”; where “X1”, “X2”, “X3” and “X4” represent different target recognition structures (or enzymes or reporter molecules)); (B) SDS-PAGE analysis of EflACE2-Ab-like-WT fusion protein eluted from Protein A affinity matrix with 0.4 M arginine (pH 4) demonstrating that a fully disulfide-linked molecule consistent with an H₂L2 Ab-like configuration was achieved (lane 1). This molecule comprises an EflACE2 polypeptide fusion to the immunoglobulin constant heavy chain (EflACE2-CH) which self assembles with an equivalent EflACE2 polypeptide fused to the immunoglobulin light chain constant domain (EflACE2-CL). Upon reduction with dithiothreitol, these two chains (i.e. EflACE2-CH (ACE2-CH) and EflACE2-CL (ACE2-CL); lane 2) are resolved separately.

(C, D) Provide results which show that the EflACE2-Ab-like-H429F fusion protein (H429F) strongly directs CDC of Ramos-S target cells, whereas a corresponding fusion protein with H chains of wild type sequence (WT) was ineffective. CDC of the opsonised Ramos-S cells was determined using the presence of a 1/3 dilution of normal human serum as a source of complement. This potent CDC mediated by the EflACE2-Ab-like-H429F fusion protein was not attributable to different levels of opsonisation compared to the EflACE2-Ab-like-WT fusion protein, as the binding of these to Ramos cells expressing SARS-CoV-2 spike were comparable for both proteins. Binding was determined using flow cytometric analysis using an anti-IgG secondary reagent labelled with goat anti-hIgG Fc FITC. MFI=median fluorescent intensity; background binding of the anti-Ig fluorescent conjugate shown as (conj only);

FIG. 54 shows the results of purification of the EflACE2-Ab-like-Fc-H429F fusion protein using Protein A and elution with arginine. (A) Protein A chromatography using Hitrap™ Protein A column with gradient elution from 30 mM arginine (pH 4) to 35% of 130 mM arginine (pH 4); (B) Size-exclusion chromatography (SEC) of the pooled and concentrated Protein A fractions containing EflACE2-Ab-like-Fc-H429F using a Superose 6 Increase 10/300 column with oligomeric material indicated (HMW); and (C) SDS-PAGE analysis of the pooled Protein A eluate and the pooled SEC monomeric fractions under non-reducing (without DTT, dithiothreitol) and reducing (with DTT) conditions; and

FIG. 55 provides results showing the survival of Colo205 colorectal cells in the presence of purified DR5-specific mAbs comprising IgG1 H chains with the single H429F mutation (HF) or the L234A, L235A and H429F (LA/LA/HF) mutations. The mAbs were titrated individually or mixed and titrated (BDR5-1LA/LA/HF+TDR5-1LA/LA/HF) and compared to a mixture of BDR5-1HF+TDR5-1HF wherein both mAbs in the mixture comprised IgG1 H chains with only the H429F modification; Colo205 cells, 10,000 cells per well of 96-well plate, were cultured in the presence of serial two-fold dilutions of the indicated mAbs. The individual mAbs were titrated from 1 μg/ml. For the titration of the mixtures, the starting concentration of 1 μg/ml was comprised of 0.5 μg/ml of each mAb in the mixture. Cell viability was quantitated using a CCK8 colorimetric cell viability assay, where absorbance (Abs450 nm) of cell culture supernatant is a measure of cell survival and viability. Maximum cell viability was determined by the culture of cells in the absence of antibody (no Ab) and maximum death determined by culture of cells with sodium dodecyl sulphate (SDS). Survival is represented by: [Abs450 nm of mAb or control treatment determined experimentally determined Abs450 nm−background Abs450 of cell culture medium].

### DETAILED DESCRIPTION

The present disclosure is directed to immunotherapeutic proteins including one or more polypeptide comprising an Fc region component, wherein the one or more polypeptide includes an amino acid substitution (mutation) at the position corresponding to H429 (within the CH3 domain) of the amino acid sequence of human immunoglobulin G1 (IgG1) heavy (H) chain (Eu numbering).

Amino acid numbering used herein is based on the so-called Eu numbering system which relates to the sequence numbering used in the description of the human IgG1 myeloma protein called Eu (Edelman G M et al., Proc Natl Acad Sci USA 63(1):78-85, 1969). In accordance with this system, H429 of IgG1 for example (i.e. histidine at position 429) occurs at position 429 of the Eu sequence. Thus, in any immunoglobulin molecule such as an antibody or fragment thereof or an antibody-like molecule, the amino acid in a given position number relates to, or corresponds to, the corresponding amino acid residue position number in the Eu sequence.

The heavy (H) chain of immunoglobulins such as IgG1 is a modular, multifunctional, but monomeric, polypeptide. The prototypical immunoglobulin or antibody structure (with an H429 mutation may be considered as consisting of a dimeric protein comprising two monomeric heavy (H) chain polypeptides each optionally associated with a light (L) chain and thus occurring in an H₂L₂ format (see FIG. 2). However, other forms of immunoglobulin molecules may be formed, including where one heavy chain (H1) associates with one light (L1) chain in an H₁L₁ format, and other forms where two heavy (H) chains may form dimers in the absence of light (L) chains to produce an H₂ formatted heavy chain complex (FIG. 2). When a light (L) and heavy (H) chain is associated in the H₁L₁ format, the molecule can dimerise in this configuration to yield the typical immunoglobulin structure designated as H2L₂. This is exemplified by the typical human IgG class of immunoglobulins wherein the two heavy (H) chains are covalently bonded to each other, and a light (L) chain is covalently bonded to each heavy (H) chain.

The dimeric form of H₁L₁ immunoglobulin molecules (i.e. H₂L₂ proteins) is the fundamental structural “unit” of all human Ig classes (i.e. IgG, IgE, IgD, IgA and IgM) and indeed, is also the basis of most mammalian immunoglobulin classes noting that exceptions to this format are known (e.g. camelid immunoglobulins can form a heavy chain dimer without light chains (abbreviated as H₂)). Thus, for example, human immunoglobulin G (IgG) molecules exist physiologically in solution as a single H₂L2 unit. However, other covalently bonded, higher-order oligomers of the basic H₂L₂ unit do exist in nature and are common, particularly, for IgM and IgA.

For instance, IgM can form covalent pentameric or hexameric rings of the H₂L₂ format with each H₂L₂ unit having disulphide bonds to adjacent H₂L₂ units to form the pentameric oligomer (H₂L₂)₅ or hexameric oligomer (H₂L₂)₆ (Eskeland T and T B Christensen, Scand J Immunol 4(3):217-228, 1975), and notably, it is the hexameric form of IgM which is the most potent effector of the classical complement pathway (which is one of the two major effector systems of the innate immune system initiated by immunoglobulins), activated by the avid binding of the soluble hexameric protein C1q (Eskeland T and T B Christensen, 1975 supra; Randall T D et al., Proc Nat Acad Sci USA 89:962-966, 1992; Hughey C T et al., J Immunol 161:4091-4097, 1998; and Randall T D et al., Eur J Immunol 20:1971-1979, 1990). IgM is also well recognised as the most potent agglutinin by virtue of its covalent oligomerisation which results in the presence of 10-12 specific, and identical, antigen binding variable domains in each IgM pentamer or hexamer. Since an individual antigen recognition structure (e.g. a Fab fragment) has a defined affinity for a target antigenic site or epitope with a strength defined by its monovalent binding to one target structure, the presence of multiple antigen recognition structures in the one molecule (e.g. the 12 antigen binding variable domains of each IgM hexamer) confers stronger or more avid binding of the oligomeric immunoglobulin. Thus, oligovalent binding arises from the combined strength of the individual antigen recognition interactions of the IgM oligomer and its target antigenic epitopes. By comparison, the avidity of IgG (which has two antigen recognition structures (Fabs) per H₂L₂ unit) arises only from the interaction of these two antigen recognition structures.

Immunoglobulins can also be regarded as modular, multifunctional proteins in which the target recognition structure is connected by a flexible linker to a function-activating structure (i.e. the antigen recognition structure provided by the V domain of the Fab is linked by a flexible hinge to the Fc region), and wherein, in the case of atypical immunoglobulin, each H chain and each L chain is comprised of different domains which can be considered structural or functional modules (FIG. 2). An H chain is particularly comprised of an antigen target recognition domain (V_{H} domain) of variable sequence followed by a series of constant domains that are unique to the heavy chain of an Ig class such as, for example, IgG or IgA, and sequence analysis shows that these constant domains are related between immunoglobulin classes (see FIGS. 3 and 4). The V_{H} domain is thus followed by a first constant domain (CH1). This first constant domain is connected by a flexible polypeptide that acts a linker known as the hinge region, to a second constant domain (CH2) which is followed, in turn, by a third constant domain (CH3), and thus a typical H chain consists of discrete structural molecules from the amino terminus (i.e. NH₂-terminus V_{H}-CH1-hinge-CH2-CH3 (FIG. 2)) and is exemplified by the human IgG1 H chain sequence (SEQ ID NOS: 3 and 114) and is conserved across all human heavy chains (FIG. 3, 4) exemplified by human IgG3 (SEQ ID NOS: 4 and 115), IgG4 (SEQ ID NO: 5 and 116), IgG2 (SEQ ID NOS: 6 and 117), IgAQ1 (SEQ ID NOS: 7 and 118), IgA2 (SEQ ID NOS: 8 and 119), as well as IgE (SEQ ID NO: 9), IgM (SEQ ID NO: 10) and IgD (SEQ ID NO: 11) and mammalian immunoglobulins generally. Similarly, a light (L) chain is comprised of a variable antigen target recognition domain (V_{L} domain) followed by a constant domain unique to the light chain class.

Given their modular nature, immunoglobulins provide a versatile platform for the creation of a diverse range of immunotherapeutic or diagnostic molecules, including bivalent antibodies such as a classical hybridoma-derived mAb, heavy-chain antibodies comprising dimers of heavy chains (e.g. camelid antibodies; Hamers-Casterman C et al., Nature 363:446, 1993), antibody-like (Ab-like) molecules (including some fusion proteins comprising at least a CH3 domain (or at least a CH4 domain)) and other immunoglobulin derivatives known to those skilled in the art as summarised in the “Periodic Table of Antibodies”; https://absoluteantibody.com/periodic-table-of-antibodies/#, the entire disclosure of which is herein incorporated by reference). Other specific examples include antibodies and antibody fragments such as single chain Fv antibody fragments, asymmetric bi-specific antibodies (WO 2012/058768), strand-exchange engineered domain (SEED or Seed-body) which are asymmetric and bispecific antibody-like molecules (WO2007110205); dual variable domain immunoglobulin (U.S. Pat. No. 7,612,181), knobs-into-holes antibody formats (WO 1998/050431), duobody molecules (WO 2011/131746), IgG-like bispecific (Shen J et al., J Immunol Methods 318(1-2):65-74, 2007), and fusion proteins comprising an Fc or Fc region component such as scFv-fusions and dual scFv-fusions.

In this specification, a number of terms are used which are well known to those skilled in the art. Nevertheless, for the purposes of clarity, a number of these terms are hereinafter defined.

As used herein, the term “antibody” is to be understood as including (unless specifically indicated as otherwise) polyclonal antibodies (pAb), monoclonal antibodies (mAb), chimeric antibodies, humanised antibodies, antibody mixtures (e.g. recombinant polyclonal antibodies) such as those generated, for example, methods well known to those skilled in the art for producing multiple antibodies with different specificities from a single host cell line (e.g. Oligoclonics® technology: Merus BV, Utrecht, The Netherlands) or a transgenic animal. Further, it is to be understood that an antibody may be of any immunoglobulin class (i.e. isotype) or allotype. Thus, for example, an antibody as disclosed herein may be of an isotype selected from the well-known immunoglobulin isotypes, or comprise components from more than one isotype (e.g. as may be the case with some chimeric antibody types). Also, an antibody may comprise “mixed” chains; for example, an antibody comprised of a H₂ or H₂L₂ unit where the H chains are different (i.e. H^{x} and H^{y} (H^{x}H^{y} or L^{x}H^{x}H^{y}L^{y}).

The term “antibody-like molecule” (Ab-like molecule) is to be understood as referring to proteins which are not antibodies and minimally comprise a target recognition structure (e.g. a receptor or ligand such as an antigen recognition sequence (e.g. a variable (V) domain or complementarity-determining region (CDR) of an immunoglobulin) linked to at least a CH3 domain (or a CH4 domain) of an immunoglobulin heavy (H) chain (providing the molecule with an “H-like” chain), and includes, for example, receptor fusion proteins comprising at least a CH3 domain (or at least a CH4 domain) of an H chain. Examples of Ab-like molecules include those mentioned above (such as those depicted in FIG. 2, FIG. 53A and/or The Periodic Table of Antibodies). In some examples, an H-like chain comprising at least a CH3 domain is considered to comprise an Fc region component and may be, for example, a complete Fc region, or merely a CH3 domain, or a CH2-CH3 or CH3-CH2 component. In other examples, such an H-like chain may further comprise, for example, an immunoglobulin hinge sequence. The H-like chain may enable dimerisation, such that an antibody-like molecule may be provided, for example, in a homodimeric or heterodimeric forms. As depicted in FIG. 53, in some embodiments, an Ab-like molecule may comprise a H₂L₂ format and may comprise, for example, fusion proteins comprising the same or different target recognition structures (or enzymes and/or reporter molecules) such as “X1 X1 X1 X1”, “X1 X1 X1 X2”, “X1 X1 X2 X2”, “X1 X1 X2 X3” or “X1 X2 X3 X4”, where X1, X2, X3 and X4 represent different target recognition structures (or enzymes or reporter molecules).

The term “fusion protein” is well known to those skilled in the art and refers to a protein expressed from a DNA construct comprising two or more open reading frames in a desired order, such that the protein may be regarded as a hybrid or chimeric protein. In some simple examples, the fusion protein comprises a protein (or fragment) of interest linked (“fused”) to the amino-terminus (N-terminus) or carboxyl-terminus (C-terminus) of a partner polypeptide such as a carrier protein (e.g. human serum albumin, HSA). As used herein, the term “fusion protein” will also be understood as referring to certain types of an antibody-like molecule such as a fusion protein comprising a target recognition structure (e.g. a receptor or ligand such as an antigen recognition sequence (e.g. a variable (V) domain or complementarity-determining region (CDR) of an immunoglobulin)) linked to at least a CH3 domain (or at least a CH4 domain) of an immunoglobulin heavy (H) chain (see examples shown in FIG. 2). Such fusion proteins may comprise, for example, mixed Fc regions (e.g. in a manner similar to that described above for antibodies (e.g. bispecific antibodies) and antibody-like molecules such that the fusion protein comprises, for example, an Fc fragment wherein the Fc regions (chains) are different (e.g. Fclx and Fc2y), and wherein each of the Fc regions may optionally be linked to different target recognition structures (e.g. ligand 1 and ligand 2). Those skilled in the art will understand that the target or antigen recognition structure may be linked to a polypeptide comprising at least a CH3 domain (or an equivalent CH4 domain of an IgE or IgM) that may, for example, comprise an entire H-chain or comprise an H-like chain which may comprise, for example, a complete Fc region, or merely a CH3 domain, or a CH2-CH3 or a CH3-CH2 component. In other examples, such an H-like chain may further comprise, for example, an immunoglobulin hinge sequence and/or a CH1 domain. A CH1 domain may, for example, provide a suitable site for linking the target or antigen recognition structure.

The term “Fc fragment” (Fc or Fc portion), as used herein, refers to a dimer formed by covalent and/or non-covalent interactions between parts of the immunoglobulin heavy (H) chain (i.e. a dimer formed between two Fc regions of the H chain (which each comprise CH2 and CH3 domains of a heavy chain and optionally a hinge sequence)), and which is responsible for much of the activation of the immune effector functions of immunoglobulins: particularly the cell-based effector responses initiated by antibodies and cell surface-located Fc receptors such as killing of target cells by antibody dependent cell-mediated cytotoxicity (ADCC) or antibody-dependent cell-mediated phagocytosis (ADCP) or trogocytosis, and which also leads to the modulation and inhibition of the activity of innate and adaptive immune cells by inhibitory Fc receptors; and the effector responses of the innate immune system initiated by antibodies through activation of the complement system via the classical pathway of complement system, a cascade of proteins found in blood or biological fluids that is important in the destruction of pathogens and involves the killing of targets by direct lysis through complement-dependent cytotoxicity (CDC) and/or by killing via phagocytosis of targets through specific receptors of the components of complement (C′ADCP). The Fc fragment also provides a site of association between immunoglobulin molecules permitting the assembly (self-association) of immunoglobulin molecules into higher order oligomers (e.g. through covalent bonding between domains in the Fc region of the H chain of one immunoglobulin with an Fc region of an H chain of an adjacent immunoglobulin (such as seen in, for example, pentameric and hexameric forms of IgM) and non-covalent self-association such as that seen in IgG when bound to antigen and which, among other properties, leads to oligomerisation including hexamer formation (Diebolder C A et al., Science 343(6176):1260-1263, 2014) and to certain effector functions).

As used herein, the term “Fc region component” is to be understood as referring to a part of the Fc region of an immunoglobulin heavy (H) chain comprising at least a CH3 domain (or at least a CH4 domain), where H429 is located, but preferably comprising a CH2 and CH3 domain and optionally further comprising an immunoglobulin hinge sequence (which may, in turn, comprise all or a portion of the lower hinge, core hinge and upper hinge sequences), that is capable of forming (e.g. by dimerisation) an Fc fragment or Fc-like fragment. An “Fc-like fragment” is to be understood as referring to an Fc fragment-like structure, but which comprises fragments or components of the Fc region (e.g. the CH3 domain (or CH4 domain) alone or a CH3 domain in combination with a CH2 domain and optionally further comprising an immunoglobulin hinge sequence (which may, in turn, comprise all or a portion of the lower hinge, core hinge and upper hinge sequences).

As used herein, the term “treating” includes prophylaxis as well as the alleviation of established symptoms of a disease or condition. As such, the act of “treating” a disease or condition therefore includes: (1) preventing or delaying the appearance of clinical symptoms of the disease or condition developing in a subject suffering from, or predisposed to, the disease or condition; (2) inhibiting the disease or condition (i.e. arresting, reducing or delaying the development of the disease or condition or a relapse thereof, in case of a maintenance treatment, or at least one clinical or subclinical symptom thereof); and (3) relieving or attenuating the disease or condition (i.e. causing regression of the disease or condition or at least one of clinical or subclinical symptom thereof).

As used herein, the phrase “manufacture of a medicament” includes the use of one or more immunotherapeutic protein as defined in the first aspect directly as the medicament or in any stage of the manufacture of a medicament comprising one or more immunotherapeutic protein as defined in the first aspect.

The term “effective amount” is an amount sufficient to effect beneficial or desired clinical results. An effective amount can be administered in one or more administrations. Typically, an effective amount is sufficient for treating a disease or condition or otherwise to palliate, ameliorate, stabilise, reverse, slow or delay the progression of a disease or condition. By way of example only, an effective amount of an immunotherapeutic protein such as a mutant IgG1 antibody may comprise between about 0.1 and about 250 mg/kg body weight per day, more preferably between about 0.1 and about 100 mg/kg body weight per day and, still more preferably between about 0.1 and about 25 mg/kg body weight per day. However, notwithstanding the above, it will be understood by those skilled in the art that an effective amount may vary and depend upon a variety of factors including the age, body weight, sex and/or health of the subject being treated, the activity of the particular immunotherapeutic protein, the metabolic stability and length of action of the particular immunotherapeutic protein, the route and time of administration of the particular immunotherapeutic protein, the rate of excretion of the particular immunotherapeutic protein and the severity of, for example, the disease or condition being treated.

As shown in the Examples provided hereinafter, it has been found that a mutant antibody including a point mutation at the H429 position may confer significant functional changes. For example, an IgG mutant with an H429F substitution shows an enhanced ability to activate complement-dependent cytotoxicity (CDC) in complement assays (i.e. assays of complement function). Also, it has been found that a fusion protein comprising an Fc region component with a point mutation at position 429 (i.e. H429F) fused to an angiotensin-converting enzyme 2 (ACE2) ectodomain (which may act as a “decoy” to block viral interaction and cellular entry of coronaviruses to host cells) may provide an enhanced ability to provide an antiviral effect through CDC of infected cells, while a similar fusion protein with an H429Y substitution displays enhanced virus neutralisation with little or no complement activation (since immunotherapeutic proteins comprising an H429Y modified Fc component shows abrogated FcγR binding and activation, especially with FcγRIIIa). While not wishing to be bound by theory, it is considered that these effects may be due to oligomerisation of the proteins into oligomers through the self-association of the antibodies/fusion proteins either in solution or upon binding to a relevant target molecule (e.g. an antigen to which an antibody mutant is directed) through “on target” oligomerisation.

Further in this regard, it is to be noted that while monomeric IgG1 and IgG3 can deliver CDC, the level of CDC can be regarded as low or “poor” by comparison to that achieved with naturally pentameric or hexameric IgM. Moreover, IgG antibodies such as IgG1 and IgG3, unlike IgM, are also comparatively poor agglutinins by virtue of their bivalency. Thus, by enabling oligomerisation (by possibly enhancing the known weak inherent capacity of the Fc of an immunoglobulin such as IgG to self-associate, especially after the immunoglobulin has bound to an antigen (i.e. “on target” oligomerisation), an immunotherapeutic protein according to the present disclosure may, by forming oligomers, provide a more optimal platform for complement system activation and other functions enhanced by self-association. This oligomerisation may, particularly and/or optimally involve the formation of a hexamer; and such hexamerisation has been visualised in the crystallographic structure of anti-HIV antibody b12 (Saphire E O et al., Science 293:1155-1159, 2001), wherein CH3 residues form an interface between adjacent IgG:IgG molecules, so forming a hexamer that optimally presents binding sites for the six globular head domains of the C1q subunit which initiates the activation of the classical complement pathway.

Moreover, many cellular molecules, including cell surface molecules, require substantial clustering by a ligand (which may be soluble or another cell surface molecule) to induce a signal that consequently induces a cellular response, and thus the dimerisation that will typically be achieved with an antibody, may not be sufficient to induce a signal that leads to a meaningful cellular response. However, it is known that the strength of the signal can be increased by increasing clustering of the target molecule through, for example, approaches involving the addition of other entities that cross-link the ligand (Chenoweth A C et al., Immunol Cell Biol 98:287-304, 2020), and thus, in the case of an antibody (such as a mAb) bound to its target molecule, such “super cross-clustering” or “hyper-clustering” of the target molecule can be achieved by the use of additional anti-immunoglobulin antibodies that cross-link the mAb that is bound to the target molecule. While not wishing to be bound by theory, it is considered that proteins such as a mutant antibody including an H429F point mutation as mentioned above, through self-association upon binding to a target molecule, may similarly achieve super cross-clustering or hyper-clustering” of the target molecule. Depending upon the nature of the target molecule, such clustering might induce an enhanced signalling response that may lead, for example, to cell proliferation (e.g. where the target molecule is, for example, CD3 or CD28), the stimulation of an inhibitory pathway to inhibit or reduce cell responses (e.g. immune checkpoints; reviewed in Chenoweth et al., supra 2020), or the stimulation of a pathway that induces cell death such as, for example, programmed cell death including apoptosis (see, for example, Ashkenazi A., Nat Rev Drug Discov 7(12):1001-1012, 2008). Stimulation of cellular pathways by mAbs for the development of therapeutic molecules is an approach being used to treat a range of diseases including cancers, inflammation and autoimmune diseases, infections, cardiovascular diseases and others (Ashkenazi, supra 2008).

Thus, in a first aspect, the present disclosure provides an immunotherapeutic protein comprising one or more immunoglobulin heavy chain polypeptide comprising an Fc region component comprising at least a CH3 domain (or at least a CH4 domain), wherein said one or more polypeptide includes an amino acid substitution at a position corresponding to H429 of the amino acid sequence of human IgG1 heavy chain (Eu numbering).

In some embodiments, the immunotherapeutic protein of the first aspect comprises a dimeric immunotherapeutic protein comprising first and second immunoglobulin heavy chain polypeptides comprising an Fc region component comprising CH2 and CH3 domains (so that the first and second polypeptides may form (e.g. by dimerisation) an Fc fragment or Fc-like fragment), wherein the Fc region component of at least one of said first and second polypeptides comprises an amino acid substitution at a position corresponding to H429 of the amino acid sequence of human IgG1 heavy chain (Eu numbering). Accordingly, the immunotherapeutic protein of such embodiments may be an immunoglobulin molecule such as, for example, an antibody or an antibody-like molecule such as a dimeric polypeptide comprising a pair of single-chain Fv polypeptides linked via Fc fragments (i.e. scFv-Fc) or a minibody (i.e. a protein comprising a pair of scFv polypeptides linked via CH3 domains (see the discussion of multivalent scFv-Fc and minibodies in, for example, Olafsen T et al., Generation of Antibody Fragments and Their Derivatives, Antibody Engineering Second edition, pp 69-84, 2010)).

In other embodiments, the immunotherapeutic protein of the first aspect comprises a partner polypeptide linked to an Fc region component comprising at least a CH3 domain (or at least a CH4 domain), wherein the Fc region component comprises an amino acid substitution at a position corresponding to H429 of the amino acid sequence of human IgG1 heavy chain (Eu numbering). Accordingly, the immunotherapeutic protein of such embodiments may be provided in the form of a fusion protein or protein conjugate. Those skilled in the art will understand that in a fusion protein, the partner polypeptide will be covalently linked (i.e. “fused”) to the Fc component (i.e. as a fusion partner) via a peptide bond or linker sequence (e.g. a short peptide linker sequence such as an immunoglobulin hinge sequence or a well-known glycine-serine linker such as GGGGS) at the N- or C-terminus of the fusion partner (i.e. Fc component), whereas in a protein conjugate, the partner polypeptide will be covalently or non-covalently linked to the Fc component (i.e. as a conjugate partner) through a chemical linkage such as a disulphide bond (e.g. through one or more cysteine (C) residue) or cross-linker compound such as a homobifunctional cross-linker such as disuccinimidyl suberate (DSS) (e.g. bis(sulfosuccinimidyl)suberate (BS³); Thermo Fisher Scientific, Waltham, MA, United States of America) or disuccinimidyl tartrate (DST) to link amine groups or a heterobifunctional cross-linker such as m-maleimidobenzoyl-N-hydroxysuccinimide ester (MDS) and N-(ε-maleimidocaproloxy) succinimide ester (EMCS), or by other non-covalent bonding such as hydrogen bonding. Where the Fc component is a conjugate partner, the immunotherapeutic protein conjugate may be considered as a cross-linked protein, and the Fc component may be conjugated to the partner polypeptide at the N- or C-terminus, but otherwise at any other suitable site on the partner polypeptide. Alternatively, the Fc component (as a conjugate partner) may be conjugated to the partner polypeptide at the N- or C-terminus of the Fc component or otherwise at any other suitable site on the Fc component (e.g. within CH1 or the upper hinge sequence if these are included in the Fc region component).

The Fc region component (also referred to hereinafter as the “Fc component”) of the immunotherapeutic protein may be derived from one or more immunoglobulin type (e.g. an IgG or IgA) and may comprise a full length (i.e. “complete”) Fc region such as, for example, a heavy (H) chain polypeptide fragment corresponding to that generated by papain digestion (i.e. wherein the polypeptide is cleaved within the upper hinge sequence to generate an Fc region comprising the constant heavy domain 2 (CH2; amino acid A231 to K340 of the human IgG1 heavy chain polypeptide (Eu numbering)), constant heavy domain 3 (CH3; amino acid G341 to G446 or K447 of the human IgG1 heavy chain polypeptide (Eu numbering)) and lower hinge sequence (also known as hinge proximal sequence of CH2; amino acids P232 to P238 (Eu numbering)) and core hinge sequence (amino acids C226 to C229) and similar heavy chain polypeptide fragments that may be prepared through digestion of an immunoglobulin heavy chain polypeptide with plasmin and human neutrophil elastase (NHE). Further examples of suitable Fc region components may comprise fragments of the heavy chain polypeptide which comprise, in addition to the CH2 and CH3 domains and the lower and core hinge sequences, all or part of the upper hinge sequence and constant heavy domain 1 (CH1). On the other hand, other suitable Fc region components may comprise fragments of the heavy chain polypeptide which comprise only the CH3 domain (e.g. amino acid G341 to G446 or K447 of the human IgG1 heavy chain polypeptide (Eu numbering)) or a fragment thereof. In addition, suitable Fc region components may comprise heterogeneous (“hybrid”) CH3 domains such as strand exchange engineered domain (SEED) forms of a CH3 domain comprising fragments derived from the IgG1 CH3 domain and other proteins such as IgA (Davies et al., Prot Eng Des Sel 23(4):195-202, 2009).

In some embodiments, the Fc region component is derived from a human immunoglobulin heavy chain polypeptide (e.g. such as those shown in FIGS. 3 and 4).

In other embodiments, the Fc region component is derived from an IgG heavy chain polypeptide, preferably an IgG1 or IgG3 (e.g. human IgG1 or IgG3) heavy chain polypeptide, and more preferably, an IgG1.

The Fe region component comprises an amino acid substitution at a position corresponding to H429 of the amino acid sequence of the human IgG1 heavy chain polypeptide (Eu numbering). Where the Fc region component is derived from another immunoglobulin type or isotype (or from an immunoglobulin from another species), it will be appreciated that those skilled in the art can readily determine a position corresponding to H429 of the human IgG1 heavy chain polypeptide IgG1 by, for example, routine sequence alignments (e.g. as shown in FIGS. 3 and 4).

Included among the suitable mutations at position 429 are:
H→X, where X is selected from tyrosine (H429Y), phenylalanine (H429F), tryptophan (H429W), glutamate (H429E), aspartate (H429D), glutamine (H429Q), serine (H429S), asparagine (H429N), and threonine (H429T).

Some preferred amino acid substitutions at the position corresponding to H429 of the amino acid sequence of the human IgG1 heavy chain polypeptide include the H→Y (e.g. H429Y) substitution and the H→F (e.g. H429F) substitution.

The amino acid substitution at the position corresponding to H429 of the amino acid sequence of the human IgG1 heavy chain polypeptide may be the only mutation in the Fc component. However, in some embodiments, the Fc component may comprise one or more further mutation (e.g. amino acid substitution). For example, the Fc component may comprise one or more sequence mutation known to those skilled in the art (see, for example, the examples listed in Table 1 of Wang X et al., Protein Cell 9(1):63-73, 2018; the entire disclosure of which is herein incorporated by reference) such as, for example: mutations that may modulate FcγR binding (e.g. S239D/1332E of IgG1 (Eu numbering) which increase FcγRIIIa binding); mutations to improve antibody-dependent cellular cytotoxicity (ADCC) such as S239D/1332E (Lazar G A et al., Proc Natl Acad Sci USA 103(11):4005-4010, 2006), opsonic phagocytosis (e.g. G236A/S239D/1332E; Richards J O et al., Mol Cancer Ther 7:2517-2527, 2008) or complement activation (e.g. S267E/H268F/S324T; Moore G L et al., MAbs 2:181-189, 2010), or decrease effector function (e.g. IgG1: L234A/L235A; IgG4: F234A/L235A; Xu D et al., Cell Immunol 200(1):16-26, 2000); mutations that may increase inhibition via FcγRIIb co-engagement (S267E/L328F; Chu S Y et al., Mol Immunol 45(15):3926-3933, 2008); and mutations which confer enhanced binding to the neonatal Fc receptor (FcRn) such as M252Y/S254T/T256E (Dall'Acqua W F et al., J Immunol 169(9):5171-5180, 2002) to increase in vivo half-life and thereby improve pharmacokinetics (PK).

Other mutations that may be included in the Fc region component include mutations to enhance complement activation such as an amino acid mutation at a position(s) corresponding to K447 of the amino acid sequence of the human IgG1 heavy chain polypeptide (Eu numbering); in particular, K447X, where X is selected from null (i.e. K447del; an amino acid deletion or truncation of the Fc component), and glutamate (i.e. K447E) (see van der Bremer E T J et al., mAbs 7(4):672-680, 2015). Further, the Fc region component may also include mutations which modulate glycosylation (e.g. a mutation at a position corresponding to Asn297 of the amino acid sequence of the human IgG1 heavy chain polypeptide (Eu numbering) such as N297A, N297Q or N297G (Wang et al., 2018 supra) to provide a site with modified glycosylation (e.g. the lack of glycan at position 297) to abolish FcγR and complement C1 binding and/or activation.

Alternatively, the Fc region component may also be treated to achieve modified glycosylation by producing the immunotherapeutic protein in the presence of kifunensine (a mannosidase inhibitor which prevents normal maturation of the N-linked glycan including core fucosylation of the N-linked glycan); a modification that specifically enhances activity via FcγRIIIa. Further, an Fc component lacking core fucosylation of the Asn297 glycan can also be achieved by culturing host cells expressing the immunotherapeutic protein with inhibitors of fucosylation (e.g. 2-fluoro peracetylated fucose, or similar) or by the expression of enzymes that modify glycosylation pathways (e.g. GDP-6-deoxy-D-lyxo-4-hexulose reductase; Neha M et al., J Biotech 5:100015, 2020) or by modification of these pathways by gene suppression (e.g. siRNA silencing of the α-1,6-Fucosyltransferase fucosyl transferase gene FUT8; Imai-Nishiya H et al., BMC Biotechnol 7:84, 2007) or knock-out (Yamane-Ohnuki N et al., Biotechnol Bioeng 87:614-622, 2004.)

The immunotherapeutic protein of the first aspect may be monomeric, dimeric or oligomeric in solution (e.g. in physiological saline at neutral pH such as physiological pH of about 7.4).

For example, in some embodiments, the immunotherapeutic protein comprises a monomer in physiological saline, wherein each monomer comprises one copy of the Fc region component, while in other embodiments, the immunotherapeutic protein comprises a dimer in physiological saline wherein the Fc region components self-associate (i.e. to form the dimer) either by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003). Thus, in some embodiments, the Fc region component comprises a core hinge sequence to enable the immunotherapeutic protein to self-associate (i.e. to form a dimer) through the formation of inter-chain disulphide bonds between one or more cysteine (C) residues of the core hinge sequence of two Fc region components, while in some other embodiments, the Fc region component comprises a CH3 domain to enable CH3:CH3 self-association by non-covalent interactions, or Fc region component comprises a CH3 domain and CH2 domain to enable self-association by CH2:CH2 and CH3:CH3 non-covalent interactions. It is to be noted that in the Examples and Figures hereinafter, dimeric forms of the immunotherapeutic protein are considered to be single molecules (i.e. each comprising two copies of the protein dimerised through the Fc region components) and are referred to as monomers/monomeric.

In other embodiments, the immunotherapeutic protein comprises an oligomer in physiological saline (at neutral pH such as physiological pH of about 7.4), wherein the immunotherapeutic protein comprises an amino acid substitution at the position corresponding to H429 of the amino acid sequence of the human IgG1 heavy chain polypeptide, which enables self-association of the Fc or Fc-like fragment comprising the mutated Fc components into soluble oligomeric forms in physiological saline (e.g. oligomeric forms comprising, for example, 3 copies, 4 copies, 5 copies, 6 copies or 12 copies assembled from, for example, dimeric forms of the immunotherapeutic protein, such that, in some particular embodiments, the oligomeric form of the immunotherapeutic protein may comprise six dimeric proteins (i.e. a hexameric form) comprising, in total, 12 copies of the immunotherapeutic protein). In such embodiments, the amino acid substitution at the position corresponding to H429 of the amino acid sequence of the human IgG1 heavy chain polypeptide may be:
H429X′, where X¹ is selected from tyrosine (i.e. H429Y), methionine, isoleucine, leucine, tryptophan and valine;
   but preferably, the immunotherapeutic protein comprises an H→Y (i.e. H429Y) substitution.

In yet other embodiments, the immunotherapeutic protein may form an oligomer upon binding to a relevant target through “on target” oligomerisation. Such oligomerisation may occur with an immunotherapeutic protein that is monomeric or dimeric in physiological saline (at neutral pH such as physiological pH of about 7.4), and comprises an amino acid substitution at the position corresponding to H429 of the amino acid sequence of the human IgG1 heavy chain polypeptide which enables on target oligomerisation into oligomeric forms (e.g. forms comprising 3 copies, 4 copies, 5 copies, 6 copies or 12 copies (e.g. a hexamer of dimeric forms) of the immunotherapeutic protein). In such embodiments, the amino acid substitution at the position corresponding to H429 of the amino acid sequence of the human IgG1 heavy chain polypeptide may be:
H429X², where X² is selected from phenylalanine (i.e. H429F), glutamate, glutamine and serine;
   but preferably, the immunotherapeutic protein comprises an H→F (i.e. H429F) substitution.

Alternatively, oligomeric forms of the immunotherapeutic protein of the first aspect may be produced by employing other techniques well known to those skilled in the art such as, for example, the use of Fc multimeric forms (Stradomers™) comprising linked multimerisation domain (MD) sequences from the hinge region of human IgG2 or the isoleucine zipper (ILZ) to the N- or C-terminus of murine IgG2a (Fitzpatrick E A et al., Front Immunol 11, article 496, 2020), and the use of multimerisation sequences from IgM (Melcheil et al., Sci Rep 1:124 doi:10.1038/srep0012, 2011), or docking and dimerisation sequences from unrelated proteins such as cyclic adenosine monophosphate-dependent protein kinase and A-kinase anchoring proteins (Rossi E A et al., Bioconjug Chem 23(3):309-323, 2012).

It has further been found that the immunotherapeutic protein of the first aspect may show enhanced binding of the neonatal Fc receptor (FcRn) by virtue of an amino acid substitution at a position corresponding to H429 of the amino acid sequence of human IgG1 heavy chain (Eu numbering). It is anticipated that this will mean that the immunotherapeutic protein will show a greater in vivo half-life (i.e. relative to an equivalent immunotherapeutic protein with no mutation at the H429 position) and thereby improved pharmacokinetics (PK), since FcRn is known to “recycle” antibodies so as to control how long they last in the body (Ward E S and R J Orber, Trends Pharmacol Sci 39(10):892-904, 2018).

Dimeric Immunotherapeutic Proteins (e.g. Immunoglobulin Molecules)

The immunotherapeutic protein of the first aspect may be a dimeric immunotherapeutic protein comprising an immunoglobulin molecule, wherein the immunoglobulin molecule comprises first and second immunoglobulin heavy chain polypeptides which each comprise an Fc region component comprising at least a CH3 domain (or at least a CH4 domain). The Fc region components of the first and second immunoglobulin heavy chain polypeptides may self-associate (i.e. to form the dimer) either by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003), or otherwise be linked by, for example, a cross-linker compound such as those mentioned above (e.g. by cross-linking the CH3 domains of the Fc region components). As such, the immunotherapeutic protein may be an immunoglobulin molecule such as, for example, an antibody or antibody derivative such as an scFv-Fc (wherein a dimer of scFv polypeptides is formed through dimerisation of Fc region components), minibody (wherein a dimer of scFv polypeptides is formed through linking CH3 domains/CH4 domains), or any of the other suitable Fc-containing antibodies or derivatives known to those skilled in the art (e.g. as summarised in the “Periodic Table of Antibodies” mentioned above).

Accordingly, the dimeric immunotherapeutic protein may further comprise at least one antigen recognition structure or, in other words, antigen binding region (e.g. an scFv or an antigen binding region comprising variable domains V_{L} and V_{H} such as a Fab fragment). The antigen binding region(s) may specifically bind to an antigen or epitope of therapeutic significance; for example, a cancer-associated antigen such as a cancer antigen present on the surface of a cancerous cell (e.g. a cell surface antigen differentially expressed and/or present in cancer cells such as the CD20, CD38 and CD52 antigens found on the surface of CLL cells, and mucins (e.g. MUC-1) or carbohydrate (e.g. Lewis X) overexpressed in some breast and pancreatic cancers), an autoantigen (e.g. an autoantigen associated with SLE or multiple sclerosis (MS)), an allergen (e.g. bee venom), an antigen associated with other inflammatory diseases such as immune complex vasculitis, an antigen from a transplanted tissue or organ or an antigen of an infectious agent such as an antigen of a bacterial, yeast, parasite or viral pathogen (e.g. an antigen of the SARS-CoV-2 virus, Middle East respiratory syndrome coronavirus (MERS-CoV), respiratory syncytial virus (RSV) or dengue virus). In other examples, the antigen binding region(s) may specifically bind to a cell surface molecule known to induce cell proliferation (i.e. the cell surface molecule may be, for example, CD3 or CD28) and/or stimulate an inhibitory pathway to inhibit or reduce cell responses (i.e. the cell surface molecule may be, for example, an immune checkpoint molecule such as 4-1BB (CD137), cluster of differentiation (CD40, CD154), OX40 receptor (TNFRSF4, CD134), tumour necrosis factor receptor type II (TNFR2, CD120b), glucocorticoid-induced TNFR-related protein (GITR, TNFRSF18, CD357), cluster of differentiation 27 (CD27), T cell immunoglobulin and mucin-domain containing-3 (TIM-3), B and T lymphocyte attenuator (BTLA, CD272), lymphocyte activation gene-3 (LAG3, CD223), cytotoxic T-lymphocyte-associated protein 4 (CTLA4, CD152), inducible T-cell costimulatory (ICOS, CD278), cluster of differentiation 28 (CD28), TT cell immunoreceptor with Ig and ITIM domains (IGIT, Vstm3), programmed death-ligand 1 (PDL-1, CD274) and programmed cell death protein 1 (PD-1, CD279); and wherein oligomerisation of the immunotherapeutic protein may bring about enhanced cross-linking (e.g. super-clustering) and, in turn, enhanced signalling to induce, for example, enhanced cell proliferation or inhibited/reduced cell responses (i.e. through enhanced stimulation of an inhibitory pathway). In yet other examples, the antigen binding region(s) may specifically bind to a cell surface molecule known to induce a cellular response selected from stimulation of an inhibitory pathway to inhibit or reduce responses. In still yet other examples, the antigen binding region(s) may specifically bind to a cell surface molecule which induces cell death when engaged by a ligand or other molecules such as agonistic mAbs. Such cell surface molecules include, for example, those of the TNF-receptor superfamily (also known as TNFRSF) including tumour necrosis factor receptor 1 (TNFR1, TNFRSF lA, CD120a), Fas (CD95, APO-1), Death Receptor 3 (DR3, TNFRSF25), Death Receptor 4 (DR4, CD261, TNFRSF1OA, TRAILR1), Death Receptor 5 (DR5, CD262, TNFRSF1 OB, TRAILR2) and Death Receptor 6 (CD358, TNFRSF21). For example, under physiological conditions, the binding of TNF-related apoptosis-inducing ligand (TRAIL) to its receptor, DR5 (Carneiro B A et al., Nat Rev Clin Oncol 17(7):395-417, 2020) or the binding of the FAS ligand to its receptor, FAS (CD95) on the cell surface induces oligomerisation (Leukocyte and Stromal Cell molecules: The CD Markers, by Zola H et al., page 195, John Wiley & Sons, 2007) that initiates signalling leading to cell death. Other molecules (not related to the TNFRSF) which can induce apoptotic signals leading to cell death when cross-linked by, for example, mAbs or fragments thereof include CD38 (see Gambles M T et al., Molecules 26(15):4658, 2021), CD20 (see Shan D et al., Cancer Immunol Immunother 48:673-683, 2000; and Cardarelli P M et al., Cancer Immunol Immunother 51:15-24, 2002) and CD52 (see Rowan W et al., Immunology 95:427-436, 1998). These molecules can also be advantageously targeted by a dimeric immunotherapeutic protein such that oligomerisation may bring about enhanced cross-linking and induction of apoptotic signals to bring about cell death as may be desired in a target cell.

In some embodiments, an immunoglobulin molecule according to the present disclosure may comprise two antigen binding regions, each of which specifically binds to different antigens or epitopes.

In some embodiments, an immunoglobulin molecule according to the present disclosure that is an antibody, may be of, for example, an IgD, IgE or IgM isotype, but preferably, will be of an IgA or IgG isotype, such as an antibody of any of the human IgA1, IgA2, IgG1, IgG2, IgG3 and IgG4 sub-types.

The first and second immunoglobulin heavy chain polypeptides of a dimeric immunotherapeutic protein comprising an immunoglobulin molecule may be the same (i.e. a homodimer) or different (i.e. a heterodimer).

In some embodiments, an immunoglobulin molecule according to the present disclosure that is an antibody, may be an antibody that forms oligomers either through oligomerisation into oligomers (e.g. hexamers) through self-association of antibodies either in solution (e.g. in physiological saline at neutral pH such as physiological pH of about 7.4) or upon binding to a relevant target (e.g. an antigen to which an antibody mutant is directed) through “on target” oligomerisation.

For example, and while not wishing to be bound by theory, it is considered that an immunoglobulin molecule that is an antibody which includes H429Y amino acid substitution in the Fc region component, may form oligomers in solution (e.g. in physiological saline at physiological pH) which can lead to an overall increase in the strength of binding (for example, enhanced avidity) to a target binding partner (e.g. a cancer antigen, antigen of a bacterial or viral pathogen, or other soluble target molecule or molecular complex). Thus, in the context of an antibody directed against a cancer antigen present on the surface of a cancerous cell, an antibody according to the present disclosure (in the form of an oligomer in solution) may show enhanced ability to bind to the cancerous cells leading to their destruction by, for example, CDC by complement or by phagocytosis by macrophages and/or ADCC; and in the context of an antibody directed against a soluble target molecule or molecular complex, an antibody according to the present disclosure (in the form of an oligomer in solution) may show enhanced ability to bind to the soluble target molecule or molecular complex leading to their removal by phagocytosis by macrophages.

On the other hand, it is considered that an immunoglobulin molecule that is an antibody which includes an H429F amino acid substitution in the Fc region component, may form oligomers upon binding to a relevant target such that the Fc components assembled “on target” present a stabilised optimal platform for binding with, for example, enhanced avidity to the C1q complement protein complex, and thereby lead to complement activation and, in turn, complement-dependent cytotoxicity (CDC). Thus, in the context of an antibody directed against a cancer antigen present on the surface of a cancerous cell, an antibody according to the present disclosure may show enhanced capability to activate complement on the cancerous cells leading to their destruction.

In some embodiments, an immunoglobulin molecule according to the present disclosure may be provided or used as a first immunoglobulin molecule with a first antigen binding region directed to a first antigen, in combination with a second immunoglobulin molecule (according to the present disclosure) with a second antigen binding region directed to a second antigen. By way of example only, in such a combination, the first antigen may be cancer antigen present on the surface of a cancerous cell (e.g. CD38 found on the surface of CLL cells) and the second antigen may be a death receptor (e.g. DR5). Oligomerisation of the first and second immunoglobulin molecules may lead to the formation of hetero-oligomers such as hetero-hexamers wherein the different cell surface targets are incorporated into the cluster which may lead to, for example, the enhanced induction of apoptotic signals to bring about cell death (e.g. where a death receptor has been targeted) with target cell specificity achieved via binding of, for example, a cancer antigen present on a cancerous cell.

### Fusion Conjugate Immunotherapeutic Proteins

The immunotherapeutic protein of the first aspect may comprise a fusion protein or protein conjugate comprised of a partner polypeptide linked to an Fc region component comprising at least a CH3 domain (or at least a CH4 domain).

A fusion protein or protein conjugate according to the present disclosure may be monomeric, dimeric or oligomeric. For example, in some embodiments, the fusion protein or protein conjugate comprises a monomer, wherein each monomer comprises one copy of the partner polypeptide (or a fragment thereof) and one copy of the Fc region component, while in other embodiments, the immunotherapeutic protein comprises a dimer wherein the Fc region components self-associate (i.e. to form the dimer) either by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003). In such a dimer, the immunotherapeutic protein comprises two Fc region components (associated to one another to form, for example, an Fc fragment or Fc-like fragment) and two fused/conjugated partner polypeptides (or two fragments thereof), and as such may be considered as being bivalent in respect of the partner polypeptide (or fragment thereof). The two fused/conjugated partner polypeptides (or two fragments thereof) of a dimeric fusion protein or protein conjugate may be the same (i.e. a homodimer) or different (i.e. a heterodimer). In some embodiments of such a dimer, as depicted in FIG. 53A, the fusion protein is antibody-like (Ab-like) and may have, for example a H₂ format or H₂L₂ format (where the monomeric fusion proteins are produced with a light (L) chain, which in turn may be optionally fused/conjugated with a partner polypeptide (such as a target recognition structure)). As can be readily appreciated, each target recognition structure of an Ab-like molecule with a H₂ or H₂L₂ format may be the same or different such as “X1 X1 X1 X1”, “X1 X1 X1 X2”, “X1 X1 X2 X2”, “X1 X1 X2 X3” or “X1 X2 X3 X4”, where X1, X2, X3 and X4 each represent a different target recognition structure. Also, one or more of the target recognition structures may be replaced with an alternative partner polypeptide type such as an enzyme(s) or reporter molecule(s).

The partner polypeptide may provide the immunotherapeutic protein with a beneficial function and/or characteristic.

In some embodiments, the partner polypeptide may be a cell surface receptor polypeptide (or a fragment thereof) or a co-receptor polypeptide (or fragment thereof).

For example, the partner polypeptide may be a cell surface molecule such as a cell surface receptor polypeptide (or a fragment thereof) that is capable of binding to a structural protein of a virus such that the immunotherapeutic protein may act as a “decoy” to block viral interaction and cellular entry of a virus to a host cell. Accordingly, some examples of such cell surface receptor polypeptides (or fragments thereof) that may comprise the partner polypeptide include the angiotensin-converting enzyme 2 (ACE2) ectodomain (ACE2 being the cellular entry receptor for SARS-CoV-2), nucleolin (the cellular entry receptor for RSV), dipeptidyl peptidase 4 (DPP4, CD26), Hsp70 (the cellular entry receptor for Japanese encephalitis virus), hepatitis A virus cellular receptor 1 (HAVCR1/TIM-1; the cellular entry receptor for Hepatitis A virus and Ebola virus), cluster of differentiation 155 (CD 155; the cellular entry receptor for poliovirus), Glucose transporter 1 (GLUT1; the cellular entry receptor for human T cell leukaemia virus 1), Proto-oncogene tyrosine-protein kinase MER (MERTK; a host factor that promotes swine fever virus entry), TYRO3 Protein Tyrosine Kinase (TYRO3; a cell surface protein associated with lymphocytic choriomeningitis (LMCV) infection), AXL (a cell surface protein associated with lymphocytic choriomeningitis (LMCV) infection) and the cluster of differentiation 4 receptor (CD4 receptor; the cellular entry receptor for human immunodeficiency virus (HIV)), to name just a few. In embodiments where the partner polypeptide is a cell surface receptor polypeptide (or a fragment thereof) that is capable of binding to a structural protein of a virus, a point mutation at the position of the Fc region component corresponding to H429 of the amino acid sequence of human IgG1 heavy chain (Eu numbering) may, for example, provide the immunotherapeutic protein with an enhanced ability to provide an antiviral effect through CDC of infected cells (e.g. where the mutation is H429F) or confer upon the immunotherapeutic protein enhanced virus neutralisation (e.g. where the mutation is H429Y). In other examples, the partner polypeptide may be a ligand for a cell surface molecule known to induce cell proliferation (i.e. the cell surface molecule may be, for example, CD3 or CD28) and/or stimulate an inhibitory pathway to inhibit or reduce cell responses (i.e. the cell surface molecule may be, for example, an immune checkpoint molecule such as 4-1BB, CD40, OX40, TNFR2, GITR, CD27, TIM-3, BTLA, LAG3, CTLA4, ICOS, CD28, TIGIT, PDL-1 and PD-1; and wherein oligomerisation of the immunotherapeutic protein may bring about enhanced cross-linking (e.g. super cross-clustering) and, in turn, enhanced signalling to induce, for example, enhanced cell proliferation or inhibited/reduced cell responses (i.e. through enhanced stimulation of an inhibitory pathway). In yet other examples, the partner polypeptide may be a ligand for a cell surface molecule which induces cell death when engaged by a ligand or other molecules such as agonistic mAbs (e.g. the abovementioned cell surface molecules of the TNFRSF (e.g. TNFR1, Fas, DR3, DR4, DR5 and DR6) and other molecules such as CD38, CD20 (Shan et al., supra 2000; and Cardarelli et al., supra 2002) and CD52 (Rowan et al., supra 1998). Thus, in some embodiments, the immunotherapeutic protein of the first aspect may comprise a dimeric fusion protein or protein conjugate comprised of, for example, TRAIL (i.e. the ligand for DR4 or DR5) as the partner polypeptide linked to an Fc region component comprising at least a CH3 domain which includes an H429F amino acid substitution. Upon binding of the ligand to the cell surface molecule, oligomerisation of an immunotherapeutic protein of these embodiments may bring about enhanced cross-linking and induction of apoptotic signals to bring about cell death as may be desired in a target cell.

Where the partner polypeptide is a co-receptor polypeptide (or fragment thereof), the co-receptor polypeptide (or fragment thereof) may be, for example, C—X—C chemokine receptor type 4 (CXCR4), C—C chemokine receptor type 5 (CCR5)(co-receptors of the CD4 receptor which bind to the HIV viral glycoprotein gp120 and enable HIV to fuse with the host cell membrane), tetraspanin and occludin (which are co-receptors required to enable infection by Hepatitis C virus (HCV)), and Gas6 (which is a ligand of receptors (such as AXL and TYRO3) which together bind to phosphatidylserine displayed on viruses, including West Nile virus, Zika virus and Ebola virus among others, and facilitates host cell entry of such viruses), among many others.

In another example, the partner polypeptide may be a cell surface receptor polypeptide (or a fragment thereof) that is capable of binding to a ligand for the cell surface receptor. Accordingly, some examples of such polypeptides (or fragments thereof) include the cytotoxic T-lymphocyte-associated protein 4 (CTLA4) (or a soluble extracellular fragment thereof). CTLA4 functions as an immune checkpoint and downregulates immune responses. An immunotherapeutic protein comprising a fusion protein or protein conjugate comprised of a CTLA4 partner polypeptide (e.g. a CTLA4-Fc component fusion protein) may, by forming a dimer or oligomer, show enhanced binding to the CTLA4 ligand and thereby act as a decoy to produce various therapeutic effects for the potential treatment of tumours (e.g. melanoma and colorectal cancer) and various autoimmune diseases such as SLE and rheumatoid arthritis (RA). Further examples of suitable cell surface receptor polypeptides (or a fragment thereof such as an ectodomain) that are capable of binding to a ligand for the cell surface receptor, include other immune checkpoints (e.g. PD1) and other cytokine receptors such as interleukin-1 receptor (IL-1R), interleukin-6 receptor (IL-6R), tumour necrosis factor receptor-2 (TNFR2, also known as CD120b) or a receptor for a cytokine of the TGF-β superfamily (see the review in Czajkowsky D M et al., Mol Med 4(10):1015-1028, 2012). An immunotherapeutic protein comprising a fusion protein or protein conjugate comprised of IL-1R (or a fragment thereof) may potentially be used in an anti-IL-1 therapy for treatment of, for example, type 2 diabetes, and an immunotherapeutic protein comprising a fusion protein or protein conjugate comprised of IL-6R (or a fragment thereof) may potentially be used in an anti-IL-6 therapy for treatment of, for example, tumours and RA. An immunotherapeutic protein comprising a fusion protein or protein conjugate comprised of TNF-R2 or an ectodomain thereof may potentially be used in a treatment of RA or other inflammatory disease or condition.

While not wishing to be bound by theory, it is considered that a fusion protein or protein conjugate according to the present disclosure (whether in monomeric or dimeric form) which includes an H429Y amino acid substitution in the Fc region component may form oligomers in solution (e.g. in physiological saline at neutral pH such as physiological pH of about 7.4) that can lead to an overall increase in the strength of binding (for example, enhanced avidity) to a target binding partner (e.g. a structural protein of a virus or a ligand for a cell surface receptor, or other soluble target molecule or molecular complex). Thus, in the context of a fusion or conjugate protein which comprises a cell surface receptor polypeptide or a fragment thereof that is capable of binding to a structural protein of a virus, resulting in enhanced avidity of binding to the virus to provide enhanced virus neutralisation (possibly conferred by cross-linking and/or aggregating viral particles (virions)). However, it is considered that in at least some embodiments, such soluble oligomers are unable to substantially bind to Fc receptors and thus may be capable of virus neutralisation with little or no complement activation.

On the other hand, and again not wishing to be bound by theory, it is considered that a fusion protein or protein conjugate (whether in monomeric or dimeric form) which includes an H429F amino acid substitution in the Fc region component, may form oligomers upon binding to a relevant target (e.g. a structural protein of a virus or a ligand for a cell surface receptor) such that the Fc components assembled “on target” present a stabilised optimal arrangement for binding (with, for example, enhanced avidity) to the C1q complement protein complex, and thereby lead to complement activation and, in turn, enhanced complement-based effector functions such as complement-dependent cytotoxicity (CDC).

Immunotherapeutic proteins according to the present disclosure may be produced in accordance with any of the standard methodologies known to those skilled in the art. For instance, those skilled in the art can readily prepare an immunotherapeutic fusion protein by generating a construct, using standard molecular biology techniques, which comprises a polynucleotide sequence(s) encoding the fusion protein, introducing the construct into a suitable host cell (e.g. a human kidney (HEK) host cell or derivative thereof such as Expi293 cells (Thermo Fisher Scientific) for expression of the fusion protein, culturing the host cells according to standard culturing protocols and recovering the expressed fusion protein from the culture supernatant using, for example, any of the known suitable methodologies for purification (e.g. affinity chromatography (e.g. Protein A), ion exchange chromatography (IEX), size exclusion chromatography (SEC) and combinations thereof). Similar methodologies can be used to prepare an immunotherapeutic protein which is an immunoglobulin molecule such as an antibody or an Ab-like molecule. That is, those skilled in the art can readily prepare a mutant antibody (i.e. an antibody including an amino acid substitution at a position corresponding to H429 of the amino acid sequence of human IgG1 heavy chain (Eu numbering) by generating a construct(s) which comprises a polynucleotide sequence(s) encoding the variable heavy (V_{H}) and light (V_{L}) region sequence of a suitable antibody (e.g. one including an antigen binding region that binds to an antigen of interest) and a constant heavy (CH) region from, for example, an IgG1 antibody, and incorporate into the CH3 region-encoding polynucleotide sequence by standard molecular biology techniques such as site-directed mutagenesis, polynucleotide sequence changes to encode a mutation at H429 (e.g. H429F and H429Y). As with the preparation of an immunotherapeutic fusion protein, the construct(s) can be introduced into a suitable host cell (e.g. a human kidney (HEK) host cell or derivative thereof), cultured according to standard culturing protocols and the expressed mutant antibody purified from the culture supernatant using, for example, any of the known suitable methodologies for purification such as affinity chromatography. Further, it has been found that where affinity chromatography (e.g. Protein A) is used in the purification, especially where the immunotherapeutic protein is in the form of an Ab-like molecule, the use of mild elution conditions such as the use of an elution buffer comprising a low concentration of arginine (e.g. less than 130 mM) and at less than or equal to pH 5, is advantageous so as to suppress the formation of aggregates. This can be achieved by, for example, standard liquid chromatography systems that can deliver a buffer gradient to the column, in this case, a linear gradient to, for example, 35% of 130 mM arginine (pH 4.0).

In some preferred embodiments, the recovery of an expressed immunotherapeutic protein according to the present disclosure is:
(i) preferably conducted under conditions of mildly acidic pH (e.g. a pH of less than neutral pH such as pH 6.5, preferably pH 5.5, and more preferably, pH 5.0) where it is desired that the immunotherapeutic protein be provided in a monomeric form; or
(ii) where it is desired that the immunotherapeutic protein be provided in a oligomeric form (e.g. as a hexamer), preferably conducted under conditions of substantially neutral pH (e.g. a pH in the range of 7.0 to 8.5, preferably 7.5 to 8.0, or more preferably, at physiological pH of about 7.4); or (iii) preferably conducted using a method comprising affinity chromatography using an elution buffer comprising a low concentration of arginine (e.g. less than 130 mM) and at less than or equal to pH 5.0 (preferably about pH 4.0), especially where it is desired that the immunotherapeutic protein be provided as an antibody-like molecule.

In some particular embodiments, the recovery of an expressed immunotherapeutic protein according to the present disclosure comprises recovery by size exclusion chromatography (SEC), for example under conditions of mildly acidic pH for the production of monomeric forms of the immunotherapeutic protein, or under substantially neutral pH for the production of the immunotherapeutic protein in oligomeric forms. The SEC may, if desired, follow a recovery stage comprising ion exchange chromatography (IEX).

In a second aspect, the present disclosure provides the use of an immunotherapeutic protein as defined in the first aspect, for treating or preventing a disease or condition in a subject, wherein the disease or condition may be selected from, for example, autoimmune diseases and conditions, other inflammatory diseases, infectious diseases and proliferative diseases.

In a third aspect, the present disclosure provides the use of an immunotherapeutic protein as defined in the first aspect, in the manufacture of a medicament for treating or preventing a disease or condition, wherein the disease or condition is selected from autoimmune diseases and conditions, other inflammatory diseases, infectious diseases and proliferative diseases.

In a fourth aspect, the present disclosure provides a method for treating or preventing a disease or condition, comprising administering to the subject an effective amount of an immunotherapeutic protein as defined in the first aspect, wherein the disease or condition is selected from autoimmune diseases and conditions, other inflammatory diseases, infectious diseases and proliferative diseases.

Among the diseases and conditions that may be treated or prevented by the method of the fourth aspect, are autoimmune diseases and conditions such as SLE and MS, other inflammatory diseases (e.g. immune complex vasculitis), infectious diseases and proliferative diseases (especially solid tumours such as breast cancers), blood cancers such as lymphoproliferative disorders (LPDs) including leukaemias (e.g. acute lymphoblastic leukaemia (ALL) and chronic lymphocytic leukaemia (CLL)), adenocarcinomas and lymphomas, as well as multiple myeloma (MM) and X-linked proliferative disease.

The method of the fourth aspect of the present disclosure will be typically applied to the treatment of a disease or condition in a human subject. However, the subject may also be selected from, for example, livestock animals (e.g. cows, horses, pigs, sheep and goats), companion animals (e.g. dogs and cats) and exotic animals (e.g. non-human primates, tigers, elephants etc).

In some embodiments, wherein the immunotherapeutic protein comprises a fusion protein comprising a partner polypeptide linked to an Fc region component comprising at least a CH3 domain (or at least a constant heavy domain 4 (CH4) domain) and said partner polypeptide is a cell surface receptor polypeptide (or a fragment thereof) that is capable of binding to a structural protein of a virus such that the immunotherapeutic protein may act as a “decoy” to block viral interaction and cellular entry of a virus to a host cell, the method of the fourth aspect may further comprise administering an antibody directed against the said virus (i.e. target virus). For example, where the immunotherapeutic protein comprises an ACE2 polypeptide (or a fragment thereof) which binds with the RBD of the CoV-2 spike protein, the antibody may be selected from antibodies that are, for example, broadly neutralising coronavirus mAbs (ie bNmAbs which can neutralise multiple coronavirus types or strains), broadly reactive coronavirus mAbs (ie mAbs which may not be neutralising but can bind with multiple coronavirus types or strains), broadly neutralising SARS-CoV-2 mAbs (ie bNmAbs which can neutralise multiple SARS-CoV-2 strains) and broadly reactive SARS-CoV-2 mAbs, broadly neutralising coronavirus spike stem specific mAbs, broadly reactive coronavirus spike stem specific mAbs, are broadly neutralising SARS-CoV-2 spike stem specific mAbs, and broadly reactive SARS-CoV-2 spike stem specific mAbs. In some specific embodiments, the antibody may be, for example, targeted to an epitope of a SARS-CoV-2 structural protein other than the spike protein (S) such as the envelope protein (E), the membrane protein (M) or the nucleocapsid protein (N). In some other specific embodiments, the antibody may be targeted to, for example, an epitope on the spike protein (S) but at a site distinct from the RBD. More generally, where the immunotherapeutic protein comprises a cell surface receptor polypeptide other than an ACE2 polypeptide, or a co-receptor polypeptide, or a fragment thereof, the antibody directed against the target virus may be selected from antibodies that are, for example, broadly neutralising against a class/family of viruses (e.g. human immunodeficiency viruses (HIV)), or which are broadly reactive against a class/family of viruses, broadly neutralising of strains of a specific virus type (e.g. bNmAbs which can neutralise multiple HIV-1 strains), and mAbs which are broadly reactive to a specific virus type. As shown hereinafter, it has been found that an immunotherapeutic protein of the present disclosure and an antibody directed against the target virus may synergistically cooperate to enhance CDC killing of cells (e.g. virus-infected cells).

Similarly, where the immunotherapeutic protein comprises a fusion protein comprising a partner polypeptide linked to an Fc region component comprising at least a CH3 domain (or at least a constant heavy domain 4 (CH4) domain) and said partner polypeptide is, for example, directed at a target of therapeutic significance (e.g. the partner polypeptide is a cell surface receptor polypeptide (or a fragment thereof) that is capable of binding to CTLA4 which functions as an immune checkpoint and downregulates immune responses), the method of the fourth aspect may further comprise administering an antibody directed against the said target (e.g. an antibody which binds to CTLA4) to, for example, provide an enhanced response such as enhanced CDC killing of cells (e.g. cancer cells).

Where the method of the fourth aspect does comprise administering an antibody in addition to a fusion protein (i.e. as described in the preceding two paragraphs), preferably the antibody comprises an Fc region component comprising an amino acid substitution at the position corresponding to H429 of the amino acid sequence of the human IgG1 heavy chain polypeptide (EU numbering) such as, for example, an H429X² amino acid substitution, where X² is selected from phenylalanine (H429F), glutamate (H429E), glutamine (H429Q) and serine (H429S).

In a fifth aspect, the present disclosure provides a pharmaceutical composition or medicament comprising an immunotherapeutic protein as defined in the first aspect, and a pharmaceutically acceptable carrier, diluent and/or excipient.

The immunotherapeutic protein may be administered in combination with one or more additional agent(s) for the treatment of the particular disease or condition being treated. For example, in the context of treating proliferative diseases, the immunotherapeutic protein may be used in combination with other agents for treating cancer (including, for example, antineoplastic drugs such as cis-platin, gemcitabine, cytosine arabinoside, doxorubicin, epirubicin, taxoids including taxol, topoisomerase inhibitors such as etoposide, cytostatic agents such as tamoxifen, aromatase inhibitors (e.g. as anastrozole) and inhibitors of growth factor function (e.g. antibodies such as the anti-erbB2 antibody trastuzumab (Herceptin™)). In some embodiments, the immunotherapeutic protein may be administered with one or more additional agent(s) which may also be an immunotherapeutic protein(s) according to the present disclosure. For example, where a first immunotherapeutic protein according to the present disclosure comprises an ACE2 polypeptide (or a fragment thereof) which binds with the RBD of the CoV-2 spike protein, then the second immunotherapeutic protein of the present disclosure may be an antibody directed against the target CoV-2 virus, particularly one targeted to an epitope of a different structural protein (e.g. the envelope protein (E), the membrane protein (M) or the nucleocapsid protein (N)) or on the same structural protein (i.e. the spike protein (S)) but at a site distinct from the RBD. As shown hereinafter, it has been found that a combination of such immunotherapeutic proteins may synergistically cooperate to enhance CDC killing of cells (e.g. virus-infected cells).

Where used in combination with other agents, the immunotherapeutic protein can be administered in the same pharmaceutical composition or in separate pharmaceutical compositions. If administered in separate pharmaceutical compositions, the immunotherapeutic protein and the other agent(s) may be administered simultaneously or sequentially in any order (e.g. within seconds or minutes or even hours (e.g. 2 to 48 hours)).

The immunotherapeutic protein may be formulated into a pharmaceutical composition with a pharmaceutically acceptable carrier, diluent and/or excipient. Examples of suitable carriers and diluents are well known to those skilled in the art, and are described in, for example, Remington's Pharmaceutical Sciences, Mack Publishing Co., Easton, PA 1995. Examples of suitable excipients for the various different forms of pharmaceutical compositions described herein may be found in the Handbook of Pharmaceutical Excipients, 2^{nd} Edition, (1994), Edited by A Wade and P J Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising an immunotherapeutic protein as defined in the first aspect may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, and even dyes may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising an immunotherapeutic protein as defined in the first aspect will typically be adapted for intravenous or subcutaneous administration. As such, a pharmaceutical composition may comprise solutions or emulsions which may be injected into the subject, and which are prepared from sterile or sterilisable solutions. A pharmaceutical composition may be formulated in unit dosage form (i.e. in the form of discrete portions containing a unit dose, or a multiple or sub-unit of a unit dose).

The immunotherapeutic protein, uses and pharmaceutical composition of the present disclosure are hereinafter further described with reference to the following, non-limiting examples.

### EXAMPLES

### Example 1 ACE-2-Fc Fusion Proteins Comprising an H429 Mutation and Activity Analysis

### Methods and Materials

### Constructs and Fusion Proteins

The amino acid sequence of the human ACE2 polypeptide is available from the European Nucleotide Archive (ENA, European Molecular Biology Laboratory) at accession no. BAB40370. The ectodomain of the protein (amino acids 19 to 740; shown as SEQ ID NO: 1 in Table 1) is comprised of a catalytic domain and a collectrin domain. Different forms of the ACE2 ectodomain were produced and studied in this example; particularly, a truncated (tr) ACE2 ectodomain comprising amino acids 19 to 615 of the mature ACE2 polypeptide (named trACE2 shown as SEQ ID NO: 2 in Table 1) and excluding the collectrin domain, a full length (fl) ACE2 ectodomain to (flACE2; comprising amino acids 19 to 740 of the mature ACE2 polypeptide; SEQ ID NO: 1 in Table 1), and an enhanced flACE2 ectodomain (EflACE2) comprising a triple mutation within the ACE2 polypeptide that has been reported to improve its binding affinity to the S protein (Chan et al., 2020 supra). These proteins were produced as fusion proteins with an Fc region component derived from human IgG1 to generate trACE2-Fc, flACE2-Fc and EflACE2-Fc according to standard techniques (see Table 2).

For example, a construct encoding the trACE2 ectodomain in pcDNA3.4 (Thermo Fisher Scientific) was prepared by joining a polynucleotide sequence encoding the trACE2 ectodomain to a synthetic sequence encoding a linker and a sequence encoding human IgG1 Fc (particularly, IgG1 Fc having the amino acid sequence of accession no. AXN93652.1 (immunoglobulin gamma 1 constant region, partial [Homo sapiens]; National Center for Biotechnology Information (NCBI) database). For the generation of a flACE2-Fc expression construct, a KpnI digestion of the trACE2 construct was conducted followed by insertion of a codon-optimised polynucleotide sequence encoding the ACE2 collectrin domain (GeneArt, Thermo Fisher Scientific). For the generation of an EflACE2-Fc expression construct, a synthetic polynucleotide sequence equivalent to that encoding flACE2-Fc, but with three mutations (i.e. T27Y, L79T and N330Y; sACE2.v2.4 described by Chan et al., 2020 supra) was used. In addition, variants of the fusion proteins were produced that incorporated H429F and H429Y mutations in the Fc component introduced using cleavage at a unique AfeI site within the IgG Fc-encoding sequence, and the subsequent insertion of appropriate mutagenic oligonucleotides using NEBuilder (New England Biolabs, Ipswich, MA, United States of America) according to the manufacturer's instructions.

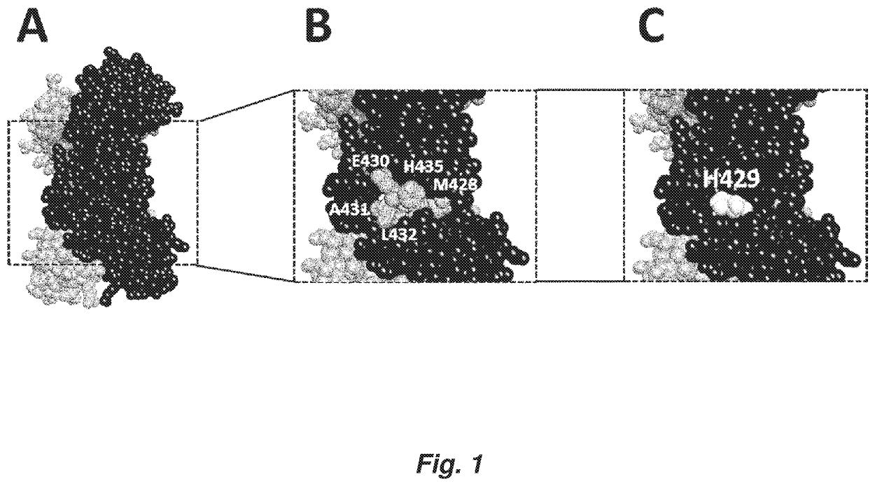

The H429 residue occupies a “buried” site within the IgG1 Fc structure (see FIG. 1) and is occupied by a histidine (His/H) residue, which is also found in the corresponding position of the Fc fragment of all other human immunoglobulin classes (FIGS. 3, 4), and accordingly is found in the corresponding position of, for example, human IgG1, IgG2, IgG3 and IgG4, as well as primate IgG subclasses and some mouse IgG subclasses. For example, the structure published in 1981 of human IgG1-Fc (PDB: 1Fc1; Deisenhofer, 1981 supra) indicates that H429 is not a surface accessible residue (FIG. 1), and in a space-filled representation of the Fc, H429 is only “visible” when overlaying residues are rendered in a non-space filling manner, as shown in FIG. 1C. Also, analysis of residues neighbouring H429 indicates that H429 lies beneath the side-chains of these residues and calculation of accessible surface area (ASA) for the Fc residues indicates that H429 is solvent inaccessible (0% ASA (Å²) FIG. 1C). H429 is also not a surface accessible residue in other Fc structures including the Fc within the structure of the anti-HIV mAb (PDB: 1HZH; Saphire et al., supra) which forms a hexameric ring structure, but H429 does not form part of the interface. Similarly, analysis of the Fc structure of the anti-Lewis Y mAb (Ramsland et al., J Immunol 187(6):3208-3217, 2011) showed that the H429 is also buried in this antibody.

TABLE 1

| SEQ ID NO: 1 | | | | | | | |
|---|---|---|---|---|---|---|---|
| | 20 | 30 | 40 | 50 | 60 | 70 | 80 |
| | ETGST | IEEQAKTFLD | KFNHEAEDLF | YQSSLASWNY | NTNITEENVQ | NMNNAGDKWS | AFLKEQSTLA |
| 90 | 100 | 110 | 120 | 130 | 140 | 150 | 160 |
| QMYPLQEIQN | LTVKLQLQAL | QQNGSSVLSE | DKSKRLNTIL | NTMSTIYSTG | KVCNPDNPQE | CLLLEPGLNE | IMANSLDYNE |
| 170 | 180 | 190 | 200 | 210 | 220 | 230 | 240 |
| RLWAWESWRS | EVGKQLRPLY | EEYVVLKNEM | ARANHYEDYG | DYWRGDYEVN | GVDGYDYSRG | QLIEDVEHTF | EEIKPLYEHL |
| 250 | 260 | 270 | 280 | 290 | 300 | 310 | 320 |
| HAYVRAKLMN | AYPSYISPIG | CLPAHLLGDM | WGRFWTNLYS | LTVPFGQKPN | IDVTDAMVDQ | AWDAQRIFKE | AEKFFVSVGL |
| 330 | 340 | 350 | 360 | 370 | 380 | 390 | 400 |
| PNMTQGFWEN | SMLTDPGNVQ | KAVCHPTAWD | LGKGDFRILM | CTKVTMDDEL | TAHHEMGHIQ | YDMAYAAQPF | LLRNGANEGE |
| 410 | 420 | 430 | 440 | 450 | 460 | 470 | 480 |
| HEAVGEIMSL | SAATPKHLKS | IGLLSPDFQE | DNETEINFLL | KQALTIVGTL | PFTYMLEKWR | WMVFKGEIPK | DQWMKKWWEM |
| 490 | 500 | 510 | 520 | 530 | 540 | 550 | 560 |
| KREIVGVVEP | VPHDETYCDP | ASLFHVSNDY | SFIRYYTRTL | YQFQFQEALC | QAAKHEGPLH | KCDISNSTEA | GQKLENMLRL |
| 570 | 580 | 590 | 600 | 610 | 620 | 630 | 640 |
| GKSEPWTLAL | ENVVGAKNMN | VRPLLNYFEP | LFTWLKDQNK | NSFVGWSTDW | SPYADQSIKV | RISLKSALGD | RAYEWNDNEM |
| 650 | 660 | 670 | 680 | 690 | 700 | 710 | 720 |
| YLFRSSVAYA | MRQYFLKVKN | QMILFGEEDV | RVANLKPRIS | FNFFVTAPKN | VSDIIPRTEV | EKAIRMSRSR | INDAFRLNDN |
| 730 | 740 | 750 | 760 | 770 | 780 | 790 | 800 |

Fc (EU numbering)

| | | | | | | | |
|---|---|---|---|---|---|---|---|
| SLEFLGIQPT | LGPPNQPPVS | GGGGSTHTCP | PCPAPELLGG | PSVFLFPPKP | KDTLMISRTP | EVTCVVVDVS | HEDPEVKENW |
| 810 | 820 | 830 | 840 | 850 | 860 | 870 | 880 |
| YVDGVEVHNA | KTKPREEQYN | STYRVVSVLT | VLHQDWLNGK | EYKCKVSNKA | LPAPIEKTIS | KAKGQPREPQ | VYTLPPSREE |
| 890 | 900 | 910 | 920 | 930 | 940 | 950 | 960 |
| MTKNQVSLTC | LVKGFYPSDI | AVEWESNGQP | ENNYKTTPPV | LDSDGSFFLY | SKLTVDKSRW | QQGNVFSCSV | MHEALHNHYT |
| 970 | | | | | | | |
| QKSLSLSPGK | | | | | | | |

Comments: SEQ ID NO: 1 is the predicted sequence of the mature “flACE2-Fc” fusion
protein. The full length (fl) ACE2 ectodomain comprises the catalytic domain and
the collectrin (or neck) domain and sequence proximal to the transmembrane region.
The N-terminus sequence ETG is predicted to remain following cleavage of the
following underlined leader sequence (MGILPSPGMPALLSLVSLLSVLLMGCVAETG; SEQ ID NO: 120)
upon secretion. The numbering above the fusion protein sequence utilises the numbering
of ACE2, amino acid residues S19 to S740, then a linking sequence (GGGGS; SEQ ID NO:
121), followed by a human IgG1 Fc comprising T223-K447 (according to the Eu numbering
convention, labeled immediately above the C-terminus Fc sequence) and includes the CH2
and CH3 domains. The IgG1 Fc amino acid, H429, is shown in bold and underlined
(Eu numbering). The ACE2 ectodomain sequence matches that of Accession no. BAB40370.
Also, a variant immunotherapeutic protein ″EflACE2-Fc″ was constructed whereby, the
amino acids at positions 27, 79 and 330 were mutated to enhance binding affinity to
CoV-2 S as follows: T27Y, L79T and N330Y (Chan et al., 2020 supra). The IgG1 Fc
sequence matches the immunoglobulin gamma 1 constant region, partial [Homo sapiens]
sequence of Accession no. AXN93652.1.

| SEQ ID NO: 2 | | | | | | | |
|---|---|---|---|---|---|---|---|
| | 20 | 30 | 40 | 50 | 60 | 70 | 80 |
| | ETGST | IEEQAKTFLD | KFNHEAEDLF | YQSSLASWNY | NTNITEENVQ | NMNNAGDKWS | AFLKEQSTLA |
| 90 | 100 | 110 | 120 | 130 | 140 | 150 | 160 |
| QMYPLQEIQN | LTVKLQLQAL | QQNGSSVLSE | DKSKRINTIL | NTMSTIYSTG | KVCNPDNPQE | CLLLEPGLNE | IMANSLDYNE |
| 170 | 180 | 190 | 200 | 210 | 220 | 230 | 240 |
| RLWAWESWRS | EVGKQLRPLY | EEYVVLKNEM | ARANHYEDYG | DYWRGDYEVN | GVDGYDYSRG | QLIEDVEHTF | EEIKPLYEHL |
| 250 | 260 | 270 | 280 | 290 | 300 | 310 | 320 |
| HAYVRAKLMN | AYPSYISPIG | CLPAHLLGDM | WGRFWTNLYS | LTVPFGQKPN | IDVTDAMVDQ | AWDAQRIFKE | AEKFFVSVGL |
| 340 | 350 | 360 | 370 | 380 | 390 | 400 | 410 |
| PNMTQGFWEN | SMLTDPGNVQ | KAVCHPTAWD | LGKGDFRILM | CTKVTMDDEL | TAHHEMGHIQ | YDMAYAAQPF | LLRNGANEGE |
| 410 | 420 | 430 | 440 | 450 | 460 | 470 | 480 |
| HEAVGEIMSL | SAATPKHLKS | IGLLSPDFQE | DNETEINFLL | KQALTIVGTL | PFTYMLEKWR | WMVFKGEIPK | DQWMKKWWEM |
| 490 | 500 | 510 | 520 | 530 | 540 | 550 | 560 |
| KREIVGVVEP | VPHDETYCDP | ASLFHVSNDY | SFIRYYTRTL | YQFQFQEALC | QAAKHEGPLH | KCDISNSTEA | GQKLENMLRL |
| 570 | 580 | 590 | 600 | 610 | 620 | 630 | 640 |
| | | | | | | 230 | 240 |
| GKSEPWTLAL | ENVVGAKNMN | VRPLLNYFEP | LFTWLKDQNK | NSFVGWSTDW | SPYADGSGSG | SGTHTCPPCP | APELLGGPSV |
| 650 | 660 | 670 | 680 | 690 | 700 | 710 | 720 |
| 250 | 260 | 270 | 280 | 290 | 300 | 310 | 320 |
| FLFPPKPKDT | LMISRTPEVT | CVVVDVSHED | PEVKENWYVD | GVEVHNAKTK | PREEQYNSTY | RVVSVLTVLH | QDWLNGKEYK |
| 730 | 740 | 750 | 760 | 770 | 780 | 790 | 800 |
| 330 | 340 | 350 | 360 | 370 | 380 | 390 | 400 |
| CKVSNKALPA | PIEKTISKAK | GQPREPQVYT | LPPSREEMTK | NQVSLTCLVK | GFYPSDIAVE | WESNGQPENN | YKTTPPVLDS |
| 810 | 820 | 830 | 840 | 847 | | | |
| 410 | 420 | 430 | 440 | 447 | | | |
| DGSFFLYSKL | TVDKSRWQQG | NVFSCSVMHE | ALHNHYTQKS | LSLSPGK | | | |

Comments: SEQ ID NO: 2 is the predicted sequence of the mature “trACE2-Fc” fusion
protein. The N-terminus sequence ETG is predicted to remain following cleavage of
the following underlined leader sequence (MGILPSPGMPALLSLVSLLSVLLMGCVAETG; SEQ ID NO: 120)
upon secretion. The numbering above the fusion protein sequence utilises the numbering of
ACE2, amino acid residues S19 to D615, then a linking sequence (GSGSGSG; SEQ ID NO: 122),
followed by a human IgG1 Fc, and which, according to the Eu numbering convention,
comprises T223-K447. The IgG1 Fc amino acid, H429, is shown bold and underlined. The
truncated ACE2 ectodomain sequence matches that of Accession no. BAB40370. The IgG1 Fc
sequence matches the immunoglobulin gamma 1 constant region, partial [Homo sapiens]
sequence of Accession no. AXN93652.1.

Expression of the fusion proteins was conducted using transient transfection of Expi293 cells (Thermo Fisher Scientific). All expressed fusion proteins were first purified from the culture supernatant by ion exchange chromatography (IEX) followed by additional purification by size exclusion chromatography (SEC). Particularly, the supernatant of the Expi293 cells transiently transfected for the expression of the respective ACE2-Fc fusion protein (where the Fc region was according to the wild type (WT) hIgG1 Fc mentioned in the preceding paragraph) was extensively dialysed against 10 mM Tris-HCl pH 8.0 and applied to a High-Q column (BioRad Laboratories, Hercules, CA, United States of America). Bound proteins were eluted with a linear gradient to buffer A containing 0.4 M NaCl. Fractions were examined by SDS-PAGE, and those fractions containing the flACE2-Fc WT fusion protein were pooled and concentrated using a 30 kDa cut-off filtration device (Pall Corporation, Port Washington, NY, United States of America) and separated by size exclusion chromatography (SEC) using a Superose 6 column (GE Life Sciences, Chicago, IL, United States of America).

The SARS-CoV-2 RBD-Ig and RBD AviTag have been described previously. The RBD AviTag was biotinylated in situ using Expi293BirA cells (Wines B D et al., J Immunol 197(4):1507-1516, 2016).

Lamelli native PAGE (N-PAGE), 150V, 2.5 h, 4° C., was according to Wines B D et al., J Immunol 162(4):2146-2153, 1999).

### Virus Neutralisation Assays

Antiviral titre was determined using SARS-CoV-2 (CoV/Australia/VICO1/2020) in a microneutralisation assay as described previously (Juno J A et al., Nat Med 26(9):1428-1434, 2020).

### Bio-Layer Interferometry

Measurements of the affinity of the ACE2-Fc fusion proteins for the CoV-2 S RBD were performed on the Octet RED96e (FortéBio, Fremont, CA, United States of America). All assays were performed at 25° C. using anti-human IgG Fc capture (AHC) biosensor tips (FortéBio) in kinetics buffer (phosphate buffered saline (PBS) pH 7.4 supplemented with 0.1% (w/v) bovine serum albumin (BSA) and 0.05% (v/v) TWEEN-20). After a 60 second (60s) biosensor baseline step, the fusion proteins (20 mg/mL) were loaded onto anti-human IgG Fc capture (AHC) biosensors by submerging sensor tips for 200s and then washing in kinetics buffer for 60s. For most of the fusion proteins, association measurements were performed by dipping into a two-fold dilution series of SARS-CoV-2 RBD from 16-250 or 500 nM for 180s and then measuring dissociation in kinetics buffer for 180s. For EflACE2-Fc WT, a two-fold dilution series of 2-31 or 63 nM was used. The biosensor tips were regenerated five times using a cycle of 5s in 10 mM glycine pH 1.5 and 5s in kinetics buffer, and baseline drift was corrected by subtracting the average shift of a fusion protein-loaded sensor not incubated with SARS-CoV-2 RBD, and an unloaded sensor incubated with SARS-CoV-2 RBD. Curve fitting analysis was performed with Octet Data Analysis 10.0 software using a global fit 1:1 model to determine K_{D} values and kinetic parameters. Curves that could not be fitted were excluded from the analyses. Kinetic constants reported were representative of two independent experiments.

ACE2-Fc Fusion Proteins and Recombinant Dimeric rsFcγR Binding by Flow Cytometry

The ACE2-Fc fusion proteins or rituximab (a chimeric mAb targeted to CD20), at 5 μg/ml, or the indicated concentrations were incubated with Ramos cells expressing transfected spike protein (Ramos-S cells; Lee W S et al., medRxiv doi:10.1101/2020.12.13.20248143, 2020) at 5×10⁶ cells/ml in 25 μl of PBS containing 0.5% (w/v) BSA and 1 mM glucose (PBS/BSA/G), for 30 minutes on ice, and then washed twice with PBS/BSA/G, incubated with PE or FITC conjugated anti-human IgG-Fc for 30 minutes on ice, before being washed again and resuspended in 25 μl of PBS/BSA/G.

Evaluation of binding of dimeric recombinant soluble FcγR (rsFcγR) was performed as previously described (Wines et al., 2016 supra). The ACE2-Fc opsonised Ramos-S cells or rituximab-opsonised cells were resuspended in 0.5 μg/ml of biotinylated dimeric rsFcγRIIa (H131 allelic form) or dimeric rsFcγRIIIa (V158 allelic form) or BSA/PBS/G and incubated for 30 minutes on ice followed by 1/500 streptavidin-APC (or anti-hIgG-Fc FITC for confirmation of ACE2-Fc opsonisation) for 20 minutes on ice. The cells were washed, resuspended in PBS/BSA/G and analysed on a Canto™ II flow cytometer (Becton Dickinson, Franklin Lakes, NJ, United States of America).

### Complement Dependent Cytotoxicity (CDC) Assay

CDC was measured using Ramos-S cells opsonised with ACE2-Fc function proteins or the control mAb, rituximab, then incubation with human serum as a source of complement. Thus, Ramos-S cells were first incubated with a fusion protein or rituximab as above (5×10⁶ cells/ml in 25 μl of PBS/BSA/G for 30 minutes on ice), then washed before resuspending in 1/3 diluted normal human serum for 30 minutes at 37° C. Cells were washed twice with PBS and the dead cells were then enumerated by staining with 1/500 Zombie Green (from a Zombie Green Fixable Viability kit according to the manufacturer's instruction, BioLegend, San Diego, CA, United States of America) before fixing with 2% paraformaldehyde in PBS and analysis on a Canto™ II flow cytometer (Becton Dickinson).

Complement Fixation Immunoassay for ACE2-Fc Fusion Proteins Ninety-six well flat-bottom MaxiSorp Nunc plates (Thermo Fisher Scientific) were coated with 5 μg/ml Avidin in PBS overnight, blocked, and then incubated with either a serial two-fold dilution or single concentration (2.5 μg/ml) of biotinylated RBD (Hartley et al., Science Immunology 5(54) doi:10.1126/sciimmunol.abf8891, 2020) in 0.1% casein for 1 hour at RT. The ACE2-Fc fusion proteins were then added over the indicated concentration range. To measure C1q fixation, the plates were incubated with 10 μg/ml purified human C1q (Merck Millipore, Burlington, MA, United States of America) for 30 minutes at RT followed by 1/2000 dilution of rabbit anti-C1q IgG (Kurtovic L et al., BMC Med 17:45 2019) for 1 hour at RT. In experiments to measure C5b-C9 fixation, the plates were incubated with 10% fresh human serum for 30 minutes at RT followed by 1/2000 dilution of rabbit anti-C5b-C9 (Merck Millipore) for 1 hour at RT washed and then incubated with goat anti-rabbit IgG conjugated to HRP (Merck Millipore) at 1/2000 dilution for 1 hour at RT followed by TMB substrate (Life Technologies Corporation, Carlsbad, CA, United States of America) for 15-20 minutes at RT. Reactivity was stopped using 1 M sulfuric acid and absorbance was measured at OD450 nm. Test samples and reagents were prepared in PBS supplemented with 0.1% (w/v) casein and plates washed three times between each step using PBS containing 0.05% (v/v) TWEEN-20. Samples were tested in duplicate and corrected for background reactivity using negative control wells from which ACE2-Fc proteins were omitted. The mean and SEM from independent experiments are shown.

### RBD Variant Multiplex Assay

A custom multiplex array was produced with the SARS-1 S 1 subunit (ACROBiosystems, Newark, DE, United States of America), SARS-Cov-2 S1 and HCoV NL63 S1 and S2 subunits (Sino Biological Inc., Beijing, China), NL63 S trimer (BPS Bioscience, San Diego, CA, United States of America), and a hexahistidine-tagged RBD WT protein (amino acids 19-613) and 21 variants identified from the GISAID RBD surveillance repository that had been expressed from pcDNA3 in Expi293 cells and purified by affinity chromatography. Bead coupling, washing steps, and data acquisition on a FlexMap3D™ analyser (Luminex Corporation, Austin, TX, United States of America) was as previously described (Lee et al., 2020 supra). Briefly, direct binding of the ACE2-Fc fusion proteins trACE2-Fc, flACE2-Fc and EflACE2-Fc at 0.5 to 250 nM, were detected using 25 μl of 1.3 μg/ml anti-human IgG R-Phycoerythrin Conjugate (SouthemBiotech, Birmingham, AL, United States of America). Data were fitted as 3-parameter agonist versus response curves (r²>0.85) to determine each EC₅₀. In a competition assay, RBD- or S1-coupled beads were simultaneously incubated with 20l of 25 μg/ml of biotinylated, AviTagged ACE2 in the presence of the various unbiotinylated ACE2-Fc fusion protein “competitors”, at 1 to 280 nM for 2 hour at RT. The binding of biotinylated ACE2 (aa 19 to 615) was performed using, first, Streptavidin, R-Phycoerythrin Conjugate (SAPE) (Thermo Fisher Scientific) at 4 μg/ml (1 hour), followed by 10 μg/ml of R-Phycoerythrin, Biotin-XX Conjugate (Thermo Fisher Scientific) (1 hour).

### FcγRIIIa-NF-κB-RE Nanoluciferase Reporter Assay

FcγRIIIa-NF-κB-RE nanoluciferase reporter assays were conducted using IA1.6/FcR-γ/FcγRIIIa V158 cells expressing a NF-κB response element-driven nanoluciferase (NanoLuc, pNL3.2.NF-κB-RE[NlucP/NF-κB-RE/Hygro], Promega Corporation, Madison, WI, United States of America), and was performed essentially as previously described (Lee et al., 2020 supra). Briefly, Ramos cells expressing Spike-IRES-orange2 were used as target cells and were incubated with agonists and the FcγRIIIa/NF-κB-RE reporter cells for 5 hours before measurement of induced nanoluciferase with Nano-Glo substrate (Promega Corporation).

### Results and Discussion

### ACE2-Fc Fusion Protein Construction and Production

A series of ACE2-Fc fusion proteins (Table 2) were produced and analysed for their capacity to neutralise SARS-CoV-2 infection and mediate Fc-dependent effector functions normally attributed to the mechanisms of action of antibodies.

TABLE 2

| | | | IgG1 Fc |
|---|---|---|---|
| | ACE2 | ACE2 | mutation/ |
| | ectodomain form | mutation/ | modification |
| Protein name | (sequence) | modification | (Eu numbering) |
| trACE2 | Truncated ACE2 | Not mutated | N/A* |
| | (amino acids 19-615) | | |
| trACE2-Fc WT | Truncated ACE2 | Not mutated | Not modified |
| | (amino acids 19-615) | | |
| trACE2-Fc- | Truncated ACE2 | Not mutated | His 429 Phe |
| H429F | (amino acids 19-615) | | |
| trACE2-Fc- | Truncated ACE2 | Not mutated | His 429 Tyr |
| H429Y | (amino acids 19-615) | | |
| trACE2-Fc-kif | Truncated ACE2 | Not mutated/ | Modified |
| | (amino acids 19-615) | modified | glycan at |
| | | N-glycans | Asn 297 |
| flACE2-Fc WT | Full length ACE2 | Not mutated | Not modified |
| | (amino acids 19-740) | | |
| flACE2-Fc- | Full length ACE2 | Not mutated | His 429 Phe |
| H429F | (amino acids 19-740) | | |
| flACE2-Fc- | Full length ACE2 | Not mutated | His 429 Tyr |
| H429Y | (amino acids 19-740) | | |
| EflACE2-Fc WT | Full length ACE2 | Thr 27 Tyr | Not modified |
| | (amino acids 19-740) | Leu 79 Thr | |
| | | Asn 330 Tyr | |
| EflACE2-Fc- | Full length ACE2 | Thr 27 Tyr | His 429 Phe |
| H429F | (amino acids 19-740) | Leu 79 Thr | |
| | | Asn 330 Tyr | |
| EflACE2-Fc- | Full length ACE2 | Thr 27 Tyr | His 429 Tyr |
| H429Y | (amino acids 19-740) | Leu 79 Thr | |
| | | Asn 330 Tyr | |

*N/A: Not applicable as no Fc present (i.e. truncated ectodomain only)

In an attempt to enhance the avidity of binding to the SARS-CoV-2 spike protein or to confer and improve Fc-dependent effector functions, three versions of the ACE2 ectodomain were fused to an Fc region of IgG1 either unmodified or altered by mutation (i.e. substitution of histidine 429 with phenylalanine (H429F) or tyrosine (H429Y)) or modified glycosylation (i.e. lacking core fucose; trACE2-Fc-kif).

### ACE2-Fc Fusion Protein Purification

Proteins were produced in Expi293 cells and purified by (an) ion exchange (IEX) then by size exclusion chromatography (SEC) at pH 7.4 (FIG. 5). All of the fusion proteins showed a similar IEX purification profile to that of flACE2-Fc-WT (FIG. 5A) which contained a major elution peak (peak *; FIG. 5A), and SDS-PAGE analysis of the collected fractions showed a single major species of about 270 kDa (FIG. 5B). Except for those fusion proteins comprising the H429Y mutation, SEC analysis and purification confirmed the presence of a major monomeric species (FIG. 5C) (NB. The monomer species are considered to be single molecules (i.e. monomer molecules) comprising two (dimerised) copies of the respective ACE2-Fc fusion protein) which were collected for further analysis, and only trace quantities of higher molecular weight oligomers and other impurities were apparent for example for flACE2-Fc-WT (FIG. 5). Indeed, the presence of a similar monomeric species was also evident following the IEX purification of all fusion proteins with unmodified Fc-WT or an Fc region component including an H429F modification (not shown). For the fusion proteins comprising the H429Y mutation, SEC analysis (FIG. 5D) (i.e. of the major IEX peak from flACE2-Fc H429Y mutant, not shown), and also of the major IEX peaks from the trACE2-Fc H429Y and EflACE2-Fc H429Y proteins (not shown)), revealed the presence of significant quantities of oligomeric (Yₒₗᵢ) and monomeric (Yₘₙ) species, which were subsequently evaluated separately for functional activity.

### Binding of ACE2-Fc Fusion Proteins RBD of SARS-CoV-2 S by ELISA

The binding of the trACE2-Fc WT, flACE2-Fc WT and EflACE2-Fc WT fusion proteins to SARS-CoV-2 receptor binding domain (RBD) (FIG. 6A-C) were similar overall (i.e. EC₅₀ 0.35 nM, 0.27 nM and 0.25 nM respectively; FIG. 6D) with the enhanced intrinsic affinity of the EflACE2-Fc being less apparent with binding to this bivalent form of RBD, which was presented in fusion with mouse IgG1 Fc (RBD-Ig). The binding activity of the various fusion proteins with mutated Fc components were also equivalent, excepting the monomeric flACE2-Fc-H429Y which was of slightly lower affinity (EC₅₀ 0.48 nM) (FIG. 6D) and the other H429Y Fc variants likewise trending to a lower level of affinity (FIG. 6).

### Oligomerisation of the ACE2-Fc H429Y Fusion Protein is pH Dependent

Oligomerisation of fusion proteins including a mutated H429Y Fc component was examined by SEC separation at pH 5.0 of the flACE2-Fc-H429Y prepared by IEX. In contrast to SEC at pH 7.4 (FIG. 5D and FIG. 7A), SEC at pH 5.0 (FIG. 7B) revealed a greater proportion of monomer Yₘ which N-PAGE showed was purified to homogeneity (FIG. 7C: lane 1 cf lane 2). The purified monomeric flACE2-Fc-H429Yₘₙ, pH 5.0, was re-analysed by dialysis at pH 7.4 followed by both N-PAGE (FIG. 7C) and by SEC at pH 7.4 (FIG. 7D). Re-exposure to pH 7.4 yielded a mix of oligomer Yₒₗᵢ and monomer Yₘₙ species (FIG. 7C; lane 2 pH 5.0 cf lane 5 pH 7.4) indicating some equilibrium between these forms occurs at neutral pH. The flACE2-Fc-H429Yₘₙ prepared at pH 5.0 showed equivalent binding to RBD-Ig as the other flACE2-Fc WT fusion proteins and Fc variants (FIG. 7E). It is also clear that the prevalence of oligomers is related to the tyrosine substitution of histidine 429 as this was not observed in the fusion proteins with phenylalanine-substituted H429F Fc region component.

### Evaluation of Virus Neutralisation Potency

The antiviral activities of the ACE2-Fc fusion proteins was determined in a micro-neutralisation assay of SARS-CoV-2 infection of Vero cells (FIG. 8) where the EC₅₀ endpoint corresponds to neutralisation of ˜99% of the inoculum virions (Khoury D S et al., Nat Rev Immunol 20(12):727-738, 2020).

The SARS-CoV-2 neutralisation endpoint of the truncated ectodomain, trACE2 (2.70 μM), was improved ˜10-fold by fusion to the wild type Fc region of IgG1 (trACE2-Fc WT, 283 nM) (FIG. 8). The improved potency is consistent with improved avidity of binding to SARS-CoV-2 spike RBD because of ACE2-Fc bivalency resulting from fusion of ACE2 ectodomains to the IgG Fc region and was also similar to that of flACE2-Fc WT. The EflACE2-Fc-WT showed a further ˜20-fold improvement (11 nM) over the flACE2-Fc WT and trACE2-Fc WT fusion proteins and ˜200-fold more than un-fused trACE2.

Analysis of the Fc modifications revealed several interesting differences. First, the H429Y mutation in trACE2-Fc and flACE2-Fc improved their virus neutralisation potency (FIG. 8); this was surprising as the neutralisation function of the ACE2-Fc fusion proteins occurs well away from the modifications at the H429 position (i.e. in the fusion proteins, the ACE2 polypeptide is expected to neutralise the virus, but the H429 residue is distal to the ACE2 being present in the CH3 domain of the Fc component). Secondly, it was found that the neutralisation activity (21.9 nM) of the oligomeric form of trACE2-Fc-H429Yₒₗᵢ isolated by SEC at pH 7.4 was enhanced 13-fold over the trACE2-Fc WT. Both the oligomer, Yₒₗᵢ, and the monomer, Yₙₘ, forms of flACE2-Fc-H429Y comprising two (dimerised) copies of the ACE2-Fc fusion protein (FIG. 5B) (endpoints of 10.0 and 20.9 nM respectively; FIG. 8) showed greater potency than the flACE2-Fc WT (124 nM) fusion protein and was similar to the viral neutralisation achieved with EflACE2-Fc WT (10.6 nM), but EflACE2-Fc-H429Yₒₗᵢ was seemingly more potent (4 nM) which represents a neutralisation activity of the EflACE2-Fc WT (10.6 nM) that is over 200-fold greater than that of the unfused trACE2, or in the case of EflACE2-Fc-H429Y (4 nM), over 600 fold greater (FIG. 8). Thus, the Fc-H429Y mutation increased SARS-CoV-2 neutralisation in the trACE2-Fc and flACE2-Fc fusion protein formats, and also trended towards a greater level of potency when combined with the triple mutation of the ACE2 in the intrinsically higher affinity EflACE2-Fc.

The phenylalanine substitution of histidine 429 (H429F) of the ACE2-Fc fusion proteins did not enhance neutralisation.

Interaction of ACE2-Fc Fusion Proteins with FcγR

The interaction of FcγRIIa and FcγRIIIa with the ACE2-Fc fusion proteins was evaluated by flow cytometry using ACE2-Fc opsonised Ramos-S cells and dimeric recombinant soluble FcγR (Wines et al., 2016 supra). The trACE2-Fc, flACE2-Fc and EflACE2-Fc fusion proteins all bound to FcγRIIa and FcγRIIIa (FIG. 9A, 9B), however, the variant proteins comprising a mutated H429Y Fc component largely ablated binding to both of these Fc receptor types.

### FcγRIIIa Activation by ACE2-Fc Fusion Proteins

Antibody dependent cytotoxicity (ADCC) and Fc-dependent clearance of viruses are important antiviral effector mechanisms that may play a protective role during SARS-CoV-2 infection (Li D et al., bioRxiv doi: 10.1101/2020.12.31.424729, 2021; and Shafer A et al., J Exp Med 218(3):e20201993, 2021). Thus, in this experimentation, the ability of the trACE2-Fc, flACE2-Fc and EflACE2-Fc fusion proteins and variant proteins to activate FcγRIIIa was evaluated. It was found that Ramos-S cells opsonised with ACE2-Fc fusion proteins comprising the wild type (WT) Fc region all initiated FcγRIIIa activation (FIG. 10). However, it was surprising that the flACE2-Fc fusion protein induced a greater level of activation than trACE2-Fc indicating that the inclusion of the collectrin domain in the ACE2 component of the fusion protein substantially, and surprisingly, improves the potency of FcγRIIIa activation by the ACE2-Fc fusion protein. Further, it was found that the increased affinity that the EflACE2-Fc fusion protein has for the SARS-CoV-2 spike protein, did not alter the level of FcγRIIIa activation which was equivalent to that of flACE2-Fc (FIG. 10D).

Production of the trACE2-Fc fusion protein in the presence of kifunensine (van Berkel P H C et al., Biotechnol Bioeng 105(20:350-357, 2010), that is trACE2-Fc-kif also improved a modest level of FcγRIIIa activation shown by trACE2-Fc to a level comparable to that of the flACE2-Fc and EflACE2-Fc fusion proteins (FIG. 10D) and approaching that of the therapeutic anti-CD20 mAb, rituximab, on the CD20⁺ Ramos-S cells (FIG. 10A,10D). Kifunensine, a mannosidase inhibitor, prevents normal N-linked glycosylation, including core fucosylation and, in immunoglobulins, the lack of fucose on the heavy chain glycan at Asn297 is known to improve FcγRIIIa binding and activation (Ferrara C et al., Proc Nat Acad Sci USA 108(31):12669-12674, 2011). Thus, it was hypothesised that similar treatment of the flACE2-Fc and EflACE2-Fc fusion proteins, or amino acid residue substitution to increase affinity for FcγRIIIa (Wang et al., 2018 supra), would be likely to further improve their FcγRIII activating potency.

Modification of the trACE2-Fc, flACE2-Fc and EflACE2-Fc fusion proteins by including an H429F Fc component mutation did not affect FcγRIII activation by opsonised Ramos-S cells (FIG. 10). In contrast, the inclusion of the mutated H429Y Fc component in all ACE2-Fc fusion proteins ablated FcγRIIIa activation of cells, which was consistent with the abovementioned failure to bind to Fc receptors, especially FcγRIIIa (FIG. 9B). Thus, while enhancing virus neutralisation, the H429Y modified Fc component in the trACE2-Fc, flACE2-Fc and EflACE2-Fc fusion proteins were inactive in FcγR binding (FIG. 9) and in activating cells through FcγRIIIa (FIG. 10). The hierarchy of FcγRIIIa activation by the fusion proteins was flACE2-Fc WT=EflACE2-Fc >trACE2-Fc emphasising that the presence of the collectrin domain is a key component in optimal activation of FcγRIIIa by the ACE2-Fc fusion proteins.

### Complement Fixation and Activation, and Lysis of SARS-CoV-2 Spike Cells by ACE2 Fc Proteins

The ACE2-Fc fusion proteins comprising mutated Fc region components were examined for their capacity to fix complement components C1q and C5-C9 in an ELISA-based analysis using avidin-immobilised RBD-biotin (FIG. 11A-F), and importantly to mediate complement-dependent killing of cells expressing SARS-CoV-2 spike protein (FIG. 11G). When the RBD was limiting (FIG. 11A, 11B), the activity of the fusion proteins comprising a mutated H429F Fc component was surprisingly enhanced over the counterparts comprising a wild type Fe region. Additionally, the binding of C5b-9 (FIG. 11E, 11F) which forms the membrane attack complex on cells, was equivalent for the fusion proteins comprising a mutated H429F Fc region component. Further, despite their oligomerisation (FIGS. 6 and 7), a feature associated with superior CoV-2 neutralisation activities (FIG. 8), both the trACE2-Fc-H429Y and flACE2-Fc-H429Y fusion proteins showed little difference in C1q or C5-9 fixation over the counterparts comprising a wild type Fc region (FIG. 11A-F). Moreover, it was apparent that the trACE2-Fc fusion protein was more potent in fixing complement C1q than flACE2-Fc (cf FIG. 11C, 11E with 11D, 11F); due to the presence of the collectrin dimerisation domain in flACE2-Fc possibly reducing the segmental flexibility of the fusion protein and thereby negatively affecting complement C1q binding. Notably however, this difference was much reduced by the inclusion of the H429F Fc mutation of the trACE2-Fc-H429F and flACE2-Fc-H429F fusion proteins; particularly evident in the fixation of the C5b-C9 complex (FIG. 11E, 11F).

Surprisingly, the fusion proteins comprising a mutated H429F Fc region component, were the only highly active ACE2-Fc fusion proteins in serum complement-dependent cytotoxicity (CDC) of Ramos-S cells (FIG. 11G). Despite the capacity of the trACE2-Fc WT, as well as the trACE2-Fc-kif fusion proteins and flACE2-Fc WT, to fix C1q and C5b-9 in the ELISA assay (FIG. 11A, 11C, 11E), these could not induce complement mediated cell death nor could EflACE2-Fc WT despite higher affinity for the SARS-CoV-2 spike protein. The trACE2-Fc-H429Yₘₙ fusion protein was also active in complement mediated killing albeit weakly.

Since it is well known that the activation of the complement cascade can also lead to, for example, the phagocytosis of cells, microbes or particles opsonised with C1q (or other complement fragments such as, for example C3b or C3bi; Ricklin D et al., Immunol Rev 274(1):33-58, 2016) which bind to specific cell surface receptors such as, for example, CR1 or CR3 (Vandendriessche S et al., Front Cell Dev Biol 9:624025, 2021) on phagocytic cells, it is considered that the observed enhancement of Fc-dependent complement lysis by the ACE2-Fc fusion proteins including an H429Y mutation, will also be reflected in similar enhancements in complement-dependent phagocytosis of ACE2-Fc-coated targets (e.g. virions) through the complement receptors.

### Conclusion

It has been found that immunotherapeutic proteins in the form of fusion proteins comprising an angiotensin converting enzyme 2 (ACE2) polypeptide (or a fragment thereof) fused to an Fc region component comprising an H429 mutation provides considerable potential for the treatment or prevention of coronavirus infection. Selection of, for example, a full length or truncated (i.e. fragment) of ACE2, and various modifications of the Fc component may also enable considerable “tuning” of the antiviral agent to modify the action(s) by which the antiviral effect is achieved. For example, by including an Fc component in the immunotherapeutic protein comprising an H429Y substitution, an oligomeric immunotherapeutic protein may be produced which shows increased virus neutralisation, and abrogated FcγR binding and activation. On the other hand, by including an Fc component in the immunotherapeutic protein comprising an H429F substitution, an antiviral effect including complement-dependent cytotoxicity (CDC) of spike protein-expressing cells (e.g. infected cells) may be achieved. Thus, these unique Fc mutations enable complementary approaches to tuning the function of the Fc region component that may aid in the development of ACE2-Fc fusion proteins and other fusion proteins of therapeutic significance by allowing the selection of desired functional profiles.

### Example 2 Chimeric Anti-TNP IgG1 Antibodies Comprising an H429 Mutation and Activity Analysis

### Methods and Materials

### Production of Anti-TNP Human IzG and Mutant Anti-TNP Human IgG Plasmid Constructs

Chimeric anti-TNP human IgG antibody constructs consisting of the variable heavy (V_{H}) and light (V_{L}) region sequence of the mouse anti-trinitrophenyl (anti-TNP) antibody TIB142 and the sequence from the constant heavy (CH) region from human IgG subclasses have been described previously in detail—hIgG1 (Patel D et al., J Immunol 184(11):6283-6292, 2010), hIgG2 and hIgG4 (Wines et al., 2016 supra). All chimeric antibody sequences were subcloned into the pCR3vector. Mutations for H429F, H429Q, H429E, H429S, H433A and H435A substitutions were made into the cDNA sequence encoding the Fc region component using standard molecular biology techniques.

### Expression and Production of IgG Antibodies by Expi293 Cells

The IgG antibodies were produced in Expi293 human embryonic kidney cells as described previously (Wines et al., 2016 supra). Briefly, Expi293 cells were maintained in Expi293 Expression Medium (Gibco, Waltham, MA, United States of America) for both cell growth and protein production. Cells were transfected simultaneously with the IgG heavy chain plasmid (15 μg) and light chain plasmid (15 g) diluted in Opti-MEM I Reduced-Serum Medium (Gibco) using the Expifectamine transfection kit (Life Technologies) then cultured for four days. Culture supernatants were clarified by centrifugation and filtered through a 0.2 μm filter after which the IgGs were purified by affinity chromatography using a Hi-Trap HP Protein A column (GE Healthcare Life Sciences, Marlborough, MA, United States of America) and eluted with 0.1 M citric acid, pH 3.5, followed by neutralisation with 1M Tris-HCl, pH 9.0 and dialysation against PBS pH 7.5. Aggregates were removed by subsequent gel filtration on a Superose 6 10/300GL column (GE Healthcare Life Sciences) and monomeric IgG peak fractions collected. The antigen binding activity of all antibody preparations was tested on BSA-TNP by ELISA as described (Wines et al, 2016 supra).

Evaluation of Antigen Binding Activity by Monoclonal Anti-TNP mAbs

The anti-TNP mAbs were evaluated by ELISA for antigen binding activity by binding to TNP haptenylated BSA (TNP-BSA) as previously described in Wines et al., 2016 supra.

ELISA Immunoassay for Detection of Complement Fixation by mAbs

Ninety-six well flat-bottom MaxiSorp Nunc plates (Thermo Fisher Scientific) were coated with 20 μg/ml TNP-BSA in PBS overnight at 4° C. The following morning, plates were blocked with 0.1% (w/v) casein in PBS for 2 hours at 37° C. and then incubated with anti-TNP mAbs (concentrations between 4 and 0.125 g/ml) for 2 hours at RT. To measure C1q fixation, the TNP:anti-TNP plates were incubated with 10 g/ml purified human C1q (Merck Millipore) for 30 minutes at RT followed by 1/2000 dilution of rabbit anti-C1q IgG (Kurtovic L et al., BMC Med 17:45 2019) for 1 hour at RT. In experiments to measure C5b-C9 fixation, the TNP:anti-TNP plates were incubated with 10% fresh human serum for 30 minutes at RT followed by a 1/2000 dilution of rabbit anti-C5b-C9 (Merck Millipore) for 1 hour at RT, before washing and then incubation with goat anti-rabbit IgG conjugated to HRP (Merck Millipore) at 1/2000 dilution for 1 hour at RT followed by TMB substrate (Life Technologies) for 15-20 minutes at RT. Reactivity was stopped using 1M sulfuric acid and absorbance was measured at OD450 nm. Test samples and reagents were prepared in 0.1% (w/v) casein in PBS, and plates washed three times between each step using 0.05% (v/v) Tween20 in PBS. Samples were tested in duplicate and corrected for background reactivity using negative control wells from which antibodies were omitted.

### Results and Discussion

Anti-TNP Antibody Constructs The TNP-WT mAb used in this example comprised either of two human heavy chain isotypes; first, an IgG1 isotype polypeptide (SEQ ID NO: 32) comprising, in the order of the N-terminus to the C-terminus, the TNP-specific V_{H} domain of mouse monoclonal antibody TIB142 fused to CH1-hinge-CH2-CH3 domains of human IgG1, encoded by the cDNA (SEQ ID NO: 33) (Patel D et al., J Immunol 184: 6283-6292, 2010); and secondly, an IgG2 isotype polypeptide (SEQ ID NO: 34) comprised, in the order of N-terminus to the C-terminus, the TNP-specific V_{H} domain of mouse monoclonal antibody TIB142 fused to CH1-hinge-CH2-CH3 domains of human IgG2, encoded by the codon-optimised cDNA with the sequence shown as SEQ ID NO: 35. The TNP-specific light chain polypeptide (SEQ ID NO: 36) comprised the TNP-specific V_{L} domain of mAb TIB142 fused to the human kappa chain constant domain, encoded by the codon-optimised DNA with the sequence shown as SEQ ID NO: 37.

### Modification of the IgG Enhances Complement Fixation

When antibodies, such as mAbs, are bound to their target antigen the classical pathway of complement may be activated leading to physiological effects including target cell destruction by complement dependent lysis (CDC) or complement mediated antibody dependent phagocytosis (C′ADCP) by specialised phagocytic cells through specific receptors for complement proteins and fragments. Activation of this major amplifying effector system of innate immunity is critically dependent on, and initiated by, the binding of the hexameric complement protein C1q which is part of the C1 complex. This binding of C1q by the antibodies bound to their target antigens leads ultimately to the formation of the membrane attack complex (MAC) comprising the other complement components (C5b, C6, C7, C8, C9).

Since the H429F Fc region component mutations conferred potent CDC in the ACE2-Fc fusion proteins (see Example 1), the effects of these (FIG. 12) and several other mutations (FIG. 13) were evaluated in human IgG1 under varied conditions of antigen density or input mAb concentration. The Fc component mutations were examined in an ELISA for complement fixation in the context of an intact (chimeric) IgG1 antibody with mouse V domains recognising the TNP hapten and human IgG1 constant domains. Surprisingly, replacement of histidine at position 429 in the TNP-IgG1-H429F mAb resulted in enhanced C1q binding compared to the C1q binding of the unmodified TNP-IgG1-WT mAb (FIG. 12). Next, the TNP-specific mAbs were evaluated for generation of the membrane attack complex proteins (FIG. 12). Again, the TNP-IgG1-H429F mAb showed enhanced complement activation as seen by the increased formation of the MAC proteins (C5b, C6, C7, C9), and importantly, this is entirely consistent with its observed enhanced capacity to bind C1q conferred by the H429F mutation. Thus, modification at position 429 can unexpectedly alter the properties of antibodies. This enhanced function is surprising as amino acid 429 is buried within the CH3 domain of IgG1 and is distant from the complement C1q binding surface in CH2 of the H chain and from the binding site for the leukocyte Fc receptors (Hogarth P M and Pietersz G A. Nature Reviews Drug Discovery 11:311-331, 2012; Chenoweth et al., 2020 supra).

The H429F mutation in the IgG1 antibody enhanced both C1q (FIG. 12A) and C5b-C9 fixation (FIG. 12B). The complement enhanced activities of the mutant IgG1 antibody comprising an H429F mutation, particularly in fixing C1q, were most apparent when lower densities of TNP:BSA antigen were opsonised by the mutated IgG (FIG. 12). Thus, in the context of both the ACE2-Fc fusion proteins and in intact IgG1 antibodies, the H429F Fc component mutation enhances complement activation.

The antigen binding activity of the anti-TNP mAbs, TNP-IgG1-WT, TNP-IgG2-WT and the modified (mutant) mAb, TNP-IgG1-H429F, were evaluated by ELISA for binding to TNP haptenylated BSA (TNP-BSA) as described (Wines et al., 2016 supra). All of the mAbs showed substantially equivalent binding activity to TNP-BSA (FIG. 12C) indicating that the enhanced complement activation by the Fc modified anti-TNP antibodies was due to the Fc mutation and not to differences in antigen binding activity.

### Conclusion

The data obtained in this example showed that the IgG-H429F mutant was capable of enhanced C1q and C5b-C9 fixation to enable increased complement activation and can therefore be expected to be capable of inducing useful complement-based effector functions.

### Example 3 Production of Modified Forms of Therapeutic Antibodies Comprising an H429 Mutation and Analysis

In this example, the effects of modifying position H429 (i.e. by amino acid substitution) on the properties of antibodies was evaluated using a number of unrelated chimeric mAbs containing distinct antigen binding variable domains which bind to unrelated epitopes in a range of different target molecules on different cell types. These antibodies were “based” upon various commercially used, and in some cases, clinically significant mAbs. In particular, the experimentation was directed at determining the possible functional effects of amino acid substitution at position 429 in antibodies bearing the V domains of the anti-HER2 mAbs trastuzumab and pertuzumab; anti-CD20 mAbs bearing the V domains of rituximab or 11B8; and the V domain of the anti-CD38 mAb daratumumab.

### Methods and Materials

### Antibodies and Antibody Constructs

DNA sequences obtained by RT-PCR or synthetic DNA corresponding to the immunoglobulin variable and constant sequences were assembled by standard molecular biology techniques including ligation and Gibson assembly (NEBuilder, New England Biolabs) or as complete synthetic DNAs to encode entire immunoglobulin H and L chains. These sequences were utilised in expression vectors such as pcDNA3.1, pcDNA 3.4 (Thermo Fisher Scientific) and pCIneo (Promega Corporation).

The unmodified mAbs used in this example were formatted on human IgG heavy chains and produced with a human kappa light chain. The unmodified mAbs include the specific V_{H} and V_{L} domains of the indicated mAb and are referred to as the wild type (WT) forms; for instance, the “WT” trastuzumab antibody used in this example, comprised the wild type (WT) HER2-specific V_{H} and V_{L} domains of trastuzumab as previously described (https://go.drugbank.com/drugs/DB00072).

More particularly, the trastuzumab-WT mAb used in this example comprised the HER2-specific heavy chain polypeptide described at https://go.drugbank.com/drugs/DB00072 (the amino acid sequence of which is provided as SEQ ID NO: 12) comprising, in the order of N-terminus to C-terminus, the HER2-specific V_{H} domain of trastuzumab fused to CH1-hinge-CH2-CH3 domains of human IgG1 and encoded by a codon-optimised DNA with the sequence shown as SEQ ID NO: 13. Similarly, the polypeptide of the anti-HER2 mAb trastuzumab light chain is as described at https://go.drugbank.com/drugs/DB00072 (SEQ ID NO: 14) and comprises the HER2-specific V_{L} domain of trastuzumab fused to a human kappa constant domain and is encoded by a codon-optimised DNA with the sequence shown as SEQ ID NO: 15.

The 11B8-WT mAb used in this example comprised a previously described CD20 specific heavy chain polypeptide (U.S. Pat. No. 8,529,902) (SEQ ID NO: 16) comprising, in the N-terminus to C-terminus order, the CD20-specific V_{H} domain of 11B8 fused to CH1-hinge-CH2-CH3 domains of human IgG1, and is encoded by a codon-optimised DNA with the sequence shown as SEQ ID NO: 17. Similarly, the polypeptide of the anti-CD20 mAb 11B8 light chain was as previously described (SEQ ID NO: 18) and comprised the CD-20 specific V_{L} domain of 11B8 fused to human kappa constant domain and is encoded by a codon-optimised DNA with the sequence shown as SEQ ID NO: 19.

The daratumumab-WT mAb used in this example comprised a previously described CD38-specific heavy chain polypeptide (https://go.drugbank.com/drugs/DB09331) (SEQ ID NO: 20) comprising, in the order N-terminus to C-terminus, the CD38-specific V_{H} domain of daratumumab mAb fused to CH1-hinge-CH2-CH3 domains of human IgG1, and is encoded by the codon-optimised DNA with the sequence shown as SEQ ID NO: 21. Similarly the polypeptide of the anti-CD38 mAb daratumumab light chain was as previously described at https://go.drugbank.com/drugs/DB09331 (SEQ ID NO: 22) comprising the CD38-specific V_{L} domain of the daratumumab mAb fused to human kappa constant domain is encoded by the codon-optimised DNA with the sequence shown as SEQ ID NO: 23.

The pertuzumab-WT mAb used in this example comprised a HER2-specific heavy chain polypeptide as previously described at https://go.drugbank.com/drugs/DB06366 (SEQ ID NO: 24) comprising, in the order of the N-terminus to the C-terminus, the HER2-specific V_{H} domain fused to CH1-hinge-CH2-CH3 domains of human IgG1, and is encoded by the codon-optimised DNA with the sequence shown as SEQ ID NO: 25. Similarly, the polypeptide of the anti-HER2 mAb trastuzumab light chain was as previously described at https://go.drugbank.com/drugs/DB06366 (SEQ ID NO: 26) comprising the HER2-specific V_{L} domain of the pertuzumab mAB fused to human kappa constant domain and is encoded by the codon-optimised DNA with the sequence shown as SEQ ID NO: 27.

The rituximab-WT mAb used in this example comprised a CD20-specific heavy chain polypeptide (as described at https://go.drugbank.com/drugs/DB00073) (SEQ ID NO: 28) comprising in the order N-terminus to C-terminus, the CD20-specific V_{H} domain of rituximab fused to CH1-hinge-CH2-CH3 domains of human IgG1, and is encoded by the codon-optimised DNA with the sequence shown as SEQ ID NO: 29. Similarly, the polypeptide of the anti-CD20 mAb rituximab light chain as previously described at https://go.drugbank.com/drugs/DB000723 (SEQ ID NO: 30) comprising the CD20-specific V_{L} domain of rituximab fused to human kappa constant domain, and is encoded by the codon-optimised DNA with the sequence shown as SEQ ID NO: 31.

Synthesis of Unmodified and Mutated Heavy Chains Antibody expression vectors were generated by standard methods that are known to those skilled in the art. Briefly, antibody expression vectors consisted of a synthetic polynucleotide sequence, encoding the antibody heavy chain or light chain, appropriately placed within plasmids such as, for example, pcDNA3 and pcDNA3.4 (Thermo Fisher Scientific). Expression vectors for antibodies of different specificities were produced by cleavage at restriction sites at the boundaries of the existing variable domain (V_{H} or V_{L}). A new synthetic DNA encoding the new V domain and flanked by sequences (e.g. 25 nucleotides) that were homologous to the cleaved vector were then incorporated by reaction with NEBuilder (New England Biolabs) according to the manufacturer's instructions.

Fc variants were produced by synthesis of synthetic polynucleotide sequences encoding the variant or by cleavage of the Fc encoding sequence of antibody expression plasmids with appropriate restriction enzymes and the incorporation of new mutagenic synthetic DNA by reaction using NEBuilder (New England Biolabs) according to the manufacturer's instructions.

Expression of Antibody Constructs Expression of the antibodies was conducted using transient transfection of Expi293F cells (Thermo Fisher Scientific). Expi293F cells were cultured in EXPI expression media (Life Technologies) and, 24 hours prior to transfection cells, were split to a concentration of 2×10⁶ viable cells/ml. On the day of transfection, 7.5×10⁷ viable log phase cells were centrifuged and resuspended in 25 ml of pre-warmed, antibiotic-free Expi293 Expression Media and maintained at 37° C. until transfection. Transfection of the cells was then performed at RT using the Life Technologies Expifectamine Transfection Kit as follows. Eighty microlitres of ExpiFectamine 293 reagent was diluted with 1.5 ml Opti-MEM-I Reduced Serum Medium (Gibco) and incubated for 5 minutes at RT. Thirty μg of DNA (15 μg of H chain DNA and 15 μg of L chain DNA) was diluted in 1.5 ml of Opti-MEM-I reduced serum medium then added to the diluted ExpiFectamine reagent, incubated for 20-30 minutes at RT and then added dropwise to the Expi293F cell suspension which was then cultured at 37° C. for 16 to 18 hours at which time 150 μL of the manufacturer's Enhancer 1 and 1.5 ml of Enhancer 2 were added and the cells cultured for a further four days at 37° C.

Cell cultures were harvested and centrifuged at 2500 rpm for 20-30 minutes, and the supernatant filtered with a 0.2 μm high flow filter (Sartorius AG, Gottingen, Germany) prior to purification. The presence of expected antibody in the supernatant was confirmed by SDS-PAGE.

Protein a Affinity Purification of mAbs

The mAbs were purified from the supernatant of the transfected Expi293F cells by Protein A affinity chromatography. Briefly, Hi-trap™ Protein A high-performance columns (GE Healthcare Life Sciences) were washed and equilibrated in binding buffer (20 mM NaH₂PO₄ pH 7.0) and the cell culture supernatant was loaded, the columns washed with binding buffer to baseline OD280 nm and bound antibody eluted with 0.1M sodium citrate tribasic dihydrate (pH 3.5) and 1 ml fractions collected and neutralised immediately with 1M Tris-HCl pH 9.0 and fractions containing the antibody, pooled.

Size Exclusion Chromatography (SEC) of mAbs

Following Protein A affinity purification, size exclusion chromatography (SEC) was used to further purify and characterise the antibodies. The Protein A purified antibodies were concentrated to an OD280 nm of 6-8 using a 30 kDa molecular weight cut-off centrifuge concentrator device (Merck-Millipore). Superose 6 10/300GL columns (GE Healthcare Life Sciences) were equilibrated in PBS pH 7.2, then the concentrated Protein A affinity purified antibody was loaded and separated at a flow rate of 0.5 ml/min in PBS and 0.5 ml fractions collected. For some mAbs, SEC was performed at pH 5.0, thus the concentrated Protein A purified mAb was dialysed overnight against buffer (100 mM sodium citrate, 100 mM NaCl, pH 5.0), then applied to a Superose 6 10/300 column pre-equilibrated in the same buffer. The dialysed antibody was applied to the Superose column SEC at a flow rate of 0.5 ml/min and 0.5 ml fractions collected from the column.

### Evaluation of Antigen Binding by Monoclonal Antibodies

Purified antibodies were tested for antigen binding prior to functional analysis. The mAbs recognising cell surface antigens were tested for antigen recognition by flow cytometry as previously described in Trist et al., J Immunol 192(2):792-803, 2014. The anti-CD20 mAbs based on rituximab or 11B8 and the anti-CD38 mAb based on daratumumab were tested for binding on Ramos lymphoma cells expressing CD20 and CD38. The anti-HER2 mAbs based on the trastuzumab and pertuzumab mAbs were tested on the HER2 expressing ovarian cancer cell line SK-OV-3. Briefly, mAbs were titrated by serial two-fold dilution in 25 μl of FACS buffer (PBS with 0.5% (w/v) BSA). Twenty-five microlitres of target cells at the concentration of 5×10⁶/ml were then added to the titrated mAb and incubated for 30 minutes on ice. Cells were then washed twice in FACS buffer, resuspended with 50 μL of anti-human IgG(Fab′)₂-Alexa 647 conjugate and incubated for 30 minutes on ice, washed twice in cold FACS buffer and then resuspended in 200 μl FACS buffer. The cells were analysed by flow cytometry using a BD FACSCanto™ II flow cytometer.

Flow Cytometric Detection of C1q Binding to Cells Opsonised with Monoclonal Antibody

C1q binding to antibody-opsonised cells was evaluated by flow cytometry. Ramos lymphoma cells or SK-OV-3 adenocarcinoma cells (5×10⁶/ml in FACS buffer) were incubated with serial dilutions of anti-CD20 rituximab-based or anti-HER2 trastuzumab-based antibodies respectively on ice for 30 minutes. Cells were washed twice in FACS buffer, resuspended in normal human serum diluted 1/3 in FACS buffer and incubated on ice for a further 30 minutes. The cells were then washed twice in cold FACS buffer and bound C1q was detected by resuspending the cells in rabbit antiserum detecting human C1q (1:500 dilution) and incubated for a further 30 minutes on ice. The cells were then washed twice in cold FACS buffer, resuspended in phycoerythrin-conjugated donkey antisera detecting rabbit antibody for 30 minutes, washed a further two times and resuspended in 200 μL of ice-cold FACS buffer, before being analysed on a BD FACSCanto™ II flow cytometer (Becton Dickinson).

### Complement Dependent Lysis of Cells

CDC was measured by flow cytometry using the Zombie Green Fixable Viability kit (BioLegend) following opsonisation of target cells with mAb. The mAb of interest was serially two-fold diluted starting at the initial concentration indicated on the figures, in 25 μl of PBS/BSA/G (PBS containing 0.5% (w/v) BSA and 1 mM glucose) or Lebowitz-15 (L-15) medium (containing 0.5% (w/v) BSA and lacking phenol red). Thus, for each WT mAb or modified mAb (i.e. H429-mutant mAb), or combinations thereof, 25 μl of target cells, at a concentration of 5×10⁶/ml in 25 μl of PBS/BSA/G, were then added to the titrated mAb and incubated for 30 minutes on ice. Cells were then washed twice in buffer and resuspended in 50 μl of complement (normal human serum (NHS) thawed and diluted 1:3 in buffer immediately before use), and incubated for 30 minutes at 37° C. Following the incubation, the cells were washed twice in cold buffer without BSA then resuspended in 50 μl Zombie Green (prepared in DMSO according to the manufacturer's instructions; BioLegend) diluted 1/500 in PBS and incubated for a further 30 mins on ice, protected from light. These cells were then washed once in buffer, resuspended in 50 μl of 2% paraformaldehyde/BSA/PBS/G and incubated for 30 minutes on ice, then washed once in PBS/BSA and re-suspended in 200 μl PBS/BSA/G. The cells were analysed by flow cytometry using a BD FACSCanto™ II flow cytometer (Becton Dickinson).

### MAb Cooperation to Enhance C1q Binding and CDC

Ramos cells, at 5×10⁵ cells/ml in 25 μl of PBS/BSA/G, were incubated with two-fold diluted rituximab-WT in the presence of 0.025 μg/ml daratumumab-WT or rituximab-H429F with two-fold diluted rituximab-H429F, in the presence of 0.025 μg/ml daratumumab-H429F mutant, or 0.5 μg/ml 11B8-H429F mutant, for 30 minutes at 37° C. in the presence of 1/3 dilution of normal human serum, washed twice with PBS, incubated with 1/500 Zombie Green for 30 minutes on ice washed again with PBS/BSA/G and fixed with 2% paraformaldehyde. The cells were washed, resuspended in PBS/BSA/G and analysed on a BD FACSCanto™ II flow cytometer (Becton Dickinson).

### Results and Discussion

### Antigen Binding by Monoclonal Antibodies

The antigen binding capacity of the purified antibodies used in this example were tested and confirmed prior to functional analysis.

The purified mAbs detecting cell surface antigens were also tested by flow cytometry for binding to either antigen positive Ramos cells (CD20⁺ CD38⁺) or SK-OV-3 cells (HER2⁺) and all of the mAbs showed readily detectable levels of antigen binding (FIG. 14). Moreover, within each group, the modified mAbs carrying a mutation in the IgG heavy chain, showed similar binding activity to that of the unmodified (WT) form (e.g. the anti-CD20 mAbs rituximab-WT and the rituximab-H429F and rituximab-H429Y mutants showed equivalent homogeneous binding to CD20 expressing Ramos cells). Similarly, the anti-CD38 daratumumab-WT mAb and its CH3-modified mutant, daratumumab-H429F, as well as the anti-CD20 11B8-WT and its CH3-modified mutants all gave binding profiles equivalent to their respective wild type forms (FIG. 14). Also, the anti-HER2 mAb trastuzumab-WT and its modified CH3-mutant form, trastuzumab-H429F and trastuzumab-H429Y, showed readily detectable and equivalent binding to SK-OV-3 adenocarcinoma cells as did pertuzumab-WT and its modified CH3 mutants (FIG. 14).

The effects of modifications at position 429 on antibody properties were further investigated using mAbs that bind to cell surface molecules. It was found, in particular, that substitution of H429 alters the physical characteristics of antibodies and selectively promotes antibody oligomerisation. For example, using mutants of the rituximab antibody; that is, rituximab-H429F mAb and rituximab-H429Y mAb, wherein the introduced tyrosine and phenylalanine amino acids are structurally similar, it was found that while the rituximab-H429F mAb and rituximab-H429Y mAb both eluted from Protein A affinity columns as a single homogeneous peak with elution characteristics like those of the unmodified rituximab-WT IgG (FIG. 15A), purification by size exclusion chromatography (SEC) at pH 7.2 (FIG. 16A) showed that the rituximab-H429Y mAb IgG exhibited a surprisingly different SEC profile (i.e. as compared to the profile for rituximab-WT and rituximab-H429F antibodies. That is, the rituximab-H429Y mAb SEC profile revealed two distinct peaks, one IgG peak (right of the vertical line in FIG. 16A) which was coincident with a single IgG peak in the SEC of rituximab-WT and rituximab-H429F mAbs, and a second peak considered to be containing preformed oligomers (IgG (oli)) of rituximab-H429Y (see FIG. 16A, left of the vertical line) and indicating an equilibrium between the formation of oligomeric and non-oligomeric forms. Whilst the presence of both oligomeric IgG and non-oligomeric IgG in the rituximab-H429Y mAb was surprising and different from the rituximab-WT IgG, it was even more surprising that this differed from what was observed with the rituximab-H429F mAb. Thus, the different biophysical properties of the rituximab-H429Y and rituximab-H429F mAbs is determined only by the presence of a hydroxyl in the tyrosine at position 429 of the rituximab-H429Y mAb.

Using SDS-PAGE, it was revealed that the oligomeric (IgGₒₗᵢ) and non-oligomeric (IgG) forms observed in the SEC IgG of rituximab-H429Y mAb behaved identically to each other and to the rituximab-WT IgG and rituximab-H429F mAb IgG. That is, despite its oligomeric nature, the rituximab-H429Y IgGₒₗᵢ migrated, under non-reducing conditions as a single ˜150 kDa IgG species identical to its non-oligomeric rituximab-H429Y IgG (H₂L2) form indicating, importantly, that the oligomerisation of the mAb, facilitated by the H429Y mutation was non-covalent in nature (FIG. 17A). Moreover, this clearly demonstrated that the rituximab-H429Y can exist at pH 7.2 as both IgG (IgG, H₂L2) and as preformed oligomers of IgG (IgGₒₗᵢ) which arise from non-covalent association between the IgG heavy chains carrying the H429Y mutation. Further analysis under reducing conditions, by reduction in dithiotheitol (DTT), showed that these non-covalent oligomers of rituximab-H429Y as well as the non-oligomeric rituximab-H429Y IgG, resolved into the expected ˜50 kDa heavy (H) chain and light ˜25 kDa (L) chain species identical to the SDS-PAGE characteristics of rituximab-WT and the rituximab-H429F mutant.

Thus, the H429Y modification confers new characteristics on rituximab wherein, in solution at pH 7.2, the mAb exists as pre-formed, non-covalent oligomers of IgG which exist in equilibrium with single IgG molecules. In contrast, rituximab-H429F and rituximab-WT mAbs only exist in solution as the single IgG species. Thus, the choice of amino acid at position H429 unpredictably affects the physical properties of rituximab IgG.

It was also found that these effects of mutation at position H429 is independent of the V domains. That is, equivalent evaluation of H429 substitution in trastuzumab, a mAb unrelated to rituximab, and which detects a cell surface molecule (i.e. HER2) structurally distinct from CD20, achieved similar results (see FIGS. 15B, 16B and 17B). In particular, the elution of the trastuzumab-WT IgG from the Protein A affinity matrix with citrate buffer, pH 3.0, yielded a homogeneous single peak (FIG. 15B), as did elution of the trastuzumab-H429F and trastuzumab-H429Y mutant mAbs (the single IgG peak being coincident with the peak obtained of the unmodified trastuzumab-WT IgG mAb). Then, by size exclusion chromatography (SEC) at pH 7.2 (FIG. 16B), it was found that the unmodified trastuzumab-WT and the trastuzumab-H429F mAbs each contained, the expected, single major IgG species, while the trastuzumab-H429Y mAb, in contrast, showed two distinct peaks (FIG. 16B). The first trastuzumab-H429Y IgG peak was coincident with the single IgG peaks observed for trastuzumab-WT mAb and trastuzumab-H429F mAb (FIG. 16B, right of vertical line) and the second trastuzumab-H429Y IgG peak contained oligomeric IgG (IgGₒₗᵢ) (FIG. 16B, left of vertical line). In addition, SDS-PAGE (FIG. 17B) showed that under non-reducing conditions (i.e. without disulphide bond reduction), the IgG peak of the trastuzumab-WT and trastuzumab-H429F mAbs each migrated at the expected 150 kDa mass. And, in the case of the trastuzumab-H429Y mAb, SDS-PAGE (FIG. 17B) showed that under non-reducing conditions both the oligomeric trastuzumab-H429Y (IgGₒₗᵢ) and the non-oligomeric (IgG H₂L2) species of trastuzumab-H429Y IgG migrated identically as a single 150 kDa species. This clearly demonstrated that the trastuzumab-H429Y can exist at pH 7.2 as both single IgG and as preformed oligomers of IgG arising from non-covalent association between the IgG heavy chains carrying the H429Y mutation.

Thus, the amino acid substitution at position 429 in the trastuzumab heavy chain conferred the same properties as those observed for the equivalent substitution in rituximab-based mAbs, and accordingly, the effects on the physical characteristics of the mutated mAb (particularly of oligomerisation) facilitated by H249Y mutation are independent of antibody specificity, molecular target, epitope and the V_{H} and V_{L} domains.

### Preformed Oligomeric and Non-Oligomeric H429Y IgG Antibodies Exhibit Enhanced CDC

While not wishing to be bound by theory, it is considered that oligomerisation, particularly hexamerisation, of mAbs provides the optimal basis for C1q binding and activation of the complement cascade that leads to complement-dependent effector responses (e.g. phagocytosis or killing of target cells by complement-dependent cytotoxicity (CDC)). Such oligomers/hexamers can either form in solution or on the target (ie “on target” oligomerisation or assembly).

The effect on antibody effector function arising from the H429Y modification of the CH3 domain of the IgG heavy chain was evaluated by complement dependent cytotoxicity assays (CDC) (see FIG. 18). By way of example, the effect of the H429 mutation on CDC potency was determined for the oligomeric and non-oligomeric forms of rituximab-H429Y using CD20 positive Ramos lymphoma cells as target cells. Thus, both the oligomeric rituximab-H429Y (IgG (oli)) and the non-oligomeric, IgG (H₂L2) forms, separated by SEC at pH 7.2 (FIG. 18A) were evaluated for CDC potency and compared to the CDC of the unmodified rituximab-WT IgG. Ramos cells were incubated in the presence of serial dilutions of the rituximab-WT IgG or non-oligomeric rituximab-H429Y IgG (H₂L₂) (p1 fraction of FIG. 18A) or oligomeric rituximab-H429Y IgG H₂L₂ species (p2 fraction of FIG. 18A). Normal human serum (diluted 1/3) was then added as a source of complement and the proportion of Ramos cells killed by CDC at each mAb concentration was determined by flow cytometry (FIG. 18B). The non-oligomeric rituximab-H429Y IgG (H₂L₂) (the p1 fraction), demonstrated a surprising enhancement in CDC potency compared to the unmodified rituximab-WT. The non-covalent oligomeric IgG form of rituximab-H429Y (IgGₒₗᵢ) (the p2 fraction) also showed enhancement of CDC compared to rituximab-WT. Interestingly, both the oligomeric p2 form and the non-oligomeric p1 form exhibited similarly enhanced CDC potency which is consistent with the H429Y oligomers forming hexamers in solution at pH 7.2 prior to binding the target cell or in the case of non-oligomeric forms IgG (H₂L2) forming hexamers on the target cell surface after antigen binding and thereby providing the optimal Fc configuration for C1q binding and thus enhanced complement activation as observed in the mAb-H429Y forms of IgG.

Thus, the substitution of H429 with tyrosine not only alters the physical characteristics of mAbs permitting oligomerisation in solution but also enhances complement dependent killing of target cells whether the IgG is oligomerised in solution or on the target.

The Oligomerisation of mAbs Including an H429Y Mutation is Controlled by Altering the pH

The nature of the non-covalent oligomerisation of the mAbs including an H429Y modification in the CH3 domain was investigated further by altering the pH of the buffer (environment) (FIG. 18C). In particular, the trastuzumab-H429Y mutant mAb (as purified by Protein A affinity chromatography) was dialysed either into buffer at pH 7.2 and then subjected to SEC also at pH 7.2, or dialysed into citrate buffer at pH 5.0 and then subjected to SEC at pH 5.0. At pH 7.2, both oligomeric and non-oligomeric IgG was present but lowering the pH of the buffer completely reversed the formation of IgG hexameric oligomers and only the IgG (H₂L2) species was apparent (FIG. 18C). Accordingly, the H429Y modification of the CH3 domain facilitates pH-sensitive, non-covalent oligomerisation of mAbs and thereby the oligomerisation/hexamerisation in solution or on the target that enhances effector potency of complement. Moreover, the capacity to control oligomerisation by alteration of pH may be useful the manufacture of immunoglobulins carrying this modification.

### The H429F Mutation Enhances the Activation of the Serum Complement Cascade

The extent of the effect on C1q binding by modification of the CH3 domain at the H-chain position 429 was investigated by flow cytometry on cells treated with the unrelated mAbs recognising CD20 or HER2 and bearing the H429F mutation in the CH3 of their heavy chains. The results are shown in FIG. 19A-D. C1q binding was evaluated on CD20 Ramos cells treated with either rituximab-WT mAb or with rituximab-H429F mAb (FIG. 19A, B). Similarly, C1q binding was also measured on HER2 positive SK-OV-3 cells treated with trastuzumab-WT mAb or trastuzumab-H429F mAb (FIG. 19C, D). C1q bound detectably above background controls to Ramos cells treated with rituximab-WT mAb (FIG. 19A) and to SK-OV-3 cells treated with trastuzumab-WT (FIG. 19C). Importantly, C1q binding was enhanced to both the rituximab-H429F treated Ramos cells (FIG. 19B) and to trastuzumab-H429F treated SK-OV-3 cells (FIG. 19D) compared to the rituximab-WT and trastuzumab-WT. Thus, the substitution of H429 in the CH3 domain of the heavy chain enhanced C1q binding in these unrelated anti-CD20 and anti-HER2 mAbs. It is also therefore clear that this functional enhancement by H429 modification is independent of the antibody variable region and of the molecular target and the epitope detected. Moreover, enhanced C1q binding is consistent with enhanced antibody dependent activation of complement in the classical complement pathway which leads to the development of the MAC that leads to cell lysis via CDC.

Accordingly, since the binding of C1q component of the C1 complex initiates the classical complement cascade that leads to the lysis of cells, the effect of the enhanced C1q binding on the CDC killing potency was also determined using the rituximab-H429F mAb and compared to the killing potency of unmodified rituximab-WT (FIG. 19E). In particular, the proportion of Ramos cells killed by CDC was evaluated by flow cytometry at each of the indicated mAb concentrations (FIG. 19E). Like the surprising enhancement of C1q binding conferred by the H429F modification of the CH3 domain in the rituximab-H429F mAb (FIG. 19A compared to 19B), CDC killing potency (EC₅₀) was also dramatically improved (>10-fold) compared to CDC by the unmodified rituximab-WT (FIG. 19E). This surprising improvement in Cq binding and in CDC killing potency by the H429F mutation is consistent with oligomerisation (particularly hexamerisation) of the antibody on the target surface, and appears to provide the optimal configuration of the mAb Fc for the binding of C1q which is itself a hexamer, as exemplified by the analysis of the rituximab-H429F mAb.

The Enhanced Complement Activation of mAbs with H429 Mutation is Epitope Independent

As discussed above, it was found that an H429 substitution in the anti-CD20 rituximab mAb strongly increased its CDC activity; to evaluate whether the enhancement of antibody function can apply to a different epitope within a single molecular target, complement activation was investigated using a second anti-CD20 mAb. The results are shown in FIGS. 20-22.

The type-II anti-CD20 mAb, 11B8 exhibits naturally poor CDC activity, but importantly also detects an epitope distinct from type-I anti-CD20 mAb rituximab (Meyer S et al., Br J Haematol 180(6):808-820, 2018). The 11B8-WT mAb and a mutant thereof including H429F substitution were produced as described above and purified separately by Protein A affinity chromatography (FIG. 20). A single homogeneous IgG peak was obtained for the 11B8-WT and 11B8-H429F mAbs (FIG. 20) indicating that the modifications had not altered the purification characteristics of the modified mAbs compared to the unmodified 11B8-WT mAb. Also, size exclusion chromatography demonstrated that the 11B8-WT mAb contained a single non-oligomeric IgG (H₂L2) species (FIG. 21A) as did the 11B8-H429F mAb. This was confirmed by SDS-PAGE analysis (FIG. 21B) that revealed the expected 150 kDa IgG species, prior to disulphide bond reduction for the wild type and mutant mAbs that resolved to the ˜50 kDa heavy chain and 25 KD light chain species following reduction in DTT (FIG. 21B).

The CDC potency of 11B8 WT was also compared to that of the H429 modified antibody 11B8-H429F (FIG. 22). The 11B8-WT and 11B8-H429F were titrated in the presence of normal human serum as a source of complement and the percent killing of Ramos cells evaluated for each mAb concentration. It was found that the 11B8-WT mAb failed to induce appreciable CDC but in contrast, the Fc modified 11B8-H429F mAb mediated potent CDC (FIG. 22). Thus, the CDC potency is enhanced in two distinct and unrelated mAbs, rituximab-H429F and 11B8-H429F and (compare FIGS. 19E and 22), which indicates that improvement of CDC by modification of the H429 position can be achieved in different mAbs targeting distinct epitopes within the same target molecule. This also indicates that the enhanced CDC achieved by the CH3 modification is also independent of the variable domain of the modified antibody.

### The Enhancing Effect of H429F is Independent of Antibody, Molecular Target and Cell Type

The enhanced complement activating potency conferred by H429 substitution to other mAbs was evaluated in an antibody, daratumumab, recognising a fourth and unrelated molecular target, namely CD38 (de Weers M et al., J Immunol 2011; 186:1840-1848, 2011; Overdijk m B et al., MAbs 7:311-321, 2015), which is a cell surface molecule that is structurally unrelated to CD20 or HER2. The daratumumab-WT mAb was formatted as a human IgG1 and kappa light chain mAb as described above. A mutant of this antibody was engineered wherein the H429 residue was replaced with phenylalanine to create the daratumumab-H429F mAb. In each case, the daratumumab-based IgG mAbs showed equivalent characteristics and eluted from the Protein A affinity column as a single homogeneous peak (FIG. 23). Further purification by SEC yielded a single IgG (H₂L₂) peak for the daratumumab-WT mAb and an equivalent peak also for the daratumumab-H429F mutant mAb (FIG. 24A). Moreover, SDS-PAGE confirmed that the SEC purified mAbs comprised the expected 150 kDa IgG species (prior to disulphide bond reduction), and the ˜50 kDa heavy chain and 25 kDa light chain species following reduction in DTT (FIG. 24B).

The CDC potency of the daratumumab-H429F mAb was compared to daratumumab-WT mAb (FIG. 25). Each mAb was titrated individually by serial two-fold dilution in the presence of normal human serum as a source of complement. The percent killing of CD38 expressing Ramos cells was evaluated for each mAb concentration and is shown in FIG. 25. Both the daratumumab-WT and daratumumab-H429F mAbs achieved ˜80% killing of Ramos lymphoma cells, however the daratumumab-H429F mAb exhibited greater CDC potency, ˜15-fold higher EC50. The improved CDC potency of the daratumumab-H429F mAb was then investigated using CD38 positive cells KMS-12-PE myeloma cells that resist CDC (FIG. 26A). The daratumumab-WT mAb showed barely detectable CDC of KMS-12-PE cells above the background control lysis (˜20%) in the presence only of complement (no mAb C′ only), even at concentrations 5-times greater (5 μg/ml) than that (1 μg/ml) required to maximally kill Ramos lymphoma cells (˜80% lysis; see FIG. 25). In contrast, potent CDC of the KMS-12-PE cells was mediated by the daratumumab-H429F mAb (FIG. 26A). In a further experiment, CDC potency was evaluated on the CD38 expressing SUP-15 acute lymphoblastic leukaemia (ALL) cells. As can be seen from FIG. 26B, the SUP-15 cells also resisted CDC killing by the daratumumab-WT mAb, but were readily killed by the daratumumab-H429F mutant mAb. Thus, substitution at position 429 of the CH3 domain not only enhances CDC against certain targets but can also rescue potent CDC against lysis-resistant targets. Moreover, the enhanced CDC potency against the CD38 target (which is structurally distinct from CD20 detected by rituximab and 11B8, and HER2 detected by trastuzumab), also indicates that the improved efficacy is independent of target and of the epitope detected and thus also of the V domain.

H429 Substitution Facilitates Functional Synergy Between mAbs Directed at Distinct Epitopes

Functional synergy between mAbs and mediated by the H429 modification was investigated by determining the extent of binding of C1q in mixtures of mAbs (trastuzumab and pertuzumab) targeting separate epitopes in HER2.

The purification characteristics of the pertuzumab-WT mAb and a mutant wherein H429 had been replaced with phenylalanine (i.e. pertuzumab-H429F mAb) are shown in (FIGS. 27 and 28A). For each mAb, a single equivalent IgG peak was obtained from Protein A affinity chromatography (FIG. 27), and when further purified by size exclusion chromatography, single homogeneous non-oligomeric IgG species were observed (FIG. 28A, right of vertical line). SDS-PAGE analysis (FIG. 28B) confirmed that these peaks contained a 150 kDa IgG (H₂L2) species that resolved into ˜50 kDa heavy chains and ˜25 kDa light chains after reduction (FIG. 28B).

Cooperation between the unrelated anti-HER2 mAbs, trastuzumab-WT and pertuzumab-WT, which detect distinct epitopes in HER2 was determined by evaluating the extent of C1q binding in mixtures of the mAbs which was quantitated by flow cytometry (FIG. 29A). Ovarian cancer cells, SK-OV-3, treated with a mixture of equal concentrations of the HER2 mAbs, trastuzumab-H429F or pertuzumab-H429F containing the H429F modification showed enhanced C1q binding (MFI=10,877) over that observed when the mAbs were used individually at the same concentrations (FIG. 29A) pertuzumab-H429F (MFI=798) or trastuzumab-H429F (MFI=1739).

The cooperative enhancement of C1q binding was further evaluated (FIG. 29B) by titration of the individual anti-HER2 mAbs or pairwise mixtures of the mAbs at two-fold serial dilutions from a starting concentration of 5p g/ml for each mAb alone or in a mixture of the mAbs at a 1:1 ratio starting at 2.5 μg/ml:2.5 μg/ml of each mAb in the mixture (FIG. 29B). The trastuzumab-H429F and pertuzumab-H429F acted synergistically to further enhance complement activation particularly when the concentration of mAbs was limiting (e.g. at 1.25 μg/ml; FIG. 29B indicated by arrow). C1q binding by the mixture of 1.25 μg/ml trastuzumab-H429F and 1.25 μg/ml Pertuzumab-H429F (MFI=6327) was greater than the C1q binding by the individual mAbs used alone at the same or twice the concentration (i.e. trastuzumab-H429F MFI=603 at 1.25 μg/ml, or 1101 at 2.5 μg/ml, and pertuzumab-H429F MFI=457 at 1.25 μg/ml, or 658 at 2.5 μg/ml) and greater than a mixture of the WT mAbs at the same 1:1 concentrations (e.g. 1.25 μg/ml trastuzumab-WT and 1.25 μg/ml pertuzumab-WT MFI=522) and even greater than the wild type mAbs used alone (i.e. trastuzumab-WT MFI=258 at 1.25 μg/ml, or 405 at 2.5 g/ml) and pertuzumab-WT MFI=424 at 1.25 μg/ml, or MFI=483 at 2.5 μg/ml). This improved complement activation potency further indicates that greater functional potency is achievable by cooperative synergy in mixtures of CH3 domain-modified mAbs recognising distinct epitopes.

Modification of the CH3 Domain Enables mAbs of Distinct Specificity to Act Synergistically in CDC Independent of Target

The cooperative synergy of mAbs leading to greater functional potency as reflected in the enhanced C1q binding (FIG. 29) was investigated for enhanced CDC lysis of targets using mixtures of mAbs detecting either two distinct molecular structures (i.e. CD20 and CD38) or two distinct epitopes within the same molecular structure, CD20. The results are shown in FIG. 30.

Cooperation in CDC between antibodies detecting distinct molecular structures, and distinct epitopes, was determined using a mixture of rituximab-WT or rituximab-H429F mAbs that target CD20 in pairwise combination with daratumumab-WT or daratumumab-H429F mAbs which target the unrelated surface molecule, CD38. No cooperation in CDC was apparent when the rituximab-WT mAb was titrated in the presence of 0.25 μg/ml daratumumab-WT mAb (FIG. 30A); that is, the CDC observed at any concentration of rituximab-WT was no greater than the baseline CDC in the presence of 0.25 μg/ml daratumumab-WT alone (0 μg/ml rituximab, FIG. 30A) or at any concentration of the rituximab-WT alone (i.e. titrated in the absence of daratumumab-WT). However, in contrast to the unmodified wild type mAbs, the rituximab-H429F and daratumumab-H429F mAbs acted to enhance CDC cell killing (FIG. 30B). In particular, the cooperation between the mAbs in mediating CDC was readily observed in the rituximab-H429F concentration range of 0.5 μg/ml-0.125 μg/ml, when used with 0.025 μg/ml of the daratumumab-H429F mAb (NB. the enhancement is indicated by the up-arrows in FIG. 30B). CDC mediated by the rituximab-H429F mAb with the daratumumab-H429F mAb was considerably greater than with either of the H429F mAbs alone (rituximab-H429F or 0.025 μg/ml daratumumab; enhancement is indicated by the up-arrows in FIG. 30B).

Functional cooperativity between mAbs detecting distinct epitopes, but within the same target molecule, was investigated in mixtures of the different CD20 mAbs. The results are shown in FIG. 30C. Titration of the rituximab-H429F mAb in the presence (0.5 μg/ml) or absence of 11B8-H429F mAb revealed that the mAbs acted synergistically. The cooperation between the mAbs was readily detected when the concentration of both mAbs was limiting (0.25 μg/ml-0.031p g/ml rituximab-H429F and 0.05 μg/ml 11B8-H429F), where CDC was greater than with either mAb alone (enhancement indicated by the up-arrows in FIG. 30C).

Thus, mAb cooperation and synergy by modification of the antibody H chain at position 429, particularly H429F, is broad in its effect. It facilitates greater functional potency through cooperation and functional synergy in mixtures of mAbs irrespective of the epitope detected by the individual mAbs (i.e. whether the epitopes are present on the same or distinct molecular targets).

### Example 4 B Lymphocyte Killing by an Anti-CD20 Therapeutic Antibody with an H429 Mutation

Monoclonal antibodies are used in the treatment of inflammatory disease, such as autoimmune disease, by targeting normal (i.e. non-malignant) cells. For example, the anti-CD20 mAb Rituximab is used for the treatment of inflammatory disease (Lee D S W et al., Nat Rev Drug Discov 20:179-199, 2021) by targeting normal B lymphocytes known to express CD20.

### Methods and Materials

Isolation of Leukocytes from Human Peripheral Blood

Peripheral blood mononuclear cells (PBMC) were isolated from anticoagulated venous blood (Vacutainer ACD-A Becton Dickinson) by centrifugation on a Ficoll gradient. Purified cells from the plasma/ficoll interface were washed in flow cytometry buffer (L-15 medium lacking phenol red and containing 0.5% BSA (L15-BSA)) and resuspended in L15-BSA to a concentration of 5×10⁶/ml.

### Complement Dependent Lysis of Cells

The CDC killing of normal peripheral blood B lymphocytes in PBMC, mediated by WT and mutated anti-CD20 mAbs was measured by flow cytometry as described above using the Zombie Green Fixable Viability kit (BioLegend) following opsonisation of cells with mAb.

CDC was performed in 96-well plates. Cells (25 μl, 5×10⁶/ml) were reacted with equal volume of mAb in L15-BSA for 30 minutes on ice, then washed (diluted in 100-200 W of buffer and centrifuged (at 200×g for 5 minutes, 4° C.) twice in L15-BSA. The IgG-opsonised cells were then resuspended in 50 μl of human serum diluted 1/3 in L15-BSA, as a source of complement, and incubated at 37° C. for 30 minutes. The treated cells were then washed once in L15-BSA, resuspended in 50 μl anti-CD19-APC antibody (BioLegend) in L15-BSA and incubated for a further 30 minutes on ice. Following two washes in L-15 lacking BSA, cells were then resuspended in 50 l of Zombie Green (1/500 dilution in protein-free L-15 medium or PBS according to the manufacturer's instructions) and incubated on ice for 30 minutes. The cells were washed once in L15-BSA and the cells fixed by resuspension in 2% paraformaldehyde in buffer for 30 minutes on ice and finally washed once and then resuspended in 200 μl L15-BSA for flow cytometry analysis. B lymphocytes were identified by staining for CD19 and the proportion (%) of dead B lymphocytes specifically killed by CDC was enumerated as the percentage of CD19⁺ Zombie green⁺ cells compared to the background control of CD19⁺ Zombie green⁺ cells in PBMC samples treated with the negative control mAbs trastuzumab-WT or trastuzumab-H429F antibodies.

### Results and Discussion

The CDC killing of normal B lymphocytes by rituximab-H429F was investigated by flow cytometry and was compared to that of the unmodified rituximab-WT (FIG. 31). The rituximab-H429F showed greater potency, killing over 85% of B cells (FIG. 31B) compared to the much lower killing by the unmodified rituximab-WT which killed only 30% of the peripheral blood B lymphocytes (FIG. 31A). As HER2 is not expressed on B lymphocytes there was no CDC of the B lymphocytes (FIG. 31C, D) by the negative control anti-HER2 mAbs, trastuzumab-WT or trastuzumab-H429F.

### Example 5 H429F Modification Improves the Function of Immunoglobulins of Other Types

Histidine 429 is conserved in the equivalent position in all human Ig classes and subclasses (FIG. 3 and FIG. 4); that is, in the CH3 domain of all IgG subclasses and IgA subclasses (FIG. 3), in IgD (FIG. 4), and also in IgE, and IgM where the CH4 domain is the equivalent of the CH3 domain of IgG (FIG. 4). The effect of modification of H429 on the function of other immunoglobulins was evaluated using human IgG3 and human IgG4 as examples.

### Methods and Materials

### Antibodies and Antibody Constructs

The mAbs used in this example comprised heavy chains of the IgG3 or IgG4 subclasses. The IgG3 heavy chain used herein contains three amino acid substitutions, N392K, M397V and R435H, introduced to avoid aggregation of purified antibodies (Saito S et al., Prot Sci 28(5):doi:10.1002/pro, 2019).

The rituximab-IgG3 CD20-specific heavy chain polypeptide (SEQ ID NO: 38) comprised in the order of the N-terminus to the C-terminus, the CD20-specific V_{H} domain of rituximab (https://go.drugbank.com/drugs/DB00073) fused to CH1-hinge-CH2-CH3 domains of an aggregation resistant human IgG3, and is encoded by the codon-optimised DNA with the sequence shown as SEQ ID NO: 39. Similarly, the polypeptide of the anti-CD20 mAb rituximab light chain as previously described (SEQ ID NO: 30) comprising the CD20-specific VL domain of rituximab fused to human kappa constant domain (https://go.drugbank.com/drugs/DB00073), and is encoded by the codon-optimised DNA with the sequence shown as SEQ ID NO:31.

The rituximab-IgG4-WT formatted CD20-specific heavy chain polypeptide (SEQ ID NO: 40) comprised in the order of the N-terminus to the C-terminus, the CD20-specific V_{H} domain of rituximab fused to CH1-hinge-CH2-CH3 domains of human IgG4, and is encoded by the codon-optimised DNA with the sequence shown as SEQ ID NO: 41. Similarly, the polypeptide of the anti-CD20 mAb rituximab light chain as previously described by reference (SEQ ID NO: 30) comprising the CD20-specific VL domain of rituximab fused to human kappa constant domain and is encoded by the codon-optimised DNA with the sequence shown as SEQ ID NO:31.

Modification to the mAb heavy chains were produced by synthesis of an entire antibody chain codon-optimised polynucleotide sequence including the specific modification to encode for, for example, H429F (GeneArt; Thermo Fisher Scientific), and antibody expression vectors were generated by standard methods known to those skilled in the art. Briefly, antibody expression vectors consisted of the synthetic polynucleotide sequence, encoding the unmodified antibody heavy chain or with the modification/mutation or light chain, appropriately placed within an expression plasmid such as, for example, pcDNA3.4 (Thermo Fisher Scientific).

### Expression of Antibody Constructs

Expression of the antibodies was conducted using transient transfection of Expi293F cells (Thermo Fisher Scientific) under the same conditions as described for rituximab and the therapeutic mAbs described elsewhere in this specification. As for all mAbs, the cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE electrophoresis by the method of Laemmli.

### Protein a Affinity Purification of Mab

The mAbs were purified from supernatant of the transfected Expi293F cells by Protein A affinity chromatography as described herein.

Size Exclusion Chromatography (SEC) Purification of mAbs

Following Protein A affinity purification, size exclusion chromatography (SEC) was used to further purify and characterise the antibodies as described herein.

Evaluation of Antigen Binding by Monoclonal Antibodies (mAbs)

Purified antibodies were tested for antigen binding on CD20 positive Ramos lymphoma cells by flow cytometry prior to functional analysis.

### Complement Dependent Lysis of Cells

CDC mediated by the mAbs was measured by flow cytometry as described herein using the Zombie Green Fixable Viability kit (BioLegend) following opsonisation of Ramos cells with mAb.

### Results and Discussion

The anti-CD20 mAb rituximab was formatted with unmodified aggregation-resistant IgG3 heavy chains (rituximab-IgG3), aggregation-resistant IgG3 heavy chain comprising the H429F mutation (rituximab-IgG3-H429F), wild type IgG4 heavy chains (rituximab-IgG4-WT) or IgG4 heavy chain comprising the H429F modification (rituximab-IgG4-H429F).

The rituximab-IgG3 mAb was formatted as an aggregation resistant human IgG3 and kappa light chain mAb and a mutant of this antibody was engineered wherein the H429 residue was replaced with phenylalanine to create the rituximab-IgG3(KVH)-H429F mAb. A Rituximab-IgG4-WT mAb was also produced and formatted as a human IgG4 and kappa light chain mAb. In each case, the rituximab IgG3 and IgG4 mAbs showed equivalent characteristics and eluted from the Protein A affinity column as a single homogeneous peak. Further purification by SEC yielded a single IgG (H₂L2) peak for each of the mAbs. Moreover, SDS-PAGE confirmed that the SEC-purified mAbs comprised the expected intact IgG species (prior to disulphide bond reduction), and the ˜50 kDa heavy chain and 25 kDa light chain species following reduction in dithiothreitol.

The CDC potency of the rituximab-IgG3-H429F was evaluated on Ramos cells by flow cytometry and compared to the potency of rituximab-IgG3-WT (FIG. 32A). Each mAb was titrated by serial two-fold dilution in the presence of normal human serum as a source of complement. The percentage killing of CD20-expressing Ramos cells was evaluated at each mAb concentration shown in FIG. 32A. The rituximab-IgG3-H429F mAb showed greater CDC potency than the rituximab-IgG3 mAb, particularly where the concentration of the mAb was limiting. The results are important as they demonstrate that the effects of H429 modification can be successfully incorporated into antibodies containing mutations located elsewhere in the heavy chain (e.g. the N392K, M397V and R435H mutations that overcome aggregation of the IgG). Further, the R435H mutation is known to enhance the in vivo half-life of IgG3, and thus the enhanced CDC by H429F mutation (FIG. 32A) demonstrates that H429 modification can be successfully incorporated into antibodies with other mutations affecting their in vivo half-life.

The CDC potency of rituximab-IgG4-H429F was also evaluated on Ramos cells and compared to the potency of rituximab-IgG4-WT (FIG. 32B). The unmodified rituximab-IgG4-WT failed to kill Ramos cells, but surprisingly, the H429 modified rituximab-IgG4-H429F mAb mediated readily detectable CDC.

Clearly, functional effects arising from H429F modification are not limited to human IgG1 and can be applied more broadly to other immunoglobulin types. This is significant as histidine 429 is conserved at the equivalent position in all human immunoglobulins (IgG, IgA, IgD, IgE and IgM; see FIG. 3, FIG. 4) and in the immunoglobulins of other mammals (e.g. primates).

### Example 6 CDC Killing by Antibodies with an H429 Mutation is Also Dependent on Complement

### CDC by the Mutated Antibodies is Dependent on Both Antibody and Complement

Ramos cells were opsonised with mAbs targeting different molecular targets, CD20 and CD38 or targeting epitopes within the same molecular target (CD20). CDC of Ramos cells using daratumumab-H429F or 11B8-H429F were evaluated in the presence of complement (mAb and human complement), in the presence of mAb but in the absence of complement (mAb only) or in the absence of mAb but in the presence of complement (complement only or C′ only) (FIG. 33). CDC lysis of the Ramos cells was only apparent when both mAb and complement were present. Cells opsonised only with mAb (i.e. in the absence of complement), showed little lysis under the conditions of the CDC assay. Similarly, non-opsonised cells showed little lysis when incubated only with complement. Thus, the lysis of the opsonised cells in the CDC assay was dependent on both antibody and complement.

### Example 7 H429 Modification Confers Enhanced CDC on Monoclonal Antibodies

Isatuximab is a monoclonal antibody that recognises the human CD38 cell surface molecule and which exhibits functional properties that are distinct from those of several other anti-CD38 mAbs such as daratumumab (Deckert J et al., Clin Cancer Res 20:4574-4583, 2014). Isatuximab inhibits the ADP-ribosyl cyclase activity of the CD38 molecule and detects an epitope distinct from daratumumab that inhibits the enzyme activity poorly (Deckert J et al., 2014 supra). The effects of H429 modification on the CDC activity of the functionally distinct isatuximab on cells that are resistant to CDC killing were evaluated.

### Methods and Materials

### Antibodies and Antibody Constructs

The isatuximab-WT mAb used in this example comprised a CD38-specific heavy chain polypeptide with the amino acid sequence shown as SEQ ID NO: 42 comprising, in the order of the N-terminus to the C-terminus, the CD38-specific V_{H} domain of isatuximab mAb fused to CH1-hinge-CH2-CH3 domains of human IgG1, and is encoded by the codon-optimised DNA with the sequence shown as SEQ ID NO: 43. The polypeptide of the anti-CD38 mAb isatuximab light chain comprised the amino acid sequence shown as SEQ ID NO: 44 (comprising the CD38-specific VL domain of the isatuximab mAb fused to human kappa constant domain) and is encoded by the codon-optimised DNA with the sequence shown as SEQ ID NO: 45.

### Synthesis of Unmodified and Mutated Heavy Chains

Antibody expression vectors were generated by standard methods known to those skilled in the art. Briefly, antibody expression vectors consisted of a synthetic polynucleotide sequence, encoding the antibody heavy chain or light chain, appropriately placed within plasmids such as, for example, pcDNA3 and pcDNA3.4 (Thermo Fisher Scientific). Expression vectors for antibodies of different specificities were produced by cleavage at restriction sites at the boundaries of the existing variable domain (V_{H} or V_{L}). A new synthetic DNA encoding the new V domain and flanked by sequences (e.g. 25 nucleotides) that were homologous to the cleaved vector were then incorporated by reaction with NEBuilder (New England Biolabs) according to the manufacturer's instructions.

Fc variants were produced by synthesis of synthetic polynucleotide sequences encoding the variant or by cleavage of the Fc encoding sequence of antibody expression plasmids with appropriate restriction enzymes and the incorporation of new mutagenic synthetic DNA by reaction using NEBuilder (New England Biolabs) according to the manufacturer's instructions.

### Expression of Antibody Constructs

Expression of the antibodies was conducted using transient transfection of Expi293F cells (Thermo Fisher Scientific) under the same conditions as described above in relation to therapeutic mAbs described elsewhere in this specification. As for all mAbs, the cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE.

Purification of mAbs and Evaluation of Antigen Binding

The mAbs were purified from supernatant of the transfected Expi293F cells by Protein A affinity chromatography as described herein. Following Protein A affinity purification, size exclusion chromatography (SEC) was used to further purify and characterise the antibodies as described herein. Purified antibodies were then tested for antigen binding on CD38 positive SUP-15 acute lymphoblastic leukaemia (ALL) cells by flow cytometry prior to functional analysis.

Complement Dependent Lysis of Cells CDC mediated by the mAbs was measured by flow cytometry as described herein using the Zombie Green Fixable Viability kit (BioLegend) following opsonisation of SUP-15 acute lymphoblastic leukaemia (ALL) cells with mAb.

### Results and Discussion

The isatuximab-WT mAb was formatted as a human IgG1 and kappa light chain mAb as described above. A mutant of this antibody was engineered wherein the H429 residue was replaced with phenylalanine to create the isatuximab-H429F mAb. In each case, the isatuximab-based mAbs showed equivalent characteristics and eluted from the Protein A affinity column as a single homogeneous peak. Further purification by SEC also yielded a single IgG (H₂L2) peak for each of the mAbs. Moreover, SDS-PAGE confirmed that the SEC-purified mAbs comprised the expected intact IgG species (prior to disulphide bond reduction), and the ˜50 kDa heavy chain and 25 kDa light chain species following reduction in dithiothreitol.

The CDC potency of the isatuximab-H429F mAb was compared to isatuximab-WT mAb (FIG. 34). Each mAb was titrated individually by serial two-fold dilution in the presence of normal human serum as a source of complement. The percent CDC potency (% killing) was evaluated on the CD38 expressing SUP-15 acute lymphoblastic leukaemia (ALL) cells. As can be seen from FIG. 34, the SUP-15 cells resisted CDC killing by the isatuximab-WT mAb, but were readily killed by the isatuximab-H429F mutant mAb. Thus, substitution at position 429 of the CH3 domain not only enhances CDC against certain cell targets but can also confers potent CDC against targets that resist lysis. Importantly, the results reveal that the lysis of resistant targets can be achieved by H429 modification of unrelated mAbs that also detect distinct epitopes—isatuximab-H429F (FIG. 34) and daratumumab-H429F (FIG. 26). Additionally, the results also indicate that improved CDC efficacy can be conferred on mAbs that have other functional attributes such as the ability to inhibit target molecule function, in this example the ability of isatuximab to inhibit ADP-ribosyl cyclase action of CD38.

### Example 8 Production of Anti-Death Receptor Antibodies with an H429 Mutation

### Methods and Materials

The mAbs used in this example are listed in Table 3 below. The mAbs are designated as BDR5 and TDR5 and detect two distinct epitopes of the human death receptor 5 (DR5; Overdijk M B et al., Mol Cancer Ther 19:2126-2138, 2020) and were produced in the H₂L₂ format with heavy chains of the human IgG1, IgG2 or IgA2 subclass and their appropriate light chain of the human kappa class.

TABLE 3
DR5-specific mAbs detecting different epitopes

| | | Mutation at | |
|---|---|---|---|
| | Epitope | position 429 in | Ig H-chain backbone |
| mAb Name | detected | the H chain | (CH1-hinge-CH2-CH3) |
| BDR5-1WT | a | Wild type | IgG1 delK447 |
| BDR5-1HF | a | H429F | IgG1 delK447 |
| BDR5-2WT | a | Wild type | IgG2 delK447 |
| BDR5-2HF | a | H429F | IgG2 delK447 |
| TDR5-1WT | b | Wild type | IgG1 delK447 |
| TDR5-1HF | b | H429F | IgG1 delK447 |
| TDR5-2WT | b | Wild type | IgG2 delK447 |
| TDR5-2HF | b | H429F | IgG2 delK447 |
| BDR5-A2WT | a | Wild type | IgA delK447-Y465 |
| BDR5-A2HF | a | H429F | IgA delK447-Y465 |
| TDR5-A2WT | b | Wild type | IgA delK447-Y465 |
| TDR5-A2HF | b | H429F | IgA delK447-Y465 |

NOTE:
The H chain backbones are shown in FIG. 3, but for IgG are truncated at G446 (i.e. delK447) and thus lack the C-terminus lysine residue. The IgA H chains are truncated at G446 (i.e. delK447-Y465) and lack the 9 C-terminus amino acids. Numbering of IgG and IgA is based on the sequence alignment in FIG. 3 and FIG. 4.

Generation of DR5-Specific Antibody Constructs with IgG Heavy Chains

Immunoglobulin variable and constant region sequences were assembled by standard molecular biology techniques as complete synthetic DNA molecules to encode entire immunoglobulin H and L chains. These molecules were utilised in the expression vector pcDNA 3.4 (Thermo Fisher Scientific).

The unmodified wild type mAbs used in this example are based on the sequences of two distinct mAbs which specifically bind to the human DR5 molecule (see the DR5-specific mAbs described in U.S. Pat. No. 10,882,913; the entire disclosure of which is herein incorporated by reference). The mAbs were produced as monoclonal IgG antibodies in the H₂L₂ format.

The unmodified DR5-specific mAbs that were generated include the specific VH and VL domains of the mAb indicated in Table 3 and are referred to as the wild type (WT) forms; for instance, the “wild type” BDR5-1WT mAb antibody used in this example, comprised DR5-specific heavy and light chains wherein the DR5-specific heavy chain of the IgG1 subclass comprised the HC-hDR5-01-G56T sequence described previously but lacking the C-terminal lysine (see SEQ ID NO: 36 of U.S. Pat. No. 10,882,913). The DR5-specific kappa light chain comprised the polypeptide sequence of the LC-hDR5-01 described previously (see SEQ ID NO: 39 of U.S. Pat. No. 10,882,913). In other cases, the unmodified antibody was created by fusing the V_{H} domain of the DR5-specific antibody to the wild type heavy (H) chain constant domains of a particular immunoglobulin subclass or class; for instance, unmodified H chains of the BDR5-2WT mAb comprised the DR5-specific V_{H} domain polypeptide sequence of the HC-hDR5-01-G56T sequence described previously (i.e. the V_{H} domain from SEQ ID NO: 36 of U.S. Pat. No. 10,882,913) fused to the constant domain of wild type IgG2 H chain lacking the C-terminal lysine. The DR5-specific kappa light chain of BDR5-2WT mAb comprised the polypeptide sequence of the LC-hDR5-01 described previously (i.e. the LC from SEQ ID NO: 39 of U.S. Pat. No. 10,882,913). More particularly, the BDR5-1WT antibody of the IgG1 subclass used in this example comprised the DR5-specific heavy chain polypeptide sequence of HC-hDR5-01-G56T described previously (i.e. from SEQ ID NO: 36 of U.S. Pat. No. 10,882,913), but lacking the lysine residue at the C-terminus of the H chain, the polypeptide sequence of which is provided herein as SEQ ID NO: 46, and was encoded by a codon-optimised DNA sequence (SEQ ID NO: 47). The DR5-specific kappa light chain of BDR5-1WT mAb was described previously as LC-hDR5-01 in SEQ ID NO: 39 of U.S. Pat. No. 10,882,913, and the polypeptide sequence herein is provided as SEQ ID NO: 48 and is encoded by a codon-optimised DNA with the nucleotide sequence shown as SEQ ID NO: 49.

Separately, the DR5-specific BDR5-2WT antibody was formatted as a human IgG2 antibody subclass comprising, in the order of N-terminus to C-terminus, the DR5-specific V_{H} domain from the HC-hDR5-01-G56T sequence described previously (i.e. from SEQ ID NO: 36 of U.S. Pat. No. 10,882,913) fused to CH1-hinge-CH2-CH3 domains of the human IgG2 subclass H chain lacking the lysine residue at the C-terminus of the H chain (from the polypeptide sequence provided herein as SEQ ID NO: 50) and encoded by a codon-optimised DNA sequence (SEQ ID NO: 51). The polypeptide of the DR5-specific kappa light chain of mAb BDR5-2WT is as described previously as LC-hDR5-01 (i.e. SEQ ID NO: 39 of U.S. Pat. No. 10,882,913) and comprises the polypeptide sequence provided herein as SEQ ID NO: 48 and is encoded by a codon-optimised DNA sequence of SEQ ID NO: 49). Additionally, DR5-specific antibodies denoted as TDR5 that detect an epitope that is distinct from that of the BDR5 mAbs were also generated, particularly TDR5 mAbs based on the VH domains of DR5-specific VH domain of the HC-hDR5-05 polypeptide sequence described previously (see SEQ ID No: 40 of U.S. Pat. No. 10,882,913) and produced with the DR5-specific kappa light chain having the polypeptide sequence as described previously as LC-hDR5-05 (see SEQ ID NO: 43 of U.S. Pat. No. 10,882,913). More particularly, the TDR5-1WT antibody of the IgG1 subclass used in this example comprised the DR5-specific heavy chain polypeptide sequence of HC-hDR5-05 described previously (i.e. from SEQ ID NO: 40 of U.S. Pat. No. 10,882,913) but lacking the lysine reside at the C-terminus of the H chain, the polypeptide sequence of which is provided herein as SEQ ID NO: 52, and was encoded by a codon-optimised DNA with the sequence provided as SEQ ID NO: 53. The polypeptide sequence of the DR5-specific kappa light chain of TDR5-1WT antibody is as described previously as LC-hDR5-05 in (i.e. SEQ ID NO: 43 of U.S. Pat. No. 10,882,913) and is provided as SEQ ID NO: 54 and is encoded by a codon-optimised DNA sequence (SEQ ID NO: 55).

Separately, a TDR5-2WT antibody was formatted as a human IgG2 antibody comprising, in the order of N-terminus to the C-terminus, the DR5-specific VH domain from the HC-hDR5-05 sequence described previously (i.e. from SEQ ID NO: 40 of U.S. Pat. No. 10,882,913), fused to the CH1-hinge-CH2-CH3 domains of the human IgG2 subclass lacking the lysine residue at the C-terminus of the H chain (see the polypeptide sequence provided as SEQ ID NO: 56) and encoded by a codon-optimised DNA sequence (SEQ ID NO: 57). The polypeptide of the DR5-specific kappa light chain of mAb TDR5-2WT is as described previously as LC-hDR5-05 in (i.e. SEQ ID NO: 43 of U.S. Pat. No. 10,882,913) and comprises the DR5-specific kappa light chain of TDR5-1WT antibody (SEQ ID NO: 54) and is encoded by a codon-optimised DNA sequence (SEQ ID NO: 55).

Generation of DR5-specific antibody constructs with IgA H chains

DNA sequences corresponding to the immunoglobulin variable and constant region sequences of the DR5 mAbs were assembled by standard methodologies well known to those skilled in the art as complete synthetic nucleotide sequences to encode the entire immunoglobulin H chain and the immunoglobulin L chain. These sequences were utilised in expression vectors such as pcDNA 3.4 (Thermo Fisher Scientific).

The unmodified mAbs used in this example were based on VH and VL chain domains of the DR5-specific mAbs described in U.S. Pat. No. 10,882,913 and were formatted on human IgA heavy chains and human kappa light chains (Table 3).

The unmodified IgA mAbs include the specific VH and VL domains of the indicated mAb (Table 3) and are referred to as the wild type (WT) forms; for instance, the “wild type” BDR5-A2WT mAb used in this example, comprised the DR5-specific VH domain polypeptide sequence from within the HC-hDR5-01-G56T sequence described previously (SEQ ID NO: 36 of U.S. Pat. No. 10,882,913) and the DR5-specific kappa light chain comprising the polypeptide sequence of LC-hDR5-01 sequence described previously (i.e. SEQ ID NO: 39 of U.S. Pat. No. 10,882,913). More particularly, the BDR5-A2WT mAb used in this example comprised the DR5-specific heavy chain polypeptide (the amino acid sequence of which is provided as SEQ ID NO: 58) comprising, in the order of the N-terminus to C-terminus, the DR5-specific VH domain of HC-hDR5-01-G56T sequence described previously (i.e. the VH portion of SEQ ID NO: 36 of U.S. Pat. No. 10,882,913) fused to CH1-hinge-CH2-CH3 domains of the human IgA2 subclass and encoded by a codon-optimised DNA (SEQ ID NO: 59). The polypeptide of the DR5-specific kappa light chain of the BDR5-A2WT mAb is as described previously as LC-hDR5-01 (i.e. SEQ ID NO: 39 of U.S. Pat. No. 10,882,913) and comprises the DR5-specific VL domain of LC-hDR5-01 fused to a human kappa constant domain light chain polypeptide sequence (SEQ ID NO: 48) and is encoded by a codon-optimised DNA sequence (SEQ ID NO: 49). Additional DR5-specific antibodies that were formatted as IgA mAbs were generated. In particular, the IgA WT mAb, TDR5-A2WT, was generated based on the VH and VL domains the DR5-specific HC-hDR5-05 sequences described in U.S. Pat. No. 10,882,913. More particularly, the TDR5-A2WT antibody used in this example comprised the DR5-specific heavy chain polypeptide (SEQ ID NO: 60) comprising, in the order of the N-terminus to C-terminus, the DR5-specific VH domain within the polypeptide sequence of HC-hDR5-05 described previously (SEQ ID NO: 40 of U.S. Pat. No. 10,882,913) fused to CH1-hinge-CH2-CH3 domains of the human IgGA2 subclass and encoded by a codon-optimised DNA sequence (SEQ ID NO: 61). The DR5-specific light chain polypeptide of the DR5-specific mAb TDR5-A2WT mAb comprised the polypeptide sequence of LC-hDR5-05 described previously (SEQ ID NO: 43 of U.S. Pat. No. 10,882,913) comprising the polypeptide sequence provided herein as SEQ ID NO: 54 and is encoded by a codon-optimised DNA sequence (SEQ ID NO: 55).

### Generation of DR5-Specific Antibody Constructs Encoding Mutated IgG and IgA Heavy Chains

MAbs comprising H chains carrying modification to the sequence at position 429, wherein histidine 429 was replaced with phenylalanine 429 (H429F) in the Fc portion of, for example, IgG1 to generate the BDR5-1HF mAb, were produced from synthetic nucleotide sequences encoding the variant by standard methodologies known to those skilled in the art, and the mutations verified by DNA sequence analysis. In the case of the human IgA H chain, the H436 as defined by the Bur numbering convention, is the equivalent of H429 position in human IgG1 (EU numbering) and referred herein as H429 as defined by the sequence alignment shown in FIG. 3 and FIG. 4. The generated antibodies were as follows:
(i) The BDR5-1HF comprised H chains of the IgG1 subclass carrying the H429F modification and comprising the polypeptide sequence shown as SEQ ID NO: 62 encoded by a codon optimised DNA sequence (SEQ ID NO: 63).
(ii) The BDR5-2HF comprised H chains of the IgG2 subclass carrying the H429F modification and comprised the polypeptide sequence as shown in SEQ ID NO: 64 which was encoded by a codon optimised DNA sequence (SEQ ID NO: 65).
(iii) The TDR5-1HF comprised H chains of the IgG1 subclass carrying the H429F modification and comprised the polypeptide sequence as shown in SEQ ID NO: 66 which was encoded by a codon optimised DNA sequence (SEQ ID NO: 67).
(iv) The TDR5-2HF comprised H chains of the IgG2 subclass carrying the H429F modification and comprised the polypeptide sequence as shown in SEQ ID NO: 68 which was encoded by a codon optimised DNA sequence (SEQ ID NO: 69).
(v) The BDR5-A2HF comprised H chains of the IgA2 subclass carrying the H429F modification and comprised the polypeptide sequence as shown in SEQ ID: 70 which was encoded by a codon optimised DNA sequence (SEQ ID NO: 71).
(vi) The TDR5-A2 HF comprised H chains of the IgA2 subclass carrying the H429F modification and comprised the polypeptide sequence as shown in SEQ ID NO: 72 which was encoded by a codon optimised DNA sequence (SEQ ID NO: 73).

All H chain Fc variants were produced with their appropriate matched human kappa L chains as follows: The BDR5 H chain variants were produced with a human kappa L chain comprised of the polypeptide sequence of LC-hDR5-01 as described previously in (i.e. SEQ ID NO: 39 of U.S. Pat. No. 10,882,913) comprising the polypeptide sequence provided as SEQ ID NO: 48 and is encoded by a codon-optimised DNA with the sequence shown as SEQ ID NO: 49. The TDR5 H chain variants were produced with a human kappa L chain comprised of the polypeptide sequence of LC-hDR5-05 described previously in (i.e. SEQ ID NO: 43 of U.S. Pat. No. 10,882,913) comprising the polypeptide sequence of SEQ ID NO: 54 and is encoded by a codon-optimised DNA with the sequence shown as SEQ ID NO: 55.

Expression of the antibodies was conducted using transient transfection of Expi293F cells (Thermo Fisher Scientific). Expi293F cells were cultured in Expi293 Expression Media (Life Technologies) and, 24 hours prior to transfection cells, were split to a concentration of 2×10⁶ viable cells/ml. On the day of transfection, 7.5×10⁷ viable log phase cells were centrifuged and resuspended in 25 ml of pre-warmed, antibiotic-free Expi293 Expression Media and maintained at 37° C. until transfection. Transfection of the cells was then performed at RT using the Expifectamine Transfection Kit (Life Technologies) as follows. Eighty microlitres of ExpiFectamine 293 reagent was diluted with 1.5 ml Opti-MEM-I Reduced Serum Medium (Gibco) and incubated for 5 minutes at RT. Thirty μg of DNA (15 μg of H chain DNA and 15 μg of L chain DNA) was diluted in 1.5 ml of Opti-MEM-I reduced serum medium and was then added to the diluted ExpiFectamine reagent, incubated for 20-30 minutes at RT and then added dropwise to the Expi293F cell suspension which was then cultured at 37° C. for 16 to 18 hours at which time 150 μL of the manufacturer's Enhancer 1 and 1.5 ml of Enhancer 2 were added and the cells cultured for a further four days at 34° C. or 37° C.

Cell cultures were harvested and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE.

Protein a Affinity Purification of IgG mAbs

The mAbs were purified from the supernatant of the transfected Expi293F cells by Protein A affinity chromatography. Briefly, Hi-trap™ Protein A high-performance columns (GE Healthcare Life Sciences) were washed and equilibrated in binding buffer (20 mM NaH₂PO₄, pH 7.0). The cell culture supernatant was loaded, the columns washed with binding buffer to baseline OD280 nm and bound antibody eluted with 0.1 M sodium citrate tribasic dihydrate (pH 3.5) and 1 ml fractions collected and neutralised immediately with 1 M Tris-HCl pH 9.0. Fractions containing the antibody were pooled.

Size Exclusion Chromatography (SEC) of IgG mAbs

Following Protein A affinity purification, size exclusion chromatography (SEC) was used to further purify and characterise the antibodies. The Protein A purified antibodies were concentrated to OD280 nm of 6-8 using 30 kDa molecular weight cut-off centrifuge concentrator device (Merck Millipore). Superose 6 10/300GL columns (GE Healthcare Life Sciences) were equilibrated in PBS, pH7.2, then the concentrated Protein A affinity purified antibody was loaded and separated at a flow rate of 0.5 ml per minute in PBS and 0.5 ml fractions collected. For some mAbs, SEC was performed at pH 5.0, thus the concentrated Protein A purified mAb was dialysed overnight against buffer (100 mM sodium citrate, 100 mM NaCl, pH 5.0), then applied to a Superose 6 10/300 column pre-equilibrated in the same buffer. The dialysed antibody was applied to the Superose column SEC at a flow rate of 0.5 ml per minute and 0.5 ml fractions collected from the column.

Evaluation of Antigen Binding by IgG or IgA mAbs

Purified antibodies were tested for DR5 antigen binding prior to functional analysis by flow cytometry as previously described in Trist et al., J Immunol 192(2):792-803, 2014 using a BD Biosciences Canto™ 11 flow cytometer (Becton Dickinson). The DR5-specific mAbs were tested for binding on, Colo205 colorectal cells, Ramos lymphoma cells and KMS-12-PE cells. Briefly, purified mAbs or culture supernatants from Expi293 cells producing the mAb, were titrated by serial two-fold dilution in 25 μl of FACS buffer (phosphate buffered saline with 0.5% (w/v) bovine serum albumin). Note, in the case of binding to KMS-12-PE cells, the mAbs were used at a single concentration. Twenty-five microlitres of target cells at the concentration of 5×10⁶/ml were then added to the mAb and incubated for 30 minutes on ice. Cells were then washed twice in FACS buffer, resuspended with 50 μl of anti-human IgG (Fc)-FITC conjugate (1/500 dilution; Chemicon Merck, Burlington, MA, United States of America) for detection of the bound IgG mAbs. After 30 minutes incubation on ice, they were washed and resuspended in 200 μl FACS buffer for analysis. The IgA mAbs were detected with 50 μl of biotin-conjugated anti-human IgA (0.5 μg/ml) (BD Pharmingen, San Diego, CA, United States of America) incubated for 30 minutes on ice, washed and resuspended in APC-conjugated streptavidin (1/500 dilution), washed twice in cold FACS buffer and then resuspended in 200 μl FACS buffer for analysis.

### Viability Assay

The capacity of antibodies to induce cell death was evaluated in a viability assay. The antibodies were tested either individually as single agents or as mixtures of two antibodies. The viability assay was performed using cell lines which were cultured routinely in culture medium consisting of RPMI 1640 medium supplemented with 10% (v/v) foetal calf serum plus 4 mM Glutamine and 100 U/ml penicillin and 0.1 mg/ml streptomycin.

The cells were harvested from cell culture, washed in culture medium by centrifugation at 200×g and resuspended in culture medium. The cells were washed again and resuspended in culture medium to a concentration of 1.2×10⁶/ml. One hundred microlitres (100 μL) of cell suspension (10,000 cells) was added to wells of 96-well flat-bottom sterile plates. Purified mAbs either as individual mAbs or mAb mixtures were added in 100 μL of medium at a single concentration or alternatively were serially, two-fold diluted in culture medium over a concentration range (10 μg/ml to 0.075 μg/ml) and 100 μL of each dilution was added to the 96-well plates previously seeded with cells along with an additional 75 μL of culture medium to bring the total final volume in each well to 200 μL. For the mAb mixtures, the total concentration of mAb was the same as in the equivalent wells treated with the single antibodies. The plates were then incubated for 48-72 hours at 37° C. in 5% CO₂, and in some experiments, maximum cell death was determined by addition of 10 μl of 1% SDS in the last hour of the incubation (i.e. at the end of the incubation period). Following the incubation period, the viability of the cells was determined using a colorimetric assay using Cell Counting Kit 8 (CCK-8; Abcam, Cambridge, United Kingdom). This assay indicates cell viability by the production of a formazan dye upon bio-reduction of the tetrazolium salt WST-8 by living cells which is directly proportional to the number of living cells. The WST-8 solution was diluted 1:2 with culture medium and 40 μl added to each well of 96-well plates. The plates were incubated at 37° C. for 2-5 hours. Colour development was determined by measuring absorbance at 450 nm (Abs450) on a Fluorostar OPTIMA (BMG LABTECH, Ortenberg, Germany).

The viability assay analysis of IgA formatted DR5 mAbs was performed as for IgG except that cell culture supernatant from Expi293 cells producing the mAbs was used as the source of mAbs and was serially diluted as for the IgG mAbs. The maximum viability control values were determined from cells cultured in 200 μL of culture medium in the absence of antibody. The control for maximum death was determined from wells seeded only with cell suspension in 200 L of culture medium but treated at the end of the incubation period with 10 μl of 1% SDS for 1 hour at 37° C. (i.e. in the last hour of the incubation period). Following the incubation period, the viability of the cells was determined using a colorimetric assay using Cell Counting Kit 8 (CCK-8; Abcam). This assay indicates cell viability by the production of a formazan dye upon bio-reduction of the tetrazolium salt WST-8 by living cells which is directly proportional to the number of living cells. The WST-8 solution was diluted 1:2 with culture medium and 40 μl added to each well of 96-well plates. The plates were incubated at 37° C. for 2-5 hours. Colour development was determined by measuring absorbance at 450 nm (Abs450) on a Fluorostar OPTIMA (BMG).

### Results

Induction of Apoptotic Cell Death by mAbs

The effect of antibody Fc modifications on the agonistic activity of monoclonal antibodies was evaluated using the induction of apoptosis by DR5-specific antibodies as an example. MAbs that recognise distinct epitopes on the human death receptor 5 (DR5, TRAILR2) cell surface molecule (Table 3) were produced and investigated for their capacity to induce cell death as determined in assays of cell survival. The mAbs, BDR5 and TDR5 are directed at two distinct epitopes on human DR5 and were formatted as H₂L2 antibodies comprising DR5-specific antibody light chains and also comprising DR5-specific H chains that lacked the C-terminus lysine but were otherwise wild type human IgG1 or human IgG2 or human IgA subclass (Table 3) or otherwise comprising Fc modified DR5-specific human IgG1, IgG2 and IgA H chains wherein the histidine at position 429 of the CH3 domain of the H chain had been replaced with phenylalanine (H429F).

Quantitation of Cell Binding by IgG Formatted DR5-Specific mAbs

Purified mAbs detecting DR5 were tested by flow cytometry for binding to DR5 positive Colo205 colorectal cells (FIG. 35) or Ramos lymphoma cells (FIG. 36). All mAbs showed readily detectable levels of target antigen binding. Moreover, the Fc modified mAbs carrying a mutation in the IgG heavy chain showed identical binding activity to that of the wild type (WT) form. For example, the IgG1 formatted BDR5-1HF which includes the H429F modification showed identical binding to Colo205 cells as unmodified wild type IgG1 BDR5-1WT (FIG. 35A). The DR5-specific mAbs BDR5-2HF and BDR5-2WT formatted as IgG2 backbone (FIG. 35B) also showed identical binding activity to each other, thereby clearly demonstrating that modification of the Fc does not affect interaction of the mAbs with their target DR5 epitope. The observation that Fc modifications at position 429 do not affect antigen binding by the modified mAbs was confirmed by the analysis of TDR5 mAbs which detect a second epitope distinct from that detected by the BDR5 mAbs. Binding by TDR5-1WT and TDR5-1HF was identical (FIG. 35C) as was the binding by the IgG2 formatted forms TDR5-2WT and TDR5-2HF (FIG. 35D). Further analysis of mAb binding to DR5 expressed on Ramos lymphoma cells showed that the binding profile of mAbs was unaffected by the Fc mutation. The IgG1 formatted mAbs BDR5-1WT and BDR5-1HF (FIG. 36A) and the IgG2 formatted BDR5-2WT and BDR5-2HF (FIG. 36B) showed identical binding to Ramos lymphoma cells. The TDR5-1WT, TDR5-1HF as IgG1 WT or H429F H chains respectively (FIG. 36C) and TDR5-2WT, TDR5-2HF as IgG2 WT and H429F H chains respectively (FIG. 36D) also bound equivalently. Thus, binding to DR5 was unaffected by H429F mutation. This was further confirmed by binding to KMS-12-PE myeloma cells, where the binding of wild type or H429F mAb was identical for each mAb (e.g. BDR5-1WT and BDR5-1HF bound identically as did the IgG2 mAbs BDR5-2WT and BDR5-2HF; FIG. 37). This again confirms that the binding to the target antigen was unaffected by H429F mutation and WT and mutant mAbs bound similarly to colorectal cells, B lymphoma cells or myeloma cells.

Evaluation of the Cell Killing Potency of DR5-Specific mAbs Comprising IgG H Chains

The capacity of the DR5 mAbs to induce death of cells of different lineages was determined in a colorimetric assay that measured the survival of cells that had been cultured in the presence of the DR5-specific antibodies (FIG. 38, FIG. 39). The relative potency of mAbs comprising heavy chains carrying the H429F modification or comprising wild type H chains was determined.

### H429F Enhances DR5 mAb Induced Killing of Colorectal Cells

Colo205 colorectal cells were incubated in the presence of 20 μg/ml of the DR5 mAbs. The TDR5-1HF mAb which comprised IgG1 H chains containing the H429F modification was a potent inducer of death of Colo205 cells, Abs450 nm=1.37 by comparison to cell survival in the absence of antibody, Abs450 nm =2.24 (FIG. 38, no ab) and was substantially more potent than its wild type counterpart TDR5-1WT, which comprised wild type IgG1 heavy chains and which exhibited little capacity to induce cell death, Abs450 nm=1.99 (FIG. 38).

The effect of H429F modification on DR5 mAb comprising IgG2 heavy chains was also determined. The TDR5-2HF mAb (20 μg/ml) comprising an IgG2 heavy chain containing the H429F modification was a more potent inducer of death of Colo205 cells, Abs450 nm=1.32 (FIG. 38) by comparison to cell survival in the absence of antibody, Abs450 nm=2.24 (FIG. 38, no ab) and was substantially more potent than its wild type counterpart TDR5-2WT which comprises wild type IgG2 heavy chains, and which exhibited little capacity to induce cell death, Abs450 nm=1.91 (FIG. 38).

Thus, H429F modification of the IgG Fc improves the capacity of anti-death receptor mAbs to induce cell death resulting in reduced cell survival. This improved potency resulting from the H429F modification is independent of human IgG subclass since the TDR5-1HF and TDR5-2HF mAbs which comprise H chains of different IgG subclasses show equivalent improved potency compared to the wild type equivalents. (FIG. 38).

The TDR5 and BDR5 mAbs detect distinct epitopes on DR5. Thus, to further determine the effects of the H429F modification on the action of anti-death receptor mAbs, the Colo205 cells were incubated in a 1:1 mixture (10 μg/ml of each mAb for a total mAb concentration of 20 μg/ml) of the TDR5-2HF mixed with BDR5-2HF mAbs which are comprised of IgG2 subclass H chains carrying the H429F modification (FIG. 38). Whilst the TDR5-2HF mAb used individually showed clear improvement in killing potency (Abs450 nm=1.32) compared to its wild type counterpart (Abs450 nm =1.91), the mixture of TDR5-2HF with BDR5-2HF showed even greater improvement in killing and indeed exhibited near-total killing of cells, Abs450 nm=0.1 (FIG. 38).

### H429F Enhances DR5 mAb Induced Killing of Lymphoma Cells

Having established the killing potency of the DR5 mAbs on colorectal Colo205 cells, mAb killing potency was then investigated on cells of a different lineage, Ramos lymphoma cells (FIG. 39).

The TDR5-1HF mAb comprising IgG1 H chains containing the H429F modification and the TDR5-2HF mAb comprising IgG2 H chains containing the H429F modification were both potent inducers of death of Ramos cells, TDR5-1HF Abs450 nm=0.39, TDR5-2HF Abs450 nm=0.42 compared to cell survival in the absence of antibody, Abs450 nm=0.65 (no mAb) and were substantially more potent than their wild type counterparts comprised of wild type IgG1 or IgG2 H chains and which exhibited little capacity to induce cell death, TDR5-1WT Abs450 nm=0.59, TDR5-2WT Abs450 nm=0.51 (FIG. 39). Thus, as was observed in the killing of colorectal cells (FIG. 38), the H429F modification of the IgG Fc improves the capacity of anti-death receptor mAbs to induce cell death of lymphoma cells resulting in reduced cell survival indicating that enhanced killing potency is not cell type dependent. Further, the improved killing of the lymphoma cells resulting from the H429F modification is independent of human IgG subclass since the TDR5-1HF and TDR5-2HF mAbs which comprise H chains of different IgG subclasses (i.e. IgG1 or IgG2) show near equivalent improvement in potency compared to their IgG1 or IgG2 wild type equivalents (FIG. 39).

Since dual-epitope targeting by mixtures of mAbs comprised H chains carrying the H429F modification showed maximum killing of Colo205 colorectal cells compared to killing by the individual mAbs or wild type mAbs (FIG. 38), the same mixture was tested for capacity to kill lymphoma cells (FIG. 39). Accordingly, Ramos cells were incubated in a 1:1 mixture of the TDR5-2HF mixed with BDR5-2HF mAbs (10 μg/ml of each mAb for a total mAb concentration of 20 μg/ml) which are comprised of IgG2 subclass H chains carrying the H429F modification (FIG. 39). Whilst the TDR5-2HF mAb used individually showed clear improvement in killing potency (Abs450 nm=0.42) compared to its wild type counterpart (Abs450 nm=0.51), the mixture of TDR5-2HF with BDR5-2HF showed even greater improvement in killing and indeed exhibited near-total killing of cells, Abs450 nm=0.03 (FIG. 39).

### Effectiveness of Dual-Epitope Targeting: Killing by DR5 mAb Mixtures Matched for IgG Subclasses

The effect of H429 modification on killing potency of mixtures of mAbs directed to different epitopes was investigated using pairwise combination of mAbs (FIG. 40) incubated with Colo205 cells. In each pairwise mixture, the BDR5 and TDR5 mAbs were present in a 1:1 ratio and both mAbs comprised the same IgG heavy chain subclass with the H429F modification (BDR5-1HF with TDR5-1HF both IgG1 or BDR5-2HF with TDR5-2HF both IgG2) and their killing potency compared to their equivalent paired mAbs of wild type H chain (BDR5-1WT with TDR5-1WT both IgG1 or BDR5-2WT with TDR5-2WT both IgG2). The mixtures were serially two-fold titrated from 1 μg/ml total mAb (BDR5 mAb 0.5 μg/ml+TDR5 mAb 0.5 μg/ml) and the effect on survival of Colo205 cells determined after 48 hours. The mixture of the BDR5-1WT with TDR5-1WT comprised of the IgG1 wild type chains failed to induce appreciable killing of Colo205 cells. Indeed, cell survival in this mixture, even at the highest concentration, 1 μg/ml, was equivalent to survival in the absence of antibody (no ab, FIG. 40). Replacing the IgG subclass did not improve potency as the mix of IgG2 BDR5-2WT with TDR5-2WT also lacked killing potency even at 1 μg/ml. Thus, the lack of potency of the WT mAb mixtures was not influenced by the IgG subclass since both IgG1 and IgG2 mixtures were equally poor. However, mixtures comprised of mAbs with heavy chains containing the H429F modification were very potent inducers of killing. Mixtures of BDR5-1HF with TDR5-1HF comprising IgG1 H chains with the H429F modification achieved levels of killing that were equivalent to control for maximum death of cells determined by cell survival in the presence of SDS detergent (SDS, FIG. 40). Further, the mAb mixture BDR5-2HF with TDR5-2HF whose mAbs comprised IgG2 H chains with the H429F modification was similarly potent as their IgG1 based equivalent mAbs mixture, BDR5-1HF with TDR5-1HF. Thus, improved potency is dependent on H429F modification but is not dependent on the IgG subclass as these showed near equivalent improvements of killing whether formatted as a human IgG1 or human IgG2 subclass H chain.

### Effectiveness of Dual-Epitope Targeting: Killing by DR5 mAb Mixtures of Distinct Specificity and IgG Subclasses

Pairwise comparisons of mAbs detecting distinct epitopes of DR5 and comprising IgG heavy chains of different IgG subclasses were undertaken to determine if the superior killing potency of mAb mixtures is determined by the nature of the IgG subclass and/or epitope individual mAbs that are combined in the mixture. In particular, pairwise mixtures in a 1:1 ratio of the mAbs were made by mixing two mAbs at equal concentration (0.5 μg/ml+0.5 μg/ml) to yield a total antiDR5 mAb concentration of 1 μg/ml in the mixture (FIG. 41). The potency of the mixtures of the DR5 mAbs was then determined by culturing Colo205 cells with serial 2-fold titrated mAb mixtures wherein the DR5 mAbs in each mixture comprised different human IgG H chain subclasses (FIG. 41).

The mixture of the BDR5-1WT mAb, which comprised wild type IgG1 heavy chains, with the TDR5-2WT mAb, which comprised wild type IgG2 heavy chains (BDR5-1WT+TDR5-2WT) showed only limited killing of Colo205 cells and only at the highest concentration (0.5 μg/ml of each mAb; i.e. 1 g/ml total mAb). In contrast, the combination of the BDR5-1HF mAb, which comprised IgG1 heavy chains containing the H429F modification, mixed with the TDR5-2HF which comprised IgG2 heavy chains containing the H429F modification (BDR5-1HF+TDR5-2HF) and mediated maximum killing comparable to levels equivalent to the SDS kill control (FIG. 41) and importantly showed greater killing potency than the mixture of their wild type mAb equivalents (BDR5-1WT+TDR5-2WT). Further, the killing potency of the mixture of the H429F modified mAbs also showed more than 10-fold greater efficacy by titration than the WT equivalents.

The reciprocal paired mixture (BDR5-2HF+TDR5-1HF) where the BDR5-2HF comprised IgG2 heavy chains containing the H429F modification and was paired with TDR5-1HF that comprised IgG1, heavy chains containing the H429F modification, also showed maximum killing levels and titre similar to the BDR5-1HF+TDR5-2HF mixture. The equivalent mixture of mAbs comprising wild type H chains (BDR5-2WT+TDR5-1WT) failed to show appreciable killing of the Colo205 cells. It is also noteworthy that the levels of killing achieved by IgG comprising H chains carrying the H429F modification was the equivalent of killing by “homogeneous” mixtures of mAbs of the same H chain subclass carrying the H429F modification for example IgG2 (BDR5-2HF with TDR5-2HF) (FIG. 40, FIG. 41).

Clearly mixtures of DR5 mAbs with wild type H chains, in any combination of pairs, showed little potency but the inclusion of H429F in the mAbs greatly increased cell killing.

Effect on Killing by Altering the Ratio of mAbs in the Mixtures

Individual mAbs containing H429F showed improved potency over their wild type counterparts when used individually (see FIG. 38, FIG. 39, FIG. 42, and FIG. 43), but greatest potency was achieved in mixtures of mAbs comprising H chains carrying the H429F modification (FIG. 40, FIG. 41). However, the mixtures used in the experiments above (FIGS. 38-41) comprised mAbs at the same concentration (i.e. a 1:1 ratio). Thus, the effect of varying the ratio of the individual mAbs in the mixtures on the viability of cells was determined.

Cell survival was determined by culture of Colo205 cells (FIG. 42) or of Ramos cells (FIG. 43) with mixtures of BDR5-1HF mAb and TDR5-1HF mAb at a final concentration of 1 μg/ml of total DR5-specific mAb as follows: 100% BDR5-1HF with 0% TDR5-1HF, 90% BDR5-1HF with 10% TDR5-1HF, 75% BDR5-1HF with 25% TDR5-1HF, 50% BDR5-1HF with 50% TDR5-1HF, 25% BDR5-1HF with 75% TDR5-1HF, 10% BDR5-1HF with 90% TDR5-1HF and 0% BDR5-1HF with 100% TDR5-1HF. Survival of Colo205 cells was unaffected or in the case of Ramos cells was little affected, in the presence of individual mAbs comprising the wild type H chains. Indeed, for Colo205 cells treatment with individual mAbs was equivalent to survival in the absence of mAbs (no ab control, FIG. 42, FIG. 43). Survival in the presence of only individual BDR5-1HF mAb, and individual TDR5-1HF mAb both of which comprised H429F modified IgG1 H chains, was modestly reduced compared to wild type. Surprisingly, however, all mixtures showed near maximum killing of Colo205 and Ramos cells, equivalent to the SDS maximum death control. Also, surprisingly, this potent killing was apparent even when one mAb was present in the mixture at as little as 10%, for example BDR5-1HF 90:10 TDR5-1HF (FIG. 42, FIG. 43). Further, the relative abundance of a mAb of one epitope specificity in the mixture did not affect potency (i.e. the BDR5-1HF: TDR5-1HF mixes were equipotent at all ratios for example, BDR5-1HF 90:10 TDR5-1HF was as potent as BDR5-1HF 10:90 TDR5-1HF; FIG. 42, FIG. 43).

Evaluation of mAbs Comprising IgA H Chains Carrying the H429 Modification

The DR5-specific mAbs used in the IgG experiments above were also produced comprising wild type IgA H chains or comprising IgA chain containing the H429F modification, and were evaluated for their capacity to induce cell death.

The DR5-specific mAbs, BDR5 and TDR5, are directed at two distinct epitopes on human DR5 and were produced according to the method above, as antibodies comprising H chains of the human IgA and, specifically, of the human IgA2 subclass as an example (Table 3). Thus, the BDR5-A2WT mAb and the TDR5-A2WT mAb comprised wild type IgA2 subclass H chains and the BDR5-A2HF mAb and the TDR5-A2HF mAb comprised IgA2 subclass H chains carrying the equivalent of the H429F modification of IgG1 (FIG. 3, Table 3); and in all cases, the H chains were paired with the specific BDR5 or TDR5 human kappa light chains. The antibodies comprising their L chains and IgA H chains were produced in Expi293 cells and the supernatant from these cultures used as a source of DR5 mAbs.

The binding of the IgA formatted DR5-specific antibodies to target cells was evaluated using indirect immunofluorescence and quantitated by flow cytometry (FIG. 44). All mAbs detected DR5 on the Colo205 cells with a level of binding that was significantly above background control levels of non-specific binding of the fluorescent conjugate to cells in the absence of antibody (no ab) or the background level of cell fluorescence (cells) (FIG. 44). Turning to the capacity of the DR5-specific mAbs (comprising the IgA H chains) to induce cell death, this was assessed by culturing Colo205 cells in the presence of serial dilutions of the mAbs (FIG. 45). The titration curves revealed that the inclusion of the H429F in the IgA heavy chain conferred potent cell death, as the mAbs comprised of an IgA heavy chain containing the H429F modification were many-fold more potent inducers of cell death than the equivalent mAbs comprising the wild type IgA heavy chain. Thus, for example, the BDR5-A2HF (EC50 >1/256 dilution) and TDR5-A2HF (EC50 >1/512) were potent inducers of killing compared to the IgA BDR5-A2WT (which showed little killing at any dilution) and TDR5-A2WT (which showed killing only at high concentration (low dilution) (EC50 ˜1/6 dilution)).

Clearly, the transformation of killing potency by the inclusion of the H429F modification in immunoglobulin heavy chains is apparent in IgA heavy chains as well as the IgG heavy chains.

Cell Specificity and Killing Potency by Mixtures of H429F Modified mAbs Detecting Different Molecular Targets

As described above, mixtures of H429F modified mAbs, where each of the H429F modified mAbs detected different epitopes on the same molecular target, showed functional improvement above their separate components at the level of C1q binding (see FIG. 29), CDC killing potency (see FIGS. 30C) and DR5-induced cell death (FIGS. 38-43). Further, improved CDC was achieved by the combination of H429 modified mAbs recognising different antigens, that is different molecular targets, such as CD20 and CD38 (FIG. 30B). Here, an experiment in mAb mediated cell death was conducted to assess the potency of mixtures of H429F modified mAbs directed at different molecular targets (e.g. CD38 and DR5). The isatuximab mAbs comprising WT or H429F mutated IgG H chains was used herein are as described in Example 7. In particular, Ramos lymphoma cells (FIG. 46) which express CD38 and DR5 were treated individually with an anti-CD38 mAb isatuximab comprising WT H chains, wild type isatuximab (Isa-WT), or with anti-DR5 mAb BDR5-1 comprising WT H chains (BDR5-1WT). The treatment with these mAbs alone resulted in little change in cell viability compared to untreated cells (A450 nm=0.720). Also, treatment with a 1:1 mixture of the Isa-WT and BDR5-1WT mAbs (Isa-WT+BDR5-1WT; FIG. 46) resulted in only a small reduction in cell viability (A450 nm=0.576). A similar reduction in viability was achieved by treatment with the individual H429F-modified mAbs, Isa-HF (A450 nm=0.550) or BDR5-1HF (A450 nm=0.564) mAbs. However, in contrast, a mixture of the Isa-HF with BDR5-1HF mAbs (Isa-HF+BDR5-1HF, FIG. 46), both of which comprise H chains carrying the H429F modification, was clearly more effective (A450 nm=0.372) in reducing cell viability by the induction of cell death.

### Discussion

The results obtained in this example clearly demonstrate that the substitution of histidine 429 in the IgG heavy chain confers increased capacity of the mAbs to induce cell death in target cells expressing a target antigen. In particular, the H429F modification of the CH3 domain was found to substantially improve the killing potency of anti-death receptor mAbs compared to mAbs with wild type H chains (i.e. lacking any modification at position 429). Importantly, the improvement in killing potency was found to be independent of the epitope on the target, independent of the IgG heavy chain subclass into which the H429 modification was made, and independent of the cellular target, and notably, the H429F modification overcame the poor killing capability of WT mAbs directed at different epitopes. In addition, it was found that the improved killing by BDR5 and TDR5 mAbs comprising the H429F modification, was achieved when these were formatted as either IgG1 and IgG2 backbones and was further increased by combining the two BDR5 and TDR5 mAbs regardless of subclass format, i.e. when the two DR5 clones were formatted as H429F modified mAbs of the same IgG subclass (IgG1 or IgG2) or when one clone was IgG1 and the second clone IgG2. In sum the effect of H429 modification of the Fc was robust, being independent of the context of IgG subclass, enhancing the receptor agonism of the mAbs, individually or in combination, as measured in these examples by DR5-induced apoptosis. Collectively, these results are very significant in that they confirm the broader utility of H429 modifications to other antibodies, antibody-like molecules and antibody derivatives, as H429 is conserved in, for example, the IgG subclasses of humans, non-human primates and mice (see the sequence comparisons provided in FIG. 3 and FIG. 4). Moreover, H429 is also found in the equivalent position in the H chains of other human Ig classes (i.e. IgA, IgE and IgM) which are structurally distinct from IgG and would not be expected to mimic the Fc-related properties attributable to the IgG Fc portion. Nonetheless, using IgA as an example, the DR5-specific mAbs used in the IgG studies were produced comprising instead wild type IgA H chains or IgA H chains containing the H429F modification, and were also found to exhibit enhanced cell killing capability.

Interestingly, the improved killing by individual mAbs carrying the H429F modification was substantially further improved by culturing target cells in a mixture of mAbs binding to different epitopes of a target antigen (e.g. different epitopes of DR5). In particular, it was found that combining TDR5-2HF and BDR5-2HF antibodies achieved a more potent level of killing than the individual mAbs (containing the H429F modification) to the extent that a mixture of the mAbs resulted in almost complete killing of the cells even at ratios of 90:10. Finally, the results of this example also showed that the improved killing achieved with mAbs including an H429 modification, whether used as individual mAbs or in mixtures, was not restricted to the target cell type and indeed, the DR5-specific antibodies, whether comprised of IgG1 or IgG2 H chains modified at H429, exhibited similar levels of enhanced killing of colorectal and lymphoma cell types.

Methods that focus killing mechanisms of action more effectively to intended target cells are desirable for improved immunotherapy with mAbs. The property of H429-modified mAbs and Fc-fusion proteins in forming oligomeric platforms (e.g. for optimal CDC) provides a route to combining mAbs with differing specificities for increased cellular target specificity. Thus, using one mAb that targets a molecule that is present on a wide range of cell types, but mixed with a mAb targeting a molecule of limited cell expression can achieve specific cell killing potency. For example, DR5 and CD38 have distinct cell and tissue distributions but they are co-expressed on some cell types particularly haemopoietic cells (Zola et al., supra 2007). The results shown in FIG. 46 demonstrate this approach and demonstrate that an individual H429F modified DR5 mAb or an individual H429F modified CD38 mAb induces modest apoptosis but lymphoma killing is potentiated by their combination where their antigens are co-expressed on the target cell. This enhancement of killing by combinations of H429F modified mAbs detecting different molecular targets may result from a number of mechanisms dependent on H429F modification of the Fc such as the formation of hetero-oligomers wherein the different cell surface targets are incorporated into the signalling cluster by virtue of the interaction between the H429F modified H chains of the different mAbs, for example, containing both CD38:anti-CD38 and DR5:anti-DR5 oligomeric complexes. Also, such potentiation may reflect the integration of signals propagated by the combined engagement and cross-linking of DR5 and CD38 by these mAbs whether homo- or hetero-oligomers or by a combination of these effects. Thus, the combined use of H429F modified mAbs that detect separate molecular target molecules combines target cell specificity with killing potency. Such a combination of effects is desirable in overcoming the often-limiting factors in therapy including lack of specificity and unwanted toxicity.

### Example 9 H429 Modification Confers Enhanced CDC on Further Example of Anti-CD38 Monoclonal Antibody

Mezagitamab is a monoclonal antibody that recognises the human CD38 cell surface molecule and which exhibits functional properties that are distinct from those of several other anti-CD38 mAbs such as daratumumab (Deckert J et al., 2014 supra). The effects of H429 modification on the CDC activity of the mezagitamab on cells that are resistant to CDC killing were evaluated.

### Methods and Materials

### Antibodies and Antibody Constructs

The mezagitamab-WT mAb used in this example comprised a CD38-specific heavy chain polypeptide with the amino acid sequence shown as SEQ ID NO: 74 comprising, in the order of the N-terminus to the C-terminus, the CD38-specific VH domain of mezagitamab mAb fused to CH1-hinge-CH2-CH3 domains of human IgG1, and which is encoded by the codon-optimised polynucleotide sequence shown as SEQ ID NO: 75. The polypeptide of the anti-CD38 mAb mezagitamab light chain comprised the amino acid sequence shown as SEQ ID NO: 76 (comprising the CD38-specific VL domain of the mezagitamab mAb fused to human lambda constant domain) and which is encoded by the codon-optimised polynucleotide sequence shown as SEQ ID NO: 77.

### Synthesis of Unmodified and Mutated Heavy Chains

Antibody expression vectors and Fc variants including the replacement of H429 with phenylalanine (H429F) in the H chains of the CD38 mAbs were generated by standard methods known to those skilled in the art and as are described elsewhere in this specification in relation to therapeutic mAbs (e.g. Example 3).

### Expression of Antibody Constructs

Expression of the antibodies was conducted using transient transfection of Expi293F cells (Thermo Fisher Scientific) under the same conditions as described above in relation to other therapeutic mAbs described in this specification. As for the mAbs, the cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE analysis.

Purification of mAbs and Evaluation of Antigen Binding

The mezagitamab-H429F and mezagitamab-WT mAbs were purified from supernatant of the transfected Expi293F cells using Protein A affinity chromatography followed by size exclusion chromatography. The SEC purified mAbs showed the expected H₂L₂ format of monomeric IgG confirmed by SDS-PAGE analysis. Following purification, binding to CD38 positive cells was confirmed by flow cytometric analysis (data not shown).

### Complement Dependent Lysis of Cells

CDC of target cells mediated by mAbs was measured by flow cytometry as described elsewhere in this specification (e.g. Example 3) using the Zombie Green Fixable Viability kit (BioLegend) following opsonisation of cells with mAbs.

### Results and Discussion

The capacity of the mezagitamab-H429F mAb to mediate CDC killing of cells was compared to CDC killing by its wild type counterpart mezagitamab-WT mAb (FIG. 47). Each mAb was titrated individually by serial two-fold dilution in the presence of normal human serum as a source of complement. The CDC potency (% killing) was evaluated on Ramos cells expressing the CD38 antigen. As can be seen from FIG. 47, the Ramos cells resisted CDC killing by the mezagitamab-WT mAb which only showed a maximum level of killing of 27.61% at the highest concentration (5 μg/ml) but were readily killed by the mezagitamab-H429F mutant mAb which showed a maximum killing percent of 89.74% and, even at a high dilution (0.16 μg/ml), showed 29% CDC killing which was similar to the maximum killing (27.61%) achieved by the wild type form. Thus, substitution at position H429 of the CH3 domain of mezagitamab not only enhances CDC against certain cell targets but can also confer potent CDC against targets that resist lysis. Importantly, the enhanced level of killing of cells conferred by the H429F modification in mezagitamab-H429F further demonstrates that H429 modification can be effectively applied to unrelated mAbs to enhance the killing of cells, including cells that are killed poorly by unmodified WT mAbs (i.e. cells showing resistance to lysis) as was seen, for example, with daratumumab-H429F (FIG. 25, FIG. 26) and isatuximab-H429F (FIG. 34).

### Example 10 H429F Modification of an Fc Region Component can Produce Hetero-Oligomers

The H429F modification of the IgG Fc enhances complement mediated cytotoxicity (CDC). The mutations K439E and S440K of IgG are known to suppress IgG hexamer formation and to inhibit CDC function, but full activity is restored when the two mutant IgGs are used together as a mixture (Diebolder C A et al., 2014 supra). Thus, the effects of the K439E and S440K mutations on the properties of H429 modified IgG were evaluated in this example using the CD20-specific mAb, rituximab.

### Methods and Materials

### Antibodies and Antibody Constructs

Used in this example are the rituximab IgG1 WT heavy chain and the H429F mutant heavy chain as described in Example 3 above. Two additional antibodies were synthesised which contained H429F plus an additional point mutation in the H chain as described in Table 4. The first additional mAb comprised H chains containing the H429F modification plus the additional mutation of K439E (rituximab-H429F/K439E). The second additional mAb comprised H chains containing the H429F modification plus the additional mutation of S440K (rituximab-H429F/S440K).

### Synthesis of Unmodified and Mutated Heavy Chains

Antibody expression constructs and Fc variants including the replacement of H429 with phenylalanine (H429F), K439 with glutamate (K439E) and S440 with lysine (S440K) in the H chains of the rituximab mAb were generated by standard methods known to those skilled in the art.

### Expression of Antibody Constructs

Expression of the antibodies was conducted using transient transfection of Expi293F cells (Thermo Fisher Scientific) under the same conditions described above for other therapeutic mAbs. As for the mAbs, the cell cultures were harvested after transfection and centrifuged at 2500 rpm for 20-30 minutes, and then the supernatant was filtered with a 0.2 μm high flow filter (Sartorius AG) prior to purification. The presence of the expected antibody in the supernatant was confirmed by SDS-PAGE.

Purification of mAbs and Evaluation of Antigen Binding

The mAbs were purified from supernatant of the transfected Expi293F cells by protein A affinity chromatography as described elsewhere such as in Example 3. Following protein A affinity purification, size exclusion chromatography (SEC) was used to further purify and characterise the antibodies as described herein. Purified antibodies were then tested for antigen binding on CD20 positive cells by flow cytometry prior to functional analysis.

### Complement Dependent Lysis of Cells

CDC of target cells mediated by mAbs was measured by flow cytometry using the Zombie Green Fixable Viability kit (BioLegend) following opsonisation of cells with mAbs.

TABLE 4
Expression constructs encoding the heavy chains of rituximab either encoded the WT C-terminus
sequence or were modified by mutation to encode the indicated mutant C-terminus sequences.

| Fc | sequence | | |
|---|---|---|---|
| mutation(s) | type | C-terminal sequence | |
| WT | amino | H⁴²⁹E A L H N H Y T Q K S L S L S P G K⁴⁴⁷ | SEQ ID NO: 96 |
| | acid DNA | CACGAAGCGCTGCACAATCATTATACTCAGAAAAGCCTGAGCTTGAGCCCGGGAAAA | SEQ ID NO: 97 |
| H429F | amino | F⁴²⁹E A L H N H Y T Q K S L S L S P G K⁴⁴⁷ | SEQ ID NO: 98 |
| | acid DNA | TTCGAGGCGCTGCACAATCATTATACTCAGAAAAGCCTGAGCTTGAGCCCGGGAAAA | SEQ ID NO: 99 |
| H429F/K439E | amino | F⁴²⁹E A L H N H Y T Q E S L S L S P G K⁴⁴⁷ | SEQ ID NO: 100 |
| | acid DNA | TTCGAAGCGCTGCACAATCATTATACTCAGGAGAGCCTGAGCTTGAGCCCGGGAAAA | SEQ ID NO: 101 |
| H429F/S440K | amino | F⁴²⁹E A L H N H Y T Q K K L S L S P G K⁴⁴⁷ | SEQ ID NO: 102 |
| | acid DNA | TTCGAAGCGCTGCACAATCATTATACTCAGAAAAAGCTGAGCTTGAGCCCGGGAAAA | SEQ ID NO: 103 |

### Results and Discussion

The rituximab-WT, rituximab-H429F and additional C-terminus mutants H429F/K439E and H429F/S440K showed equivalent elution from the protein A affinity column, and further purification by SEC also yielded a single IgG peak for each of the mAbs of 150 kDa by SDS-PAGE that comprised ˜50 kDa heavy chain and 25 kDa light chain species following reduction with dithiothreitol as described for similar therapeutic mAbs in Example 3.

The CDC potency of the rituximab-H429F was compared to rituximab-WT (FIG. 48A). Each mAb was titrated individually by serial two-fold dilution in the presence of normal human serum as a source of complement. The CDC potency (% killing) was evaluated on CD20 expressing Ramos cells. As shown by FIG. 48A, the Ramos cells were resistant to CDC killing by the rituximab-WT but were readily killed by the rituximab-H429F mutant. However, both the dual mutants, rituximab-H429F/K439E and rituximab-H429F/S440K, showed greatly diminished killing compared to the rituximab-H429F. Thus, substitution at positions 439 or 440 strongly inhibits the CDC enhancing effect of the H429F mutation. Others have shown the K439E and S440K mutations inhibit Fc:Fc interactions, oligomer formation (including hexamers) and subsequent CDC, by the introduction of charge repulsion (Diebolder C A et al., 2014 supra). However, fully enhanced CDC-activity equivalent to that of the rituximab-H429F is restored when the rituximab-H429F/K439E and rituximab-H429F/S440K mAbs are used together as a mixture. It should be noted that all mAbs gave near identical binding to CD20 (FIG. 48B). Therefore, the differences in the CDC potency conferred by the mutations K439E and S440K were not related to differences in binding to the target cells. While not wishing to be bound by theory, it is considered that by using these two mutants together as a mixture allows charge repulsion to be avoided by heterotypic interactions between K439E and S440K mutant Fes.

The full CDC activity of the combination of rituximab-H429F/K439E and rituximab-H429F/S440K as a mixture demonstrates that hetero-oligomers (e.g. hexamers) are formed efficiently. This can be exploited to produce hetero-oligomers from two mAbs with different specificities to target two different epitopes on the same antigen, or two entirely different antigens. Such heterotypic oligomers, including hetero-hexamers, are expected to have advantages for specificity, for example where effector function is desirable to be directed only, or selectively, against targets that express the two specificities recognised individually by each of the two mAbs. Moreover, the crosslinking of different receptors selected by the different specificities of two mAbs in hetero-oligomers, including hetero-hexamers, is also expected to be efficient. More heterogenous oligomers (that none-the-less may have highly selective direction of effector functions to targets or may be capable of multiple crosslinks of receptors and subsequent unique biologic responses) may also be achieved by using more than two antibody specificities, such as three distinct mAbs, or four distinct mAbs etc. (eg. a hetero-oligomer formed from a mixture of mAb1-IgG-H429F/K439E, mAb2-IgG-H429F/K439E and mAb3-IgG-H429F/S440K, or from a mixture of mAb1-IgG-H429F/K439E, mAb2-IgG-H429F/K439E, mAb3-IgG-H429F/S440K and mAb4-IgG-H429F/S440K). Other mutations that suppress Fc:Fc interactions and CDC or which can be used to form hetero-oligomers have been described in van den Bremer E T J et al., 2015 supra, and may substitute for the K439E and S440K mutations used herein.

### Example 11 Discussion of H429 Mutations in the CH3 Domain, Implications and Applications

A method of altering the functional and physical properties of the heavy (H) chain of immunoglobulins (and, in turn, the molecules comprising such H chains or a part thereof comprising at least a CH3 domain) has been identified which involves modifying the amino acids in the CH3 domain. In particular, by substitution of the histidine at position 429 (Eu numbering) within the CH3 domain with different amino acids, immunotherapeutic proteins may be produced showing unexpected and distinct effects on the physical and functional properties of the molecules such as, especially, enhancement of complement activation or mAb-induced receptor signalling as exemplified by DR5 activation, by the functional consequence of protein hexamerisation. In the context of a modified antibody according to the present disclosure, and while not wishing to be bound by theory, it is considered that oligomerisation, particularly hexamerisation, of the antibodies especially on the target (on-target assembly) provides an optimal platform for binding of the hexameric C1q complex, resulting in, for example, enhanced activation of the complement cascade that leads to complement-dependent effector responses (e.g. phagocytosis or killing of target cells by complement-dependent cytotoxicity (CDC)). Moreover, oligomerisation for the purpose of enhancing cell surface signalling of mAbs bound to targets leads to enhanced signalling responses (e.g. apoptosis and cell death through cell surface molecules able to induce death pathways). This enhancement of the DR5 signalling responses is underpinned by H429F oligomerisation and thus signal amplification induced by antibody cross-linking in other systems where signalling is achieved by antibody cross-linking; for example, antibody cross-linking of activating type receptors including the Fc receptors or antigen receptor complex of T cells and B cells or inhibitory type receptors including immune checkpoints would be expected to also be enhanced when antibodies to such cell surface molecules contain the H429F modification.

### Functional Changes

Indeed, the experimentation provided herein shows that H429 substitution can enhance the killing potency of IgG mediated by the classical complement pathway, and that antibodies bearing an H429Y or especially the H429F mutation have a greater capacity to activate complement than their wild type (WT) counterparts bearing an unmodified IgG heavy chain. Further, it was found that these modifications confer a level of enhancement of activity that is so profound that, in some instances, only the antibodies carrying a mutation at H429 are actually capable of activating complement to kill cells (i.e. the cells were otherwise resistant to killing by the unmodified wild type antibody); for example, the anti-CD20 antibody 11B8-H429F was found to potently kill lymphoma cells compared to the unmodified 11B8-WT which showed little killing capacity (FIG. 22). Similarly, the anti-CD38 antibody daratumumab-H429F kills myeloma cells and acute lymphoblastic leukaemia cells whereas the unmodified daratumumab wild type mAb does not (FIG. 26).

Further, it was significant that such enhanced functional activities (i.e. conferred by the mutation in the CH3 domain) was observed indicating that the alteration of effector functions resulting from mutation of H429 is independent of the:
(i) Variable domain (since the altered effector responses conferred by the modification of H429 were apparent in all of the antibodies detecting distinct epitopes and were independently derived and therefore unrelated, and noting that the CH3 domain is physically distant from the variable domain which recognises antigen);
(ii) Epitope (since the altered effector responses conferred by the modification of H429 were apparent in all of the antibodies detecting distinct epitopes);
(iii) Cellular target (since enhanced complement activation was observed in all of the antibodies targeting lymphoma cells, myeloma cells, adenocarcinoma cells and leukaemia cells);
(iv) Molecular target (since the functional enhancement was observed in all of the antibodies which targeted different cell surface molecules (namely HER2, CD20, CD38 and SARS-CoV-2 spike which are structurally and functionally unrelated) and also in the anti-hapten antibodies);
(v) Epitope location (since the functional effects were evident in antibodies targeting different epitopes within the one molecular target (e.g. the different epitopes in CD20 recognised by the rituximab and 11B8 mAbs, and the different epitopes in HER2 recognised by the pertuzumab and trastuzumab mAbs and the different epitopes in CD38 recognised by the daratumumab and isatuximab mAbs));
(vi) Immunoglobulin type (since the altered effector responses conferred by the modification of H429 were apparent in IgG3 and in IgG4);
(vi) Physical form of the modified antibodies (since the non-covalent oligomeric/hexameric IgG form bearing the H429Y mutation and the non-oligomeric IgG bearing the same mutation are equally potent in CDC); and
(vii) Nature of the target recognition structure and the format of the H chain (since the effects seen in the antibodies bearing an H429 substitution were also evident in the ACE2-Fc fusion proteins (e.g. the H429Y modification facilitated non-covalent pH-sensitive oligomerisation and H429F enhanced complement activation)).

### Physical Changes

All of the evaluated antibodies, irrespective of their antigenic target or the nature of the substitution at position H429, showed equivalent characteristics in protein A affinity purification to their unmodified wild type mAb counterpart (e.g. the rituximab-H429F mutant antibody behaved identically to the unmodified wild type rituximab mAb). However, depending on the amino acid used to replace the H429, size exclusion chromatography (SEC) revealed that the modified mAbs can exist in solution at pH 7.2 as preformed, non-covalent IgG oligomers (IgGₒₗᵢ) in equilibrium with non-oligomeric IgG species, and the relative proportions of these oligomeric and non-oligomeric IgG, which can be equally potent in CDC (e.g. the rituximab-429Y(oli) and the non-oligomeric rituximab H429Y IgG(H₂L2) forms both mediate enhanced CDC compared to the unmodified wild type rituximab mAb) can thereby be controlled by the pH of the buffer (e.g. lowering the purification buffer to pH 5.0 resulted in the presence of only non-oligomeric IgG compared to the higher proportion of IgGₒₗᵢ at pH 7.2) providing significant flexibility and control in the manufacture of the immunotherapeutic proteins of the disclosure.

### Cooperation and Synergy

The consequences of H429 substitution in the CH3 domain are multifaceted. For example, while such modifications can significantly enhance the potency of complement activation or receptor agonism by individual mAbs compared to their wild type counterparts, it has been found that provision of mixtures of antibodies modified at H429 may additionally enhance function by the cooperation and functional synergy between the H429 modified antibodies. In particular, it is considered that additive or synergistic co-operation in the functional activity of mAb mixtures results in even greater complement activation potency than that observed with the same H429 modified antibodies used individually and, particularly, when the concentration of each of the mAbs in the mixture was limiting. This type of cooperation was observed using mAbs detecting distinct molecular targets (e.g. the rituximab-H429F mAb detecting CD20 mixed with the daratumumab-H429F mAb detecting CD38) and in mixtures of antibodies detecting distinct epitopes within the same molecular target (e.g. the rituximab-H429F mAb mixed with 11B8-H429F binding to CD20), and found to be independent of the target cell (e.g. synergistic cooperation was observed between the pertuzumab-H429F mAb and the trastuzumab-H429F mAb on adenocarcinoma cells, as well as between the rituximab-H429F mAb and the daratumumab-H429F mAb on blood cancer cells). Such cooperation was evident in the apoptotic killing potency of anti-DR5 mAbs. These data also showed the potential to specifically form hetero-oligomers wherein the different cell surface targets are incorporated into the cluster by virtue of the interaction between the H429F modified heavy chains of the different mAbs; such hetero-oligomers, depending on context, enabling enhanced CDC or agonistic signalling leading to a cellular response such as apoptosis. Thus, the use of mixtures of mAbs with H429 substitution facilitates greater functional potency through cooperation and functional synergy irrespective of the epitope detected by the individual mAbs (i.e. whether the epitopes are present on the same or distinct molecular targets) and thereby provides the opportunity to further capitalise on the enhancement of the functional potency utility achieved by H429 modification of the immunoglobulin CH3 domain, including improving the specificity of targeting of immunotherapeutic proteins of the present disclosure by leveraging the functional cooperation apparent between different antibodies comprising H429F substitution.

Combining Other Modifications with Function-Modifying H429 Substitutions

The enhancement of complement dependent effector functions by modification at the H429 position was surprising as this amino acid residue is located in the CH3 domain whereas the interaction of C1q (with IgG) occurs primarily in the CH2 domain. Further, the interaction of an antibody with FcγR also occurs in the CH2 domain (Hogarth and Pietersz, 2012 supra; Chenoweth et al., 2020 supra) as does much of the interaction with the scavenger receptor FcRn (Dall'Acqua et al., 2002 supra). Thus, because of the modular nature of the immunoglobulin H chain (FIG. 2), it is possible to combine modifications in one domain with modifications in the same or different domains that affect antibody functions whilst retaining the oligomerisation mediated by H429 modification (e.g. an H429 substitution can be readily combined with modifications elsewhere (e.g. amino acid substitutions in the hinge and/or CH2 domain that are known to enhance or ablate one or more of Fc receptor binding and/or complement binding, or which are known to enhance or ablate antibody half-life).

Thus, in a particular application, an H429 substitution may be combined with a mutation in CH2 domain known to enhance FcγR binding (e.g. for FcγRIII binding) to improve ADCC (reviewed in Chenoweth et al., 2020 supra) in an immunotherapeutic protein of the disclosure, or alternatively (or additionally) combined with a mutation in the CH2 domain known to enhance C1q binding (Lee et al., 2017 supra) to further improve complement potency. In another particular application, an H429 substitution may be employed to enhance or decrease binding to FcRn so as to modulate the in vivo half-life of an immunotherapeutic protein. For example, in some embodiments, an H429 substitution may be combined with modifications known to enhance binding to FcRn (e.g. M252Y, S254T and/or T256E; Dall'Acqua et al., 2002 supra) so as to increase the in vivo half-life of an immunotherapeutic protein of the disclosure. Notwithstanding the modular nature of antibodies, different mutations can also be made in the same domain of the immunoglobulin chain with the resulting mutant displaying properties conferred by each of the individual mutations. For example, in some embodiments, an H429 substitution may be combined with modifications known to enhance binding to FcRn (e.g. M438L and N434S; Zalevsky J et al., Nat Biotechnol 28:157-159, 2012) so as to increase the in vivo half-life of an immunotherapeutic protein of the disclosure. In other embodiments, where the Fc region component is derived from an IgG3 immunoglobulin, an H429 substitution may be combined with the abovementioned R435H modification known to enhance the in vivo half-life of IgG3 molecules, and/or the N392K and M397V mutations (Saito S et al., 2019 supra).

Further, in some circumstances, an immunotherapeutic protein according to the present disclosure may be modified in a manner wherein the propensity to oligomerise is desirably retained (thereby enhancing the avidity of antigen binding, or in the case of fusion proteins, target binding), whilst ablating one or more effector functions such as Fc receptor interaction and/or C1q binding. As such, in the case where the immunotherapeutic protein is an antibody, the antibody will have enhanced avidity but lack immune effector functions such as ADCC or ADCP, or complement for example CDC or C′ADCP or C′ADCC. Modifications that simultaneously ablate FcγR and C1q binding are well known and include, for example, L234A, L235A or removal of the heavy chain glycan normally found at Asn 297 (Eu numbering) (Wang et al., 2018 supra). Moreover, modifications that selectively ablate FcγR and C1q binding are also well known and these can be combined with an H429 substitution so that an immunotherapeutic protein such as an antibody will have enhanced avidity but lack selected immune effector functions such as ADCC or ADCP complement, (e.g. CDC or C‘ADCP or or C’ADCC). For example, modifications in the CH2 domain or the lower hinge to selectively ablate effector function, may be used in combination with the H429F substitution to produce an immunotherapeutic protein which lacks FcγR or complement-based effector systems.

In some applications, where the immunotherapeutic protein comprises two immunoglobulin H chain polypeptides, one of the H chain polypeptides (i.e. the “first H chain polypeptide”) may comprise an Fc region component (comprising at least a CH3 domain) with an amino acid substitution at a position corresponding to H429, while other (the “second H chain polypeptide”) may comprise a different modification(s) within the CH3 domain.

### Unusual Feature(s) of the Histidine at Position 429 of the IgG Heavy Chain

The H429 residue is buried within the CH3 domain of the immunoglobulin Fc portion (FIG. 1) and so does not directly participate in the protein:protein interaction interface of the Fc observed in, for example, IgG molecule assemblies, such as that visualised in the crystallographic structure of anti-HIV antibody b12 (Saphire et al., 2001 supra) nor directly in any other Fc surface interfaces that may occur. Amino acid residues that form part of the binding interfaces in protein:protein interactions are understood to be clusters of solvent accessible surface residues that contribute to a complementary binding interaction surface and, accordingly, it is considered that residues buried below such interfaces do not directly contribute to a protein:protein interaction. However, in a general sense, inaccessible residues may indirectly affect interacting surfaces, but any effect of mutation at such inaccessible residues is not simply predictable and, moreover, predictive methods focus on interfacial surface residues.

### a Summary of Some Applications of an H429 Substitution (Part 1)—Immunoglobulin Molecules

In accordance with the present disclosure, the modification to CH3 domain at position 429 can be applied to, for example, a monomeric heavy chain (i.e. H₁ format; see FIG. 2, FIG. 53A) and thus to dimeric proteins (e.g. H chain dimers in the absence of light chain (H₂ format), or in the presence of light chain H₂L₂ format of the typical IgG structure (FIG. 2)) where the H chain dimeric molecule may be homodimeric or heterodimeric (e.g. a bi-specific antibody). Such a modification at position 429 may be made to, for example, human IgG1 or to the corresponding residue in the CH3 domain of an immunoglobulin of another IgG subclass (i.e. IgG2, IgG3 or IgG4) or an IgA subclass (i.e. IgA1 or IgA2) or another immunoglobulin isotype such as IgD, and even at the corresponding position of the equivalent CH4 domain of IgE or IgM. Moreover, the H429 modification may be present in only one CH3 domain of the dimer. Further, the H429 modification may be applied to monomeric antibody-like molecules (FIG. 2, FIG. 53A) comprising one or more immunoglobulin H chain polypeptides comprising at least a CH3 domain (or its equivalent or part thereof derived from IgA or other Ig classes), and such antibody-like monomers may form homodimeric or heterodimeric molecules wherein at least one monomer in the dimer is modified at the position corresponding to H429 of the IgG1 CH3 domain. The H429 modification may be present in only one CH3 domain of the dimer.

### a Summary of Some Applications of an H429 Substitution (Part 2)—Fusion Conjugate Proteins

In accordance with the present disclosure, the modification to CH3 domain (or CH4 domain) at position H429 can be applied to, for example, a monomeric fusion protein (i.e. as exemplified by the ACE2-Fc fusion proteins of Example 1). The H429 substitution may be applied to, for example, monomeric fusion proteins (FIG. 2) comprising a CH3 domain (or its equivalent or part thereof derived from IgA or other immunoglobulin classes). Such fusion protein monomers may form homodimeric or heterodimeric molecules wherein at least one monomer in the dimer is modified at the position corresponding to H429 of the CH3 domain. In some particular applications, a fusion protein according to the disclosure may comprise an H429Y substitution in only one of the CH3 domains. Like with immunoglobulins, such H429 modifications in fusion proteins can confer effects on various physical and effector properties. For example, enhanced complement activation leading to CDC was observed to be a characteristic of ACE2-Fc fusion proteins where the H429F-substituted Fc region component conferred strong complement activation and CDC. The characteristic of pH-sensitive oligomerisation of antibodies with the H429Y substitution was also found to be a characteristic of the ACE2-Fc fusions carrying the H429Y mutation. This indicates the importance of the H429 position in dictating the physical and enhanced effector function in diverse families of immunotherapeutic proteins, and which is clearly independent of the target recognised and also of the nature of the target recognition structure (i.e. whether an antibody V domain or a cell surface receptor or soluble receptor).

### Example 12 ACE-2-Fc (with H429F) in Combination with an Anti-SARS-CoV-2 Antibody

The effect of H429 modification on the potency of CDC killing in the context of infectious disease was evaluated using mAbs detecting the spike protein of SARS-CoV-2. In particular, the histidine 429 of CH3 was substituted with phenylalanine (i.e. H429F) in the H chain of the S2P6 monoclonal antibody which targets the S2 stem region of the SARS-CoV-2 spike (see, for example, Pinto D et al., Science 373:1109-1116, 2021).

### Methods and Materials

### Generation of Antibodies and Antibody Constructs

The S2P6-wild type (WT) mAb used in this example comprised a SARS-CoV-2 spike protein-specific heavy chain polypeptide with the amino acid sequence shown as SEQ ID NO: 78 (see Table 5) comprising, in the order of the N-terminus to the C-terminus, the spike-specific VH domain of S2P6 mAb (defined in Pinto et al., 2021 supra) fused to CH1-hinge-CH2-CH3 domains of human IgG1, and is encoded by the codon-optimised polynucleotide sequence shown as SEQ ID NO: 79 (Table 5). The polypeptide of the anti-S2P6 anti-SARS-CoV-2 light chain comprised the amino acid sequence shown as SEQ ID NO: 80 (comprising the spike-specific VL domain of the S2P6 mAb fused to human kappa constant domain) and is encoded by the codon-optimised polynucleotide sequence shown as SEQ ID NO: 81 (Table 5).

Two additional mAbs were generated, the CC40.8-WT mAb and the CV3-25-WT mAb, and used in this example. Each mAb comprised a SARS-CoV-2 spike protein-specific heavy chain polypeptide with the amino acid sequence for either CC40.8 (shown as SEQ ID NO: 82) or for CV3-25 shown as (SEQ ID NO: 83) comprising, in the order of the N-terminus to the C-terminus, the spike-specific VH domain of either CC40.8 mAb (defined in Zhou P et al., Sci Transl Med 14(637):eabi9215, 2022) or of CV3-25 (defined in Jennewein M F et al., Cell Rep 36(2):109353, 2021) fused to CH1-hinge-CH2-CH3 domains of human IgG1, and encoded by the codon-optimised polynucleotide sequence shown for CC40.8 as SEQ ID NO: 84, or for CV3-25 as SEQ ID NO: 85 (see Table 5 for the sequences). The polypeptide of the anti-spike CC40.8 light chain or of the anti-spike CV3-25 light chain comprised the amino acid sequence shown in Table 5 for CC40.4 as SEQ ID NO: 86 and for CV3-25, shown as SEQ ID NO: 87 (comprising the spike-specific VL domain of the CC40.8 mAb or of CV3-25 fused to human kappa constant domain) and are encoded by the codon-optimised polynucleotide sequence shown for CC40.8 as SEQ ID NO: 88 and for CV3-25 as the codon-optimised DNA sequence shown as SEQ ID NO: 89.

### Synthesis of Unmodified and Mutated Heavy Chains

Antibody expression vectors which included a modified Fc sequence encoding the substitution of H429 with phenylalanine (i.e. H429F) in the H chains of the S2P6, CC40.8 and CV3-25 mAbs, were generated by standard methodologies known to those skilled in the art. The respective modified H chain amino acid sequences (and encoding polynucleotide sequences) are shown in Table 5 as SEQ ID NOs: 90-95.

Expression and Purification of flACE2-Fc Fusion Proteins

The flACE2-Fc-WT and flACE2-Fc-H429F fusion proteins were produced using transient transfection in Expi293F cells as described above in Example 1. Purification from supernatant by ion exchange chromatography and size exclusion chromatography (under the same conditions described in Example 1) yielded a single fusion protein “species”.

### Expression of Anti-SARS-CoV-2 Antibody Constructs

Expression of the anti-SARS-CoV-2 spike antibodies was conducted using transient transfection of Expi293F cells (Thermo Fisher Scientific) using standard methodologies. The cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE.

Purification of mAbs and Evaluation of Antigen Binding

The anti-SARS-CoV-2 mAbs were purified from supernatant of the transfected Expi293F cells by protein A affinity chromatography then size exclusion chromatography (SEC) yielding the expected single IgG peak (i.e. for the standard H₂L₂ antibody structure) confirmed by SDS-PAGE. Prior to functional analysis, the purified flACE2-Fc-H429F or flACE2-Fc-WT fusion proteins and the purified anti-SARS-CoV-2 mAbs were tested for antigen binding on Ramos cells expressing SARS-CoV-2 spike (Ramos-S cells) by flow cytometry using a fluorescent anti-human IgG-Fc antibody (FITC-conjugated anti-hIgG-Fc, 1/500; Chemicon®, Merck KGaA, Darmstadt, Germany).

### Complement Dependent Lysis of Cells

The potency of CDC mediated by the flACE2-Fc fusion protein or the anti-SARS-CoV-2 mAbs was measured by flow cytometry using Ramos-S cells expressing the SARS-CoV-2 spike protein in a similar manner to the CDC assay described in Example 1. Human serum, diluted 1/6.4, was used as a source of complement. The Zombie Green Fixable Viability kit (BioLegend) was used following opsonisation of Ramos-S cells with the mAbs. Additionally, CDC lysis was examined in mixtures of flACE2-Fc-H429F and anti-SARS-CoV-2 mAbs comprising H chains with an H429F modification.

### Results and Discussion

H429 Modification Confers CDC by the flACE2-Fc Fusion Protein

The flACE2-Fc-H429F and flACE2-Fc-WT fusion proteins exhibited near identical binding characteristics to SARS-CoV-2 spike protein expressed on Ramos-S cells (FIG. 49A) indicating that the H429F mutation did not appreciably affect antigen binding on cells. The flACE2-Fc-H429F and flACE2-Fc-WT fusion proteins were then tested for their capacity to mediate CDC (FIG. 49B). Despite their similar binding profiles, considerable differences in the CDC potency were observed between the two fusion proteins. That is, flACE2-Fc-WT showed poor CDC of Ramos-S cells, with the maximum kill of 19.4% occurring at the highest concentration (2.5 μg/ml flACE2-Fc-WT) and rapid titration to background (2.5 μg/ml-0.6 μg/ml). In contrast, the flACE2-Fc-H429F fusion protein mediated a potent level of cell killing over a larger concentration range (2.5 μg/ml-0.156 μg/ml) compared to flACE2-Fc-WT, and exhibited a maximum kill of 73.8%. This is consistent with the results of ELISA experiments (FIG. 11) showing that flACE2-Fc-H429F demonstrated enhanced binding of C1q (FIG. 11D) and enhanced formation of the membrane attack complex (FIG. 11F) compared to its WT counterpart as was also demonstrated for trACE2-Fc-H429F (FIGS. 11C and 11E).

H429 Modification Confers CDC by Anti-SARS-CoV-2 mAbs

The anti-SARS-CoV-2 mAbs S2P6-WT, CC40.8-WT and CV-23-WT were formatted as wild type human IgG1 and kappa light chain mAbs as described above. The H429F mutation was engineered into the H chains of each mAb to generate S2P6-H429F, CC40.8-H429F and CV-23-H429F mAbs. The binding of the purified mAbs to SARS-CoV-2 spike protein was evaluated by flow cytometry (FIG. 49C). The S2P6-H429F mAb and S2P6-WT mAb showed near identical binding properties. Similarly, the CC40.8-H429F and CV3-25-H429F mAbs showed very similar binding profiles to their respective wild type counterparts, indicating that the H429F modification of the heavy chain does not affect interaction of the mAbs with the SARS-CoV-2 spike antigen.

The CDC potency of the S2P6-H429F and CC40.8-H429F mAbs, relative to their wild type counterparts S2P6-WT and CC40.8-WT, was also determined (see FIG. 49D). It was found that the S2P6-WT mAb showed no CDC of Ramos-S cells at any concentration above the background control of lysis in the absence of antibody but in the presence of only complement (i.e. no mAb C′ only; FIG. 49D). However, the S2P6-H429F mAb showed potent killing. Similarly, the CC40.8-H429F mAb also showed readily detectable killing above background though not as great as the potency of S2P6-H429F.

Modification of the CH3 Domain Enables and Fusion Proteins and mAbs of Distinct Specificity to Act Synergistically in CDC

Combinations of the ACE2-Fc fusion protein and anti-SARS-CoV-2 mAbs were investigated for potential synergy leading to greater functional potency (i.e. by determination of enhanced CDC lysis of targets) (see FIGS. 50A and 50B). In particular, cooperation in CDC between the fusion protein (which binds with the RBD of the spike protein) and anti-SARS-CoV-2 mAbs (which detect epitopes in the stem region of the spike protein), was determined using pairwise combinations of flACE2-Fc with the various anti-SARS-CoV-2 mAbs.

The combination of flACE2-Fc-WT with the S2P6-WT mAb failed to show any CDC above background. Moreover, no lysis was detected when the S2P6-WT was titrated in the presence of a fixed concentration (1 μg/ml) (flACE2-Fc-WT; FIG. 50A). In contrast, functional cooperativity was readily apparent between the flACE2-Fc-H429F fusion protein and the S2P6-H429F mAb (FIG. 50A). The S2P6-H429F mAb, when titrated alone, mediated potent CDC of Ramos-S cells in the absence of flACE2-Fc-H429F. The flACE2-Fc H429F at the limiting concentration of 1 μg/ml, mediated detectable CDC (23.43% killing). Remarkably, the level of CDC mediated at all concentrations of S2P6-H429F in the presence of a fixed concentration (1 μg/ml) of flACE2-Fc-H429F, was considerably greater than with the S2P6-H429F mAb alone or of the flACE2-Fc-H429F alone (FIG. 50A). The cooperation between the flACE2-Fc-H429F fusion protein and the S2P6-H429F mAb was also readily detected when the concentration of the mAb was limiting (0.03 μg/ml-0.125 μg/ml) shown by the arrows in FIG. 50A. The cooperation was also evaluated between flACE2-Fc-H429F and two additional anti-SARS-CoV-2 spike mAbs, namely CC40.8 and CV3-25 (FIG. 50B). CDC was determined for the mAbs alone (at 2.5 g/ml of mAb), flACE2-Fc-H429F alone and in pairwise mixtures. Increased CDC was evident in the combination of the flACE2-Fc-H429F fusion protein and the CC40.8-H429F mAb compared to the CDC mediated by either alone. Similarly, the combination of flACE2-Fc-H429F with the CV3-25-H429F mAb mediated an increased level of CDC compared to the killing by either alone (FIG. 50B). Clearly, the surprising cooperation between the fusion protein and monoclonal antibodies can occur broadly and is not restricted to a single combination of proteins. More remarkable is that such cooperation was achieved between two proteins of distinct molecular formats (i.e. a fusion protein comprising a receptor (ACE2) fused to an Fe region component with a H429F modification synergistically cooperating with a monoclonal antibody to achieve greater functional potency).

### Example 13 H429F in an ACE2-Fc Protein Confers CDC Independent of the ACE2 Format

Experimentation was conducted to determine the effect of H429F modification on CDC potency using ACE2-Fc fusion proteins comprising structurally distinct forms of the ACE2 polypeptide. In particular, three ACE2-Fc fusion proteins were evaluated wherein the ACE2 component comprised either the truncated ACE2 ectodomain comprising only the catalytic domain of the ectodomain and excluding the collectrin domain (trACE2-Fc), or the full length ACE2 ectodomain (flACE2-Fc) or the enhanced full length ACE2 ectodomain comprising a triple mutation within the ACE2 polypeptide that has been reported to improve its binding affinity to the receptor binding domain (RBD) of SARS-CoV-2 spike protein (EflACE2-Fc) (Chan et al., 2020 supra).

### Methods and Materials

The ACE2-Fc fusion proteins (trACE2-Fc, flACE2-Fc and EflACE2-Fc) comprising the different structural forms of the ACE2 polypeptide were generated with either a wild type Fc region component or an Fc region component including a H429F mutation substantially as described above in Example 1. CDC potency was evaluated by flow cytometry using the Zombie Green method and using a 1/4 dilution of human serum as a source of complement.

### Results and Discussion

The binding of the ACE2-Fc fusion proteins to Ramos-S cells expressing the SARS-CoV-2 spike protein was determined by flow cytometry and the results are shown in FIG. 51. The trACE2-Fc-H429F and its wild type counterpart, trACE2-Fc-WT, showed near identical binding to SARS-CoV-2 spike protein (5.1 nM and 5.3 nM respectively; FIG. 51A). Similarly, flACE2-Fc-H429F and EflACE2-Fc-H429F showed near identical binding to their wild type counterparts flACE2-Fc-WT and EflACE2-Fc-WT (FIGS. 51B and 51C respectively). Thus, H429F modification does not significantly impact on the strength of binding of the fusion proteins to the target protein SARS-CoV-2 spike.

The EC50 values showed that the affinity enhanced EflACE2-Fc fusion proteins with either a wild type Fc region component or an Fc region component with the H429F mutation, showed the greatest level of binding to Ramos-S cells. Interestingly, the trACE2-Fc fusion protein, which includes only the ACE2 catalytic domain and lacks the collectrin domain found in the flACE2, showed stronger binding (EC₅₀ 4.4 nM) to SARS-CoV-2 spike than the flACE2-Fc fusion proteins (EC₅₀ 6.6 nM) (FIG. 51).

Despite the near identical binding of each set of ACE2-Fc fusion proteins, CDC was greatly enhanced when the Fc region component included the H429F mutation (FIG. 52). Thus, trACE2-Fc-H429F (FIG. 52A), flACE2-Fc-H429F (FIG. 52B) and EflACE2-Fc-H429F (FIG. 52C) mediated potent CDC but, in contrast to their wild type counterparts, trACE2-Fc-WT, flACE2-Fc-WT and EflACE2-Fc-WT which mediated little or no CDC.

The enhancement of CDC achieved by the H429F modification of ACE2-Fc fusion proteins was independent of the nature of the ACE2 polypeptide as all of the ACE2-Fc-H429F forms mediated potent CDC. Interestingly, the hierarchy of CDC potency observed was that EflACE2-Fc-H429F was more potent than trACE2-Fc-H429F, which was more potent than flACE2-Fc-H429F. It is noteworthy that ELISA experiments investigating C1q binding (FIG. 11) by ACE2-Fc fusion proteins also demonstrated that trACE2-Fc-H429F showed a greater capacity to bind C1q and form the membrane attack complex (C5b-9) compared to flACE2-Fc-H429F, thereby indicating that enhancement of C1q binding correlates with the relative CDC potency of fusion proteins, in this case ACE2-Fc. An extension of this observation is that the quantitation of C1q binding to H chains or Fc region components, or of the formation of the membrane attack complex, such as by using ELISA-based methods, may provide a rapid screen for the selection of antibodies, antibody-like molecules, Fc-fusion proteins and the like for their potential to mediate CDC and thus, for the discrimination between the levels of CDC killing potency mediated by such proteins.

### Example 14 Production of an IgG-Like ACE2-Fc Fusion Protein

This example sought to produce an immunotherapeutic protein (comprising an Fc region component with an amino acid substitution at position H429 of the amino acid sequence of human IgG1 heavy chain) having a different structure to those of the preceding examples. That is, the immunotherapeutic protein in this case may be considered as an example of an antibody-like (Ab-like) molecule; more particularly, an Ab-like fusion protein.

Ab-like proteins are based on the archetypal immunoglobulin structure (see FIGS. 2 and 53A), but have one or more target recognition structure (not of immunoglobulin origin; that is, not a typical VL or VH domain) replacing the variable domains of the heavy and light chains. In this example, the affinity enhanced full length ACE2 polypeptide (EflACE2) was used in the production of an EflACE2-Ab-like fusion protein comprising a polypeptide complex assembled into an H₂L2-like protein, wherein there are two heavy chain-like polypeptides each comprising EflACE2 linked to an Fc region component (i.e. an H chain) and additionally, two light chain-like polypeptides each comprising EflACE2 linked to a constant (C) domain of a light chain shown. A diagrammatical representation of this molecule is shown in the middle panel of FIG. 53A.

### Methods and Materials

### Constructs

The mature EflACE2-Ab-like-WT fusion protein used in this example comprised a heavy chain polypeptide shown as SEQ ID NO: 104 comprising the EflACE2 ectodomain comprising the entire ectodomain fused, in the order the N-terminus to the C-terminus, to CH1-hinge-CH2-CH3 domains of wild type human IgG1, and is encoded by the codon-optimised polynucleotide sequence shown as SEQ ID NO: 105. Similarly, the mature polypeptide of the light chain shown as SEQ ID NO: 106 comprised the EflACE2 ectodomain fused to the N-terminus of a human kappa constant domain, and is encoded by the codon-optimised polynucleotide sequence shown as SEQ ID NO: 107. The mature EflACE2-Ab-like-H429F fusion protein comprised a heavy chain polypeptide with an amino acid sequence shown as SEQ ID NO: 108 with an Fc region component with the H429F modification, and is encoded by the codon-optimised polynucleotide sequence shown as SEQ ID NO: 109.

The EflACE2-Ab-like-WT, comprising an IgG1-WT H chain, expression construct was assembled using NEBuilder (New England Biolabs) by the appropriate insertion of a synthetic DNA fragment encoding linking sequence and a human IgG1-CH1 domain (GeneArt; Thermo Fisher Scientific) into the EflACE2-Fc-WT expression vector cleaved at the sequence encoding the linker between the EflACE2 and Fc region component sequences. In a similar approach, the EflACE2-Fc-WT expression vector was cleaved to excise the fragment encoding the Fc region component sequences and a synthetic DNA encoding a linking sequence and a human constant light kappa domain was incorporated. The mutated H429F H chain variant was produced as described in Example 1. Thus, the expression constructs consisted of several synthetic polynucleotide sequences that, joined together, encoded the EflACE2-Ab-like fusion protein heavy chain or light chain, within the expression plasmid pcDNA3.4 (Thermo Fisher Scientific).

### Expression and Purification

Expression of the Ab-like fusion proteins was conducted using transient transfection of Expi293F cells (Thermo Fisher Scientific) using the conditions described hereinbefore in Example 1.

The fusion proteins were purified to homogeneity by Protein A affinity chromatography under gentle elution conditions according to the following protocol:
(i) On Day 1, the transfected Expi293F cells were incubated at 37° C. After the addition of enhancers, the cells were then incubated at 34° C., 125 μm, and 8% CO₂ atmosphere. Six days after transfection, the supernatant was harvested by centrifugation at 3000×g for 15 minutes. The supernatant was collected into a fresh tube and spun again for an additional 30 minutes. The supernatant was then filtered through a 0.22 μm filter.
(ii) The EflACE2-Ab-like-H429F fusion protein was purified using a 1 mL Hitrap™ Protein A column (Cytiva Life Sciences, Marlborough, MA, United States of America), with all steps at a flowrate of 1 ml/min. In a preferred method, the column was first equilibrated with 20 mM phosphate buffer, pH 7.0. The filtered supernatant was loaded onto the Protein A column using a BioLogic LP low-pressure chromatography system (Bio-Rad, Hercules, CA, United States of America). After loading was completed, the column was washed with 20 Column Volumes (CV) of 20 mM phosphate buffer pH 7.0. The column was further washed with 5CV of Buffer A—30 mM Arginine pH 4.0. The bound proteins were eluted using a shallow linear gradient to 35% buffer B—130 mM arginine, pH 4.0 performed over 20 CV. Fractions of 1 mL were collected, neutralised using 1M Tris-HCl pH 9.0, and concentrations determined using the 0.1% (mg/ml) extinction coefficient at 280 nm of 1.80 (derived using protparam at https://web.expasy.org/protparam/). Fractions 15, 20, 24, 25 and 27 across the elution peak were analysed using size exclusion chromatography (SEC). Because of the gentle elution conditions of this shallow arginine gradient, all of these tested fractions (i.e. 15, 20, 24, 25 and 27) showed approximately the same monomer to oligomer ratio of ˜85:15% and were free of large molecular weight aggregates. Thus, all fractions (8-34 in FIG. 54A) of the EFlACE2-Ab-like fusion protein eluted from the Protein A column were pooled and concentrated using a 30 kDa cut-off filtration device (Merck) and separated by SEC using a Superose 6 Increase 10/300 GL column (Cytiva Life Sciences). The pooled and concentrated EflACE2-Ab-like fusion protein eluted from the Protein A column had a similar SEC profile (FIG. 54B) to that of the individual fractions 15, 20, 24, 25 and 27 (data not shown). SDS-PAGE analysis indicated both the Protein A pooled eluate and the SEC-derived monomeric fraction comprised a heavy (H) and light (L) chain on reduction, and under non-reducing conditions comprised a fully disulfide-bonded, high molecular weight species consistent with a H₂L₂ composition (FIG. 54C).

### Complement Dependent Cytotoxicity (CDC) Assay and Spike Protein Binding

CDC potency was evaluated by flow cytometry using the Zombie Green method in a similar manner to the CDC assay described in Example 1. Binding of the EflACE2-Ab-like proteins to SARS-CoV-2 spike protein was evaluated by flow-cytometry using Ramos-S cells as described herein in Example 1.

### Results and Discussion

### Production and Purification of the Ab-Like Fusion Proteins

As described above, ACE2-Fc fusion proteins in Example 1 can be appropriately purified by sequential ion exchange (IEX) chromatography and size exclusion chromatography (see FIG. 5). An alternative to the use of the IEX step for the purification for the Ab-like proteins described in this example, is the use of Protein A affinity chromatography which is a highly specific affinity purification and is appropriate for at-scale production in an industrial setting, and with the use of arginine in the conditions for elution of the proteins from the Protein A column, aggregate formation in the eluted proteins may be suppressed (see, for example, Arakawa T et al., Protein Expr Purif 36(2):244-248, 2004; 2004 et al., Ejima D et al., Anal Biochem 345(2):250-257, 2005; and Shukla D et al., J Phys Chem B 115(11):2645-2654, 2010). Purification of ACE2-fusion proteins containing an Fc region component used elution by a low concentration of arginine, less than 130 mM, and at less than or equal to pH 5. The appropriate low arginine concentration can be achieved by standard liquid chromatography systems that can deliver a buffer gradient to the column. For example, in a preferred method of purifying EflACE2-Ab-like-H429F, purification is achieved through binding to Protein A under standard conditions followed by washing with five column volumes of Buffer A—30 mM arginine (pH 4.0) and elution with a linear gradient to 35% of 130 mM arginine (pH 4.0). Elution under other conditions, for example 100 mM sodium citrate buffer pH 3.0, may result in the elution of ACE2-Fc or ACE2-Ab-like fusion proteins containing some aggregates. However, this preferred method of the use of arginine under the disclosed conditions herein, yielded EflACE2-Ab-like protein from the Protein A chromatography that was demonstrated to be highly pure by SDS-PAGE, and was also largely monomeric (˜85%) as determined by SEC (i.e. relatively free of aggregates). Particularly, FIG. 53B shows that the EflACE2-Ab-like-WT protein was obtained as a pure, fully disulfide-bonded H₂L₂ protein that, on reduction with dithiothreitol, generated equal amounts of EflACE2-H and EflACE2-L chains. FIG. 54C shows that the EflACE2-Ab-like-H429F protein was also obtained as a pure, fully disulfide-bonded H₂L₂ protein that, on reduction with dithiothreitol, generated equal amounts of EflACE2-H and EflACE2-L chains.

### Target Recognition and CDC Potency of Ab-Like Fusion Protein

The CDC potency of the EflACE2-Ab-like-WT and EflACE2-Ab-like-H429F fusion proteins was determined by flow cytometry analysis (FIG. 53C). The EflACE2-Ab-like-WT failed to mediate detectable CDC killing of Ramos-S cells—indeed, lysis was similar to the background lysis (5.1%) of the cells in the complement only control. In contrast, the EflACE2-Ab-like-H429F fusion protein showed that it mediated powerful CDC killing of the SARS-CoV-2 spike expressing Ramos-S cells. These differences in CDC potency were not attributable to differences in binding of targets by the WT and H429F forms of the proteins. Analysis by flow cytometry, of the binding of the EflACE2-Ab-like proteins to Ramos-S cells expressing SARS-CoV-2 spike protein demonstrated that the EflACE2-Ab-like-WT and the EflACE2-Ab-like-H429F showed comparable levels of binding to the cells (FIG. 53D).

Clearly the modification of H429 of the heavy chain enhanced the CDC function of the Ab-like proteins. Thus, modification at position 429 of the immunoglobulin-H chain (e.g. mutations such as H429F or H429Y) are versatile modifications that enhance the functional utility of different classes of polypeptides that are derived from immunoglobulins (i.e. the function enhancing effects mediated by H429 mutation are apparent in antibodies, antibody-like proteins and in Fc-fusion proteins). Moreover, while the EflACE2-Ab-like fusion protein of this example comprises four polypeptide chains all of which comprise the same target recognition structure (i.e. EflACE2), it is considered that an Ab-like fusion protein may otherwise comprise heterologous polypeptides such that may each comprise a different target recognition structure (i.e. if formatted as an H₂L₂ complex, the Ab-like protein may comprise 1, 2, 3 or 4 different target recognition structures depending on the combinations of H and L chains comprising the different target recognition structures; see FIG. 53A, right panel). Additionally, Ab-like proteins formatted in an H₂ configuration (i.e. without associated light chains) may comprise the same or different target recognition structures in the two chains. The H chains of such molecules may be modified by mutation of H429 (e.g. H429F) to alter their effector responses in the same way as the CDC potency of EflACE2-Ab-like-H429F was enhanced here. Finally, it may be noted that the large size of the EflACE2-Ab-like-H429F molecule (>500 kDa) might have been predicted to impose steric restraints on the function of the Fc portion, even with the H429F mutation, but clearly this was not the case and indeed H429F mutation conferred potent CDC and can therefore be considered as representing a rigorous example of the capacity of the H429F modification of an Fc region component to confer improved effector function in Fc-fusion proteins.

### Example 15 H429F Modification Confers Enhanced Function in Antibodies with Mutations in the CH2 Domain Inactivating FcγR Binding and/or Complement Dependent Effector Function

H429F modification of the Fc region component was further investigated in heavy chains comprising additional mutations that alter the capacity of an antibody to activate immune effector functions, such as Fc receptor binding and/or complement dependent functions. In particular, the antibodies in this example included modifications to the hinge or the CH2 domain of the IgG H chain known to increase or decrease immune effector function (see Wang X et al., 2018 supra; and Lee C-H et al., Nat Immunol 18:889-898, 2017). Such modifications can confer properties broadly, for example, on all FcγR functions as well as complement activation or confer selective properties on the antibody such as enhancement or impairment of function of individual FcγR or of selectively altered complement function. It is important to note that these modifications in the hinge or CH2 domain of antibody (e.g. IgG) are distant from the CH3 domain that includes the H429. Since a feature of an immunoglobulin H chain is the modular domain structure, it is possible that the properties conferred on Ig H chains or Fc region components by H429 modification may be unaffected by mutations in CH2 domain that affect immune effector functions such as FcγR binding or complement activation or antibody half-life relating to FcRn binding (Dall'Acqua W F et al., J Biol Chem 281:23514-23524, 2002). That is, functional properties of the H429 modified Ig-heavy chain containing molecules such as antibodies, Ab-like molecules and fusion proteins may be further modified for enhanced or reduced functions by mutations in, for example, the CH1, the hinge or CH2 regions.

Alteration of immune effector function of IgG can be achieved by mutation of the IgG residues L234 and/or L235 (Eu numbering) which are located in the hinge proximal region of CH2 also known as lower hinge. One of the most widely used mutational strategies to inactivate immune effector functions mediated by IgG1 is the substitution of these residues with alanine (i.e. L234A and L235A) also known as LA/LA (Wang X et al., 2018 supra). The L234A, L235A mutations inactivate interactions with all cell surface FcγR and similarly inactivate the antibody-dependent complement cascade (Chenoweth A C et al., 2020 supra, and Wang X et al., 2018 supra). In this example, the effect of combining in the one H chain, a modified CH2 domain with a modified CH3 domain including the H429F modification was examined. In particular, death receptor mAbs BDR5 and TDR5 were formatted on human IgG1 backbone and synthesised such that each H chain of the mAbs contained the L234A, L235A and H429F mutations (i.e. referred to herein as BDR5-1LA/LA/HF and TDR5-1LA/LA/HF) and evaluated for effects on signal amplification in assays of apoptosis.

### Methods and Materials

### Antibodies and Antibody Constructs

The mAbs used in this example are based on the sequences of two distinct mAbs which specifically bind to the human DR5 molecule as described in Example 8. The DR5-specific H chain of the BDR5-1LA/LA/HF mAb comprised H chains of the human IgG1 subclass containing the L234A, L235A and H429F mutations and lacking the C-terminus lysine (delK447). Its amino acid sequence is shown as SEQ ID NO: 110, and is encoded by the codon-optimised polynucleotide sequence shown as SEQ ID NO: 111. The DR5-specific kappa light chain of BDR5-1LA/LA/HF mAb comprises the DR5-specific kappa light chain of the BDR5 mAb described in Example 8 and the polypeptide and encoding polynucleotide sequences are shown as SEQ ID NO: 48 and 49 respectively. Turning to the DR5-specific H chain of the TDR5-1LA/LA/HF mAb, this is comprised of H chains of the human IgG1 subclass containing the L234A, L235A and H429F mutations and lacking the C-terminus lysine (delK447), its amino acid sequence is shown as SEQ ID NO: 112, and is encoded by the codon-optimised polynucleotide sequence shown as SEQ ID NO: 113. The DR5-specific kappa light chain of TDR5-1LA/LA/HF mAb comprises the DR5-specific kappa light chain of the TDR5 mAb as described in Example 8; the polypeptide and encoding polynucleotide sequences are shown as SEQ ID NO: 54 and SEQ ID NO: 55 respectively. The other mAbs used herein comprising WT IgG1(delK447) H chains (i.e. BDR5-1WT and TDR5-1WT) or comprising H429F modified IgG1 chains (delK447) (i.e. BDR5-1H429F and TDR5-1H429F) were as described above in Example 8.

### Synthesis of Unmodified and Mutated Heavy Chains

Antibody expression constructs and Fc variants including the replacement of H429 with phenylalanine (H429F), the replacement of L234 with alanine and of L235 with alanine (L234A, L235A or LA/LA) were generated by standard methods known to those skilled in the art and are as described elsewhere in this specification in relation to therapeutic mAbs such as in Example 3.

### Expression of Antibody Constructs

Expression of the antibodies was conducted using transient transfection of Expi293F cells (Thermo Fisher Scientific) under the same conditions as has been described above. As for all mAbs, the cell cultures were harvested after transfection and centrifuged at 2500 rpm for 20-30 minutes, and the supernatant filtered with a 0.2 μm high flow filter (Sartorius AG) prior to purification.

Purification of mAbs and Evaluation of Antigen Binding

The BDR5-1LA/LA/HF and TDR5-1LA/LA/HF mAbs were purified from supernatant of the transfected Expi293F cells using Protein A affinity chromatography as described above (e.g. Example 8) and the mAbs showed the expected H₂L₂ format of monomeric antibodies as confirmed by SDS-PAGE analysis.

### Viability Assay

The capacity of antibodies to induce cell death was evaluated in a viability assay as described in Example 8. The antibodies were tested either individually as single agents or as mixtures of two antibodies. The viability assay was performed using Colo205 cells which were cultured routinely in culture medium consisting of RPMI 1640 medium supplemented with foetal calf serum (10% v/v) plus 4 mM glutamine and 100 U/ml penicillin and 0.1 mg/ml streptomycin. The cells were harvested from cell culture, washed in culture medium by centrifugation at 200×g and resuspended in culture medium. The cells were then washed again and resuspended in culture medium to a concentration of 1.2×10⁶/ml. One hundred microlitres (100 μL) of cell suspension (10,000 cells) was added to the wells of 96-well flat-bottom sterile plates. Where the mAbs were used in mixtures, the total concentration of mAb was the same as in the equivalent wells treated with the single antibodies. The plates were then incubated for 48 hours at 37° C. in 5% CO₂. The maximum viability control values were determined from cells cultured in 200 μl of culture medium in the absence of antibody. The control for maximum death was determined from wells seeded only with cell suspension in 200 μl of culture medium but treated in the last two hours of the incubation period (i.e. at the end of the incubation period) with 10 μl of 1% SDS. Following the incubation period, the viability of the cells was determined using a colorimetric assay using Cell Counting Kit 8 (CCK-8 using the tetrazolium salt, WST-8) (Abcam) and the plates incubated at 37° C. for 2-5 hours. Colour development was determined by measuring absorbance at 450 nm (Abs450) on a Fluorostar OPTIMA (BMG).

### Results and Discussion

The effect of H429 modification on the apoptosis killing potency of mAbs comprising heavy chains with additional mutations was determined. The BDR5-1LA/LA/HF mAb and the TDR5-1LA/LA/HF mAb were used individually and were also used in combination (BDR5-1LA/LA/HF+TDR5-1LA/LA/HF) (see FIG. 55). It was clear that when used individually, the BDR5-1LA/LA/HF mAb mediated a reduction in survival of the Colo205 cells compared to survival in the absence of antibody. Similarly, the TDR5-1LA/LA/HF mAb reduced cell survival when used individually. The greatest killing potency was obtained when the mAbs were used in combination which achieved levels of killing that were equivalent to the control for maximum death of cells determined by cell survival in the presence of SDS detergent. Even at concentrations as little as 0.6 μg/ml, the mixture of BDR5-1LA/LA/HF and TDR5-1LA/LA/HF mAbs mediated near complete cell death (FIG. 55). It is important to note that the cell death induced by the mixture of BDR5-1LA/LA/HF and TDR5-1LA/LA/HF mAbs which lack immune effector function, is comparable to the killing potency of the mixture of BDR5-1HF and TDR5-1HF (FIG. 55) which contain only the H429F mutation and retain the unmutated L234 and L235 residues.

The increased apoptosis achieved by the BDR5-1LA/LA/HF and TDR5-1LA/LA/HF mAbs when used alone is also consistent with the increased apoptosis by BDR5-1HF and TDR5-1HF mAbs, when used as single agents (see FIG. 38 and FIG. 42) where the BDR5-1HF and TDR5-1HF mAbs mediated improved apoptosis when formatted either on IgG1 or IgG2 backbone. Thus, the LA/LA/HF variants would likewise be expected to also mediate enhanced function on IgG2 backbones.

It is clear that the induction of apoptosis death by anti-DR5 antibodies either used alone or in combination is conferred or enhanced by modification of H429 (FIG. 55, and also FIGS. 38 and 39) and the most potent apoptosis is achieved when the mAbs are used in combination. Importantly, the H429F enhancement of apoptosis was retained despite the presence of mutations elsewhere in the heavy chain, which in this case are known to inactivate both Fc receptor activation and complement activation (i.e. the effects of CH2 modification is independent of the effects of H429 modification). Thus, modifications of CH2 which impact these functions do not affect the unique oligomerisation properties conferred on an IgG Fc region by H429F modification which, in this case, is manifest by the enhanced signalling via DR5 induced by H429F mutation resulting in enhanced apoptotic cell death.

In addition to the LA/LA mutations that impair effector functions of IgG, other mutations in CH2 are well known to alter effector function (An Z et al., MAbs 1:572-579, 2009) including mutation of N297 of the IgG heavy chain which eliminates N-glycosylation of the IgG heavy chain and thereby inactivates effector functions (Walker M R et al., Biochem J 259:347-353, 1989). Additionally, effector functions have been modified in the other human IgG subclasses by mutation of their lower hinge sequences—IgG3 is identical to IgG1 (LLGG) and LA/LA inactivates its effector function. Moreover, IgG2 and IgG4 (An Z et al., 2009 supra; and Xu D et al., 2000 supra), which already show reduced effector function and an impaired capacity to activate the complement cascade, can also be mutated to inactivate their immune effector functions (Wang X et al., 2018 supra). Thus, the data showing the function enhancing effects of H429 mutation and the effects resulting from alterations to CH2 to affect immune effector function together indicate that H429 modification of CH3 could also be applied with modifications in CH2 that increase function such as increasing FcγR activation (Lazar G A et al., 2006 supra) or complement activation (Lee C-H et al., 2017 supra; and Wang X et al., 2018 supra) or both or FcRn binding (Dall'Acqua W F et al., 2016 supra). Further, while in this example, mutations were made in separate domains, multiple mutations can frequently be made in the same domain of the immunoglobulin chain with the resulting mutant displaying properties conferred by each of the individual mutations. For example, in some embodiments, an H429 substitution (i.e. in CH3) may be combined with modifications known to enhance binding to FcRn (e.g. M438L and N434S; Zalevsky J et al., 2012 supra), that are also in CH3, so as to increase the in vivo half-life of an immunotherapeutic protein of the disclosure. Further, while in this example, the effects of the various mutations on signal amplification were evaluated by apoptosis, it is to be understood that enhancement of signal amplification may also be observed by, for example, other means or outcomes from increased signalling-dependent biological effects on the cell, such as cellular responses (including receptor-dependent cellular responses such as cell activation), inhibition of cell activation or function, and cytokine release.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the immunotherapeutic proteins, uses and pharmaceutical composition disclosed herein are not restricted by the particular application(s) described. Neither are the proteins, uses and pharmaceutical composition restricted in their preferred embodiment(s) with regard to the particular elements and/or features described or depicted herein. It will also be appreciated that the proteins, uses and pharmaceutical composition disclosed herein are not limited to the embodiment or embodiments disclosed, but are capable of numerous rearrangements, modifications and substitutions without departing from the scope of the disclosure as set forth and defined by the following claims.

TABLE 5
SEQUENCES

SEQ ID NO: 3 IgG1 H chain CH1-hinge-CH2-CH3 (Protein)
>J00228_IgG1
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQQGNVFSC
SVMHEALHNHYTQKSLSLSPGK
SEQ ID NO: 4 IgG3 H chain (NB hinge 4 repeat form) (Protein)
>X03604-IGHG3
ASTKGPSVFPLAPCSRSTSGGTAALGCLVKDYFPEPVTVSWNSGALTSGVHTFPAVLQSSGLYSLSSVVTVPSSSLG
TQTYTCNVNHKPSNTKVDKRVELKTPLGDTTHTCPRCPEPKSCDTPPPCPRCPEPKSCDTPPPCPRCPEPKSCDTPP
PCPRCPAPELLGGPSVFLFPPKPKDTLMISRTPEVTCVVVDVSHEDPEVQFKWYVDGVEVHNAKTKPREEQYNSTER
VVSVLTVLHQDWLNGKEYKCKVSNKALPAPIEKTISKTKGQPREPQVYTLPPSREEMTKNQVSLTCLVKGFYPSDIA
VEWESSGQPENNYNTTPPMLDSDGSFFLYSKLTVDKSRWQQGNIFSCSVMHEALHNRFTQKSLSLSPGK
SEQ ID NO: 5 IgG4 H chain (Protein)
>K01316-IGHG4
ASTKGPSVFPLAPCSRSTSESTAALGCLVKDYFPEPVTVSWNSGALTSGVHTFPAVLQSSGLYSLSSVVTVPSSSLG
TKTYTCNVDHKPSNTKVDKRVESKYGPPCPSCPAPEFLGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLTVDKSRWQEGNVFSCSVM
HEALHNHYTQKSLSLSLGK
SEQ ID NO: 6 IgG2 H chain (Protein)
>J00230-IGHG2
ASTKGPSVFPLAPCSRSTSESTAALGCLVKDYFPEPVTVSWNSGALTSGVHTFPAVLQSSGLYSLSSVVTVPSSNFG
TQTYTCNVDHKPSNTKVDKTVERKCCVECPPCPAPPVAGPSVFLFPPKPKDTLMISRTPEVTCVVVDVSHEDPEVQF
NWYVDGVEVHNAKTKPREEQFNSTFRVVSVLTVVHQDWLNGKEYKCKVSNKGLPAPIEKTISKTKGQPREPQVYTLP
PSREEMTKNQVSLTCLVKGFYPSDIAVEWESNGQPENNYKTTPPMLDSDGSFFLYSKLTVDKSRWQQGNVFSCSVMH
EALHNHYTQKSLSLSPGK
SEQ ID NO: 7 IgA1 H chain (Protein)
>J00220_IGHA1
ASPTSPKVFPLSLCSTQPDGNVVIACLVQGFFPQEPLSVTWSESGQGVTARNFPPSQDASGDLYTTSSQLTLPATQC
LAGKSVTCHVKHYTNPSQDVTVPCPVPSTPPTPSPSTPPTPSPSCCHPRLSLHRPALEDLLLGSEANLTCTLTGLRD
ASGVTFTWTPSSGKSAVQGPPERDLCGCYSVSSVLPGCAEPWNHGKTFTCTAAYPESKTPLTATLSKSGNTFRPEVH
LLPPPSEELALNELVTLTCLARGESPKDVLVRWLQGSQELPREKYLTWASRQEPSQGTTTFAVTSILRVAAEDWKKG
DTFSCMVGHEALPLAFTQKTIDRLAGKPTHVNVSVVMAEVDGTCY
SEQ ID NO: 8 IgA2 H chain (Protein)
>J00221_IGHA2
ASPTSPKVFPLSLDSTPQDGNVVVACLVQGFFPQEPLSVTWSESGQNVTARNFPPSQDASGDLYTTSSQLTLPATQC
PDGKSVTCHVKHYTNPSQDVTVPCPVPPPPPCCHPRLSLHRPALEDLLLGSEANLTCTLTGLRDASGATFTWTPSSG
KSAVQGPPERDLCGCYSVSSVLPGCAQPWNHGETFTCTAAHPELKTPLTANITKSGNTFRPEVHLLPPPSEELALNE
LVTLTCLARGESPKDVLVRWLQGSQELPREKYLTWASRQEPSQGTTTFAVTSILRVAAEDWKKGDTFSCMVGHEALP
LAFTQKTIDRLAGKPTHVNVSVVMAEVDGTCY
SEQ ID NO: 9 IgE CH4 (Protein)
>IGHE_CH4_J00222
GPRAAPEVYAFATPEWPGSRDKRTLACLIQNFMPEDISVQWLHNEVQLPDARHSTTQPRKTKGSGFFVESRLEVTRA
EWEQKDEFICRAVHEAASPSQTVQRAVSVNPGK
SEQ ID NO: 10 IgM CH4 (Protein)
>IGHM_CH4_X57331
GVALHRPDVYLLPPAREQLNLRESATITCLVTGFSPADVFVQWMQRGQPLSPEKYVTSAPMPEPQAPGRYFAHSILT
VSEEEWNTGETYTCVVAHEALPNRVTERTVDKSTGKPTLYNVSLVMSDTAGTCY
SEQ ID NO: 11 IgD CH3 (Protein)
>IGHD_K02879
AAQAPVKLSLNLLASSDPPEAASWLLCEVSGFSPPNILLMWLEDQREVNTSGFAPARPPPQPRSTTFWAWSVLRVPA
PPSPQPATYTCVVSHEDSRTLLNASRSLEVSYVTDHGPMK
SEQ ID NO: 12 Trastuzumab heavy chain (Protein)
EVQLVESGGGLVQPGGSLRLSCAASGENIKDTYIHWVRQAPGKGLEWVARIYPTNGYTRYADSVKGRFTISADTSKN
TAYLQMNSLRAEDTAVYYCSRWGGDGFYAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGK*
SEQ ID NO: 13 Trastuzumab heavy chain DNA
GAAGTGCAGCTGGTTGAATCTGGCGGCGGACTGGTTCAACCAGGCGGATCTCTGAGACTGAGCTGTGCCGCCAGCGG
CTTCAACATCAAGGACACCTACATCCACTGGGTCCGACAGGCCCCTGGCAAAGGACTTGAATGGGTCGCCAGAATCT
ACCCCACCAACGGCTACACCAGATACGCCGACTCTGTGAAGGGCAGATTCACCATCAGCGCCGACACCAGCAAGAAC
ACCGCCTACCTGCAGATGAACAGCCTGAGAGCCGAGGACACCGCCGTGTACTACTGTAGTAGATGGGGAGGCGACGG
CTTCTACGCTATGGATTATTGGGGCCAGGGCACCCTGGTCACAGTTAGCTCTGCGTCGACAAAAGGCCCTAGCGTCT
TTCCACTCGCCCCATCCTCCAAGAGCACCTCTGGGGGCACTGCGGCTTTGGGCTGCTTGGTGAAGGATTACTTCCCA
GAGCCTGTCACTGTCAGCTGGAATAGCGGGGCTCTGACCAGTGGAGTGCACACCTTTCCCGCCGTGTTACAGAGCAG
CGGCCTCTATAGCCTGAGCAGTGTGGTAACTGTGCCCTCGAGCAGCTTGGGTACCCAGACCTATATCTGCAATGTCA
ATCATAAACCCAGTAACACAAAGGTAGACAAGAAAGTCGAACCCAAATCTTGCGACAAAACTCACACATGCCCTCCT
TGCCCCGCCCCCGAACTCCTGGGGGGACCTTCGGTTTTTCTTTTTCCACCTAAACCGAAAGACACCCTGATGATCAG
CAGGACACCAGAAGTGACATGTGTCGTCGTGGACGTATCCCATGAGGACCCGGAGGTGAAGTTCAACTGGTATGTTG
ATGGTGTGGAAGTGCATAATGCAAAAACTAAGCCACGGGAGGAACAGTACAATAGCACATACAGAGTAGTTAGCGTT
CTGACTGTCCTGCACCAAGATTGGCTGAACGGGAAGGAATACAAGTGTAAGGTGAGCAACAAAGCCCTGCCCGCTCC
AATTGAGAAAACAATTTCTAAGGCCAAAGGACAGCCCCGGGAGCCACAGGTGTATACTCTGCCGCCTAGCAGGGAGG
AAATGACCAAGAACCAGGTGAGCCTGACCTGTCTGGTGAAAGGCTTCTACCCCAGCGATATCGCAGTTGAGTGGGAG
AGCAATGGCCAACCCGAGAACAACTACAAGACTACGCCCCCCGTGCTTGATAGCGACGGATCATTTTTCCTGTACTC
AAAACTGACCGTGGACAAAAGCAGATGGCAGCAGGGAAACGTTTTCAGTTGCTCAGTGATGCACGAAGCGCTGCACA
ATCATTATACTCAGAAAAGCCTGAGCTTGAGCCCGGGAAAATGA
SEQ ID NO: 14 Trastuzumab light chain (Protein)
DIQMTQSPSSLSASVGDRVTITCRASQDVNTAVAWYQQKPGKAPKLLIYSASFLYSGVPSRFSGSRSGTDFTLTISS
LQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC*
SEQ ID NO: 15 Trastuzumab light chain DNA
GATATCCAGATGACACAGAGCCCCAGCAGCCTGTCTGCCTCTGTGGGAGACAGAGTGACCATCACCTGTAGAGCCAG
CCAGGACGTGAACACAGCCGTGGCTTGGTATCAGCAGAAGCCTGGCAAGGCCCCTAAGCTGCTGATCTACAGCGCCA
GCTTTCTGTACAGCGGCGTGCCCAGCAGATTCAGCGGAAGCAGAAGCGGCACCGACTTCACCCTGACCATAAGCAGT
CTGCAGCCCGAGGACTTCGCCACCTACTACTGTCAGCAGCACTACACCACACCTCCAACCTTCGGCCAGGGTACCAA
GGTGGAAATCAAGCGGACAGTGGCCGCTCCTAGCGTGTTCATCTTTCCACCTAGCGACGAGCAGCTGAAGTCTGGCA
CAGCCTCTGTCGTGTGCCTGCTGAACAACTTCTACCCCAGAGAAGCCAAGGTGCAGTGGAAGGTGGACAACGCCCTG
CAGAGCGGCAATAGCCAAGAGAGCGTGACCGAGCAGGACAGCAAGGACTCTACCTACAGCCTGAGCAGCACCCTGAC
ACTGAGCAAGGCCGACTACGAGAAGCACAAAGTGTACGCCTGCGAAGTGACCCACCAGGGCCTTTCTAGCCCTGTGA
CCAAGAGCTTCAACCGGGGCGAGTGCTAA
SEQ ID NO: 16 11B8 heavy Chain (Protein)
EVQLVESGGGLVQPGRSLRLSCAASGFTENDYAMHWVRQAPGKGLEWVSTISWNSGSIGYADSVKGRFTISRDNAKK
SLYLQMNSLRAEDTALYYCAKDIQYGNYYYGMDVWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGK*
SEQ ID NO: 17 11B8 heavy Chain DNA
GAAGTGCAGCTGGTTGAATCTGGCGGCGGACTGGTTCAGCCTGGCAGATCTCTGAGACTGAGCTGTGCCGCCAGCGG
CTTCACCTTCAACGATTACGCCATGCACTGGGTCCGACAGGCCCCTGGAAAAGGCCTTGAATGGGTGTCCACCATCA
GCTGGAACAGCGGCTCTATCGGCTACGCCGATTCCGTGAAGGGCAGATTCACCATCTCCAGAGACAACGCCAAGAAG
TCCCTGTACCTGCAGATGAACAGCCTGAGAGCCGAGGACACAGCCCTGTACTACTGCGCCAAGGACATCCAGTACGG
CAACTACTACTACGGCATGGACGTGTGGGGCCAGGGCACACTGGTCACAGTTAGCTCTGCTAGCACAAAAGGCCCTA
GCGTCTTTCCACTCGCCCCATCCTCCAAGAGCACCTCTGGGGGCACTGCGGCTTTGGGCTGCTTGGTGAAGGATTAC
TTCCCAGAGCCTGTCACTGTCAGCTGGAATAGCGGGGCTCTGACCAGTGGAGTGCACACCTTTCCCGCCGTGTTACA
GAGCAGCGGCCTCTATAGCCTGAGCAGTGTGGTAACTGTGCCCTCGAGCAGCTTGGGTACCCAGACCTATATCTGCA
ATGTCAATCATAAACCCAGTAACACAAAGGTAGACAAGAAAGTCGAACCCAAATCTTGCGACAAAACTCACACATGC
CCTCCTTGCCCCGCCCCCGAACTCCTGGGGGGACCTTCGGTTTTTCTTTTTCCACCTAAACCGAAAGACACCCTGAT
GATCAGCAGGACACCAGAAGTGACATGTGTCGTCGTGGACGTATCCCATGAGGACCCGGAGGTGAAGTTCAACTGGT
ATGTTGATGGTGTGGAAGTGCATAATGCAAAAACTAAGCCACGGGAGGAACAGTACAATAGCACATACAGAGTAGTT
AGCGTTCTGACTGTCCTGCACCAAGATTGGCTGAACGGGAAGGAATACAAGTGTAAGGTGAGCAACAAAGCCCTGCC
CGCTCCAATTGAGAAAACAATTTCTAAGGCCAAAGGACAGCCCCGGGAGCCACAGGTGTATACTCTGCCGCCTAGCA
GGGAGGAAATGACCAAGAACCAGGTGAGCCTGACCTGTCTGGTGAAAGGCTTCTACCCCAGCGATATCGCAGTTGAG
TGGGAGAGCAATGGCCAACCCGAGAACAACTACAAGACTACGCCCCCCGTGCTTGATAGCGACGGATCATTTTTCCT
GTACTCAAAACTGACCGTGGACAAAAGCAGATGGCAGCAGGGAAACGTTTTCAGTTGCTCAGTGATGCACGAAGCGC
TGCACAATCATTATACTCAGAAAAGCCTGAGCTTGAGCCCGGGAAAATGA
SEQ ID NO: 18 11B8 Light Chain (Protein)
EIVLTQSPATLSLSPGERATLSCRASQSVSSYLAWYQQKPGQAPRLLIYDASNRATGIPARFSGSGSGTDFTLTISS
LEPEDFAVYYCQQRSNWPITFGQGTRLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC*
SEQ ID NO: 19 11B8 Light Chain DNA
GAAATTGTGCTGACACAGAGCCCCGCCACACTGTCACTTTCTCCAGGCGAAAGAGCCACACTGAGCTGCAGAGCCAG
CCAGAGCGTGTCCTCTTACCTGGCCTGGTATCAGCAGAAGCCTGGACAGGCTCCCCGGCTGCTGATCTACGATGCCA
GCAATAGAGCCACCGGCATTCCCGCCAGATTTTCTGGCTCTGGAAGCGGCACCGACTTCACCCTGACCATAAGCAGC
CTGGAACCTGAGGACTTCGCCGTGTACTACTGCCAGCAGAGAAGCAACTGGCCCATCACCTTTGGCCAGGGTACCAG
ACTGGAAATCAAGCGGACAGTGGCCGCTCCTAGCGTGTTCATCTTTCCACCTAGCGACGAGCAGCTGAAGTCTGGCA
CAGCCTCTGTCGTGTGCCTGCTGAACAACTTCTACCCCAGAGAAGCCAAGGTGCAGTGGAAGGTGGACAACGCCCTG
CAGAGCGGCAATAGCCAAGAGAGCGTGACCGAGCAGGACAGCAAGGACTCTACCTACAGCCTGAGCAGCACCCTGAC
ACTGAGCAAGGCCGACTACGAGAAGCACAAAGTGTACGCCTGCGAAGTGACCCACCAGGGCCTTTCTAGCCCTGTGA
CCAAGAGCTTCAACCGGGGCGAGTGCTAA
SEQ ID NO: 20
Daratumumab Heavy Chain (Protein)
EVQLLESGGGLVQPGGSLRLSCAVSGFTENSFAMSWVRQAPGKGLEWVSAISGSGGGTYYADSVKGRFTISRDNSKN
TLYLQMNSLRAEDTAVYFCAKDKILWFGEPVFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGK*
SEQ ID NO: 21 Daratumumab Heavy Chain DNA
GAAGTTCAGCTGCTGGAATCTGGCGGCGGACTGGTTCAACCAGGCGGATCTCTGAGACTGAGCTGTGCCGTGTCCGG
CTTCACCTTCAACAGCTTCGCCATGAGCTGGGTCCGACAGGCCCCTGGAAAAGGCCTTGAATGGGTGTCCGCCATCT
CTGGCTCTGGCGGAGGCACATATTACGCCGACTCTGTGAAGGGCAGATTCACCATCAGCCGGGACAACAGCAAGAAC
ACCCTGTACCTGCAGATGAACAGCCTGAGAGCCGAGGATACCGCCGTGTACTTCTGCGCCAAGGACAAGATCCTTTG
GTTCGGCGAGCCCGTGTTCGATTATTGGGGCCAGGGCACCCTGGTCACAGTTAGCTCTGCTAGCACAAAAGGCCCTA
GCGTCTTTCCACTCGCCCCATCCTCCAAGAGCACCTCTGGGGGCACTGCGGCTTTGGGCTGCTTGGTGAAGGATTAC
TTCCCAGAGCCTGTCACTGTCAGCTGGAATAGCGGGGCTCTGACCAGTGGAGTGCACACCTTTCCCGCCGTGTTACA
GAGCAGCGGCCTCTATAGCCTGAGCAGTGTGGTAACTGTGCCCTCGAGCAGCTTGGGTACCCAGACCTATATCTGCA
ATGTCAATCATAAACCCAGTAACACAAAGGTAGACAAGAGAGTCGAACCCAAATCTTGCGACAAAACTCACACATGC
CCTCCTTGCCCCGCCCCCGAACTCCTGGGGGGACCTTCGGTTTTTCTTTTTCCACCTAAACCGAAAGACACCCTGAT
GATCAGCAGGACACCAGAAGTGACATGTGTCGTCGTGGACGTATCCCATGAGGACCCGGAGGTGAAGTTCAACTGGT
ATGTTGATGGTGTGGAAGTGCATAATGCAAAAACTAAGCCACGGGAGGAACAGTACAATAGCACATACAGAGTAGTT
AGCGTTCTGACTGTCCTGCACCAAGATTGGCTGAACGGGAAGGAATACAAGTGTAAGGTGAGCAACAAAGCCCTGCC
CGCTCCAATTGAGAAAACAATTTCTAAGGCCAAAGGACAGCCCCGGGAGCCACAGGTGTATACTCTGCCGCCTAGCA
GGGAGGAAATGACCAAGAACCAGGTGAGCCTGACCTGTCTGGTGAAAGGCTTCTACCCCAGCGATATCGCAGTTGAG
TGGGAGAGCAATGGCCAACCCGAGAACAACTACAAGACTACGCCCCCCGTGCTTGATAGCGACGGATCATTTTTCCT
GTACTCAAAACTGACCGTGGACAAAAGCAGATGGCAGCAGGGAAACGTTTTCAGTTGCTCAGTGATGCACGAAGCGC
TGCACAATCATTATACTCAGAAAAGCCTGAGCTTGAGCCCGGGAAAATGA
SEQ ID NO: 22 Daratumumab Light Chain (Protein)
EIVLTQSPATLSLSPGERATLSCRASQSVSSYLAWYQQKPGQAPRLLIYDASNRATGIPARFSGSGSGTDFTLTISS
LEPEDFAVYYCQQRSNWPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC*
SEQ ID NO: 23 Daratumumab Light Chain DNA
GAAATTGTGCTGACACAGAGCCCCGCCACACTGTCACTTTCTCCAGGCGAAAGAGCCACACTGAGCTGCAGAGCCAG
CCAGAGCGTGTCCTCTTACCTGGCCTGGTATCAGCAGAAGCCTGGACAGGCTCCCCGGCTGCTGATCTACGATGCCA
GCAATAGAGCCACCGGCATTCCCGCCAGATTTTCTGGCTCTGGAAGCGGCACCGACTTCACCCTGACCATAAGCAGC
CTGGAACCTGAGGACTTCGCCGTGTACTACTGCCAGCAGAGAAGCAACTGGCCTCCTACCTTTGGCCAGGGTACCAA
GGTGGAAATCAAGCGGACAGTGGCCGCTCCTAGCGTGTTCATCTTTCCACCTAGCGACGAGCAGCTGAAGTCTGGCA
CAGCCTCTGTCGTGTGCCTGCTGAACAACTTCTACCCCAGAGAAGCCAAGGTGCAGTGGAAGGTGGACAACGCCCTG
CAGAGCGGCAATAGCCAAGAGAGCGTGACCGAGCAGGACAGCAAGGACTCTACCTACAGCCTGAGCAGCACCCTGAC
ACTGAGCAAGGCCGACTACGAGAAGCACAAAGTGTACGCCTGCGAAGTGACCCACCAGGGCCTTTCTAGCCCTGTGA
CCAAGAGCTTCAACCGGGGCGAGTGCTAA
SEQ ID NO: 24 Pertuzumab heavy chain (Protein)
EVQLVESGGGLVQPGGSLRLSCAASGFTFTDYTMDWVRQAPGKGLEWVADVNPNSGGSIYNQRFKGRFTLSVDRSKN
TLYLQMNSLRAEDTAVYYCARNLGPSFYFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK*
SEQ ID NO: 25 Pertuzumab heavy chain DNA
GAAGTGCAGCTGGTTGAATCTGGCGGCGGACTGGTTCAACCAGGCGGATCTCTGAGACTGAGCTGTGCCGCCAGCGG
CTTCACCTTCACCGACTACACAATGGACTGGGTCCGACAGGCCCCTGGCAAAGGACTTGAATGGGTCGCCGACGTGA
ACCCCAATAGCGGCGGCAGCATCTACAACCAGCGGTTCAAGGGCAGATTCACCCTGAGCGTGGACAGAAGCAAGAAC
ACCCTGTACCTGCAGATGAACAGCCTGAGAGCCGAGGACACCGCCGTGTACTACTGCGCCAGAAATCTGGGCCCCAG
CTTCTACTTCGATTATTGGGGCCAGGGCACCCTGGTCACAGTTAGCTCTGCTAGCACAAAAGGCCCTAGCGTCTTTC
CACTCGCCCCATCCTCCAAGAGCACCTCTGGGGGCACTGCGGCTTTGGGCTGCTTGGTGAAGGATTACTTCCCAGAG
CCTGTCACTGTCAGCTGGAATAGCGGGGCTCTGACCAGTGGAGTGCACACCTTTCCCGCCGTGTTACAGAGCAGCGG
CCTCTATAGCCTGAGCAGTGTGGTAACTGTGCCCTCGAGCAGCTTGGGTACCCAGACCTATATCTGCAATGTCAATC
ATAAACCCAGTAACACAAAGGTAGACAAGAAAGTCGAACCCAAATCTTGCGACAAAACTCACACATGCCCTCCTTGC
CCCGCCCCCGAACTCCTGGGGGGACCTTCGGTTTTTCTTTTTCCACCTAAACCGAAAGACACCCTGATGATCAGCAG
GACACCAGAAGTGACATGTGTCGTCGTGGACGTATCCCATGAGGACCCGGAGGTGAAGTTCAACTGGTATGTTGATG
GTGTGGAAGTGCATAATGCAAAAACTAAGCCACGGGAGGAACAGTACAATAGCACATACAGAGTAGTTAGCGTTCTG
ACTGTCCTGCACCAAGATTGGCTGAACGGGAAGGAATACAAGTGTAAGGTGAGCAACAAAGCCCTGCCCGCTCCAAT
TGAGAAAACAATTTCTAAGGCCAAAGGACAGCCCCGGGAGCCACAGGTGTATACTCTGCCGCCTAGCAGGGAGGAAA
TGACCAAGAACCAGGTGAGCCTGACCTGTCTGGTGAAAGGCTTCTACCCCAGCGATATCGCAGTTGAGTGGGAGAGC
AATGGCCAACCCGAGAACAACTACAAGACTACGCCCCCCGTGCTTGATAGCGACGGATCATTTTTCCTGTACTCAAA
ACTGACCGTGGACAAAAGCAGATGGCAGCAGGGAAACGTTTTCAGTTGCTCAGTGATGCACGAAGCGCTGCACAATC
ATTATACTCAGAAAAGCCTGAGCTTGAGCCCGGGAAAATGA
SEQ ID NO: 26 Pertuzumab light chain (Protein)
DIQMTQSPSSLSASVGDRVTITCKASQDVSIGVAWYQQKPGKAPKLLIYSASYRYTGVPSRFSGSGSGTDFTLTISS
LQPEDFATYYCQQYYIYPYTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC*
SEQ ID NO: 27 Pertuzumab light chain DNA
GATATCCAGA5TGACACAGAGCCCCAGCAGCCTGTCTGCCTCTGTGGGAGACAGAGTGACCATCACCTGTAAAGCCA
GCCAGGACGTGTCCATCGGCGTGGCATGGTATCAGCAGAAGCCTGGCAAGGCCCCTAAGCTGCTGATCTACAGCGCC
AGCTACAGATACACCGGCGTGCCCAGCAGATTTTCTGGCAGCGGCTCTGGCACCGACTTCACCCTGACCATATCTAG
CCTGCAGCCTGAGGACTTCGCCACCTACTACTGCCAGCAGTACTACATCTACCCCTACACCTTCGGCCAGGGTACCA
AGGTGGAAATCAAGCGGACAGTGGCCGCTCCTAGCGTGTTCATCTTTCCACCTAGCGACGAGCAGCTGAAGTCTGGC
ACAGCCTCTGTCGTGTGCCTGCTGAACAACTTCTACCCCAGAGAAGCCAAGGTGCAGTGGAAGGTGGACAACGCCCT
GCAGAGCGGCAATAGCCAAGAGAGCGTGACCGAGCAGGACAGCAAGGACTCTACCTACAGCCTGAGCAGCACCCTGA
CACTGAGCAAGGCCGACTACGAGAAGCACAAAGTGTACGCCTGCGAAGTGACCCACCAGGGCCTTTCTAGCCCTGTG
ACCAAGAGCTTCAACCGGGGCGAGTGCTAA
SEQ ID NO: 28 Rituximab Heavy Chain (Protein)
QVQLQQPGAELVKPGASVKMSCKASGYTFTSYNMHWVKQTPGRGLEWIGAIYPGNGDTSYNQKFKGKATLTADKSSS
TAYMQLSSLTSEDSAVYYCARSTYYGGDWYFNVWGAGTTVTVSAASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSPGK*
SEQ ID NO: 29 Rituximab Heavy Chain DNA
CAGGTTCAGCTGCAACAGCCTGGCGCCGAGCTTGTGAAACCTGGCGCCTCTGTGAAGATGAGCTGCAAGGCCAGCGG
CTACACCTTCACCAGCTACAACATGCACTGGGTCAAGCAGACCCCTGGCAGAGGCCTGGAATGGATCGGAGCCATCT
ATCCCGGCAACGGCGACACCTCCTACAACCAGAAGTTCAAGGGCAAAGCCACACTGACCGCCGACAAGAGCAGCAGC
ACAGCCTACATGCAGCTGAGCAGCCTGACCAGCGAAGATAGCGCCGTGTACTACTGCGCCAGAAGCACCTATTACGG
CGGCGACTGGTACTTCAACGTGTGGGGAGCTGGCACCACCGTGACAGTTTCTGCCGCTAGCACAAAAGGCCCTAGCG
TCTTTCCACTCGCCCCATCCTCCAAGAGCACCTCTGGGGGCACTGCGGCTTTGGGCTGCTTGGTGAAGGATTACTTC
CCAGAGCCTGTCACTGTCAGCTGGAATAGCGGGGCTCTGACCAGTGGAGTGCACACCTTTCCCGCCGTGTTACAGAG
CAGCGGCCTCTATAGCCTGAGCAGTGTGGTAACTGTGCCCTCGAGCAGCTTGGGTACCCAGACCTATATCTGCAATG
TCAATCATAAACCCAGTAACACAAAGGTAGACAAGAAAGTCGAACCCAAATCTTGCGACAAAACTCACACATGCCCT
CCTTGCCCCGCCCCCGAACTCCTGGGGGGACCTTCGGTTTTTCTTTTTCCACCTAAACCGAAAGACACCCTGATGAT
CAGCAGGACACCAGAAGTGACATGTGTCGTCGTGGACGTATCCCATGAGGACCCGGAGGTGAAGTTCAACTGGTATG
TTGATGGTGTGGAAGTGCATAATGCAAAAACTAAGCCACGGGAGGAACAGTACAATAGCACATACAGAGTAGTTAGC
GTTCTGACTGTCCTGCACCAAGATTGGCTGAACGGGAAGGAATACAAGTGTAAGGTGAGCAACAAAGCCCTGCCCGC
TCCAATTGAGAAAACAATTTCTAAGGCCAAAGGACAGCCCCGGGAGCCACAGGTGTATACTCTGCCGCCTAGCAGGG
ATGAACTGACCAAGAACCAGGTGAGCCTGACCTGTCTGGTGAAAGGCTTCTACCCCAGCGATATCGCAGTTGAGTGG
GAGAGCAATGGCCAACCCGAGAACAACTACAAGACTACGCCCCCCGTGCTTGATAGCGACGGATCATTTTTCCTGTA
CTCAAAACTGACCGTGGACAAAAGCAGATGGCAGCAGGGAAACGTTTTCAGTTGCTCAGTGATGCACGAAGCGCTGC
ACAATCATTATACTCAGAAAAGCCTGAGCTTGAGCCCGGGAAAATGA
SEQ ID NO: 30 Rituximab Light Chain (Protein)
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC*
SEQ ID NO: 31 Rituximab Light Chain DNA
CAAATTGTGCTGTCTCAGAGCCCCGCCATCCTGAGTGCTTCTCCAGGCGAGAAAGTGACCATGACCTGCAGAGCCAG
CAGCAGCGTGTCCTACATCCACTGGTTCCAGCAGAAGCCTGGCAGCAGCCCTAAGCCTTGGATCTACGCCACAAGCA
ATCTGGCCAGCGGCGTGCCAGTCAGATTTTCTGGCTCTGGCAGCGGCACCAGCTACAGCCTGACAATCAGCAGAGTG
GAAGCCGAGGATGCCGCCACCTACTACTGTCAGCAGTGGACCAGCAATCCTCCTACCTTTGGCGGCGGTACCAAGGT
GGAAATCAAGCGGACAGTGGCCGCTCCTAGCGTGTTCATCTTTCCACCTAGCGACGAGCAGCTGAAGTCTGGCACAG
CCTCTGTCGTGTGCCTGCTGAACAACTTCTACCCCAGAGAAGCCAAGGTGCAGTGGAAGGTGGACAACGCCCTGCAG
AGCGGCAATAGCCAAGAGAGCGTGACCGAGCAGGACAGCAAGGACTCTACCTACAGCCTGAGCAGCACCCTGACACT
GAGCAAGGCCGACTACGAGAAGCACAAAGTGTACGCCTGCGAAGTGACCCACCAGGGCCTTTCTAGCCCTGTGACCA
AGAGCTTCAACCGGGGCGAGTGCTAA
SEQ ID NO: 32 A TNP IgG1 heavy chain (protein)
EVQLQESGPSLVKPSQTLSLTCSVTGDSITSGYWNWIRQFPGNKLEYMGFINYSGNTYYNPSLRSRISITRDTSKNQ
YFLHLNSVTTEDTATYYCARANWDVFAYWGQGTLVTVSAASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK*
SEQ ID NO: 33 A TNP-IgG1 heavy chain DNA
GAGGTGCAGCTTCAGGAGTCAGGACCTAGCCTCGTGAAACCTTCTCAGACTCTGTCCCTCACATGTTCTGTCACTGG
CGACTCCATCACCAGTGGTTACTGGAACTGGATCCGGCAATTCCCAGGGAATAAACTTGAGTACATGGGTTTCATAA
ATTACAGTGGTAACACTTACTACAATCCATCTCTCAGAAGTCGAATCTCCATCACTCGAGACACATCCAAGAACCAG
TACTTCCTGCACTTGAATTCTGTGACTACTGAGGACACAGCCACATATTACTGTGCAAGGGCTAACTGGGACGTCTT
TGCTTACTGGGGCCAAGGGACTCTGGTCACTGTCTCTGCAGCCTCCACCAAGGGCCCATCGGTCTTCCCCCTGGCAC
CCTCCTCCAAGAGCACCTCTGGGGGCACAGCGGCCCTGGGCTGCCTGGTCAAGGACTACTTCCCCGAACCGGTGACG
GTGTCGTGGAACTCAGGCGCCCTGACCAGCGGCGTGCACACCTTCCCGGCTGTCCTACAGTCCTCAGGACTCTACTC
CCTCAGCAGCGTGGTGACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCCCACCGTGCCCAGCACCT
GAACTCCTGGGGGGACCGTCAGTCTTCCTCTTCCCCCCAAAACCCAAGGACACCCTCATGATCTCCCGGACCCCTGA
GGTCACATGCGTGGTGGTGGACGTGAGCCACGAAGACCCTGAGGTCAAGTTCAACTGGTACGTGGACGGCGTGGAGG
TGCATAATGCCAAGACAAAGCCGCGGGAGGAGCAGTACAACAGCACGTACCGTGTGGTCAGCGTCCTCACCGTCCTG
CACCAGGACTGGCTGAATGGCAAGGAGTACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTGCCCCCATCCCGGGATGAGCTGACCAAGA
ACCAGGTCAGCCTGACCTGCCTGGTCAAAGGCTTCTATCCCAGCGACATCGCCGTGGAGTGGGAGAGCAATGGGCAG
CCGGAGAACAACTACAAGACCACGCCTCCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGCATGAGGCTCTGCACAACCACTACACGC
AGAAGAGCCTCTCCCTGTCTCCGGGTAAATGA
SEQ ID NO: 34 A TNP IgG2 heavy chain (protein)
EVQLQESGPSLVKPSQTLSLTCSVTGDSITSGYWNWIRQVPGNKLEYMGFINYSGNTYYNPSLRSRISITRDTSKNQ
YFLHLNSVTTEDTATYYCARANWDVFAYWGQGTLVTVSAASTKGPSVFPLAPCSRSTSESTAALGCLVKDYFPEPVT
VSWNSGALTSGVHTFPAVLQSSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQVSLTCLVKGFYPSDIAVEWESNGQPENN
YKTTPPMLDSDGSFFLYSKLTVDKSRWQQGNVFSCSVMHEALHNHYTQKSLSLSLGK
SEQ ID NO: 35 A TNP IgG2 heavy chain (DNA)
GAGGTGCAGTTGCAGGAATCTGGTCCCAGTCTAGTTAAGCCCAGCCAGACACTGAGCCTGACATGTAGTGTGACCGG
TGACAGCATTACAAGCGGCTACTGGAACTGGATCAGACAGGTGCCAGGAAACAAACTCGAATACATGGGGTTCATCA
ATTACAGTGGCAATACTTACTATAATCCCAGCCTGAGAAGCAGAATTTCTATAACCAGAGACACCAGCAAAAACCAG
TACTTTCTGCACCTGAACAGCGTAACAACGGAAGACACCGCCACCTATTACTGCGCCAGGGCCAACTGGGATGTGTT
CGCATACTGGGGCCAGGGCACTCTGGTGACGGTGAGCGCCGCGTCGACAAAAGGCCCTAGCGTCTTTCCACTCGCCC
CATGTTCAAGAAGCACCAGCGAGTCAACTGCGGCTTTGGGCTGCTTGGTGAAGGATTACTTCCCAGAGCCTGTCACT
GTCAGCTGGAATAGCGGGGCTCTGACCAGTGGAGTGCACACCTTTCCCGCCGTGTTACAGAGCAGCGGCCTCTATAG
CCTGAGCAGTGTGGTAACTGTGCCCTCGAGCAATTTTGGTACCCAGACCTATACATGCAATGTCGATCATAAACCCA
GTAACACAAAGGTAGACAAGACAGTCGAAAGAAAATGCTGTGTGGAGTGCCCTCCTTGCCCCGCCCCCCCAGTGGCC
GGGCCTTCGGTTTTTCTTTTTCCACCTAAACCGAAAGACACCCTGATGATCAGCAGGACACCAGAAGTGACATGTGT
CGTCGTGGACGTATCCCATGAAGACCCGGAGGTGCAATTCAACTGGTATGTTGATGGTGTGGAAGTGCATAATGCAA
AAACTAAGCCACGGGAGGAACAGTTCAATAGCACATTCAGAGTAGTTAGCGTTCTGACTGTCGTGCACCAAGATTGG
CTGAACGGGAAGGAATACAAGTGTAAGGTGAGCAACAAAGGTCTGCCCGCTCCAATTGAGAAAACAATTTCTAAGAC
CAAAGGACAGCCCCGGGAGCCACAGGTGTATACTCTGCCGCCTAGCAGGGAGGAAATGACCAAGAACCAGGTGAGCC
TGACCTGTCTGGTGAAAGGCTTCTACCCCAGCGATATCGCAGTTGAGTGGGAGAGCAATGGCCAACCCGAGAACAAC
TACAAGACTACGCCCCCCATGCTTGATAGCGACGGATCATTTTTCCTGTACTCAAAACTGACCGTGGACAAAAGCAG
ATGGCAGCAGGGAAACGTTTTCAGTTGCTCAGTGATGCACGAAGCgCTGCACAATCATTATACTCAGAAAAGCCTGA
GCTTGAGCTTAGGAAAATGA
SEQ ID NO: 36 A TNP-light chain (protein)
DIVMSQSPSSLAVSVGEKVTMSCKSSQSLLYSSNQKNYLAWYQRKPGQSPKLLIYWASTRESGVPDRFTGSGSGTDF
TLTISSVKAEDLAVYYCQHYYSSPYTFGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
SEQ ID NO: 37 A TNP-light chain (DNA)
GACATAGTGATGTCACAGAGCCCAAGCAGCCTTGCAGTGTCTGTTGGTGAGAAGGTGACCATGAGCTGCAAGTCAAG
CCAGAGCCTGCTCTATAGCAGCAATCAGAAGAACTATTTGGCCTGGTATCAGAGAAAACCCGGCCAGAGCCCTAAGC
TCCTTATTTATTGGGCTAGCACACGCGAGTCCGGCGTGCCGGACAGATTTACCGGCAGCGGTAGCGGCACCGATTTT
ACTTTGACCATCTCGTCTGTAAAGGCCGAAGACCTGGCAGTGTATTACTGTCAACATTATTACTCATCTCCCTACAC
TTTCGGAGGGGGGACCAAACTGGAAATTAAGCGTACGGTTGCTGCCCCTTCTGTCTTCATCTTCCCTCCCAGCGATG
AACAGCTGAAAAGTGGGACAGCGAGTGTAGTGTGCCTGCTAAACAATTTTTACCCACGGGAGGCCAAAGTGCAGTGG
AAAGTGGACAACGCTCTGCAAAGTGGAAATTCTCAGGAGTCCGTCACAGAGCAGGACTCGAAGGACAGCACTTACAG
CTTGTCAAGCACCCTGACGCTGAGCAAAGCCGATTACGAGAAGCACAAGGTGTACGCATGCGAAGTTACGCACCAAG
GACTGAGTAGCCCAGTCACAAAGAGCTTCAACAGGGGGGAATGT
SEQ ID NO: 38 Rituximab IgG3 heavy chain (protein)
QVQLQQPGAELVKPGASVKMSCKASGYTFTSYNMHWVKQTPGRGLEWIGAIYPGNGDTSYNQKFKGKATLTADKSSS
TAYMQLSSLTSEDSAVYYCARSTYYGGDWYFNVWGAGTTVTVSAASTKGPSVFPLAPCSRSTSGGTAALGCLVKDYF
PEPVTVSWNSGALTSGVHTFPAVLQSSGLYSLSSVVTVPSSSLGTQTYTCNVNHKPSNTKVDKRVELKTPLGDTTHT
CPRCPEPKSCDTPPPCPRCPEPKSCDTPPPCPRCPEPKSCDTPPPCPRCPAPELLGGPSVFLFPPKPKDTLMISRTP
EVTCVVVDVSHEDPEVQFKWYVDGVEVHNAKTKLREEQYNSTFRVVSVLTVLHQDWLNGKEYKCKVSNKALPAPIEK
TISKTKGQPREPQVYTLPPSREEMTKNQVSLTCLVKGFYPSDIAVEWESNGQPENNYKTTPPVLDSDGSFFLYSKLT
VDKSRWQQGNIFSCSVMHEALHNHYTQKSLSLSPGK
SEQ ID NO: 39 Rituximab IgG3 heavy chain (DNA)
CAGGTTCAGCTGCAACAGCCTGGCGCCGAGCTTGTGAAACCTGGCGCCTCTGTGAAGATGAGCTGCAAGGCCAGCGG
CTACACCTTCACCAGCTACAACATGCACTGGGTCAAGCAGACCCCTGGCAGAGGCCTGGAATGGATCGGAGCCATCT
ATCCCGGCAACGGCGACACCTCCTACAACCAGAAGTTCAAGGGCAAAGCCACACTGACCGCCGACAAGAGCAGCAGC
ACAGCCTACATGCAGCTGAGCAGCCTGACCAGCGAAGATAGCGCCGTGTACTACTGCGCCAGAAGCACCTATTACGG
CGGCGACTGGTACTTCAACGTGTGGGGAGCTGGTACCACCGTGACAGTTTCTGCCGCTAGCACAAAAGGCCCTAGCG
TCTTTCCACTCGCCCCATGTTCTAGAAGCACCTCTGGGGGCACTGCGGCTTTGGGCTGCTTGGTGAAGGATTACTTC
CCAGAGCCTGTCACTGTCAGCTGGAATAGCGGGGCTCTGACCAGTGGAGTGCACACCTTTCCCGCCGTGTTACAGAG
CAGCGGCCTCTATAGCCTGAGCAGTGTGGTAACTGTGCCCTCGAGCAGCTTGGGCACCCAGACCTATACCTGCAATG
TCAATCATAAACCCAGTAACACAAAGGTAGACAAGCGGGTCGAACTGAAAACCCCCCTGGGCGACACCACCCACACC
TGTCCTAGATGCCCCGAGCCCAAGTCCTGCGATACCCCTCCACCTTGCCCCAGGTGCCCTGAGCCTAAGAGCTGTGA
TACCCCCCCTCCCTGCCCTCGGTGTCCAGAGCCTAAGTCTTGTGACACCCCACCCCCATGTCCAAGGTGCCCCGCCC
CCGAACTCCTGGGGGGACCTTCGGTTTTTCTTTTTCCACCTAAACCGAAAGACACCCTGATGATCAGCAGGACACCA
GAAGTGACATGTGTCGTCGTGGACGTATCCCATGAGGACCCGGAGGTGCAGTTCAAGTGGTATGTTGATGGTGTGGA
AGTGCATAATGCAAAAACTAAGCTGCGGGAGGAACAGTACAATAGCACATTCAGAGTAGTTAGCGTTCTGACTGTCC
TGCACCAAGATTGGCTGAACGGGAAGGAATACAAGTGTAAGGTGAGCAACAAAGCCCTGCCCGCTCCAATTGAGAAA
ACAATTTCTAAGACCAAAGGACAGCCCCGGGAGCCACAGGTGTATACTCTGCCGCCTAGCAGGGAAGAGATGACCAA
GAACCAGGTGAGCCTGACCTGTCTGGTGAAAGGCTTCTACCCCAGCGATATCGCAGTTGAGTGGGAGAGCAATGGCC
AACCCGAGAACAACTACAAGACTACGCCCCCCGTGCTTGATAGCGACGGATCATTTTTCCTGTACTCAAAACTGACC
GTGGACAAAAGCAGATGGCAGCAGGGAAACATCTTCAGTTGCTCAGTGATGCACGAAGCGCTGCACAATCATTATAC
TCAGAAAAGCCTGAGCTTGAGCCCGGGAAAA
SEQ ID NO: 40 Rituximab IgG4 heavy chain (protein)
QVQLQQPGAELVKPGASVKMSCKASGYTFTSYNMHWVKQTPGRGLEWIGAIYPGNGDTSYNQKFKGKATLTADKSSS
TAYMQLSSLTSEDSAVYYCARSTYYGGDWYFNVWGAGTTVTVSAASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPCPSCP
APEFLGGPSVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHNHYTQKSLSLSLGK
SEQ ID NO: 41 Rituximab IgG4 heavy chain (DNA)
CAGGTTCAGCTGCAACAGCCTGGCGCCGAGCTTGTGAAACCTGGCGCCTCTGTGAAGATGAGCTGCAAGGCCAGCGG
CTACACCTTCACCAGCTACAACATGCACTGGGTCAAGCAGACCCCTGGCAGAGGCCTGGAATGGATCGGAGCCATCT
ATCCCGGCAACGGCGACACCTCCTACAACCAGAAGTTCAAGGGCAAAGCCACACTGACCGCCGACAAGAGCAGCAGC
ACAGCCTACATGCAGCTGAGCAGCCTGACCAGCGAAGATAGCGCCGTGTACTACTGCGCCAGAAGCACCTATTACGG
CGGCGACTGGTACTTCAACGTGTGGGGAGCTGGtACCACCGTGACAGTTTCTGCCGCTAGCACAAAAGGCCCTAGCG
TCTTTCCACTCGCCCCATgtTCCAgaAGCACCTCTGAAAGTACTGCGGCTTTGGGCTGCTTGGTGAAGGATTACTTC
CCAGAGCCTGTCACTGTCAGCTGGAATAGCGGGGCTCTGACCAGTGGAGTGCACACCTTTCCCGCCGTGTTACAGAG
CAGCGGCCTCTATAGCCTGAGCAGTGTGGTAACTGTGCCCTCGAGCAGCTTGGGcACCaAGACCTATAcaTGCAATG
TCgAcCATAAACCCAGTAACACAAAGGTAGACAAGcgAGTCGAAAGCAAGTACGGCCCCCCCTGCCCTAGCTGCCCC
GCCCCCGAAtTtCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCAgGAGGACCCGGAGGTGcAGTTCAACTGGTATGTTGATGGTG
TGGAAGTGCATAATGCAAAAACTAAGCCACGGGAGGAACAGTtCAATAGCACATACAGAGTAGTTAGCGTTCTGACT
GTCCTGCACCAAGATTGGCTGAACGGGAAGGAATACAAGTGTAAGGTGAGCAACAAAGgCCTGCCCagTagcATTGA
GAAAACAATTTCTAAGGCCAAAGGACAGCCCCGGGAGCCACAGGTGTATACTCTGCCGCCTAGCcaaGAgGAAaTGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAcggCT
GACCGTGGACAAAAGCAGATGGCAGgAGGGAAACGTTTTCAGTTGCTCAGTGATGCACGAAGCGCTGCACAATCATT
ATACTCAGAAAAGCCTGAGCTTGAGCttaGGAAAA
SEQ ID NO: 42 Isatuximab heavy chain (Protein)
QVQLVQSGAEVAKPGTSVKLSCKASGYTFTDYWMQWVKQRPGQGLEWIGTIYPGDGDTGYAQKFQGKATLTADKSSK
TVYMHLSSLASEDSAVYYCARGDYYGSNSLDYWGQGTSVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 43 Isatuximab heavy chain DNA
CAGGTTCAGCTGGTTCAGTCTGGCGCCGAGGTGGCCAAACCTGGCACATCTGTGAAGCTGAGCTGCAAGGCCAGCGG
CTACACCTTCACCGACTACTGGATGCAGTGGGTCAAGCAGAGGCCTGGACAGGGCCTCGAATGGATCGGCACAATCT
ATCCTGGCGACGGCGATACAGGCTACGCCCAGAAGTTTCAGGGCAAAGCCACACTGACCGCCGACAAGAGCAGCAAG
ACCGTGTACATGCACCTGAGCAGCCTGGCCTCTGAGGATAGCGCCGTGTACTATTGCGCCAGAGGCGACTACTACGG
CAGCAACAGCCTGGATTATTGGGGCCAGGGCACCTCCGTCACAGTTAGCTCTGCTAGCACAAAAGGCCCTAGCGTCT
TTCCACTCGCCCCATCCTCCAAGAGCACCTCTGGGGGCACTGCGGCTTTGGGCTGCTTGGTGAAGGATTACTTCCCA
GAGCCTGTCACTGTCAGCTGGAATAGCGGGGCTCTGACCAGTGGAGTGCACACCTTTCCCGCCGTGTTACAGAGCAG
CGGCCTCTATAGCCTGAGCAGTGTGGTAACTGTGCCCTCGAGCAGCTTGGGTACCCAGACCTATATCTGCAATGTCA
ATCATAAACCCAGTAACACAAAGGTAGACAAGAAAGTCGAACCCAAATCTTGCGACAAAACTCACACATGCCCTCCT
TGCCCCGCCCCCGAACTCCTGGGGGGACCTTCGGTTTTTCTTTTTCCACCTAAACCGAAAGACACCCTGATGATCAG
CAGGACACCAGAAGTGACATGTGTCGTCGTGGACGTATCCCATGAGGACCCGGAGGTGAAGTTCAACTGGTATGTTG
ATGGTGTGGAAGTGCATAATGCAAAAACTAAGCCACGGGAGGAACAGTACAATAGCACATACAGAGTAGTTAGCGTT
CTGACTGTCCTGCACCAAGATTGGCTGAACGGGAAGGAATACAAGTGTAAGGTGAGCAACAAAGCCCTGCCCGCTCC
AATTGAGAAAACAATTTCTAAGGCCAAAGGACAGCCCCGGGAGCCACAGGTGTATACTCTGCCGCCTAGCAGGGAGG
AAATGACCAAGAACCAGGTGAGCCTGACCTGTCTGGTGAAAGGCTTCTACCCCAGCGATATCGCAGTTGAGTGGGAG
AGCAATGGCCAACCCGAGAACAACTACAAGACTACGCCCCCCGTGCTTGATAGCGACGGATCATTTTTCCTGTACTC
AAAACTGACCGTGGACAAAAGCAGATGGCAGCAGGGAAACGTTTTCAGTTGCTCAGTGATGCACGAAGCGCTGCACA
ATCATTATACTCAGAAAAGCCTGAGCTTGAGCCCGGGAAAATGA
SEQ ID NO: 44 Isatuximab light chain (Protein)
DIVMTQSHLSMSTSLGDPVSITCKASQDVSTVVAWYQQKPGQSPRRLIYSASYRYIGVPDRFTGSGAGTDFTFTISS
VQAEDLAVYYCQQHYSPPYTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 45 Isatuximab light chain DNA
GATATCGTGATGACACAGAGCCACCTGAGCATGAGCACCAGCCTGGGAGATCCCGTGTCCATCACCTGTAAAGCCAG
CCAGGATGTGTCCACCGTGGTGGCCTGGTATCAGCAGAAGCCTGGCCAGTCTCCTCGGCGGCTGATCTACTCTGCCA
GCTACAGATATATCGGCGTGCCCGACAGATTCACCGGATCTGGCGCTGGCACCGACTTCACCTTTACCATCAGCTCT
GTGCAGGCCGAGGACCTGGCCGTGTACTATTGTCAGCAGCACTACAGCCCTCCATACACCTTTGGCGGCGGTACCAA
GCTGGAAATCAAGCGGACAGTGGCCGCTCCTAGCGTGTTCATCTTTCCACCTAGCGACGAGCAGCTGAAGTCTGGCA
CAGCCTCTGTCGTGTGCCTGCTGAACAACTTCTACCCCAGAGAAGCCAAGGTGCAGTGGAAGGTGGACAACGCCCTG
CAGAGCGGCAATAGCCAAGAGAGCGTGACCGAGCAGGACAGCAAGGACTCTACCTACAGCCTGAGCAGCACCCTGAC
ACTGAGCAAGGCCGACTACGAGAAGCACAAAGTGTACGCCTGCGAAGTGACCCACCAGGGCCTTTCTAGCCCTGTGA
CCAAGAGCTTCAACCGGGGCGAGTGCTAA
SEQ ID NO: 46 BDR5-1WT heavy chain (protein) (HC-hDR5-01-G56T SEQ ID NO:
36 of US10882913)
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPG
SEQ ID NO: 47 BDR5-1WT heavy chain (DNA)
GAAGTGCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAATGGATCGGCAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGATTATTGGGGCCAGGGTACCCTGGTCACAGTTAGCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGCACCCAGACCTATATCTGCAATGTCAATCATA
AACCCAGTAACACAAAGGTAGACAAGAGAGTCGAACCCAAATCTTGCGACAAAACTCACACATGCCCTCCTTGCCCC
GCCCCCGAACTCCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGCACGAAGCGCTGCACAATCATT
ATACTCAGAAAAGCCTGAGCTTGAGCCCGGGA
SEQ ID NO: 48 BDR5 light chain (protein) (LC-hDR5-01SEQ ID NO: 39 of US10882913)
EIVMTQSPATLSVSPGERATLSCRASQSISNNLHWYQQKPGQAPRLLIKFASQSITGIPARFSGSGSGTEFTLTISS
LQSEDFAVYYCQQGNSWPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 49 BDR5 LC light chain (DNA)
GAGATTGTGATGACACAGAGCCCCGCCACACTGTCCGTTTCTCCAGGCGAAAGAGCCACACTGAGCTGCAGAGCCAG
CCAGAGCATCAGCAACAACCTGCACTGGTATCAGCAGAAGCCCGGACAGGCTCCCAGACTGCTGATCAAGTTTGCCA
GCCAGTCCATCACAGGCATCCCCGCCAGATTTTCTGGCAGCGGCTCTGGCACCGAGTTCACCCTGACAATCAGCAGC
CTGCAGAGCGAGGACTTCGCCGTGTACTATTGCCAGCAGGGCAACAGCTGGCCCTACACCTTCGGCCAGGGTACCAA
GCTGGAAATCAAGCGGACAGTGGCCGCTCCTAGCGTGTTCATCTTTCCACCTAGCGACGAGCAGCTGAAGTCTGGCA
CAGCCTCTGTCGTGTGCCTGCTGAACAACTTCTACCCCAGAGAAGCCAAGGTGCAGTGGAAGGTGGACAACGCCCTG
CAGAGCGGCAATAGCCAAGAGAGCGTGACCGAGCAGGACAGCAAGGACTCTACCTACAGCCTGAGCAGCACCCTGAC
ACTGAGCAAGGCCGACTACGAGAAGCACAAAGTGTACGCCTGCGAAGTGACCCACCAGGGCCTTTCTAGCCCTGTGA
CCAAGAGCTTCAACCGGGGCGAGTGC
SEQ ID NO: 50 BDR5-2WT H chain (Protein) (VH ONLY FROM HC-hDR5-01-G56T SEQ
ID NO: 36 of US10882913)
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASTKGPSVFPLAPCSRSTSESTAALGCLVKDYFPEP
VTVSWNSGALTSGVHTFPAVLQSSGLYSLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEVHNAKTKPREEQFNSTFRVVSVLTVVHQ
DWLNGKEYKCKVSNKGLPAPIEKTISKTKGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYTQKSLSLSPG
SEQ ID NO: 51 BDR5-2WT heavy chain (DNA)
GAAGTGCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAATGGATCGGCAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGACTATTGGGGCCAGGGTACCCTGGTCACAGTTAGCTCTGCTAGCACAAAAGGCCCTAGCGTCTTTCCAC
TCGCCCCATGTTCAAGAAGCACCAGCGAGTCAACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAATTTTGGCACCCAGACCTATACATGCAATGTCGATCATA
AACCCAGTAACACAAAGGTAGACAAGACAGTCGAAAGAAAATGCTGTGTGGAGTGCCCTCCTTGCCCCGCCCCCCCA
GTGGCCGGGCCTTCGGTTTTTCTTTTTCCACCTAAACCGAAAGACACCCTGATGATCAGCAGGACACCAGAAGTGAC
ATGTGTCGTCGTGGACGTATCCCATGAAGACCCGGAGGTGCAATTCAACTGGTATGTTGATGGTGTGGAAGTGCATA
ATGCAAAAACTAAGCCACGGGAGGAACAGTTCAATAGCACATTCAGAGTAGTTAGCGTTCTGACTGTCGTGCACCAA
GATTGGCTGAACGGGAAGGAATACAAGTGTAAGGTGAGCAACAAAGGTCTGCCCGCTCCAATTGAGAAAACAATTTC
TAAGACCAAAGGACAGCCCCGGGAGCCACAGGTGTATACTCTGCCGCCTAGCAGGGAGGAAATGACCAAGAACCAGG
TGAGCCTGACCTGTCTGGTGAAAGGCTTCTACCCCAGCGATATCGCAGTTGAGTGGGAGAGCAATGGCCAACCCGAG
AACAACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAGAAAA
GCCTGAGCTTGAGCCCGGGA
SEQ ID NO: 52 TDR5-1WT heavy chain (protein) (HC-hDR5-05 (SEQ ID NO: 40 of
US10882913)
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPG
SEQ ID NO: 53 TDR5-1WT heavy chain (DNA)
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGCCAGGGTACCCTGGTCACAGTTAGCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGCACCCAGACCTATATCTGCAATGTCAATCATA
AACCCAGTAACACAAAGGTAGACAAGAGAGTCGAACCCAAATCTTGCGACAAAACTCACACATGCCCTCCTTGCCCC
GCCCCCGAACTCCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGCACGAAGCGCTGCACAATCATT
ATACTCAGAAAAGCCTGAGCTTGAGCCCGGGA
SEQ ID NO: 54 TDR5 light chain (protein) (LC-hDR5-05 SEQ ID NO: 43 of US10882913)
DIQLTQSPSSLSASVGDRVTITCSASSSVSYMYWYQQKPGKAPKPWIYRTSNLASGVPSRFSGSGSGTDFTLTISSL
QPEDFATYYCQQYHSYPPTFGGGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
SEQ ID NO: 55 TDR5 light chain (DNA)
GATATCCAGCTGACACAGAGCCCTAGCAGCCTGTCTGCCTCTGTGGGCGACAGAGTGACCATCACATGTAGCGCCAG
CAGCAGCGTGTCCTACATGTACTGGTATCAGCAGAAGCCCGGCAAGGCCCCTAAGCCTTGGATCTACAGAACCAGCA
ATCTGGCCAGCGGCGTGCCCAGCAGATTTTCTGGTTCTGGCAGCGGCACCGACTTCACCCTGACCATATCTAGCCTG
CAGCCTGAGGACTTCGCCACCTACTACTGCCAGCAGTACCACAGCTACCCTCCAACCTTTGGCGGCGGTACCAAGGT
GGAAATCAAGCGGACAGTGGCCGCTCCTAGCGTGTTCATCTTTCCACCTAGCGACGAGCAGCTGAAGTCTGGCACAG
CCTCTGTCGTGTGCCTGCTGAACAACTTCTACCCCAGAGAAGCCAAGGTGCAGTGGAAGGTGGACAACGCCCTGCAG
AGCGGCAATAGCCAAGAGAGCGTGACCGAGCAGGACAGCAAGGACTCTACCTACAGCCTGAGCAGCACCCTGACACT
GAGCAAGGCCGACTACGAGAAGCACAAAGTGTACGCCTGCGAAGTGACCCACCAGGGCCTTTCTAGCCCTGTGACCA
AGAGCTTCAACCGGGGCGAGTGC
SEQ ID NO: 56 TDR5-2WT heavy chain (protein) (VH ONLY FROM HC-hDR5-05 SEQ
ID NO: 40 of US10882913)
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASTKGPSVFPLAPCSRSTSESTAALGCLVKDYFPEP
VTVSWNSGALTSGVHTFPAVLQSSGLYSLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEVHNAKTKPREEQFNSTFRVVSVLTVVHQ
DWLNGKEYKCKVSNKGLPAPIEKTISKTKGQPREPQVYTLPPSREEMTKNQVSLTCLVKGFYPSDIAVEWESNGQPE
NNYKTTPPMLDSDGSFFLYSKLTVDKSRWQQGNVFSCSVMHEALHNHYTQKSLSLSPG
SEQ ID NO: 57 TDR5-2WT heavy chain (DNA)
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGACAGGGTACCCTCGTGACCGTTAGCTCTGCTAGCACAAAAGGCCCTAGCGTCTTTCCAC
TCGCCCCATGTTCAAGAAGCACCAGCGAGTCAACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAATTTTGGCACCCAGACCTATACATGCAATGTCGATCATA
AACCCAGTAACACAAAGGTAGACAAGACAGTCGAAAGAAAATGCTGTGTGGAGTGCCCTCCTTGCCCCGCCCCCCCA
GTGGCCGGGCCTTCGGTTTTTCTTTTTCCACCTAAACCGAAAGACACCCTGATGATCAGCAGGACACCAGAAGTGAC
ATGTGTCGTCGTGGACGTATCCCATGAAGACCCGGAGGTGCAATTCAACTGGTATGTTGATGGTGTGGAAGTGCATA
ATGCAAAAACTAAGCCACGGGAGGAACAGTTCAATAGCACATTCAGAGTAGTTAGCGTTCTGACTGTCGTGCACCAA
GATTGGCTGAACGGGAAGGAATACAAGTGTAAGGTGAGCAACAAAGGTCTGCCCGCTCCAATTGAGAAAACAATTTC
TAAGACCAAAGGACAGCCCCGGGAGCCACAGGTGTATACTCTGCCGCCTAGCAGGGAGGAAATGACCAAGAACCAGG
TGAGCCTGACCTGTCTGGTGAAAGGCTTCTACCCCAGCGATATCGCAGTTGAGTGGGAGAGCAATGGCCAACCCGAG
AACAACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAGAAAA
GCCTGAGCTTGAGCCCGGGA
SEQ ID NO: 58 BDR5-A2WT heavy chain (protein) (VH ONLY FROM HC-hDR5-01-
G56T SEQ ID NO: 36 of US10882913)
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASPTSPKVFPLSLDSTPQDGNVVVACLVQGFFPQEP
LSVTWSESGQNVTARNFPPSQDASGDLYTTSSQLTLPATQCPDGKSVTCHVKHYTNPSQDVTVPCPVPPPPPCCHPR
LSLHRPALEDLLLGSEANLTCTLTGLRDASGATFTWTPSSGKSAVQGPPERDLCGCYSVSSVLPGCAQPWNHGETFT
CTAAHPELKTPLTANITKSGNTFRPEVHLLPPPSEELALNELVTLTCLARGFSPKDVLVRWLQGSQELPREKYLTWA
SRQEPSQGTTTFAVTSILRVAAEDWKKGDTFSCMVGHEALPLAFTQKTIDRLAG
SEQ ID NO: 59 BDR5-A2WT heavy chain (DNA)
GAAGTTCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAGTGGATCGGAAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGACTATTGGGGCCAGGGTACCCTCGTGACCGTTAGCTCTGCTAGCCCTACAAGCCCCAAGGTGTTCCCTC
TGAGCCTGGATAGCACACCCCAGGACGGAAATGTGGTGGTGGCTTGTCTGGTGCAGGGATTCTTCCCACAAGAGCCC
CTGTCCGTGACTTGGAGCGAGAGCGGACAGAATGTGACCGCCAGAAACTTCCCACCTAGCCAGGATGCCAGCGGCGA
TCTGTACACAACAAGCAGCCAGCTGACCCTGCCTGCCACACAGTGTCCTGATGGCAAGAGCGTGACCTGCCACGTGA
AGCACTACACAAACCCCAGCCAGGACGTGACCGTGCCTTGTCCTGTTCCTCCTCCACCTCCATGCTGTCACCCTAGA
CTGAGCCTGCACAGACCCGCTCTGGAAGATCTGCTGCTGGGCTCTGAGGCCAACCTGACCTGTACACTGACCGGCCT
GAGAGATGCCTCTGGCGCCACCTTTACATGGACACCTAGCAGCGGAAAGTCCGCCGTTCAGGGACCTCCTGAGAGGG
ATCTGTGTGGCTGTTACAGCGTGTCCTCTGTGCTGCCTGGATGTGCCCAGCCTTGGAATCACGGCGAGACATTCACC
TGTACCGCCGCTCATCCCGAGCTGAAAACACCCCTGACCGCCAACATCACCAAGAGCGGCAATACCTTCAGACCCGA
AGTGCATCTGCTGCCTCCACCATCTGAAGAACTGGCCCTGAACGAGCTGGTGACCCTGACATGTCTGGCCAGGGGCT
TCAGCCCTAAGGATGTGCTCGTTAGATGGCTGCAGGGCAGCCAAGAGCTGCCCAGAGAGAAGTATCTGACCTGGGCC
AGTCGACAGGAACCTTCTCAGGGCACCACAACCTTTGCCGTGACCAGCATTCTGAGAGTGGCCGCCGAGGATTGGAA
GAAGGGCGACACCTTTAGCTGCATGGTCGGACACGAAGCGCTGCCCCTGGCTTTCACCCAGAAAACCATCGATAGAC
TGGCCGGC
SEQ ID NO: 60 TDR5-A2WT heavy chain (protein) (VH ONLY FROM HC-hDR5-05 SEQ
ID NO: 40 of US10882913)
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASPTSPKVFPLSLDSTPQDGNVVVACLVQGFFPQEP
LSVTWSESGQNVTARNFPPSQDASGDLYTTSSQLTLPATQCPDGKSVTCHVKHYTNPSQDVTVPCPVPPPPPCCHPR
LSLHRPALEDLLLGSEANLTCTLTGLRDASGATFTWTPSSGKSAVQGPPERDLCGCYSVSSVLPGCAQPWNHGETFT
CTAAHPELKTPLTANITKSGNTFRPEVHLLPPPSEELALNELVTLTCLARGESPKDVLVRWLQGSQELPREKYLTWA
SRQEPSQGTTTFAVTSILRVAAEDWKKGDTFSCMVGHEALPLAFTQKTIDRLAG
SEQ ID NO: 61 TDR5-A2WT heavy chain (DNA)
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGACAGGGTACCCTCGTGACCGTTAGCTCTGCTAGCCCTACAAGCCCCAAGGTGTTCCCTC
TGAGCCTGGATAGCACACCCCAGGACGGAAATGTGGTGGTGGCTTGTCTGGTGCAGGGATTCTTCCCACAAGAGCCC
CTGTCCGTGACTTGGAGCGAGAGCGGACAGAATGTGACCGCCAGAAACTTCCCACCTAGCCAGGATGCCAGCGGCGA
TCTGTACACAACAAGCAGCCAGCTGACCCTGCCTGCCACACAGTGTCCTGATGGCAAGAGCGTGACCTGCCACGTGA
AGCACTACACAAACCCCAGCCAGGACGTGACCGTGCCTTGTCCTGTTCCTCCTCCACCTCCATGCTGTCACCCTAGA
CTGAGCCTGCACAGACCCGCTCTGGAAGATCTGCTGCTGGGCTCTGAGGCCAACCTGACCTGTACACTGACCGGCCT
GAGAGATGCCTCTGGCGCCACCTTTACATGGACACCTAGCAGCGGAAAGTCCGCCGTTCAGGGACCTCCTGAGAGGG
ATCTGTGTGGCTGTTACAGCGTGTCCTCTGTGCTGCCTGGATGTGCCCAGCCTTGGAATCACGGCGAGACATTCACC
TGTACCGCCGCTCATCCCGAGCTGAAAACACCCCTGACCGCCAACATCACCAAGAGCGGCAATACCTTCAGACCCGA
AGTGCATCTGCTGCCTCCACCATCTGAAGAACTGGCCCTGAACGAGCTGGTGACCCTGACATGTCTGGCCAGGGGCT
TCAGCCCTAAGGATGTGCTCGTTAGATGGCTGCAGGGCAGCCAAGAGCTGCCCAGAGAGAAGTATCTGACCTGGGCC
AGTCGACAGGAACCTTCTCAGGGCACCACAACCTTTGCCGTGACCAGCATTCTGAGAGTGGCCGCCGAGGATTGGAA
GAAGGGCGACACCTTTAGCTGCATGGTCGGACACGAAGCGCTGCCCCTGGCTTTCACCCAGAAAACCATCGATAGAC
TGGCCGGC
SEQ ID NO: 62 BDR5-1HF H chain (Protein) (VH ONLY FROM HC-hDR5-01-G56T SEQ
ID NO: 36 of US10882913)
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMFEALHNHYTQKSLSLSPG
SEQ ID NO: 63 BDR5-1HF heavy chain (DNA)
GAAGTGCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAATGGATCGGCAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGATTATTGGGGCCAGGGTACCCTGGTCACAGTTAGCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGCACCCAGACCTATATCTGCAATGTCAATCATA
AACCCAGTAACACAAAGGTAGACAAGAGAGTCGAACCCAAATCTTGCGACAAAACTCACACATGCCCTCCTTGCCCC
GCCCCCGAACTCCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGTTCGAAGCGCTGCACAATCATT
ATACTCAGAAAAGCCTGAGCTTGAGCCCGGGA
SEQ ID NO: 64 BDR5-2HF H chain (Protein) (VH ONLY FROM HC-hDR5-01-G56T SEQ
ID NO: 36 of US10882913)
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASTKGPSVFPLAPCSRSTSESTAALGCLVKDYFPEP
VTVSWNSGALTSGVHTFPAVLQSSGLYSLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEVHNAKTKPREEQFNSTFRVVSVLTVVHQ
DWLNGKEYKCKVSNKGLPAPIEKTISKTKGQPREPQVYTLPPSREEMTKNQVSLTCLVKGFYPSDIAVEWESNGQPE
NNYKTTPPMLDSDGSFFLYSKLTVDKSRWQQGNVFSCSVMFEALHNHYTQKSLSLSPG
SEQ ID NO: 65 BDR5-2HF heavy chain (DNA)
GAAGTGCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAATGGATCGGCAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGACTATTGGGGCCAGGGTACCCTGGTCACAGTTAGCTCTGCTAGCACAAAAGGCCCTAGCGTCTTTCCAC
TCGCCCCATGTTCAAGAAGCACCAGCGAGTCAACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAATTTTGGCACCCAGACCTATACATGCAATGTCGATCATA
AACCCAGTAACACAAAGGTAGACAAGACAGTCGAAAGAAAATGCTGTGTGGAGTGCCCTCCTTGCCCCGCCCCCCCA
GTGGCCGGGCCTTCGGTTTTTCTTTTTCCACCTAAACCGAAAGACACCCTGATGATCAGCAGGACACCAGAAGTGAC
ATGTGTCGTCGTGGACGTATCCCATGAAGACCCGGAGGTGCAATTCAACTGGTATGTTGATGGTGTGGAAGTGCATA
ATGCAAAAACTAAGCCACGGGAGGAACAGTTCAATAGCACATTCAGAGTAGTTAGCGTTCTGACTGTCGTGCACCAA
GATTGGCTGAACGGGAAGGAATACAAGTGTAAGGTGAGCAACAAAGGTCTGCCCGCTCCAATTGAGAAAACAATTTC
TAAGACCAAAGGACAGCCCCGGGAGCCACAGGTGTATACTCTGCCGCCTAGCAGGGAGGAAATGACCAAGAACCAGG
TGAGCCTGACCTGTCTGGTGAAAGGCTTCTACCCCAGCGATATCGCAGTTGAGTGGGAGAGCAATGGCCAACCCGAG
AACAACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAGAAAA
GCCTGAGCTTGAGCCCGGGA
SEQ ID NO: 66 TDR5-1HF
heavy chain (protein) (HC-hDR5-05 (SEQ ID NO: 40 of US10882913)
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMFEALHNHYTQKSLSLSPG
SEQ ID NO: 67 TDR5-1HF heavy chain (DNA)
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGCCAGGGTACCCTGGTCACAGTTAGCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGCACCCAGACCTATATCTGCAATGTCAATCATA
AACCCAGTAACACAAAGGTAGACAAGAGAGTCGAACCCAAATCTTGCGACAAAACTCACACATGCCCTCCTTGCCCC
GCCCCCGAACTCCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGTTCGAAGCGCTGCACAATCATT
ATACTCAGAAAAGCCTGAGCTTGAGCCCGGGA
SEQ ID NO: 68 TDR5-2HF heavy chain (protein) (HC-hDR5-05 (SEQ ID NO: 40 of
US10882913)
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASTKGPSVFPLAPCSRSTSESTAALGCLVKDYFPEP
VTVSWNSGALTSGVHTFPAVLQSSGLYSLSSVVTVPSSNFGTQTYTCNVDHKPSNTKVDKTVERKCCVECPPCPAPP
VAGPSVFLFPPKPKDTLMISRTPEVTCVVVDVSHEDPEVQFNWYVDGVEVHNAKTKPREEQFNSTFRVVSVLTVVHQ
DWLNGKEYKCKVSNKGLPAPIEKTISKTKGQPREPQVYTLPPSREEMTKNQVSLTCLVKGFYPSDIAVEWESNGQPE
NNYKTTPPMLDSDGSFFLYSKLTVDKSRWQQGNVFSCSVMFEALHNHYTQKSLSLSPG
SEQ ID NO: 69 TDR5-2HF heavy chain (DNA)
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGACAGGGTACCCTCGTGACCGTTAGCTCTGCTAGCACAAAAGGCCCTAGCGTCTTTCCAC
TCGCCCCATGTTCAAGAAGCACCAGCGAGTCAACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAATTTTGGCACCCAGACCTATACATGCAATGTCGATCATA
AACCCAGTAACACAAAGGTAGACAAGACAGTCGAAAGAAAATGCTGTGTGGAGTGCCCTCCTTGCCCCGCCCCCCCA
GTGGCCGGGCCTTCGGTTTTTCTTTTTCCACCTAAACCGAAAGACACCCTGATGATCAGCAGGACACCAGAAGTGAC
ATGTGTCGTCGTGGACGTATCCCATGAAGACCCGGAGGTGCAATTCAACTGGTATGTTGATGGTGTGGAAGTGCATA
ATGCAAAAACTAAGCCACGGGAGGAACAGTTCAATAGCACATTCAGAGTAGTTAGCGTTCTGACTGTCGTGCACCAA
GATTGGCTGAACGGGAAGGAATACAAGTGTAAGGTGAGCAACAAAGGTCTGCCCGCTCCAATTGAGAAAACAATTTC
TAAGACCAAAGGACAGCCCCGGGAGCCACAGGTGTATACTCTGCCGCCTAGCAGGGAGGAAATGACCAAGAACCAGG
TGAGCCTGACCTGTCTGGTGAAAGGCTTCTACCCCAGCGATATCGCAGTTGAGTGGGAGAGCAATGGCCAACCCGAG
AACAACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAGAAAA
GCCTGAGCTTGAGCCCGGGA
SEQ ID NO: 70 BDR5-A2HF H chain (Protein) (VH ONLY FROM HC-hDR5-01-G56T SEQ
ID NO: 36 of US10882913)
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASPTSPKVFPLSLDSTPQDGNVVVACLVQGFFPQEP
LSVTWSESGQNVTARNFPPSQDASGDLYTTSSQLTLPATQCPDGKSVTCHVKHYTNPSQDVTVPCPVPPPPPCCHPR
LSLHRPALEDLLLGSEANLTCTLTGLRDASGATFTWTPSSGKSAVQGPPERDLCGCYSVSSVLPGCAQPWNHGETFT
CTAAHPELKTPLTANITKSGNTFRPEVHLLPPPSEELALNELVTLTCLARGESPKDVLVRWLQGSQELPREKYLTWA
SRQEPSQGTTTFAVTSILRVAAEDWKKGDTFSCMVGFEALPLAFTQKTIDRLAG
SEQ ID NO: 71 BDR5-A2HF heavy chain (DNA)
GAAGTTCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAGTGGATCGGAAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGACTATTGGGGCCAGGGTACCCTCGTGACCGTTAGCTCTGCTAGCCCTACAAGCCCCAAGGTGTTCCCTC
TGAGCCTGGATAGCACACCCCAGGACGGAAATGTGGTGGTGGCTTGTCTGGTGCAGGGATTCTTCCCACAAGAGCCC
CTGTCCGTGACTTGGAGCGAGAGCGGACAGAATGTGACCGCCAGAAACTTCCCACCTAGCCAGGATGCCAGCGGCGA
TCTGTACACAACAAGCAGCCAGCTGACCCTGCCTGCCACACAGTGTCCTGATGGCAAGAGCGTGACCTGCCACGTGA
AGCACTACACAAACCCCAGCCAGGACGTGACCGTGCCTTGTCCTGTTCCTCCTCCACCTCCATGCTGTCACCCTAGA
CTGAGCCTGCACAGACCCGCTCTGGAAGATCTGCTGCTGGGCTCTGAGGCCAACCTGACCTGTACACTGACCGGCCT
GAGAGATGCCTCTGGCGCCACCTTTACATGGACACCTAGCAGCGGAAAGTCCGCCGTTCAGGGACCTCCTGAGAGGG
ATCTGTGTGGCTGTTACAGCGTGTCCTCTGTGCTGCCTGGATGTGCCCAGCCTTGGAATCACGGCGAGACATTCACC
TGTACCGCCGCTCATCCCGAGCTGAAAACACCCCTGACCGCCAACATCACCAAGAGCGGCAATACCTTCAGACCCGA
AGTGCATCTGCTGCCTCCACCATCTGAAGAACTGGCCCTGAACGAGCTGGTGACCCTGACATGTCTGGCCAGGGGCT
TCAGCCCTAAGGATGTGCTCGTTAGATGGCTGCAGGGCAGCCAAGAGCTGCCCAGAGAGAAGTATCTGACCTGGGCC
AGTCGACAGGAACCTTCTCAGGGCACCACAACCTTTGCCGTGACCAGCATTCTGAGAGTGGCCGCCGAGGATTGGAA
GAAGGGCGACACCTTTAGCTGCATGGTCGGATTCGAAGCGCTGCCCCTGGCTTTCACCCAGAAAACCATCGATAGAC
TGGCCGGC
SEQ ID NO: 72 TDR5-A2HF heavy chain (protein) (HC-hDR5-05 (SEQ ID NO: 40
of US10882913)
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASPTSPKVFPLSLDSTPQDGNVVVACLVQGFFPQEP
LSVTWSESGQNVTARNFPPSQDASGDLYTTSSQLTLPATQCPDGKSVTCHVKHYTNPSQDVTVPCPVPPPPPCCHPR
LSLHRPALEDLLLGSEANLTCTLTGLRDASGATFTWTPSSGKSAVQGPPERDLCGCYSVSSVLPGCAQPWNHGETFT
CTAAHPELKTPLTANITKSGNTFRPEVHLLPPPSEELALNELVTLTCLARGFSPKDVLVRWLQGSQELPREKYLTWA
SRQEPSQGTTTFAVTSILRVAAEDWKKGDTFSCMVGFEALPLAFTQKTIDRLAG
SEQ ID NO: 73 TDR5-A2HF heavy chain (DNA)
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGACAGGGTACCCTCGTGACCGTTAGCTCTGCTAGCCCTACAAGCCCCAAGGTGTTCCCTC
TGAGCCTGGATAGCACACCCCAGGACGGAAATGTGGTGGTGGCTTGTCTGGTGCAGGGATTCTTCCCACAAGAGCCC
CTGTCCGTGACTTGGAGCGAGAGCGGACAGAATGTGACCGCCAGAAACTTCCCACCTAGCCAGGATGCCAGCGGCGA
TCTGTACACAACAAGCAGCCAGCTGACCCTGCCTGCCACACAGTGTCCTGATGGCAAGAGCGTGACCTGCCACGTGA
AGCACTACACAAACCCCAGCCAGGACGTGACCGTGCCTTGTCCTGTTCCTCCTCCACCTCCATGCTGTCACCCTAGA
CTGAGCCTGCACAGACCCGCTCTGGAAGATCTGCTGCTGGGCTCTGAGGCCAACCTGACCTGTACACTGACCGGCCT
GAGAGATGCCTCTGGCGCCACCTTTACATGGACACCTAGCAGCGGAAAGTCCGCCGTTCAGGGACCTCCTGAGAGGG
ATCTGTGTGGCTGTTACAGCGTGTCCTCTGTGCTGCCTGGATGTGCCCAGCCTTGGAATCACGGCGAGACATTCACC
TGTACCGCCGCTCATCCCGAGCTGAAAACACCCCTGACCGCCAACATCACCAAGAGCGGCAATACCTTCAGACCCGA
AGTGCATCTGCTGCCTCCACCATCTGAAGAACTGGCCCTGAACGAGCTGGTGACCCTGACATGTCTGGCCAGGGGCT
TCAGCCCTAAGGATGTGCTCGTTAGATGGCTGCAGGGCAGCCAAGAGCTGCCCAGAGAGAAGTATCTGACCTGGGCC
AGTCGACAGGAACCTTCTCAGGGCACCACAACCTTTGCCGTGACCAGCATTCTGAGAGTGGCCGCCGAGGATTGGAA
GAAGGGCGACACCTTTAGCTGCATGGTCGGATTCGAAGCGCTGCCCCTGGCTTTCACCCAGAAAACCATCGATAGAC
TGGCCGGC
SEQ ID NO: 74 mezagitamab (CD38) WT heavy chain amino acid sequence
[Sequence 21 from US8362211]
EVQLLESGGGLVQPGGSLRLSCAASGFTFDDYGMSWVRQAPGKGLEWVSDISWNGGKTHYVDSVKGQFTISRDNSKN
TLYLQMNSLRAEDTAVYYCARGSLFHDSSGFYFGHWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPGK
SEQ ID NO: 75 mezagitamab (CD38) WT heavy chain nucleotide sequence
GAAGTGCAGCTGCTGGAATCTGGCGGCGGACTTGTTCAACCAGGCGGCTCTCTGAGACTGAGCTGTGCCGCTTCCGG
CTTCACCTTCGACGATTACGGCATGAGCTGGGTCCGACAGGCCCCTGGAAAAGGCCTTGAATGGGTGTCCGACATCA
GCTGGAATGGCGGCAAGACCCACTACGTGGACTCTGTGAAGGGCCAGTTCACCATCAGCCGGGACAACAGCAAGAAC
ACCCTGTACCTGCAGATGAACAGCCTGAGAGCCGAGGACACCGCCGTGTACTATTGTGCCAGAGGCAGCCTGTTCCA
CGACAGCAGCGGCTTCTATTTTGGCCACTGGGGCCAGGGCACACTGGTCACAGTTAGCTCTGCTAGCACAAAAGGCC
CTAGCGTCTTTCCACTCGCCCCATCCTCCAAGAGCACCTCTGGGGGCACTGCGGCTTTGGGCTGCTTGGTGAAGGAT
TACTTCCCAGAGCCTGTCACTGTCAGCTGGAATAGCGGGGCTCTGACCAGTGGAGTGCACACCTTTCCCGCCGTGTT
ACAGAGCAGCGGCCTCTATAGCCTGAGCAGTGTGGTAACTGTGCCCTCGAGCAGCTTGGGTACCCAGACCTATATCT
GCAATGTCAATCATAAACCCAGTAACACAAAGGTAGACAAGAGAGTCGAACCCAAATCTTGCGACAAAACTCACACA
TGCCCTCCTTGCCCCGCCCCCGAACTCCTGGGGGGACCTTCGGTTTTTCTTTTTCCACCTAAACCGAAAGACACCCT
GATGATCAGCAGGACACCAGAAGTGACATGTGTCGTCGTGGACGTATCCCATGAGGACCCGGAGGTGAAGTTCAACT
GGTATGTTGATGGTGTGGAAGTGCATAATGCAAAAACTAAGCCACGGGAGGAACAGTACAATAGCACATACAGAGTA
GTTAGCGTTCTGACTGTCCTGCACCAAGATTGGCTGAACGGGAAGGAATACAAGTGTAAGGTGAGCAACAAAGCCCT
GCCCGCTCCAATTGAGAAAACAATTTCTAAGGCCAAAGGACAGCCCCGGGAGCCACAGGTGTATACTCTGCCGCCTA
GCAGGGAGGAAATGACCAAGAACCAGGTGAGCCTGACCTGTCTGGTGAAAGGCTTCTACCCCAGCGATATCGCAGTT
GAGTGGGAGAGCAATGGCCAACCCGAGAACAACTACAAGACTACGCCCCCCGTGCTTGATAGCGACGGATCATTTTT
CCTGTACTCAAAACTGACCGTGGACAAAAGCAGATGGCAGCAGGGAAACGTTTTCAGTTGCTCAGTGATGCACGAAG
CGCTGCACAATCATTATACTCAGAAAAGCCTGAGCTTGAGCCCGGGAAAA
SEQ ID NO: 76 mezagitamab (CD38) light chain amino acid sequence [Sequence
22 from US8362211]
QSVLTQPPSASGTPGQRVTISCSGSSSNIGDNYVSWYQQLPGTAPKLLIYRDSQRPSGVPDRFSGSKSGTSASLAIS
GLRSEDEADYYCQSYDSSLSGSVFGGGTKLTVLGQPKANPTVTLFPPSSEELQANKATLVCLISDFYPGAVTVAWKA
DGSPVKAGVETTKPSKQSNNKYAASSYLSLTPEQWKSHRSYSCQVTHEGSTVEKTVAPTECS
SEQ ID NO: 77 mezagitamab (CD38) light chain nucleotide sequence
CAATCTGTTCTGACACAGCCTCCTAGCGCCTCTGGCACACCTGGACAGAGAGTGACCATCAGCTGTAGCGGCAGCAG
CAGCAACATCGGCGACAACTACGTGTCCTGGTATCAGCAGCTGCCTGGCACAGCCCCTAAACTGCTGATCTACCGGG
ATAGCCAGAGGCCAAGCGGCGTGCCAGATAGATTTTCCGGCAGCAAGAGCGGCACATCTGCCAGCCTGGCTATCAGC
GGACTGAGATCTGAGGACGAGGCCGACTACTACTGCCAGAGCTACGATAGCAGCCTGAGCGGCTCTGTGTTTGGCGG
AGGTACCAAGCTGACCGTGCTGGGCCAACCTAAAGCCAATCCTACCGTGACACTGTTCCCTCCAAGCAGCGAAGAAC
TGCAGGCCAACAAGGCCACACTCGTGTGCCTGATCAGCGACTTTTATCCTGGCGCCGTGACCGTGGCCTGGAAGGCT
GATGGATCTCCTGTGAAAGCCGGCGTGGAAACCACCAAGCCTAGCAAGCAGAGCAACAACAAATACGCCGCCAGCAG
CTACCTGAGCCTGACACCTGAGCAGTGGAAGTCCCACAGATCCTACAGCTGCCAAGTGACCCACGAGGGCAGCACCG
TGGAAAAGACAGTGGCCCCTACCGAGTGCAGC
SEQ ID NO: 78 S2P6 IgG1 WT H chain amino acid sequence
EVQLVQSGAEVKKPGASVKVSCKASGYTFTSQYMHWVRQAPGQGLEWIGIINPSGVHTSYAQKFQGRVTLTRDTSTS
TLYMELSSLRSEDTAVYYCARGSPKGAFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGK
SEQ ID NO: 79 S2P6 IgG1 WT H chain nucleotide sequence
GAAGTTCAGCTGGTTCAGTCTGGCGCCGAAGTGAAGAAACCTGGCGCCTCTGTGAAGGTGTCCTGCAAGGCCAGCGG
CTACACCTTTACCAGCCAGTACATGCACTGGGTCCGACAGGCTCCAGGACAGGGACTCGAGTGGATCGGCATCATCA
ATCCTAGCGGCGTGCACACCAGCTACGCCCAGAAATTCCAGGGCAGAGTGACCCTGACCAGAGACACCAGCACCAGC
ACACTGTACATGGAACTGAGCAGCCTGCGGAGCGAGGATACCGCCGTGTACTATTGTGCCAGAGGCTCTCCTAAGGG
CGCCTTCGATTATTGGGGCCAGGGCACACTGGTCACCGTGTCCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGTACCCAGACCTATATCTGCAATGTCAATCATA
AACCCAGTAACACAAAGGTAGACAAGAAAGTCGAACCCAAATCTTGCGACAAAACTCACACATGCCCTCCTTGCCCC
GCCCCCGAACTCCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGCACGAAGCGCTGCACAATCATT
ATACTCAGAAAAGCCTGAGCTTGAGCCCGGGAAAA
SEQ ID NO: 80 S2P6 kappa light chain amino acid sequence
EIVMMQSPGTLSLSPGERATLSCRASQSVRSNYLAWYQQKPGQAPRLLIYGASSRATGIPDRFSGSGSGTDFTLTIS
RLEPEDFAVYYCQQYGSSPPRFTFGPGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
SEQ ID NO: 81 S2P6 kappa light chain nucleotide sequence
GAGATCGTCATGATGCAGAGCCCCGGCACACTGTCACTGTCTCCAGGCGAAAGAGCCACACTGAGCTGTAGAGCCTC
TCAGAGCGTGCGGAGCAATTACCTGGCCTGGTATCAGCAGAAGCCCGGACAGGCTCCTCGGCTGTTGATCTATGGCG
CCTCTAGCAGAGCCACTGGCATCCCCGATAGATTTTCTGGCAGCGGCTCCGGCACCGACTTCACCCTGACAATCAGC
AGACTGGAACCCGAGGACTTCGCCGTGTACTACTGTCAGCAGTACGGCAGCAGCCCTCCTCGGTTCACATTTGGCCC
TGGTACCAAGGTGGAAATCAAGCGGACAGTGGCCGCTCCTAGCGTGTTCATCTTTCCACCTAGCGACGAGCAGCTGA
AGTCTGGCACAGCCTCTGTCGTGTGCCTGCTGAACAACTTCTACCCCAGAGAAGCCAAGGTGCAGTGGAAGGTGGAC
AACGCCCTGCAGAGCGGCAATAGCCAAGAGAGCGTGACCGAGCAGGACAGCAAGGACTCTACCTACAGCCTGAGCAG
CACCCTGACACTGAGCAAGGCCGACTACGAGAAGCACAAAGTGTACGCCTGCGAAGTGACCCACCAGGGCCTTTCTA
GCCCTGTGACCAAGAGCTTCAACCGGGGCGAGTGC
SEQ ID NO: 82 CC40.8 IgG1 WT H Chain amino acid sequence
QVQEVQLLESGGGLVQPGGSLRLSCAASGFTFSSYVMTWARQAPGKGLEWVSAISGTGYTYYADSVKGRFTVSRDNS
KNTLFLQMSSLRAEDTAVYYCAITMAPVVWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNHYTQKSLSLSPGK
SEQ ID NO: 83 CV3-25 WT H chain amino acid sequence
QVQLVESGAEVKKPGESLKISCKGSGYTFTRYWIGWVRQMPGKGLEWMGIIYPGDSDTRYSPSFQGHVTISADKSIS
TAYLQWNSLKASDTAMYYCARLPQYCSNGVCQRWFDPWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSLSLSPGK
SEQ ID NO: 84 CC40.8 IgG1 WT H chain nucleotide sequence
CAAGTGCAGGAAGTGCAGCTGCTGGAATCTGGCGGAGGACTGGTTCAACCAGGCGGCTCTCTGAGACTGTCTTGTGC
CGCCAGCGGCTTCACCTTCAGCAGCTACGTTATGACCTGGGCCAGACAGGCCCCTGGCAAAGGACTTGAATGGGTGT
CCGCCATCAGCGGCACAGGCTACACCTACTACGCCGACTCTGTGAAGGGCAGATTCACCGTGTCCAGAGACAACAGC
AAGAATACCCTGTTCCTGCAGATGAGCAGCCTGAGAGCCGAGGATACCGCCGTGTACTACTGCGCCATTACAATGGC
CCCTGTCGTGTGGGGCCAGGGCACAACAGTGACAGTCAGCAGCGCTAGCACAAAAGGCCCTAGCGTCTTTCCACTCG
CCCCATCCTCCAAGAGCACCTCTGGGGGCACTGCGGCTTTGGGCTGCTTGGTGAAGGATTACTTCCCAGAGCCTGTC
ACTGTCAGCTGGAATAGCGGGGCTCTGACCAGTGGAGTGCACACCTTTCCCGCCGTGTTACAGAGCAGCGGCCTCTA
TAGCCTGAGCAGTGTGGTAACTGTGCCCTCGAGCAGCTTGGGTACCCAGACCTATATCTGCAATGTCAATCATAAAC
CCAGTAACACAAAGGTAGACAAGAAAGTCGAACCCAAATCTTGCGACAAAACTCACACATGCCCTCCTTGCCCCGCC
CCCGAACTCCTGGGGGGACCTTCGGTTTTTCTTTTTCCACCTAAACCGAAAGACACCCTGATGATCAGCAGGACACC
AGAAGTGACATGTGTCGTCGTGGACGTATCCCATGAGGACCCGGAGGTGAAGTTCAACTGGTATGTTGATGGTGTGG
AAGTGCATAATGCAAAAACTAAGCCACGGGAGGAACAGTACAATAGCACATACAGAGTAGTTAGCGTTCTGACTGTC
CTGCACCAAGATTGGCTGAACGGGAAGGAATACAAGTGTAAGGTGAGCAACAAAGCCCTGCCCGCTCCAATTGAGAA
AACAATTTCTAAGGCCAAAGGACAGCCCCGGGAGCCACAGGTGTATACTCTGCCGCCTAGCAGGGAGGAAATGACCA
AGAACCAGGTGAGCCTGACCTGTCTGGTGAAAGGCTTCTACCCCAGCGATATCGCAGTTGAGTGGGAGAGCAATGGC
CAACCCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGCACGAAGCGCTGCACAATCATTATA
CTCAGAAAAGCCTGAGCTTGAGCCCGGGAAAA
SEQ ID NO: 85 CV3-25 WT H chain nucleotide sequence
CAAGTGCAGCTGGTTGAATCTGGCGCCGAAGTGAAGAAGCCTGGCGAGAGCCTGAAGATCAGCTGCAAAGGCAGCGG
CTACACCTTCACCAGATACTGGATCGGCTGGGTCCGACAGATGCCTGGCAAAGGCCTTGAGTGGATGGGCATCATCT
ACCCCGGCGACAGCGATACCAGATACAGCCCTAGCTTTCAGGGCCACGTGACCATCAGCGCCGACAAGTCTATCAGC
ACCGCCTACCTGCAGTGGAACAGCCTGAAGGCCAGCGACACCGCCATGTACTACTGTGCCAGACTGCCCCAGTACTG
CAGCAATGGCGTTTGCCAGCGTTGGTTCGATCCTTGGGGACAGGGCACACTGGTCACCGTTAGCAGCGCTAGCACAA
AAGGCCCTAGCGTCTTTCCACTCGCCCCATCCTCCAAGAGCACCTCTGGGGGCACTGCGGCTTTGGGCTGCTTGGTG
AAGGATTACTTCCCAGAGCCTGTCACTGTCAGCTGGAATAGCGGGGCTCTGACCAGTGGAGTGCACACCTTTCCCGC
CGTGTTACAGAGCAGCGGCCTCTATAGCCTGAGCAGTGTGGTAACTGTGCCCTCGAGCAGCTTGGGTACCCAGACCT
ATATCTGCAATGTCAATCATAAACCCAGTAACACAAAGGTAGACAAGAAAGTCGAACCCAAATCTTGCGACAAAACT
CACACATGCCCTCCTTGCCCCGCCCCCGAACTCCTGGGGGGACCTTCGGTTTTTCTTTTTCCACCTAAACCGAAAGA
CACCCTGATGATCAGCAGGACACCAGAAGTGACATGTGTCGTCGTGGACGTATCCCATGAGGACCCGGAGGTGAAGT
TCAACTGGTATGTTGATGGTGTGGAAGTGCATAATGCAAAAACTAAGCCACGGGAGGAACAGTACAATAGCACATAC
AGAGTAGTTAGCGTTCTGACTGTCCTGCACCAAGATTGGCTGAACGGGAAGGAATACAAGTGTAAGGTGAGCAACAA
AGCCCTGCCCGCTCCAATTGAGAAAACAATTTCTAAGGCCAAAGGACAGCCCCGGGAGCCACAGGTGTATACTCTGC
CGCCTAGCAGGGAGGAAATGACCAAGAACCAGGTGAGCCTGACCTGTCTGGTGAAAGGCTTCTACCCCAGCGATATC
GCAGTTGAGTGGGAGAGCAATGGCCAACCCGAGAACAACTACAAGACTACGCCCCCCGTGCTTGATAGCGACGGATC
ATTTTTCCTGTACTCAAAACTGACCGTGGACAAAAGCAGATGGCAGCAGGGAAACGTTTTCAGTTGCTCAGTGATGC
ACGAAGCGCTGCACAATCATTATACTCAGAAAAGCCTGAGCTTGAGCCCGGGAAAA
SEQ ID NO: 86 CC40.8 L chain amino acid sequence
SYELTQPPSVSVSPGQTARITCSGDALPKRYAYWYQQKSGQAPILVIYEDKKRPSGIPERLSGSKSGTVATLTISGA
QVEDEADYYCYSTDSSGNHAVFGGGTQLTVLGQPKAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNRGEC
SEQ ID NO: 87 CV3-25 L chain amino acid sequence
EIVLTQSPSSVSASVGDRVTITCRASQGISSWLAWYQQKPGKAPKLLIYAASSLQSGVPSRFSGSGSGTDFTLTISS
LQPEDFATYYCQQGNSFPYTFGQGTNLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID NO: 88 CC40.8 L chain nucleotide sequence
AGCTATGAACTGACACAGCCTCCAAGCGTGTCCGTGTCTCCTGGACAGACCGCCAGAATCACATGTAGCGGAGATGC
CCTGCCTAAGAGATACGCCTACTGGTATCAGCAGAAGTCCGGACAGGCCCCTATCCTGGTCATCTACGAGGACAAGA
AGCGGCCCTCTGGCATCCCTGAGAGACTGAGCGGAAGCAAGTCTGGCACAGTGGCCACACTGACAATCTCTGGCGCC
CAGGTGGAAGATGAGGCCGACTACTACTGCTACAGCACCGACAGCTCTGGCAACCACGCTGTGTTTGGCGGAGGTAC
CCAACTGACCGTGCTGGGCCAACCTAAAGCCGCTCCTAGCGTGTTCATCTTTCCACCTAGCGACGAGCAGCTGAAGT
CTGGCACAGCCTCTGTCGTGTGCCTGCTGAACAACTTCTACCCCAGAGAAGCCAAGGTGCAGTGGAAGGTGGACAAC
GCCCTGCAGAGCGGCAATAGCCAAGAGAGCGTGACCGAGCAGGACAGCAAGGACTCTACCTACAGCCTGAGCAGCAC
CCTGACACTGAGCAAGGCCGACTACGAGAAGCACAAAGTGTACGCCTGCGAAGTGACCCACCAGGGCCTTTCTAGCC
CTGTGACCAAGAGCTTCAACCGGGGCGAGTGC
SEQ ID NO: 89 CV3-25 L chain nucleotide sequence
GAAATTGTGCTGACACAGAGCCCCAGCAGCGTGTCAGCCTCTGTGGGAGACAGAGTGACCATCACCTGTAGAGCCAG
CCAGGGCATCTCTTCTTGGCTGGCCTGGTATCAGCAGAAGCCTGGCAAGGCCCCTAAGCTGCTGATCTATGCCGCTA
GCTCTCTGCAGTCTGGCGTGCCCAGCAGATTTTCTGGCAGCGGCTCTGGCACCGACTTCACCCTGACCATATCTAGC
CTGCAGCCTGAGGACTTCGCCACCTACTATTGCCAGCAGGGCAACAGCTTCCCCTACACCTTTGGCCAGGGTACCAA
CCTGGAAATCAAGCGGACAGTGGCCGCTCCTAGCGTGTTCATCTTTCCACCTAGCGACGAGCAGCTGAAGTCTGGCA
CAGCCTCTGTCGTGTGCCTGCTGAACAACTTCTACCCCAGAGAAGCCAAGGTGCAGTGGAAGGTGGACAACGCCCTG
CAGAGCGGCAATAGCCAAGAGAGCGTGACCGAGCAGGACAGCAAGGACTCTACCTACAGCCTGAGCAGCACCCTGAC
ACTGAGCAAGGCCGACTACGAGAAGCACAAAGTGTACGCCTGCGAAGTGACCCACCAGGGCCTTTCTAGCCCTGTGA
CCAAGAGCTTCAACCGGGGCGAGTGC
SEQ ID NO: 90 S2P6 IgG1 H429F H chain amino acid sequence
EVQLVQSGAEVKKPGASVKVSCKASGYTFTSQYMHWVRQAPGQGLEWIGIINPSGVHTSYAQKFQGRVTLTRDTSTS
TLYMELSSLRSEDTAVYYCARGSPKGAFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMFEALHNHYTQKSLSLSPGK
SEQ ID NO: 91 S2P6 IgG1 H429F H chain nucleotide sequence
GAAGTTCAGCTGGTTCAGTCTGGCGCCGAAGTGAAGAAACCTGGCGCCTCTGTGAAGGTGTCCTGCAAGGCCAGCGG
CTACACCTTTACCAGCCAGTACATGCACTGGGTCCGACAGGCTCCAGGACAGGGACTCGAGTGGATCGGCATCATCA
ATCCTAGCGGCGTGCACACCAGCTACGCCCAGAAATTCCAGGGCAGAGTGACCCTGACCAGAGACACCAGCACCAGC
ACACTGTACATGGAACTGAGCAGCCTGCGGAGCGAGGATACCGCCGTGTACTATTGTGCCAGAGGCTCTCCTAAGGG
CGCCTTCGATTATTGGGGCCAGGGCACACTGGTCACCGTGTCCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGTACCCAGACCTATATCTGCAATGTCAATCATA
AACCCAGTAACACAAAGGTAGACAAGAAAGTCGAACCCAAATCTTGCGACAAAACTCACACATGCCCTCCTTGCCCC
GCCCCCGAACTCCTGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAAGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGATGAACTGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGTTCGAAGCGCTGCACAATCATT
ATACTCAGAAAAGCCTGAGCTTGAGCCCGGGAAAA
SEQ ID NO: 92 CC40.8 IgG1 H429F H chain amino acid sequence
QVQEVQLLESGGGLVQPGGSLRLSCAASGFTFSSYVMTWARQAPGKGLEWVSAISGTGYTYYADSVKGRFTVSRDNS
KNTLFLQMSSLRAEDTAVYYCAITMAPVVWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMFEALHNHYTQKSLSLSPGK
SEQ ID NO: 93 CC40.8 IgG1 H429F H chain nucleotide sequence
CAAGTGCAGGAAGTGCAGCTGCTGGAATCTGGCGGAGGACTGGTTCAACCAGGCGGCTCTCTGAGACTGTCTTGTGC
CGCCAGCGGCTTCACCTTCAGCAGCTACGTTATGACCTGGGCCAGACAGGCCCCTGGCAAAGGACTTGAATGGGTGT
CCGCCATCAGCGGCACAGGCTACACCTACTACGCCGACTCTGTGAAGGGCAGATTCACCGTGTCCAGAGACAACAGC
AAGAATACCCTGTTCCTGCAGATGAGCAGCCTGAGAGCCGAGGATACCGCCGTGTACTACTGCGCCATTACAATGGC
CCCTGTCGTGTGGGGCCAGGGCACAACAGTGACAGTCAGCAGCGCTAGCACAAAAGGCCCTAGCGTCTTTCCACTCG
CCCCATCCTCCAAGAGCACCTCTGGGGGCACTGCGGCTTTGGGCTGCTTGGTGAAGGATTACTTCCCAGAGCCTGTC
ACTGTCAGCTGGAATAGCGGGGCTCTGACCAGTGGAGTGCACACCTTTCCCGCCGTGTTACAGAGCAGCGGCCTCTA
TAGCCTGAGCAGTGTGGTAACTGTGCCCTCGAGCAGCTTGGGTACCCAGACCTATATCTGCAATGTCAATCATAAAC
CCAGTAACACAAAGGTAGACAAGAAAGTCGAACCCAAATCTTGCGACAAAACTCACACATGCCCTCCTTGCCCCGCC
CCCGAACTCCTGGGGGGACCTTCGGTTTTTCTTTTTCCACCTAAACCGAAAGACACCCTGATGATCAGCAGGACACC
AGAAGTGACATGTGTCGTCGTGGACGTATCCCATGAGGACCCGGAGGTGAAGTTCAACTGGTATGTTGATGGTGTGG
AAGTGCATAATGCAAAAACTAAGCCACGGGAGGAACAGTACAATAGCACATACAGAGTAGTTAGCGTTCTGACTGTC
CTGCACCAAGATTGGCTGAACGGGAAGGAATACAAGTGTAAGGTGAGCAACAAAGCCCTGCCCGCTCCAATTGAGAA
AACAATTTCTAAGGCCAAAGGACAGCCCCGGGAGCCACAGGTGTATACTCTGCCGCCTAGCAGGGATGAACTGACCA
AGAACCAGGTGAGCCTGACCTGTCTGGTGAAAGGCTTCTACCCCAGCGATATCGCAGTTGAGTGGGAGAGCAATGGC
CAACCCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGTTCGAAGCGCTGCACAATCATTATA
CTCAGAAAAGCCTGAGCTTGAGCCCGGGAAAA
SEQ ID NO: 94 CV3-25 IgG1 H chain H429F amino acid sequence
QVQLVESGAEVKKPGESLKISCKGSGYTFTRYWIGWVRQMPGKGLEWMGIIYPGDSDTRYSPSFQGHVTISADKSIS
TAYLQWNSLKASDTAMYYCARLPQYCSNGVCQRWFDPWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMFEALHNHYTQKSLSLSPGK
SEQ ID NO: 95 CV3-25 IgG1 H chain H429F nucleotide sequence
CAAGTGCAGCTGGTTGAATCTGGCGCCGAAGTGAAGAAGCCTGGCGAGAGCCTGAAGATCAGCTGCAAAGGCAGCGG
CTACACCTTCACCAGATACTGGATCGGCTGGGTCCGACAGATGCCTGGCAAAGGCCTTGAGTGGATGGGCATCATCT
ACCCCGGCGACAGCGATACCAGATACAGCCCTAGCTTTCAGGGCCACGTGACCATCAGCGCCGACAAGTCTATCAGC
ACCGCCTACCTGCAGTGGAACAGCCTGAAGGCCAGCGACACCGCCATGTACTACTGTGCCAGACTGCCCCAGTACTG
CAGCAATGGCGTTTGCCAGCGTTGGTTCGATCCTTGGGGACAGGGCACACTGGTCACCGTTAGCAGCGCTAGCACAA
AAGGCCCTAGCGTCTTTCCACTCGCCCCATCCTCCAAGAGCACCTCTGGGGGCACTGCGGCTTTGGGCTGCTTGGTG
AAGGATTACTTCCCAGAGCCTGTCACTGTCAGCTGGAATAGCGGGGCTCTGACCAGTGGAGTGCACACCTTTCCCGC
CGTGTTACAGAGCAGCGGCCTCTATAGCCTGAGCAGTGTGGTAACTGTGCCCTCGAGCAGCTTGGGTACCCAGACCT
ATATCTGCAATGTCAATCATAAACCCAGTAACACAAAGGTAGACAAGAAAGTCGAACCCAAATCTTGCGACAAAACT
CACACATGCCCTCCTTGCCCCGCCCCCGAACTCCTGGGGGGACCTTCGGTTTTTCTTTTTCCACCTAAACCGAAAGA
CACCCTGATGATCAGCAGGACACCAGAAGTGACATGTGTCGTCGTGGACGTATCCCATGAGGACCCGGAGGTGAAGT
TCAACTGGTATGTTGATGGTGTGGAAGTGCATAATGCAAAAACTAAGCCACGGGAGGAACAGTACAATAGCACATAC
AGAGTAGTTAGCGTTCTGACTGTCCTGCACCAAGATTGGCTGAACGGGAAGGAATACAAGTGTAAGGTGAGCAACAA
AGCCCTGCCCGCTCCAATTGAGAAAACAATTTCTAAGGCCAAAGGACAGCCCCGGGAGCCACAGGTGTATACTCTGC
CGCCTAGCAGGGATGAACTGACCAAGAACCAGGTGAGCCTGACCTGTCTGGTGAAAGGCTTCTACCCCAGCGATATC
GCAGTTGAGTGGGAGAGCAATGGCCAACCCGAGAACAACTACAAGACTACGCCCCCCGTGCTTGATAGCGACGGATC
ATTTTTCCTGTACTCAAAACTGACCGTGGACAAAAGCAGATGGCAGCAGGGAAACGTTTTCAGTTGCTCAGTGATGT
TCGAAGCGCTGCACAATCATTATACTCAGAAAAGCCTGAGCTTGAGCCCGGGAAAA
SEQ ID NO: 104 Ef1ACE2-Ig-like-WT H chain amino acid sequence
ETGSTIEEQAKYFLDKFNHEAEDLFYQSSLASWNYNTNITEENVQNMNNAGDKWSAFLKEQSTTAQMYPLQEIQNLT
VKLQLQALQQNGSSVLSEDKSKRLNTILNTMSTIYSTGKVCNPDNPQECLLLEPGLNEIMANSLDYNERLWAWESWR
SEVGKQLRPLYEEYVVLKNEMARANHYEDYGDYWRGDYEVNGVDGYDYSRGQLIEDVEHTFEEIKPLYEHLHAYVRA
KLMNAYPSYISPIGCLPAHLLGDMWGRFWTNLYSLTVPFGQKPNIDVTDAMVDQAWDAQRIFKEAEKFFVSVGLPNM
TQGFWEYSMLTDPGNVQKAVCHPTAWDLGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTLPFTYMLEKWRWMVFKGEIPKDQWMKKW
WEMKREIVGVVEPVPHDETYCDPASLFHVSNDYSFIRYYTRTLYQFQFQEALCQAAKHEGPLHKCDISNSTEAGQKL
FNMLRLGKSEPWTLALENVVGAKNMNVRPLLNYFEPLFTWLKDQNKNSFVGWSTDWSPYADQSIKVRISLKSALGDR
AYEWNDNEMYLFRSSVAYAMRQYFLKVKNQMILFGEEDVRVANLKPRISENFFVTAPKNVSDIIPRTEVEKAIRMSR
SRINDAFRLNDNSLEFLGIQPTLGPPNQPPVSGGGGSGGGGSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK
SEQ ID NO: 105 Ef1ACE2-Ig-like-WT H chain nucleotide sequence
GAAACCGGTTCCACCATTGAGGAACAGGCCAAGTACTTTTTGGACAAGTTTAACCACGAAGCCGAAGACCTGTTCTA
TCAAAGTTCACTTGCTTCTTGGAATTATAACACCAATATTACTGAAGAGAATGTCCAAAACATGaACAACGCCGGCG
ACAAGTGGAGCGCCTTCCTGAAAGAGCAGAGCACAACAGCCCAGATGTACCCTCTGCAAGAGATCCAGAACCTGACC
GTGAAGCTCCAGCTGCAGGCCCTCCAGCAGAATGGAAGCTCTGTGCTGAGCGAGGACAAGAGCAAGCGGCTGAACAC
CATCCTGAATACCATGAGCACCATCTACAGCACCGGCAAAGTGTGCAACCCCGACAATCCCCAAGAGTGCCTGCTGC
TGGAACCCGGCCTGAATGAGATCATGGCCAACAGCCTGGACTACAACGAGAGACTGTGGGCCTGGGAGTCTTGGAGA
AGCGAAGTGGGAAAGCAGCTGCGGCCCCTGTACGAGGAATACGTGGTGCTGAAGAACGAGATGGCCAGAGCCAACCA
CTACGAGGACTACGGCGACTATTGGAGAGGCGACTACGAAGTGAATGGCGTGGACGGCTACGACTACAGCAGAGGCC
AGCTGATCGAGGACGTGGAACACACCTTCGAGGAAATCAAGCCTCTGTACGAGCATCTGCACGCCTACGTGCGGGCC
AAGCTGATGAATGCTTACCCCAGCTACATCAGCCCCATCGGCTGTCTGCCTGCTCATCTGCTGGGAGACATGTGGGG
CAGATTCTGGACCAACCTGTACAGCCTGACAGTGCCCTTCGGCCAGAAACCTAACATCGACGTGACCGACGCTATGG
TGGATCAGGCTTGGGATGCCCAGCGGATCTTCAAAGAGGCCGAGAAGTTCTTCGTGTCCGTGGGCCTGCCTAATATG
ACCCAAGGCTTCTGGGAGTACTCCATGCTGACAGACCCCGGCAACGTGCAGAAAGCCGTGTGTCATCCTACCGCCTG
GGATCTCGGCAAGGGCGACTTCAGAATCCTGATGTGCACCAAAGTGACGATGGACGACTTCCTGACAGCCCACCACG
AGATGGGCCACATCCAGTACGATATGGCCTACGCCGCTCAGCCCTTCCTGCTGAGAAATGGCGCCAATGAGGGCTTC
CATGAGGCCGTGGGAGAGATCATGAGCCTGTCTGCCGCCACACCTAAGCACCTGAAGTCTATCGGACTGCTGAGCCC
CGACTTCCAAGAGGACAACGAGACAGAGATCAACTTCCTGCTCAAGCAGGCCCTGACCATCGTGGGCACACTGCCCT
TTACCTACATGCTGGAAAAGTGGCGGTGGATGGTCTTTAAGGGCGAGATCCCCAAGGACCAGTGGATGAAGAAATGG
TGGGAGATGAAGCGCGAGATCGTGGGCGTTGTGGAACCTGTGCCTCACGACGAGACATACTGCGATCCTGCCAGCCT
GTTTCACGTGTCCAACGACTACTCCTTCATCCGGTACTACACCCGGACACTGTACCAGTTCCAGTTTCAAGAGGCTC
TGTGCCAGGCCGCCAAGCACGAAGGACCTCTGCACAAGTGCGACATCAGCAACTCTACAGAGGCCGGACAGAAACTG
TTCAACATGCTGCGGCTGGGCAAGAGCGAGCCTTGGACACTGGCTCTGGAAAATGTCGTGGGCGCCAAGAACATGAA
TGTGCGGCCACTGCTGAACTACTTCGAGCCCCTGTTCACCTGGCTGAAGGACCAGAACAAGAACAGCTTCGTCGGCT
GGTCGACCGATTGGAGCCCTTATGCCGATCAGAGCATCAAAGTGCGGATCAGCCTGAAGTCTGCCCTGGGCGATAGA
GCCTACGAGTGGAACGACAACGAGATGTACCTGTTCCGGTCCAGCGTGGCATATGCTATGCGGCAGTACTTCCTGAA
AGTGAAGAACCAGATGATCCTGTTCGGCGAAGAGGACGTGCGCGTGGCCAATCTGAAGCCCAGAATCAGCTTCAACT
TCTTCGTGACAGCCCCTAAGAACGTGTCCGATATCATCCCCAGAACCGAGGTGGAAAAGGCCATCAGAATGAGCAGA
AGCCGGATCAACGACGCCTTCCGGCTGAACGACAATAGCCTGGAATTTCTGGGCATCCAGCCTACACTGGGCCCTCC
AAATCAGCCTCCTGTTAGCGGAGGAGGAGGATCTGGAGGAGGAGGATCCGCTAGCACAAAAGGCCCTAGCGTCTTTC
CACTCGCCCCATCCTCCAAGAGCACCTCTGGGGGCACTGCGGCTTTGGGCTGCTTGGTGAAGGATTACTTCCCAGAG
CCTGTCACTGTCAGCTGGAATAGCGGGGCTCTGACCAGTGGAGTGCACACCTTTCCCGCCGTGTTACAGAGCAGCGG
CCTCTATAGCCTGAGCAGTGTGGTAACTGTGCCCTCGAGCAGCTTGGGTACCCAGACCTATATCTGCAATGTCAATC
ATAAACCCAGTAACACAAAGGTAGACAAGAAAGTCGAACCCAAATCTTGCGACAAAACTCACACATGCCCTCCTTGC
CCCGCCCCCGAGCTCCTGGGGGGACCTTCGGTTTTTCTTTTTCCACCTAAACCGAAAGACACCCTGATGATCAGCAG
GACACCAGAAGTGACATGTGTCGTCGTGGACGTATCCCATGAGGACCCGGAGGTGAAGTTCAACTGGTATGTTGATG
GTGTGGAAGTGCATAATGCAAAAACTAAGCCACGGGAGGAACAGTACAATAGCACATACAGAGTAGTTAGCGTTCTG
ACTGTCCTGCACCAAGATTGGCTGAACGGGAAGGAATACAAGTGTAAGGTGAGCAACAAAGCCCTGCCCGCTCCAAT
TGAGAAAACAATTTCTAAGGCCAAAGGACAGCCCCGGGAGCCACAGGTGTATACTCTGCCGCCTAGCAGGGAGGAAA
TGACCAAGAACCAGGTGAGCCTGACCTGTCTGGTGAAAGGCTTCTACCCCAGCGATATCGCAGTTGAGTGGGAGAGC
AATGGCCAACCCGAGAACAACTACAAGACTACGCCCCCCGTGCTTGATAGCGACGGATCATTTTTCCTGTACTCAAA
ACTGACCGTGGACAAAAGCAGATGGCAGCAGGGAAACGTTTTCAGTTGCTCAGTGATGCACGAAGCGCTGCACAATC
ATTATACTCAGAAAAGCCTGAGCTTGAGCCCGGGAAAA
SEQ ID NO: 106
Ef1ACE2 light chain amino acid sequence
ETGSTIEEQAKYFLDKFNHEAEDLFYQSSLASWNYNTNITEENVQNMNNAGDKWSAFLKEQSTTAQMYPLQEIQNLT
VKLQLQALQQNGSSVLSEDKSKRLNTILNTMSTIYSTGKVCNPDNPQECLLLEPGLNEIMANSLDYNERLWAWESWR
SEVGKQLRPLYEEYVVLKNEMARANHYEDYGDYWRGDYEVNGVDGYDYSRGQLIEDVEHTFEEIKPLYEHLHAYVRA
KLMNAYPSYISPIGCLPAHLLGDMWGRFWTNLYSLTVPFGQKPNIDVTDAMVDQAWDAQRIFKEAEKFFVSVGLPNM
TQGFWEYSMLTDPGNVQKAVCHPTAWDLGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTLPFTYMLEKWRWMVFKGEIPKDQWMKKW
WEMKREIVGVVEPVPHDETYCDPASLFHVSNDYSFIRYYTRTLYQFQFQEALCQAAKHEGPLHKCDISNSTEAGQKL
FNMLRLGKSEPWTLALENVVGAKNMNVRPLLNYFEPLFTWLKDQNKNSFVGWSTDWSPYADQSIKVRISLKSALGDR
AYEWNDNEMYLFRSSVAYAMRQYFLKVKNQMILFGEEDVRVANLKPRISENFFVTAPKNVSDIIPRTEVEKAIRMSR
SRINDAFRLNDNSLEFLGIQPTLGPPNQPPVSGGGGSGGGGSRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGEC
SEQ ID NO: 107 Ef1ACE2 light chain nucleotide sequence
GAAACCGGTTCCACCATTGAGGAACAGGCCAAGTACTTTTTGGACAAGTTTAACCACGAAGCCGAAGACCTGTTCTA
TCAAAGTTCACTTGCTTCTTGGAATTATAACACCAATATTACTGAAGAGAATGTCCAAAACATGAACAACGCCGGCG
ACAAGTGGAGCGCCTTCCTGAAAGAGCAGAGCACAACAGCCCAGATGTACCCTCTGCAAGAGATCCAGAACCTGACC
GTGAAGCTCCAGCTGCAGGCCCTCCAGCAGAATGGAAGCTCTGTGCTGAGCGAGGACAAGAGCAAGCGGCTGAACAC
CATCCTGAATACCATGAGCACCATCTACAGCACCGGCAAAGTGTGCAACCCCGACAATCCCCAAGAGTGCCTGCTGC
TGGAACCCGGCCTGAATGAGATCATGGCCAACAGCCTGGACTACAACGAGAGACTGTGGGCCTGGGAGTCTTGGAGA
AGCGAAGTGGGAAAGCAGCTGCGGCCCCTGTACGAGGAATACGTGGTGCTGAAGAACGAGATGGCCAGAGCCAACCA
CTACGAGGACTACGGCGACTATTGGAGAGGCGACTACGAAGTGAATGGCGTGGACGGCTACGACTACAGCAGAGGCC
AGCTGATCGAGGACGTGGAACACACCTTCGAGGAAATCAAGCCTCTGTACGAGCATCTGCACGCCTACGTGCGGGCC
AAGCTGATGAATGCTTACCCCAGCTACATCAGCCCCATCGGCTGTCTGCCTGCTCATCTGCTGGGAGACATGTGGGG
CAGATTCTGGACCAACCTGTACAGCCTGACAGTGCCCTTCGGCCAGAAACCTAACATCGACGTGACCGACGCTATGG
TGGATCAGGCTTGGGATGCCCAGCGGATCTTCAAAGAGGCCGAGAAGTTCTTCGTGTCCGTGGGCCTGCCTAATATG
ACCCAAGGCTTCTGGGAGTACTCCATGCTGACAGACCCCGGCAACGTGCAGAAAGCCGTGTGTCATCCTACCGCCTG
GGATCTCGGCAAGGGCGACTTCAGAATCCTGATGTGCACCAAAGTGACGATGGACGACTTCCTGACAGCCCACCACG
AGATGGGCCACATCCAGTACGATATGGCCTACGCCGCTCAGCCCTTCCTGCTGAGAAATGGCGCCAATGAGGGCTTC
CATGAGGCCGTGGGAGAGATCATGAGCCTGTCTGCCGCCACACCTAAGCACCTGAAGTCTATCGGACTGCTGAGCCC
CGACTTCCAAGAGGACAACGAGACAGAGATCAACTTCCTGCTCAAGCAGGCCCTGACCATCGTGGGCACACTGCCCT
TTACCTACATGCTGGAAAAGTGGCGGTGGATGGTCTTTAAGGGCGAGATCCCCAAGGACCAGTGGATGAAGAAATGG
TGGGAGATGAAGCGCGAGATCGTGGGCGTTGTGGAACCTGTGCCTCACGACGAGACATACTGCGATCCTGCCAGCCT
GTTTCACGTGTCCAACGACTACTCCTTCATCCGGTACTACACCCGGACACTGTACCAGTTCCAGTTTCAAGAGGCTC
TGTGCCAGGCCGCCAAGCACGAAGGACCTCTGCACAAGTGCGACATCAGCAACTCTACAGAGGCCGGACAGAAACTG
TTCAACATGCTGCGGCTGGGCAAGAGCGAGCCTTGGACACTGGCTCTGGAAAATGTCGTGGGCGCCAAGAACATGAA
TGTGCGGCCACTGCTGAACTACTTCGAGCCCCTGTTCACCTGGCTGAAGGACCAGAACAAGAACAGCTTCGTCGGCT
GGTCGACCGATTGGAGCCCTTATGCCGATCAGAGCATCAAAGTGCGGATCAGCCTGAAGTCTGCCCTGGGCGATAGA
GCCTACGAGTGGAACGACAACGAGATGTACCTGTTCCGGTCCAGCGTGGCATATGCTATGCGGCAGTACTTCCTGAA
AGTGAAGAACCAGATGATCCTGTTCGGCGAAGAGGACGTGCGCGTGGCCAATCTGAAGCCCAGAATCAGCTTCAACT
TCTTCGTGACAGCCCCTAAGAACGTGTCCGATATCATCCCCAGAACCGAGGTGGAAAAGGCCATCAGAATGAGCAGA
AGCCGGATCAACGACGCCTTCCGGCTGAACGACAATAGCCTGGAATTTCTGGGCATCCAGCCTACACTGGGCCCTCC
AAATCAGCCTCCTGTTAGCGGAGGAGGAGGATCTGGAGGAGGAGGCTCGAGGACAGTGGCCGCTCCTAGCGTGTTCA
TCTTTCCACCTAGCGACGAGCAGCTGAAGTCTGGCACAGCCTCTGTCGTGTGCCTGCTGAACAACTTCTACCCCAGA
GAAGCCAAGGTGCAGTGGAAGGTGGACAACGCCCTGCAGAGCGGCAATAGCCAAGAGAGCGTGACCGAGCAGGACAG
CAAGGACTCTACCTACAGCCTGAGCAGCACCCTGACACTGAGCAAGGCCGACTACGAGAAGCACAAAGTGTACGCCT
GCGAAGTGACCCACCAGGGCCTTTCTAGCCCTGTGACCAAGAGCTTCAACCGGGGCGAGTGC
SEQ ID NO: 108 Ef1ACE2-Ig-like-H429F H chain amino acid sequence
ETGSTIEEQAKYFLDKFNHEAEDLFYQSSLASWNYNTNITEENVQNMNNAGDKWSAFLKEQSTTAQMYPLQEIQNLT
VKLQLQALQQNGSSVLSEDKSKRLNTILNTMSTIYSTGKVCNPDNPQECLLLEPGLNEIMANSLDYNERLWAWESWR
SEVGKQLRPLYEEYVVLKNEMARANHYEDYGDYWRGDYEVNGVDGYDYSRGQLIEDVEHTFEEIKPLYEHLHAYVRA
KLMNAYPSYISPIGCLPAHLLGDMWGRFWTNLYSLTVPFGQKPNIDVTDAMVDQAWDAQRIFKEAEKFFVSVGLPNM
TQGFWEYSMLTDPGNVQKAVCHPTAWDLGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTLPFTYMLEKWRWMVFKGEIPKDQWMKKW
WEMKREIVGVVEPVPHDETYCDPASLFHVSNDYSFIRYYTRTLYQFQFQEALCQAAKHEGPLHKCDISNSTEAGQKL
FNMLRLGKSEPWTLALENVVGAKNMNVRPLLNYFEPLFTWLKDQNKNSFVGWSTDWSPYADQSIKVRISLKSALGDR
AYEWNDNEMYLFRSSVAYAMRQYFLKVKNQMILFGEEDVRVANLKPRISENFFVTAPKNVSDIIPRTEVEKAIRMSR
SRINDAFRLNDNSLEFLGIQPTLGPPNQPPVSGGGGSGGGGSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMFEALHNHYTQKSLSLSPGK
SEQ ID NO: 109 Ef1ACE2-Ig-like-H429F H chain nucleotide sequence
GAAACCGGTTCCACCATTGAGGAACAGGCCAAGTACTTTTTGGACAAGTTTAACCACGAAGCCGAAGACCTGTTCTA
TCAAAGTTCACTTGCTTCTTGGAATTATAACACCAATATTACTGAAGAGAATGTCCAAAACATGaACAACGCCGGCG
ACAAGTGGAGCGCCTTCCTGAAAGAGCAGAGCACAACAGCCCAGATGTACCCTCTGCAAGAGATCCAGAACCTGACC
GTGAAGCTCCAGCTGCAGGCCCTCCAGCAGAATGGAAGCTCTGTGCTGAGCGAGGACAAGAGCAAGCGGCTGAACAC
CATCCTGAATACCATGAGCACCATCTACAGCACCGGCAAAGTGTGCAACCCCGACAATCCCCAAGAGTGCCTGCTGC
TGGAACCCGGCCTGAATGAGATCATGGCCAACAGCCTGGACTACAACGAGAGACTGTGGGCCTGGGAGTCTTGGAGA
AGCGAAGTGGGAAAGCAGCTGCGGCCCCTGTACGAGGAATACGTGGTGCTGAAGAACGAGATGGCCAGAGCCAACCA
CTACGAGGACTACGGCGACTATTGGAGAGGCGACTACGAAGTGAATGGCGTGGACGGCTACGACTACAGCAGAGGCC
AGCTGATCGAGGACGTGGAACACACCTTCGAGGAAATCAAGCCTCTGTACGAGCATCTGCACGCCTACGTGCGGGCC
AAGCTGATGAATGCTTACCCCAGCTACATCAGCCCCATCGGCTGTCTGCCTGCTCATCTGCTGGGAGACATGTGGGG
CAGATTCTGGACCAACCTGTACAGCCTGACAGTGCCCTTCGGCCAGAAACCTAACATCGACGTGACCGACGCTATGG
TGGATCAGGCTTGGGATGCCCAGCGGATCTTCAAAGAGGCCGAGAAGTTCTTCGTGTCCGTGGGCCTGCCTAATATG
ACCCAAGGCTTCTGGGAGTACTCCATGCTGACAGACCCCGGCAACGTGCAGAAAGCCGTGTGTCATCCTACCGCCTG
GGATCTCGGCAAGGGCGACTTCAGAATCCTGATGTGCACCAAAGTGACGATGGACGACTTCCTGACAGCCCACCACG
AGATGGGCCACATCCAGTACGATATGGCCTACGCCGCTCAGCCCTTCCTGCTGAGAAATGGCGCCAATGAGGGCTTC
CATGAGGCCGTGGGAGAGATCATGAGCCTGTCTGCCGCCACACCTAAGCACCTGAAGTCTATCGGACTGCTGAGCCC
CGACTTCCAAGAGGACAACGAGACAGAGATCAACTTCCTGCTCAAGCAGGCCCTGACCATCGTGGGCACACTGCCCT
TTACCTACATGCTGGAAAAGTGGCGGTGGATGGTCTTTAAGGGCGAGATCCCCAAGGACCAGTGGATGAAGAAATGG
TGGGAGATGAAGCGCGAGATCGTGGGCGTTGTGGAACCTGTGCCTCACGACGAGACATACTGCGATCCTGCCAGCCT
GTTTCACGTGTCCAACGACTACTCCTTCATCCGGTACTACACCCGGACACTGTACCAGTTCCAGTTTCAAGAGGCTC
TGTGCCAGGCCGCCAAGCACGAAGGACCTCTGCACAAGTGCGACATCAGCAACTCTACAGAGGCCGGACAGAAACTG
TTCAACATGCTGCGGCTGGGCAAGAGCGAGCCTTGGACACTGGCTCTGGAAAATGTCGTGGGCGCCAAGAACATGAA
TGTGCGGCCACTGCTGAACTACTTCGAGCCCCTGTTCACCTGGCTGAAGGACCAGAACAAGAACAGCTTCGTCGGCT
GGTCGACCGATTGGAGCCCTTATGCCGATCAGAGCATCAAAGTGCGGATCAGCCTGAAGTCTGCCCTGGGCGATAGA
GCCTACGAGTGGAACGACAACGAGATGTACCTGTTCCGGTCCAGCGTGGCATATGCTATGCGGCAGTACTTCCTGAA
AGTGAAGAACCAGATGATCCTGTTCGGCGAAGAGGACGTGCGCGTGGCCAATCTGAAGCCCAGAATCAGCTTCAACT
TCTTCGTGACAGCCCCTAAGAACGTGTCCGATATCATCCCCAGAACCGAGGTGGAAAAGGCCATCAGAATGAGCAGA
AGCCGGATCAACGACGCCTTCCGGCTGAACGACAATAGCCTGGAATTTCTGGGCATCCAGCCTACACTGGGCCCTCC
AAATCAGCCTCCTGTTAGCGGAGGAGGAGGATCTGGAGGAGGAGGATCCGCTAGCACAAAAGGCCCTAGCGTCTTTC
CACTCGCCCCATCCTCCAAGAGCACCTCTGGGGGCACTGCGGCTTTGGGCTGCTTGGTGAAGGATTACTTCCCAGAG
CCTGTCACTGTCAGCTGGAATAGCGGGGCTCTGACCAGTGGAGTGCACACCTTTCCCGCCGTGTTACAGAGCAGCGG
CCTCTATAGCCTGAGCAGTGTGGTAACTGTGCCCTCGAGCAGCTTGGGTACCCAGACCTATATCTGCAATGTCAATC
ATAAACCCAGTAACACAAAGGTAGACAAGAAAGTCGAACCCAAATCTTGCGACAAAACTCACACATGCCCTCCTTGC
CCCGCCCCCGAGCTCCTGGGGGGACCTTCGGTTTTTCTTTTTCCACCTAAACCGAAAGACACCCTGATGATCAGCAG
GACACCAGAAGTGACATGTGTCGTCGTGGACGTATCCCATGAGGACCCGGAGGTGAAGTTCAACTGGTATGTTGATG
GTGTGGAAGTGCATAATGCAAAAACTAAGCCACGGGAGGAACAGTACAATAGCACATACAGAGTAGTTAGCGTTCTG
ACTGTCCTGCACCAAGATTGGCTGAACGGGAAGGAATACAAGTGTAAGGTGAGCAACAAAGCCCTGCCCGCTCCAAT
TGAGAAAACAATTTCTAAGGCCAAAGGACAGCCCCGGGAGCCACAGGTGTATACTCTGCCGCCTAGCAGGGAGGAAA
TGACCAAGAACCAGGTGAGCCTGACCTGTCTGGTGAAAGGCTTCTACCCCAGCGATATCGCAGTTGAGTGGGAGAGC
AATGGCCAACCCGAGAACAACTACAAGACTACGCCCCCCGTGCTTGATAGCGACGGATCATTTTTCCTGTACTCAAA
ACTGACCGTGGACAAAAGCAGATGGCAGCAGGGAAACGTTTTCAGTTGCTCAGTGATGTTCGAAGCGCTGCACAATC
ATTATACTCAGAAAAGCCTGAGCTTGAGCCCGGGAAAA
SEQ ID NO: 110 BDR5-1LA/LA/H429F IgG1 H chain amino acid sequence
EVQLQQSGAEVVKPGASVKLSCKASGFNIKDTFIHWVKQAPGQGLEWIGRIDPANTNTKYDPKFQGKATITTDTSSN
TAYMELSSLRSEDTAVYYCVRGLYTYYFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKRVEPKSCDKTHTCPPCP
APEAAGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMFEALHNHYTQKSLSLSPG
SEQ ID NO: 111 BDR5-1LA/LA/H429F IgG1 H chain nucleotide sequence
GAAGTGCAGCTGCAACAGTCTGGCGCCGAGGTTGTGAAACCTGGCGCCTCTGTGAAGCTGAGCTGTAAAGCCAGCGG
CTTCAACATCAAGGACACCTTCATCCACTGGGTCAAGCAGGCCCCTGGACAGGGACTCGAATGGATCGGCAGAATCG
ACCCCGCCAACACCAACACTAAGTACGACCCCAAGTTCCAGGGCAAAGCCACCATCACCACCGACACCAGCAGCAAC
ACCGCCTACATGGAACTGAGCAGCCTGAGAAGCGAGGACACCGCCGTGTACTATTGTGTGCGGGGCCTGTACACCTA
CTACTTCGATTATTGGGGCCAGGGTACCCTGGTCACAGTTAGCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGCACCCAGACCTATATCTGCAATGTCAATCATA
AACCCAGTAACACAAAGGTAGACAAGAGAGTCGAACCCAAATCTTGCGACAAAACTCACACATGCCCTCCTTGCCCC
GCCCCCGAAGCCGCGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGTTCGAAGCGCTGCACAATCATT
ATACTCAGAAAAGCCTGAGCTTGAGCCCGGGA
SEQ ID NO: 112 TDR5-1LA/LA/H429F IgG1 H chain amino acid sequence
QVQLVQSGAEVKKPGASVKVSCKASGFNIKDTHMHWVRQAPGQRLEWIGRIDPANGNTEYDQKFQGRVTITVDTSAS
TAYMELSSLRSEDTAVYYCARWGTNVYFAYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKRVEPKSCDKTHTCPPCP
APEAAGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMFEALHNHYTQKSLSLSPG
SEQ ID NO: 113 TDR5-1LA/LA/H429F IgG1 H chain nucleotide sequence
CAGGTTCAGCTGGTTCAGTCTGGCGCCGAAGTGAAGAAACCTGGCGCCTCTGTGAAGGTGTCCTGCAAGGCCAGCGG
CTTCAACATCAAGGACACCCACATGCACTGGGTCCGACAGGCTCCAGGACAGAGACTGGAATGGATCGGCAGAATCG
ACCCCGCCAACGGCAACACCGAGTACGACCAGAAATTCCAGGGCAGAGTGACCATCACCGTGGACACATCTGCCAGC
ACCGCCTACATGGAACTGAGCAGCCTGAGAAGCGAGGACACCGCCGTGTACTACTGTGCCAGATGGGGCACCAACGT
GTACTTCGCCTATTGGGGCCAGGGTACCCTGGTCACAGTTAGCTCTGCTAGCACAAAAGGCCCTAGCGTCTTTCCAC
TCGCCCCATCCTCCAAGAGCACCTCTGGGGGCACTGCGGCTTTGGGCTGCTTGGTGAAGGATTACTTCCCAGAGCCT
GTCACTGTCAGCTGGAATAGCGGGGCTCTGACCAGTGGAGTGCACACCTTTCCCGCCGTGTTACAGAGCAGCGGCCT
CTATAGCCTGAGCAGTGTGGTAACTGTGCCCTCGAGCAGCTTGGGCACCCAGACCTATATCTGCAATGTCAATCATA
AACCCAGTAACACAAAGGTAGACAAGAGAGTCGAACCCAAATCTTGCGACAAAACTCACACATGCCCTCCTTGCCCC
GCCCCCGAAGCCGCGGGGGGACCTTCGGTTTTTCTTTTTCCACCTAAACCGAAAGACACCCTGATGATCAGCAGGAC
ACCAGAAGTGACATGTGTCGTCGTGGACGTATCCCATGAGGACCCGGAGGTGAAGTTCAACTGGTATGTTGATGGTG
TGGAAGTGCATAATGCAAAAACTAAGCCACGGGAGGAACAGTACAATAGCACATACAGAGTAGTTAGCGTTCTGACT
GTCCTGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAAATGA
CCAAGAACCAGGTGAGCCTGACCTGTCTGGTGAAAGGCTTCTACCCCAGCGATATCGCAGTTGAGTGGGAGAGCAAT
GGCCAACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGTTCGAAGCGCTGCACAATCATT
ATACTCAGAAAAGCCTGAGCTTGAGCCCGGGA

1. An immunotherapeutic protein comprising one or more immunoglobulin heavy chain polypeptide comprising an Fc region component comprising at least a constant heavy chain domain 3 (CH3) domain (or at least a constant heavy domain 4 (CH4) domain), wherein said one or more polypeptide includes an amino acid substitution at a position corresponding to H429 of the amino acid sequence of human IgG1 heavy chain (Eu numbering).

2. The immunotherapeutic protein of claim 1, wherein the Fc region component comprises a constant heavy domain 3 (CH3) and a constant heavy domain 2 (CH2) of an immunoglobulin heavy chain, optionally in combination with a lower, core and/or upper hinge sequence.

3. The immunotherapeutic protein of claim 1, wherein the Fc region component comprises a constant heavy domain 3 (CH3) only.

4. The immunotherapeutic protein of any one of claims 1 to 3, wherein the Fc region component is derived from an IgG or IgA heavy chain polypeptide.

5. The immunotherapeutic protein of any one of claims 1 to 4, wherein the immunotherapeutic protein is an immunoglobulin molecule comprising first and second immunoglobulin heavy chain polypeptides comprising an Fc region component comprising CH2 and CH3 domains so that the first and second polypeptides form, by dimerisation, an Fc fragment or Fc-like fragment.

6. The immunotherapeutic protein of claim 4, wherein the Fc region component is derived from an IgG1 heavy chain polypeptide.

7. The immunotherapeutic protein of claim 4, wherein the Fc region component is derived from an IgG2 heavy chain polypeptide.

8. The immunotherapeutic protein of claim 4, wherein the Fc region component is derived from an IgG3 heavy chain polypeptide.

9. The immunotherapeutic protein of claim 4, wherein the Fc region component is derived from an IgG4 heavy chain polypeptide.

10. The immunotherapeutic protein of claim 4, wherein the Fc region component is derived from an IgA heavy chain polypeptide.

11. The immunotherapeutic protein of claim 1, wherein the immunotherapeutic protein is an immunoglobulin molecule comprising first and second immunoglobulin heavy chain polypeptides comprising an Fc region component comprising CH2 and CH4 domains so that the first and second polypeptides form, by dimerisation, an Fc fragment or Fc-like fragment.

12. The immunotherapeutic protein of claim 5 or 11, wherein the immunoglobulin protein is an antibody, antibody-like molecule or antibody derivative.

13. The immunotherapeutic protein of claim 12, wherein the immunoglobulin protein comprises at least one antigen recognition structure which may specifically bind to an antigen or epitope of therapeutic significance.

14. The immunotherapeutic protein of claim 13, wherein the at least one antigen recognition structure specifically binds to a cancer-associated antigen, an autoantigen, an allergen, an antigen associated with an inflammatory disease, an antigen from a transplanted tissue or organ or an antigen of an infectious agent such as an antigen of a bacterial or viral pathogen.

15. The immunotherapeutic protein of claim 14, wherein the at least one antigen recognition structure specifically binds to a cancer-associated antigen selected from CD20, CD38 and CD52.

16. The immunotherapeutic protein of claim 14, wherein the at least one antigen recognition structure specifically binds to an antigen of SARS-CoV-2 virus, Middle East respiratory syndrome coronavirus (MERS-CoV), respiratory syncytial virus (RSV) or dengue virus.

17. The immunotherapeutic protein of claim 14, wherein the at least one antigen recognition structure specifically binds to an antigen of a viral pathogen selected from antigens of coronaviruses.

18. The immunotherapeutic protein of claim 13, wherein the at least one antigen recognition structure specifically binds to a cell surface molecule known to induce cell activation or cell proliferation and/or stimulate an inhibitory pathway to inhibit or reduce cell responses.

19. The immunotherapeutic protein of claim 13, wherein the at least one antigen recognition structure specifically binds to a cell surface molecule known to induce a cellular response selected from stimulation of an inhibitory pathway to inhibit or reduce responses.

20. The immunotherapeutic protein of claim 13 or 15, wherein the at least one antigen recognition structure specifically binds to an immune checkpoint molecule.

21. The immunotherapeutic protein of claim 20, wherein the immune checkpoint molecule is selected from the group consisting of 4-1BB (CD137), cluster of differentiation (CD40, CD154), OX40 receptor (TNFRSF4, CD134), tumour necrosis factor receptor type II (TNFR2, CD120b), glucocorticoid-induced TNFR-related protein (GITR, TNFRSF18, CD357), cluster of differentiation 27 (CD27), T cell immunoglobulin and mucin-domain containing-3 (TIM-3), B and T lymphocyte attenuator (BTLA, CD272), lymphocyte activation gene-3 (LAG3, CD223), cytotoxic T-lymphocyte-associated protein 4 (CTLA4, CD152), inducible T-cell costimulatory (ICOS, CD278), cluster of differentiation 28 (CD28), TT cell immunoreceptor with Ig and ITIM domains (IGIT, Vstm3), programmed death-ligand 1 (PDL-1, CD274) and programmed cell death protein 1 (PD-1, CD279).

22. The immunotherapeutic protein of claim 13, wherein the at least one antigen recognition structure specifically binds to a cell surface molecule, wherein the cell surface molecule induces cell death cell death when engaged by a ligand or the immunotherapeutic protein.

23. The immunotherapeutic protein of claim 22, wherein the cell surface molecule is selected from the group consisting of tumour necrosis receptor 1 (TNFRT), Fas (CD95), Death Receptor 3, Death Receptor 4, Death Receptor 5, Death Receptor 6, and cluster of differentiation (CD38).

24. The immunotherapeutic protein of any one of claims 12 to 23, wherein the immunotherapeutic protein is an antibody that forms oligomers through self-association in solution at physiological pH.

25. The immunotherapeutic protein of claim 24, wherein the immunotherapeutic protein is a hexamer.

26. The immunotherapeutic protein of claim 24 or 25, wherein the amino acid substitution at the position corresponding to H429 of the amino acid sequence of the human IgG1 heavy chain polypeptide is an H→Y substitution.

27. The immunotherapeutic protein of any one of claims 12 to 23, wherein the immunotherapeutic protein is an antibody that forms oligomers upon binding to a relevant target through “on target” oligomerisation.

29. The immunotherapeutic protein of claim 28, wherein the immunotherapeutic protein forms a hexamer upon binding to a relevant target through “on target” oligomerisation.

30. The immunotherapeutic protein of claim 28, wherein the amino acid substitution at the position corresponding to H429 of the amino acid sequence of the human IgG1 heavy chain polypeptide is an H→F substitution.

31. The immunotherapeutic protein of claim 28, wherein the amino acid substitution at the position corresponding to H429 of the amino acid sequence of the human IgG2 heavy chain polypeptide is an H→F substitution.

32. The immunotherapeutic protein of claim 28, wherein the amino acid substitution at the position corresponding to H429 of the amino acid sequence of the human IgG3 heavy chain polypeptide is an H→F substitution.

33. The immunotherapeutic protein of any one of claims 30 to 32, wherein the Fc region component comprises at least a constant heavy chain domain 3 (CH3) domain (or at least a constant heavy domain 4 (CH4) domain) and a constant heavy chain domain 2 (CH2) domain, and wherein said CH2 includes an L234A and/or L235A amino acid substitution.

34. The immunotherapeutic protein of any one of claims 1 to 4, wherein the immunotherapeutic protein comprises a fusion protein or protein conjugate comprising a partner polypeptide linked to an Fc region component comprising at least a CH3 domain (or at least a constant heavy domain 4 (CH4) domain).

35. The immunotherapeutic protein of claim 34, wherein the partner polypeptide is a cell surface receptor polypeptide (or a fragment thereof) or a co-receptor polypeptide (or fragment thereof).

36. The immunotherapeutic protein of claim 34, wherein the partner polypeptide is a cell surface receptor polypeptide (or a fragment thereof) that is capable of binding to a structural protein of a virus such that the immunotherapeutic protein may act as a “decoy” to block viral interaction and cellular entry of a virus to a host cell.

37. The immunotherapeutic protein of claim 34, wherein the partner polypeptide is an angiotensin-converting enzyme 2 (ACE2) ectodomain, dipeptidyl peptidase 4 (DPP4, CD26), Hsp70, hepatitis A virus cellular receptor 1 (HAVCR1/TIM-1), cluster of differentiation 155 (CD155), Glucose transporter 1 (GLUT1), Proto-oncogene tyrosine-protein kinase MER (MERTK), TYRO3 Protein Tyrosine Kinase (TYRO3), AXL or cluster of differentiation 4 receptor (CD4 receptor).

38. The immunotherapeutic protein of claim 34, wherein the partner polypeptide is a co-receptor polypeptide (or fragment thereof).

39. The immunotherapeutic protein of claim 38, wherein the co-receptor polypeptide (or fragment thereof) is C-X-C chemokine receptor type 4 (CXCR4), C—C chemokine receptor type 5 (CCR5), tetraspanin, or occludin.

40. The immunotherapeutic protein of claim 34, wherein the partner polypeptide is cytotoxic T-lymphocyte-associated protein 4 (CTLA4) (or a soluble extracellular fragment thereof), interleukin-1 receptor (IL-IR), interleukin-6 receptor (IL-6R), tumour necrosis factor receptor-2 (TNFR2, also known as CD120b) or a receptor for a cytokine of the TGF-β superfamily.

41. The immunotherapeutic protein of claim 34, wherein the partner polypeptide is a ligand for a cell surface molecule known to induce cell activation and/or proliferation and/or stimulate an inhibitory pathway to inhibit or reduce cell responses.

42. The immunotherapeutic protein of claim 41, wherein the partner polypeptide is a ligand for an immune checkpoint molecule.

43. The immunotherapeutic protein of claim 42, wherein the immune checkpoint molecule is selected from the group consisting of 4-1BB (CD137), CD40 (CD154), OX40 (TNFRSF4, CD134), TNFR2 (CD120b), GITR (TNFRSF18, CD357), CD27, TIM-3, BTLA (CD272), LAG3 (CD223), CTLA4 (CD152), ICOS (CD278), CD28, TIGIT (Vstm3), PDL-1 (CD274) and PD-1 (CD279)).

44. The immunotherapeutic protein of claim 34, wherein the partner polypeptide is a ligand for a cell surface molecule which induce cell death when engaged by the ligand.

45. The immunotherapeutic protein of claim 44, wherein the partner polypeptide is a ligand for a cell surface molecule selected from the group consisting of TNFR1, Fas, Death Receptor 3, Death Receptor 4, Death Receptor 5, Death Receptor 6, and CD38.

46. The immunotherapeutic protein of claim 45, wherein the partner polypeptide is TNF-related apoptosis-inducing ligand (TRAIL).

47. The immunotherapeutic protein of any one of claims 34 to 46, wherein the immunotherapeutic protein is a fusion protein that forms oligomers through self-association in solution at physiological pH.

48. The immunotherapeutic protein of claim 47, wherein the immunotherapeutic protein is a hexamer.

49. The immunotherapeutic protein of claim 47 or 48, wherein the amino acid substitution at the position corresponding to H429 of the amino acid sequence of the human IgG1 heavy chain polypeptide is an H→Y substitution.

50. The immunotherapeutic protein of any one of claims 34 to 46, wherein the immunotherapeutic protein is a fusion protein that forms oligomers upon binding to a relevant target through “on target” oligomerisation.

51. The immunotherapeutic protein of claim 50, wherein the immunotherapeutic protein forms a hexamer upon binding to a relevant target through “on target” oligomerisation.

52. The immunotherapeutic protein of claim 50 or 51, wherein the amino acid substitution at the position corresponding to H429 of the amino acid sequence of the human IgG1 heavy chain polypeptide is an H→F substitution.

53. The immunotherapeutic protein of any one of claims 34 to 52, wherein the immunotherapeutic protein is an antibody-like molecule in an H₂ or H₂L₂ format comprising two fusion proteins each comprising an immunoglobulin heavy chain polypeptide which comprises an Fc region component comprising at least a constant heavy chain domain 3 (CH3) domain (or at least a constant heavy domain 4 (CH4) domain) and including an amino acid substitution at a position corresponding to H429 of the amino acid sequence of human IgG1 heavy chain (Eu numbering) and a partner polypeptide linked to said Fc region component comprising at least a CH3 domain (or at least a constant heavy domain 4 (CH4) domain).

54. The immunotherapeutic protein of claim 53, wherein the partner polypeptides of the two fusion proteins are the same or different.

55. The immunotherapeutic protein of claim 53 or 54, wherein the immunotherapeutic protein is an antibody-like molecule in an H₂L₂ format and comprises two fusions proteins each comprising an immunoglobulin light chain polypeptide and a partner polypeptide linked thereto which may be the same or different.

56. The immunotherapeutic protein of any one of claims 53 to 55, wherein the partner polypeptides are each target recognition structures.

57. The use of the immunotherapeutic protein of any one of claims 1 to 56 for treating or preventing a disease or condition in a subject, wherein the disease or condition is selected from autoimmune diseases and conditions, other inflammatory diseases, infectious diseases and proliferative diseases.

58. The use of the immunotherapeutic protein of any one of claims 1 to 56 in the manufacture of a medicament for treating or preventing a disease or condition, wherein the disease or condition is selected from autoimmune diseases and conditions, other inflammatory diseases, infectious diseases and proliferative diseases.

59. A method for treating or preventing a disease or condition, comprising administering to the subject an effective amount of the immunotherapeutic protein of any one of claims 1 to 56, wherein the disease or condition is selected from autoimmune diseases and conditions, other inflammatory diseases, infectious diseases and proliferative diseases.

60. The method of claim 59, wherein the immunotherapeutic protein comprises a fusion protein comprising a partner polypeptide linked to an Fc region component comprising at least a CH3 domain (or at least a constant heavy domain 4 (CH4) domain) and said partner polypeptide is a cell surface receptor polypeptide (or a fragment thereof) that is capable of binding to a structural protein of a virus such that the immunotherapeutic protein may act as a “decoy” to block viral interaction and cellular entry of a virus to a host cell, said method further comprising administering an antibody directed against the said virus.

61. The method of claim 59, wherein the immunotherapeutic protein comprises a fusion protein comprising a partner polypeptide linked to an Fc region component comprising at least a CH3 domain (or at least a constant heavy domain 4 (CH4) domain) and said partner polypeptide is directed at a target of therapeutic significance, said method further comprising administering an antibody directed against the said target.

62. A pharmaceutical composition or medicament comprising the immunotherapeutic protein of any one of claims 1 to 56, and a pharmaceutically acceptable carrier, diluent and/or excipient.

63. The pharmaceutical composition or medicament of claim 62, wherein the carrier is saline at physiological pH.

64. The pharmaceutical composition or medicament of claim 62, wherein the carrier is saline at pH less than 6.5.

65. A pharmaceutical composition or medicament comprising the immunotherapeutic protein of claim 51 or 52, and a pharmaceutically acceptable carrier, diluent and/or excipient, wherein the pH of the composition or medicament is mildly acidic.

66. A pharmaceutical composition or medicament comprising a mixture of two or more different immunotherapeutic proteins of any one of claims 1 to 56, and a pharmaceutically acceptable carrier, diluent and/or excipient.

67. The pharmaceutical composition or medicament of claim 66, wherein the mixture of two or more immunotherapeutic proteins bind to different molecular targets.

68. The pharmaceutical composition or medicament of claim 67, wherein the immunotherapeutic proteins consist of a first immunoglobulin molecule with a first antigen recognition structure directed to a first antigen, and a second immunoglobulin molecule with a second antigen recognition structure directed to a second antigen, wherein the first antigen is a cancer antigen present on the surface of a cancerous cell and the second antigen is a death receptor.

69. The pharmaceutical composition or medicament of claim 67, wherein the immunotherapeutic proteins bind to different epitopes on a single molecular target.

70. A kit of parts comprising at least one immunotherapeutic protein of any one of claims 1 to 56.

71. A method of producing an immunotherapeutic protein of claim 1 to 56, comprising culturing a host cell comprising a construct encoding said protein under conditions suitable for the expression of said protein, and recovering the protein from culture supernatant under conditions of (i) mildly acidic pH to recover immunotherapeutic protein in a monomeric form; or (ii) substantially neutral pH to recover immunotherapeutic protein in an oligomeric form.

72. A method of producing an immunotherapeutic protein of any one of claims 1 to 56, comprising culturing a host cell comprising a construct encoding said protein under conditions suitable for the expression of said protein, and recovering the protein from culture supernatant using a method comprising affinity chromatography using an elution buffer comprising a concentration of arginine of less than 130 mM and at less than or equal to pH 5.0.

73. The method of claim 71, wherein the recovery of expressed immunotherapeutic protein comprises recovery by size exclusion chromatography (SEC).
